(19) United States
(12) Patent Application Publication
(10) Pub. No.: US 2025/0257107 A1
(43) Pub. Date: Aug. 14, 2025

(54) RECOMBINANT KERATINS AND PRODUCTION THEREOF

(71) Applicant: Geltor, Inc., Emeryville, CA (US)

(72) Inventors: Nikolay OUZOUNOV, San Ramon, CA (US); Jeffrey R. MELLIN, Richmond, CA (US); Andrew R. HAGEN, Oakland, CA (US); Krystal L. AUSTIN, Aurora, CO (US); José M. MELO BARCELOS, Emeryville, CA (US)

(21) Appl. No.: 18/856,729

(22) PCT Filed: Apr. 13, 2023
(86) PCT No.: PCT/US2023/018560

§ 371 Date: Oct. 14, 2024

Related U.S. Application Data
(60) Provisional application No. 63/330,894, filed on Apr. 14, 2022.

(51) Int. Cl. C07K 14/47; A61K 8/65; A61Q 5/12; C07K 1/14

(52) U.S. Cl. CPC C07K 14/4741; A61K 8/65; A61Q 5/12; C07K 1/14; C07K 2319/50

(57) ABSTRACT

Provided herein are recombinant keratins, and methods of production and uses thereof. Keratin polypeptides are produced together with solubility-enhancing amino acid sequences allowing for soluble expression and facile purification from recombinant systems. Recombinant keratins can be used, for example, in personal care products, such as skin care products, cosmetics, nail care products, scalp products, or hair care products.

### CROSS-REFERENCE

This application is a national phase application under 35 U.S.C. § 371 of PCT International Application No.: PCT/US2023/018560, filed on Apr. 13, 2023, which claims the benefit of U.S. Provisional Application No. 63/330,894, filed Apr. 14, 2022, the entire contents of which are incorporated herein by reference for all purposes.

### SEQUENCE LISTING

The text of the computer readable sequence listing filed herewith, titled “57607-720_601_SL”, created May 15, 2023, having a file size of 377,856 bytes, is hereby incorporated by reference in its entirety.

### BACKGROUND

Keratins are a type of intermediate filament produced by epithelial cells in vertebrates. Keratin provides mechanical strength to tissues, such as skin, hair, and nails in humans, and additionally in claws and scales in certain animals. The human genome contains about 54 different keratin genes, many of which are expressed exclusively in various compartments of hair follicles. There are about 28 types of keratins in human epithelial cells, and about 17 types of keratin proteins that are specifically located in hair.

Keratins are important for mechanical stability of both individual epithelial cells and entire tissues. Inside individual cells, keratins can be bundled into filament bundles that span the cytoplasm, forming a scaffolding network that protects the cell. They also attach to epithelial cell-cell junctions (desmosomes), to increase the stability of entire tissues. In some cases, they are also involved in non-mechanical functions, such as intracellular signaling pathways and other regulatory functions. Keratins can also be involved in such diverse functions as wound healing, apoptosis, and protection from stress. In vertebrates, keratins form hair, the outer layer of skin, horns, nails, wool, feathers, claws, and hooves.

Isolated keratins are also commercially useful, for example, in the personal care industry. The beauty and health of hair depends on the health of the outer cuticle which can be treated with keratins. As such, keratins are also useful as an additive to hair products, to result in thicker, shinier hair. Keratin addition to hair can repair damaged hair and can straighten curly hair. Keratin can be used at a keratin treatment at salons, where keratin, combined with a heat treatment, results in thicker individual hairs that are also straighter, with the effects of the procedure lasting several months.

Keratins can be used as markers for specific diseases, such as cancers. Antibodies against certain keratins are used in pathology laboratories during histopathological analysis for cancer diagnosis. Thus, isolated keratins are needed to initiate production of the antibodies.

Commercially used keratins have traditionally been isolated from animal by-products such as wool and chicken feathers. Keratins have also been isolated from horn material, such as goat horns. Preparation of keratin from animal sources is a time consuming and costly process, often requiring grinding steps, and a step of enzymatic extraction combined with addition of surfactants. The finished keratin product would also be likely to include other proteins and unwanted contaminants. Further, the keratin that is produced in this fashion has a different amino acid sequence than human keratin.

Recombinant keratins have been produced in the past. Basit et al (“Health improvement of human hair and their reshaping using recombinant keratin K31”; Biotechnology reports, 2018) produced recombinant keratin in E. coli. However, the protein was present in insoluble inclusion bodies in the bacterial cells. The production of a protein of interest in a bacterial inclusion body requires the additional time-consuming steps of breaking the cell walls, isolating the inclusion bodies, unfolding the protein from the inclusion bodies, refolding the protein, as part of any desired protein purification steps.

Keratins are not normally present in non-animal sources such as plants, yeast, and bacteria, they are typically derived from animal-based sources, such as from by-products from the meat production industry. A non-animal source of keratin polypeptides would be useful for the personal care industry. Because keratins are difficult to prepare and purify, and typically end up in bacterial inclusion bodies when made recombinantly, another way of preparing keratins is needed.

### SUMMARY

An improved method of preparing keratin polypeptides is described herein. Keratin polypeptides produced according to methods described herein have improved properties, such as purity and solubility throughout expression and purification. Resultant keratin polypeptides have a defined amino acid composition and molecular weight, providing a consistent product profile. Thus, using the methods described herein, keratins can now be efficiently produced for a variety of purposes.

In one aspect, a non-naturally occurring polypeptide is provided comprising (a) a solubility-enhancing amino acid sequence; and (b) a target amino acid sequence at least 50 amino acids in length having at least 80% sequence identity to the corresponding region of a keratin polypeptide, wherein the solubility-enhancing amino acid sequence consists of a triplet amino acid pattern of (Gly-X—Y)ₙ, wherein n=8-300, and X and Y are any amino acid residue. In some cases, for at least one (Gly-X—Y) triplet, X and Y are the same amino acid residue. In some cases, for at least one (Gly-X—Y) triplet, X and Y are different amino acid residues. In some cases, at least one (Gly-X—Y) triplet is different from another (Gly-X—Y) triplet. In some cases, at least one (Gly-X—Y) triplet is the same as another (Gly-X—Y) triplet. In some cases, the non-naturally occurring polypeptide is a fusion protein. In some cases, the solubility-enhancing amino acid sequence is fused to the N-terminus of the target amino acid sequence.

In some cases, the solubility-enhancing amino acid sequence is fused to the C-terminus of the target amino acid sequence. In some cases, the solubility-enhancing amino acid sequence comprises a first solubility-enhancing amino acid sequence fused to the N-terminus of the target amino acid sequence, and a second solubility-enhancing amino acid sequence fused to the C-terminus of the target amino acid sequence. In some cases, a ratio of the number of amino acid residues present in the solubility-enhancing amino acid sequence to the number of amino acid residues present in the target amino acid sequence is at least 0.5:1. In some cases, the keratin polypeptide is a Type I keratin or a Type II keratin. In some cases, the Type I keratin is selected from the group consisting of an epithelial keratin, a hair follicle-specific epithelial keratin, and a hair keratin. In some cases, the keratin polypeptide is a hair keratin selected from the group consisting of K31, K32, K33a, K33b, K34, K35, K36, K37, K38, K39, and K40. In some cases, the keratin polypeptide is an epithelial keratin selected from the group consisting of K9, K10, K12, K13, K14, K15, K16, K17, K18, K19, K20, K23, and K24. In some cases, the Type II keratin is selected from the group consisting of an epithelial keratin, a hair follicle-specific epithelial keratin, and a hair keratin. In some cases, the keratin polypeptide is a hair keratin selected from the group consisting of K81, K82, K83, K84, K85, and K86. In some cases, the keratin polypeptide is an epithelial keratin selected from the group consisting of K1, K2, K3, K4, K5, K6a, K6b, K6c, K7, K8, K76, K77, K78, K79, and K80. In some cases, the solubility-enhancing amino acid sequence and the target amino acid sequence are directly linked by a peptide bond. In some cases, the solubility-enhancing amino acid sequence and the target amino acid sequence are linked via a linker sequence. In some cases, the linker sequence is selected from the group consisting of a flexible linker, a rigid linker, and a cleavable linker. In some cases, the cleavable linker comprises a protease cleavage site. In some cases, the solubility-enhancing sequence comprises or consists of an amino acid sequence of any one of SEQ ID NOS: 18-31 or 119, or comprises or consists of an amino acid sequence having at least about 80% sequence identity to the amino acid sequence of any one of SEQ ID NOS: 18-31 or 119. In some cases, the target amino acid sequence comprises or consists of an amino acid sequence of any one of SEQ ID NOS: 2-12, or comprises or consists of an amino acid sequence having at least about 80% sequence identity to the amino acid sequence of any one of SEQ ID NOS: 2-12. In some cases, the non-naturally occurring polypeptide comprises or consists of an amino acid sequence of any one of SEQ ID NOS: 56-74, 76, 78, 80-82, 84, 86, 88, 90, 92, 94, 96, 102, 104, 106, 108-113, or 118, or comprises or consists of an amino acid sequence having at least about 80% sequence identity to the amino acid sequence of any one of SEQ ID NOS: 56-74, 76, 78, 80-82, 84, 86, 88, 90, 92, 94, 96, 102, 104, 106, 108-113, or 118.

In another aspect, a method of producing a non-naturally occurring polypeptide is provided, the method comprising: (a) culturing a host cell comprising a polynucleotide encoding the non-naturally occurring polypeptide of any one of the preceding; and (b) recovering the non-naturally occurring polypeptide, thereby producing the non-naturally occurring polypeptide.

In another aspect, a method of producing a target polypeptide is provided, the method comprising: (a) culturing a host cell comprising a polynucleotide encoding the non-naturally occurring polypeptide of any one of the preceding, (b) recovering the non-naturally occurring polypeptide, and (c) separating the solubility-enhancing amino acid sequence from the target amino acid sequence, thereby producing the target polypeptide.

In some embodiments, the host cell described in the method herein can be a microbial cell or a eukaryotic cell. In some embodiments, the microbial host cell can be a bacteria cell. In some embodiments, the bacteria cell can be E. coli. In some embodiments, the eukaryotic cell can be a mammalian cell.

In yet another aspect, a recombinant polypeptide is provided comprising the amino acid sequence of a full-length human keratin 31 (K31) having a total truncation of from 50 amino acids to 400 amino acids. In some cases, the total truncation is from 50 amino acids to 350 amino acids, from 50 amino acids to 300 amino acids, from 50 amino acids to 250 amino acids, from 50 amino acids to 200 amino acids, from 50 amino acids to 150 amino acids, or from 50 amino acids to 100 amino acids. In some cases, the total truncation comprises an N-terminal truncation, a C-terminal truncation, an internal truncation, or any combination thereof. In some cases, the total truncation is an N-terminal truncation. In some cases, the N-terminal truncation is from 50 amino acids to 250 amino acids, from 50 amino acids to 200 amino acids, from 50 amino acids to 150 amino acids, or from 50 amino acids to 100 amino acids. In some cases, the total truncation is a C-terminal truncation. In some cases, the C-terminal truncation is from 50 amino acids to 200 amino acids, from 50 amino acids to 150 amino acids, or from 50 amino acids to 100 amino acids. In some cases, the total truncation is both an N-terminal truncation and a C-terminal truncation. In some cases, the N-terminal truncation is from 50 amino acids to 250 amino acids, from 50 amino acids to 200 amino acids, from 50 amino acids to 150 amino acids, or from 50 amino acids to 100 amino acids, and the C-terminal truncation is from 50 amino acids to 200 amino acids, from 50 amino acids to 150 amino acids, or from 50 amino acids to 100 amino acids. In some cases, the recombinant polypeptide has less than 75% of the amino acid sequence of the full-length K31. In some cases, the recombinant polypeptide has less than 70%, less than 65%, less than 60%, less than 55%, less than 50%, less than 45%, less than 40%, less than 35%, less than 30%, less than 25%, less than 20%, or less than 15% of the amino acid sequence of the full-length K31. In some cases, the recombinant polypeptide comprises the amino acid sequence of SEQ ID NO: 11, or an amino acid sequence having at least 80% sequence identity to SEQ ID NO: 11. In some cases, the recombinant polypeptide comprises the amino acid sequence of any one of SEQ ID NOs: 7, 9, or 10; or an amino acid sequence having at least 80% sequence identity to any one of SEQ ID NOs: 7, 9, or 10. In some cases, the recombinant polypeptide consists of the amino acid sequence of any one of SEQ ID NOs: 7 or 9-11; or consists of an amino acid sequence having at least 80% sequence identity to any one of SEQ ID NOs: 7 or 9-11.

In yet another aspect, a non-naturally occurring polypeptide is provided comprising at least 9 contiguous amino acid residues of a collagen polypeptide and at least 25 contiguous amino acid residues of a keratin polypeptide, wherein the at least 9 contiguous amino acid residues of the collagen polypeptide comprise at least three (Gly-X—Y) triplet repeats.

In another aspect, a composition is provided comprising from 0.005% to 30% w/w of the non-naturally occurring polypeptide of any one of the preceding, the target polypeptide of any one of the preceding, or the recombinant polypeptide of any one of the preceding.

In another aspect, a composition is provided comprising a recombinant polypeptide, wherein the recombinant polypeptide comprises an amino acid sequence having at least 80% sequence identity to a corresponding region of a full-length keratin polypeptide having a truncation of at least 50 amino acids, wherein the recombinant polypeptide has a purity of at least 80% as measured by high-performance liquid chromatography or mass spectrometry.

In some cases, any of the preceding compositions are formulated for topical application. In some cases, any of the preceding compositions are personal care products for application to skin, hair, scalp, or nails.

In yet another aspect, a method for treating or providing a cosmetic benefit to the skin or nails of a subject is provided, the method comprising administering or applying to the skin or nails of a subject the composition of any of the preceding, thereby treating or providing the cosmetic benefit to the skin or nails of the subject. In some cases, the administering or applying results in decreasing skin damage, promoting the repair of damaged skin, protecting the skin against UV damage, protecting skin cells against the effects of exposure to urban dust, increasing viability of skin cells, increasing the viability of fibroblast cells, increasing the viability of keratinocyte cells, increasing procollagen synthesis, decreasing the production of inflammatory cytokines, repairing or strengthening the nail plate, repairing dry or damaged nails, preventing damage to nails, improving strength or growth of nails, or any combination thereof.

In yet another aspect, a method for treating or providing a cosmetic benefit to the hair of a subject is provided, the method comprising administering or applying to the hair of a subject the composition of any one of the preceding, thereby treating or providing the cosmetic benefit to the hair of the subject. In some cases, the administering or applying results in improving hair strength, improving hair shininess, smoothness, suppleness, combing performance, frizz control, sheen or feel, improving hair combability or manageability, improving hair flexibility, increasing hair strand diameter, improving viability of cell populations contributing to hair and scalp health, providing wound healing benefits for hair care, providing wound healing benefits for skin care, providing wound healing benefits for biomedicine, strengthening hair, repairing split ends, repairing hair damaged by atmospheric agents, repairing hair damaged or weakened by mechanical or chemical treatments, protecting hair structural proteins from oxidative stress, decreasing keratin denaturation, protecting hair from damage by heat, protecting hair from damage by atmospheric agents, mechanical or chemical treatments, or any combination thereof.

A method of purifying a recombinant polypeptide from a plurality of host cells is described herein. The method of purifying a recombinant polypeptide from a plurality of host cells comprises (a) providing or obtaining a mixture comprising the recombinant polypeptide and host cells or host cell lysate; (b) separating the mixture into a first soluble fraction comprising the recombinant polypeptide and a first insoluble fraction; (c) adjusting the pH of the first soluble fraction to a pH of 5 or less to generate a pH-adjusted mixture; and (d) separating the pH-adjusted mixture into a second soluble fraction comprising the recombinant polypeptide and a second insoluble fraction, wherein the recombinant polypeptide comprises at least 50% of protein in the second soluble fraction.

In some aspects, the recombinant polypeptide can comprise a solubility-enhancing amino acid sequence. In some aspects, the recombinant polypeptide does not comprise a solubility-enhancing amino acid sequence.

In some aspects, the host cell can be a microbial cell. In some aspects, the microbial cell can be a bacterial cell. In some aspects, the bacterial cell can be a gram-negative bacterium. In some aspects, the gram-negative bacterium can be E. coli. In some aspects, the separating of the mixture in step (c) can comprise centrifugation, ultracentrifugation, filtration, ultrafiltration, or a combination thereof.

In some aspects, the adjusting of the pH in step (c) can comprise adding an acid to the first soluble fraction. In some aspects, the acid can be a weak acid or a strong acid. In some aspects, the acid can be H₂SO₄. In some aspects, the adjusting of the pH in step (c) can comprise adjusting the pH of the first soluble fraction to a pH of 4 or less. In some aspects, the adjusting of the pH in step (c) can comprise adjusting the pH of the first soluble fraction to a pH of 3 or less.

In some aspects, the method of purifying a recombinant polypeptide from a plurality of host cells can further comprise a step (e) for adjusting the pH of the second soluble fraction to a pH that is lower than the pH of (c) to generate a second pH-adjusted mixture; and (f) for separating the second pH-adjusted mixture into a third soluble fraction comprising the recombinant polypeptide and a third insoluble fraction. In some aspects, the method can further comprise a step (g) for adjusting the pH of the third soluble fraction to a pH that is lower than the pH of (e) to generate a third pH-adjusted mixture; and (h) for separating the third pH-adjusted mixture into a fourth soluble fraction comprising the recombinant polypeptide and a fourth insoluble fraction.

In some aspects, a purity of the recombinant polypeptide is higher in the third soluble fraction than in the second soluble fraction; and wherein a purity of the recombinant polypeptide is higher in the fourth soluble fraction than in the third soluble fraction. In some aspects, the recombinant polypeptide, or portions thereof, is at least 55%, at least 65%, at least 70%, at least 75%, at least 80%, at least 85%, at least 90%, at least 94%, at least 95%, at least 96%, at least 97%, at least 98%, or at least 99% of protein present in the second soluble fraction.

In some aspects, the final product comprising the recombinant polypeptide is devoid of host cell debris. In some aspects, the final product comprising the recombinant polypeptide comprises at most 50%, at most 45%, at most 40%, at most 35%, at most 30%, at most 25%, at most 20%, at most 15%, at most 10%, at most 6%, at most 5%, at most 4%, at most 3%, at most 2%, or at most 1% endogenous host cell protein relative to total protein. In some aspects, the final product comprising the recombinant polypeptide is devoid of endogenous host cell protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1A and FIG. 1B are schematic diagrams showing the process used to produce the polypeptides of the present disclosure and for testing their solubility at various pH values. FIG. 1A is a diagram showing how the bacterial cells harboring genes encoding the proteins of interest were grown in shake flasks, and the polypeptides of interest were induced and secreted into the culture medium. FIG. 1B is a schematic diagram showing that after the cell culture step (FIG. 1A), the broth from the centrifuged cell culture medium was treated with an acid to reach several pH stages then centrifuged once again. Aliquots were removed at each pH stage for analysis. Proteins in both the pellet fraction (insoluble) and the supernatant fraction (soluble) were examined by SDS-PAGE, followed by protein staining with Coomassie blue, in order to determine expression and solubility at various pH values.

FIG. 2 is a photograph of SDS-PAGE gels showing expression and secretion of various human keratin polypeptides into the culture media when produced as fusion proteins with solubility-enhancing amino acid sequences of the present disclosure (left gel EFT 36); and after the culture media was pH adjusted to pH 3.0 and separated into insoluble (pellet) and soluble (supernatant) fractions (supernatant is shown in right gel EFT 36-pH 3). The keratin polypeptides remain soluble following pH treatment. The far right lane shows the keratin polypeptide expressed without the solubility-enhancing amino acid sequence, showing that it is not soluble at pH 3.

FIG. 3 is a photograph of SDS-PAGE gels showing production of human keratin type 31 polypeptides. The left gel shows a keratin type 31 polypeptide sequence expressed by itself (“K31”) and the right gel shows the same sequence expressed together with a solubility-enhancing amino acid sequence derived from a human type I, alpha 1 collagen (“HsCol1a1-27_K31”). At the end of fermentation (“BOF”) the medium containing the secreted protein was either untreated, or adjusted to either pH 5, 4, or 3. The media was centrifuged to separate the insoluble fraction as the pellet (“Pel”) and the soluble fraction as the supernatant (“Sol”) fractions, and the proteins in both fractions were analyzed. Far left lane: Protein size marker. After acid treatment at each pH tested, K31 is present in the insoluble pellet fraction after acid treatment, while HsCol1a1-27_K31 is present in the soluble supernatant fraction. Thus, the solubility-enhancing amino acid sequence was used to successfully produce and purify an otherwise insoluble keratin type 31 polypeptide sequence.

FIG. 4 is a photograph of an SDS-PAGE gel showing production of a human keratin type 31 polypeptide with an alternate solubility-enhancing amino acid sequence derived from a Gallus type 21 collagen (“GL21”). Samples were prepared and analyzed as above. Far left lane: Protein size marker. Thus, this solubility-enhancing sequence was also used to successfully produce and purify the target keratin polypeptide (“*”).

FIG. 5 is a photograph of an SDS-PAGE gel showing production of a human keratin type 31 polypeptide with another alternate solubility-enhancing amino acid sequence derived from a human type III, alpha 1 (“HsCol3a1-4”). Samples were prepared and analyzed as above. Thus, this solubility-enhancing sequence was also used to successfully produce and purify the target keratin polypeptide (“*”).

FIG. 6 is a photograph of SDS-PAGE gels showing production of a human keratin type 31 polypeptide using various lengths of solubility-enhancing amino acid sequence derived from a human type I, alpha 1 collagen (225 amino acids (C225), 196 amino acids (C196), 147 amino acids (C130), 64 amino acids (C64), and 25 amino acids (C25)). Samples were prepared and analyzed as above. Far left lanes: Protein size marker. The molecular weight and the ratio of the collagen-derived solubility-enhancing amino acid sequence to the keratin amino acid sequence (measured by amino acid lengths) (“C:K ratio”) of each keratin polypeptide is indicated below the panels. Longer solubility-enhancing amino acids were more effective at producing soluble keratin polypeptides.

FIGS. 7A and 7B are photographs of SDS-PAGE gels showing production of a human keratin type 31 polypeptide using various lengths of an alternate solubility-enhancing amino acid sequence derived from a Gallus gallus type 21 collagen (188 amino acids (GL21; FIG. 7A), 141 amino acids (GL21Cde125; FIG. 7A), 93 amino acids (GL21Cde150; FIG. 7B), and 47 amino acids (GL21Cde175; FIG. 7B)). Samples were prepared and analyzed as above. Far left lanes: Protein size marker. Longer solubility-enhancing amino acids of this type were also more effective at producing soluble keratin polypeptides.

FIGS. 8A and 8B are photographs of various gels showing production of a human keratin type 31 polypeptide of various lengths (147 amino acids (K147; FIG. 8A), 125 amino acids (K125; FIG. 8A), 100 amino acids (K100; FIG. 8A), 75 amino acids (K75; FIG. 8B), and 50 amino acids (K50; FIG. 8B)) without the use of a solubility-enhancing amino acid sequence. Samples were prepared and analyzed as above. Far left lanes: Protein size marker. These keratin polypeptides are insoluble (pellet fractions) after pH treatment at all pH levels.

FIG. 9 is a photograph of various gels showing production of the same human keratin type 31 polypeptide of various lengths as in FIGS. 8A and 8B, but with the use of a solubility-enhancing amino acid sequence derived from a human type I, alpha 1 collagen (C262). Samples were prepared and analyzed as above. Far left lane: Protein size marker. Thus, the solubility-enhancing amino acid sequence was used to successfully produce and purify otherwise insoluble keratin type 31 polypeptide sequences at each length of the target keratin polypeptide sequence.

FIG. 10 is a photograph of various gels showing production of various human keratin type 31 polypeptides. Two different lengths of a solubility-enhancing amino acid sequence derived from a human type I, alpha 1 collagen (196 amino acids (C196) or 64 amino acids (C64)) were varied with two different lengths of human keratin type 31 polypeptide sequences (100 amino acids (K100) or 50 amino acids (K50)). Samples were prepared and analyzed as above. The molecular weight and the ratio of the collagen-derived solubility-enhancing amino acid sequence to the keratin amino acid sequence (measured by amino acid lengths) (“C:K ratio”) of each keratin polypeptide is indicated below the panels. Far left lanes: Protein size marker. Higher ratios of solubility-enhancing amino acid sequence to target keratin amino acid sequence were more effective at producing soluble keratin polypeptides.

FIG. 11 is a photograph of a gel showing production of a human keratin type 31 polypeptide (K31) using an enzyme cleavable linker (quasiTEV) between a collagen-derived solubility-enhancing amino acid sequence (HsCoIa1-27) and the target keratin amino acid sequence. Samples were prepared and analyzed as above. Far left lane: Protein size marker. Following acid enrichment, a sample of the expressed keratin polypeptide was treated with AcTEV protease, resulting in separation of collagen-derived solubility-enhancing amino acid sequence and linker (HsCoIa1-27quasiTEV at 24 kDa) from the target keratin amino acid sequence (K31 at 17 kDa). Following cleavage of all the expressed keratin polypeptide, the mix of polypeptides was again separated using pH, the collagen-derived solubility-enhancing amino acid sequence and linker collagen fragment remained soluble, and the target keratin amino acid sequence now fractionated with the pellet. Thus, the solubility-enhancing amino acid sequence was used to successfully produce and purify an otherwise insoluble keratin type 31 polypeptide sequence as a fusion polypeptide, followed by successful separation and purification of the target keratin type 31 polypeptide alone.

FIG. 12 illustrates fluorescence microscopy images of Cy5-labeled, exemplary recombinant keratin fusion polypeptide (SEQ ID NO: 71) deposited on type I hair that has been bleached three times (bottom panels). Fluorescence intensity of each hair strand was quantified using ImageJ. Data are plotted above the microscopy images.

FIG. 13 illustrates cross-sectional, fluorescence microscopy images of Cy5-labeled, exemplary recombinant keratin fusion polypeptide (SEQ ID NO: 71) penetration into the cortex of type I hair that has been bleached three times (bottom panels). Hair strands were conditioned with the recombinant keratin fusion polypeptide with either 1, 3, or 5 short applications or a single 1-hour incubation. Fluorescence intensity of within the cross-section of each hair strand was quantified using ImageJ. Data are plotted above the microscopy images.

FIG. 14 illustrates both surface and cross-sectional fluorescence microscopy images of Cy5-labeled, exemplary recombinant keratin fusion polypeptide (SEQ ID NO: 71) deposited on and within type I hair that has been bleached three times (bottom panels). Hair strands were incubated with the recombinant keratin fusion polypeptide for 1 hour and subsequently underwent 0, 1, 3, or 5 shampoo cycles. Fluorescence intensity of within the cross-section and on the surface of each hair strand was quantified using ImageJ. Data are plotted above the microscopy images.

FIG. 15 illustrates the hair denaturation temperature of European medium brown hair strands after undergoing a heat damage protocol by differential scanning calorimetry. An exemplary recombinant keratin fusion polypeptide (SEQ ID NO: 71) was applied to hair tresses as a leave-on prior to heat damage; an untreated control also underwent heat damage. Control hair tresses were not conditioned with the recombinant keratin fusion polypeptide.

FIG. 16 illustrates the effect of an exemplary recombinant keratin fusion polypeptide (SEQ ID NO: 71) on hair suppleness as quantified by bending force, the force necessary to create a deformation on a hair tress. Type I tresses that were bleached three times received 0, 1, or 5 recombinant keratin fusion polypeptide applications prior to a 3-point bend test performed with the TA XT (Stable Microsystems). The charts show the average difference in bending forth, where a negative value indicates a reduction of bending force relative to untreated hair.

FIG. 17 illustrates the effect of an exemplary recombinant keratin fusion polypeptide (SEQ ID NO: 71) on style retention. Type I hair that had been bleached three times (Type I, 3× bleached hair) or unbleached type I hair (Type I, virgin) (bottom panels) were treated with the recombinant keratin fusion polypeptide and rolled in plastic curlers overnight. The style retention test was started by removing the curlers and hair tresses were imaged at the start of the test and after 24 hours (bottom panel). Length retention was defined as the ratio between the initial length and final length of the hair tresses (top panel). The final length difference between the control and treated tresses were normalized to the final length of the control tress (top panel).

FIG. 18 illustrates the effect of an exemplary recombinant keratin fusion polypeptide (SEQ ID NO: 71) on frizz control when applied to hair tresses as a leave-on. Type I hair that had been bleached three times (Type I, 3× bleached hair), unbleached type I hair (Type I, virgin), or unbleached type IV hair (Type IV, virgin) were conditioned with the recombinant keratin fusion polypeptide at varying concentrations. Hair tresses were then placed in an image acquisition system (Shuffle, Bossa Nova Vision) set at 85% relative humidity with an initial set of images taken and a final set of images taken after 24 hours. The data illustrate the average change of frizz surface relative to control (untreated) tresses in which a negative result indicates a reduction of frizz compared to control (untreated) tresses.

FIG. 19 illustrates the effect of an exemplary recombinant keratin fusion polypeptide (SEQ ID NO: 71) on frizz control when applied to hair tresses as a rinse-off. Type I hair that had been bleached three times (Type I, 3× bleached hair), unbleached type I hair (Type I, virgin), or unbleached type IV hair (Type IV, virgin) were conditioned with the recombinant keratin fusion polypeptide. Hair tresses were then placed in an image acquisition system (Shuffle, Bossa Nova Vision) set at 85% relative humidity with an initial set of images taken and a final set of images taken after 24 hours. The data illustrate the average change of frizz surface relative to control (untreated) tresses in which a negative value indicates a reduction of frizz compared to control (untreated) tresses.

FIG. 20 illustrates the effect of an exemplary recombinant keratin fusion polypeptide (SEQ ID NO: 71) on combing performance when applied to hair tresses as a leave-on or rinse-off. Type I hair that had been bleached three times (Type I, 3× bleached hair) were conditioned with the recombinant keratin fusion polypeptide. Wet and dry combing tests were performed before and after recombinant keratin fusion polypeptide application using Texture Analyzer. The data illustrate the change in combing force, at various conditions, after recombinant keratin fusion polypeptide application (relative to the baseline measurement) in which a negative value indicates a reduction of combing force after recombinant keratin fusion polypeptide application.

FIG. 21 illustrates the antioxidative properties of an exemplary recombinant keratin fusion polypeptide (SEQ ID NO: 71). A commercial oxygen antioxidant capacity (ORAC) kit (OxiSelect™ ORAC Activity Assay, Cell Biolabs, Inc.) was used to evaluate the antioxidative properties of the recombinant keratin fusion polypeptide at various concentrations. Data are expressed as the Trolox Equivalent (TE).

FIG. 22A and FIG. 22B illustrate the effect of an exemplary recombinant keratin fusion polypeptide (SEQ ID NO: 71; “KFC” herein) on cell viability in populations of cells contributing to hair and scalp care. Human fibroblasts and keratinocytes were co-incubated with the recombinant keratin fusion polypeptide at varying concentrations in a MTT viability assay. FIG. 22A illustrates human fibroblast viability in vitro with results normalized to the untreated control. FIG. 22B illustrates human keratinocyte viability in vitro with results normalized to the untreated control.

FIG. 23 illustrates the wound healing properties of an exemplary recombinant keratin fusion polypeptide (SEQ ID NO: 71) in an in vitro wound healing assay. A wound is simulated by scratching cell cultures in the absence of recombinant polypeptides, in the presence of the recombinant keratin fusion polypeptide, or in the presence of hydrolyzed animal keratin (HAK). Real time cell migration is quantified over 48 hours. FIG. 23 (right panels) illustrates representative images of cells treated with the recombinant keratin fusion polypeptide. Quantified cell migration data are shown in FIG. 23 (left panel).

FIG. 24A and FIG. 24B illustrates a comparison between an exemplary recombinant keratin fusion polypeptide (SEQ ID NO: 71) and a representative collagen in the context of hair care. FIG. 24A illustrates the effect of the exemplary recombinant keratin fusion polypeptide and a representative collagen on dry combing force. FIG. 24B illustrates the effect of the exemplary recombinant keratin fusion polypeptide and the representative collagen on frizz control for unbleached type IV hair (hair type IV, virgin) when applied as leave-on treatments.

FIG. 25A and FIG. 25B depict expression and low-pH stability of a representative fusion polypeptide (C196-K50) in a mammalian cell line (HEK293). FIG. 25A shows an SDS-PAGE gel (left) and a Western blot (right) using an antibody specific to the keratin region of the fusion polypeptide and compares expression from E. coli and HEK293 host cells. FIG. 25B is a photograph of an SDS-PAGE gel demonstrating the fusion polypeptide (arrow) and its ability to remain soluble in low pH conditions; insoluble pellet fractions (“P”) and soluble supernatant fractions (“S”) were both analyzed after pH treatment.

### DETAILED DESCRIPTION

### Definitions

The terminology used herein is for the purpose of describing particular cases only and is not intended to be limiting. As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The terms “about” or “approximately” mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual”, “patient”, or “subject” are used interchangeably herein. None of the terms require or are limited to a situation characterized by the supervision (e.g., constant or intermittent) of a health care worker (e.g., a doctor, a registered nurse, a nurse practitioner, a physician's assistant, an orderly, or a hospice worker).

As used herein, the term “comprise” or variations thereof such as “comprises” or “comprising” are to be read to indicate the inclusion of any recited feature but not the exclusion of any other features. In some embodiments of any of the compositions and methods provided herein, “comprising” may be replaced with “consisting essentially of” or “consisting of”. The phrase “consisting essentially of” is used herein to require the specified feature(s) as well as those which do not materially affect the character or function of the claimed disclosure. As used herein, the term “consisting” is used to indicate the presence of the recited feature alone.

The term “gene” as used herein generally refers to a polynucleotide that encodes a specific protein, and which may refer to the coding region alone or may include regulatory sequences preceding (5′ non-coding sequences) and following (3′ non-coding sequences) the coding sequence.

The term “recombinant” refers to nucleic acids or proteins formed by laboratory methods of genetic recombination (e.g., molecular cloning) to bring together genetic material from multiple sources, creating sequences that would not otherwise be found in the genome. A recombinant fusion protein is a protein created by combining sequences encoding two or more constituent proteins, such that they are expressed as a single polypeptide. Recombinant fusion proteins may be expressed in vivo in various types of host cells, including bacterial cells, fungal cells, mammalian cells, plant cells, etc.

As used herein, the term “solubility-enhancing amino acid sequence” refers to an amino acid sequence that is capable of increasing the solubility of a target amino acid sequence, for example, during expression, secretion and/or purification (such as, for example, by altering solubility at different ranges or types of pH, ionic strengths, salts, salt concentrations, temperature, polarity, buffers, solvents etc.). The solubility of a target amino acid sequence is enhanced when that target sequence has greater solubility when expressed together with the solubility-enhancing amino acid sequence as compared to when expressed alone (i.e., without the solubility-enhancing amino acid sequence).

As used herein, “operably linked” with reference to nucleic acid sequences, regions, elements or domains means that the nucleic acid regions are functionally related to each other. In another example, a promoter can be operably linked to nucleic acid encoding a polypeptide, whereby the promoter regulates or mediates the transcription of the nucleic acid.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, for example, Coomassie blue staining following gel electrophoresis, Lowry protein assay and Bradford protein assay.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide of interest. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in prokaryotic cells and/or eukaryotic cells, as well as those that remain episomal or those which integrate into the host cell genome.

Expression vectors can be designed for expression of a protein of interest in a variety of expression systems as are known in the art, such as those using prokaryotic cells (e.g., Gram positive bacteria, Gram negative bacteria such as E. coli, or Archaea) or eukaryotic cells (e.g., insect cells (such as e.g., Sf9 or Sf21 cells), yeast (such as e.g., Pichia, Saccharomyces or Kluyveromyces species), plants (such as whole plants, plant tissue systems or plant cell systems), algae (such as e.g., Chlamydomonas or Synechococcus species), avian cells (such as e.g., embryonic cells or fibroblasts) or mammalian cells (such as e.g., CHO, HeLa, Vero or HEK 293 cells)).

The term “promoter” or a “transcription regulatory region” refers to nucleic acid sequences that influence or promote the initiation of transcription. Promoters are typically considered to include regulatory regions, such as enhancer or inducer elements. The promoter will generally be appropriate to the host cell in which the target gene is being expressed. In general, the transcriptional and translational regulatory sequences include, but are not limited to, promoter sequences, ribosomal binding sites, transcriptional start and stop sequences, translational start and stop sequences, and enhancer or activator sequences.

The term “regulatory sequence” is intended to include promoters, enhancers and other expression control elements (e.g., polyadenylation signals) that control the transcription or translation of a gene. Regulatory sequences include those which direct constitutive expression of a nucleic acid in many types of cells, those which direct expression of the nucleic acid sequence only in certain cells (e.g., tissue specific regulatory sequences), and those which direct the expression of the nucleic acid sequence upon stimulation with a particular agent (e.g., inducible regulatory sequences). It will be appreciated by those skilled in the art that the design of the expression vector can depend on such factors as the choice of the cell to be transformed, the level of expression of protein desired, etc.

The terms “linker sequence”, “linker” or “spacer” generally refer to one to several amino acids that can be arranged between different sections of protein sequences. The two protein sequences can contain, for example, a rigid or flexible linker sequence of amino acids between the two protein sequences. Flexible linkers are generally rich in small or polar amino acids such as Gly and Ser to provide good flexibility and solubility. The flexible linker sequence can be, for example, a glycine-serine peptide sequence (Gly-Gly-Gly-Gly-Ser)˜(SEQ ID NO: 1). Rigid linkers exhibit relatively stiff structures by adopting α-helical structures or by containing multiple Pro residues. Under many circumstances, they separate the functional domains more efficiently than the flexible linkers, and useful when spatial separation preserves stability or activity. The linker sequence can be, for example, from one amino acid to about 15 amino acids in length. The linker sequence can also contain a protease cleavage site. Cleavable linkers may be useful, for example, to remove tags from recombinant proteins of interest during the purification process. Examples include enzymatically or chemically cleavable or photocleavable linkers. For example, the solubility-enhancing amino acid sequence may be joined to the target amino acid sequence via a cleavable linker, such as an enzymatically or chemically cleavable liker, and then the solubility-enhancing amino acid sequence may be separated from the target amino acid sequence using a cleavage step, such as an enzymatic incubation step, resulting in a purified target polypeptide alone.

The term “target polypeptide” broadly refers to an amino acid sequence that is of particular interest. The “target polypeptide” can take on many embodiments. For example, in some embodiments, the “target polypeptide” can be an endogenous protein. In some embodiments, the “target polypeptide” can be a recombinant protein. In some embodiments, the “target polypeptide” can be a full-length protein. In some embodiments, the “target polypeptide” can be a truncated protein. In some embodiments, the “target polypeptide” can be a fusion protein. In some embodiments, the “target polypeptide” can be a fragment of a fusion protein. In some embodiments, the “target polypeptide” can refer to a particular sequence of amino acids within a protein. In some embodiments, the “target polypeptide” can refer to a particular domain within a protein.

The term “protease cleavage site” generally refers to an amino acid sequence that is cleaved by a specific protease.

The term “histidine tag” generally refers to a 2-30 contiguous series of histidine residues on a recombinant polypeptide.

The term “secretion tag” or “signal peptide” generally refers to an amino acid sequence that recruits the host cell's cellular machinery to transport an expressed protein to a particular location or cellular organelle of the host cell.

As used herein, a “peptide” refers to a polypeptide that is from 2 to about or 40 amino acids in length.

As used herein, “polypeptide” refers to two or more amino acids covalently joined. The terms “polypeptide” and “protein” are used interchangeably herein.

As used herein, the term “fusion protein” refers to a protein composed of a plurality of polypeptide components expressed from a single nucleic acid sequence. Fusion proteins may be a combination of two, three or even four or more different proteins or polypeptides. The fusion protein can be, for example, a fusion of two or more heterologous amino acid sequences. The fusion protein can consist of a polypeptide joined with a heterologous polypeptide, which can be, for example, another protein or polypeptide, a heterologous targeting sequence, a linker, an epitope tag, a detectable fusion partner (such as a fluorescent protein), a secretion signal sequence, a purification aid (such as a His tag), a protease target site, a solubility-enhancing amino acid sequence, and the like. A fusion protein may have one or more heterologous domains added to the N-terminus, C-terminus, and or the middle (internal) portion of the protein. If two parts of a fusion protein are “heterologous”, they are not part of the same protein in its natural state. For example, a target amino acid sequence or polypeptide may be produced as a fusion protein with a solubility-enhancing amino acid sequence, and then optionally separated into individual polypeptides after production.

The term “host cell” generally refers to a cell that is engineered to express an introduced exogenous polynucleotide. The host cell can be a eukaryote or a prokaryote. The host cell can be, for example, a microbial cell, a bacterial cell, a fungal cell, a yeast cell, a mammalian cell, an algal cell, or a plant cell.

The term “keratinocyte” generally refers to a cell that produces keratins found in the epidermal layer of the skin.

The term “non-naturally occurring” as used herein refers to a gene, polypeptide, or protein, for example, a keratin polypeptide which is not normally found in nature. Non-naturally occurring keratins may be recombinantly prepared. The non-naturally occurring polypeptide comprises, e.g., a solubility-enhancing amino acid sequence and a target amino acid sequence comprising a keratin polypeptide. The keratin polypeptide may be a full-length keratin polypeptide, or a region or truncate thereof.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as any individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The term “truncation” as used herein refers to a sequence (amino acid, DNA, or RNA) that is shorter than that of a reference full-length sequence, e.g., a naturally occurring sequence. As used herein, the term “truncation” is interchangeable with the term “fragment”. The term “truncated polypeptide” as used herein generally refers to a polypeptide that is smaller than a full-length (e.g., natural) polypeptide wherein one or more portions of the full-length (e.g., natural) polypeptide is not present. The non-naturally occurring polypeptides provided herein may be truncated at the C-terminal end, the N-terminal end, truncated by removal of internal portion(s) of the full-length polypeptide sequence (e.g., an internal truncation), truncated at both the C-terminal end and the N-terminal end, or may have one or both of a C-terminal truncation and an N-terminal truncation as well as an internal truncation.

When used in reference to an amino acid position, a “truncation” is inclusive of said amino acid position. For example, an N-terminal truncation at amino acid position 100 relative to a full-length polypeptide means a truncation of 100 amino acids from the N-terminus of the full-length polypeptide (i.e., the truncated polypeptide is missing amino acid positions 1 through 100 of the full-length polypeptide). Similarly, a C-terminal truncation at amino acid position 901 of a full-length polypeptide (assuming a 1000 amino acid full-length polypeptide) means a truncation of 100 amino acids from the C-terminus (i.e., the truncated polypeptide is missing amino acid positions 901 through 1000 of the full-length polypeptide). Similarly, an internal truncation at amino acid positions 101 and 200 means an internal truncation of 100 amino acids of the full-length polypeptide (i.e., the truncated polypeptide is missing amino acid positions 101 to 200 of the full-length polypeptide).

The term “collagen-like protein” as used herein is a polypeptide or protein sequence comprising a collagenaceous repeat triplet sequence of (Gly-X—Y)ₙ, where X and Y can be any amino acids. The term encompasses variants and fragments of the collagen-like protein.

The section headings used herein are for organizational purposes only and are not to be construed as limiting the subject matter described.

### Keratin Polypeptides and Design for Recombinant Production

Keratins are a category of intermediate filaments (IFs), a superfamily of 10 nm fibers, and are typically richly alpha-helical polypeptides that intertwine in a coiled-coil fashion to form the subunit structure of 10 nm filaments. Filaments from different keratins have distinct physical properties, suggesting that differential expression is tailored to suit tissue-specific structural requirements of tensile strength, flexibility, and dynamics. Keratins are categorized as distinct from other IFs including desmin, vimentin, neurofilament proteins and glial fibrillary acid protein.

All IF proteins have a central a-helical domain, the rod, which is flanked by nonhelical head (amino-end) and tail (carboxy-end) domains. The rods of two polypeptide chains intertwine in a coiled-coil fashion. Throughout the a-helical sequences are repeats of hydrophobic amino acids, in a characteristic heptad repeat pattern, such that the first and fourth of every seven residues are frequently apolar. This provides a hydrophobic seal on the helical surface, enabling the coiling between two IF polypeptides.

Typically, in keratins, linker domains (L1, L12, L2) within the rod domain are evident, along with the clearly distinguishable heptad repeats in domains 1A, 1B, 2A, and 2B that are characteristic of α-helical coiled-coil forming IF proteins. Furthermore, subdomain 2B often contains an interruption or stutter in the heptad repeat which may play an important role in the unfolding/unwinding of the coiled-coils when they are subjected to strain.

Keratin dimers are typically heterodimers, assembled from two different related alpha-helical proteins. Assembly conditions, such as pH, and the structure of the oligomers that form IFs can differ between IFs, with keratins having signature assembly units (unit length filaments).

Several types of keratin proteins exist in the human body. Human and animal hair, for example, has two main types of keratins: type I (acidic, pI=4-6) and type II (basic, pI=6-8). Keratin is characterized by a high cysteine content, which varies from 7% to 20% of the total amino acid residues. The mechanical strength of hair is due to the presence of intramolecular and intermolecular disulfide bonds. Type I and II keratins have been found in all classes of vertebrates. Keratin-like IFs proteins have been found in lower chordates. Thread-forming keratins are another type found in several teleost fish and amphibians. 10 nm filaments very similar to IFs found in animal cells have been identified in some types of plant cells, and their main component was keratin.

Morphologically, human hair fibers are composed of three main components: the cuticle, the cortex, and the medulla. Keratin 31 is a main component of the cortex component of hair. Damage that occurs to the hair, for example, during chemical treatments, exposure to the sun, etc., can cause damage in this layer of hair. The application of keratin to the hair is now of commercial importance to the personal care and hair-care industry to repair and protect the damaged hair.

As such, keratin-based personal care and hair care products are of commercial importance. However, these keratin proteins are currently industrially sourced, for the most part, from animal by-products such as wool and chicken feathers.

Having a human keratin protein sequence that can be produced in large amounts in microbial cells, and thereafter easily and inexpensively purified, would be a useful way to obtain industrial amounts of keratin that is not animal sourced, and is vegan and sustainable, especially for use in the hair care and personal care industry.

To alleviate issues with animal-sourced products, both collagen and keratin can be produced by other eukaryotic or prokaryotic organisms. However, microbially produced keratin, in particular, is very difficult to purify in large quantities. This creates an obstacle for the prospect of obtaining large amounts of purified non-animal-sourced keratin.

In certain embodiments, the keratin polypeptides of the present disclosure may be derived from any of the 54 keratin types known in the art, as summarized in Table 1 below.

TABLE 1
Keratin Types

| Keratin Type | Type I | Type II |
|---|---|---|
| Epithelial keratins | K9, K10, K12, K13, K14, K15, | K1, K2, K3, K4, K5, K6a, |
| | K16, K17, K18, K19, K20, | K6b, K6c, K7, K8, K76, K77, |
| | K23, K24 | K78, K79, K80 |
| Hair follicle-specific epithelial | K25, K26, K27, K28 | K71, K72, K73, K74, K75 |
| keratins (root sheath) | | |
| Hair keratins | K31, K32, K33a, K33b, K34, | K81, K82, K83, K84, K85, |
| | K35, K36, K37, K38, K39, | K86 |
| | K40 | |

In preferred embodiments, the keratin may be a hair keratin, for use, e.g., in personal care products, such as hair care products. In other preferred embodiments, the keratin may be an epithelial keratin, for use, e.g., in personal care products, such as skin or nail products.

Exemplary sequences and descriptions of members of each of the 54 keratin types can be found by the UniProt Knowledgebase (KB) identifier provided in Table 2 below. UniProtKB available at www.uniprot.org/uniprot/. Other suitable keratin sequences can be readily identified in the art.

TABLE 2
Exemplary Human Keratins

| UniProtKB | | | | |
|---|---|---|---|---|
| Identifier | Entry name | Protein names | Gene names | Length |
| P35527 | K1C9_HUMAN | Keratin, type I cytoskeletal 9 | KRT9 | 623 |
| | | (Cytokeratin-9) (CK-9) (Keratin-9) | | |
| | | (K9) | | |
| P13645 | K1C10_HUMAN | Keratin, type I cytoskeletal 10 | KRT10 KPP | 584 |
| | | (Cytokeratin-10) (CK-10) (Keratin- | | |
| | | 10) (K10) | | |
| Q99456 | K1C12_HUMAN | Keratin, type I cytoskeletal 12 | KRT12 | 494 |
| | | (Cytokeratin-12) (CK-12) (Keratin- | | |
| | | 12) (K12) | | |
| P13646 | K1C13_HUMAN | Keratin, type I cytoskeletal 13 | KRT13 | 458 |
| | | (Cytokeratin-13) (CK-13) (Keratin- | | |
| | | 13) (K13) | | |
| P02533 | K1C14_HUMAN | Keratin, type I cytoskeletal 14 | KRT14 | 472 |
| | | (Cytokeratin-14) (CK-14) (Keratin- | | |
| | | 14) (K14) | | |
| P19012 | K1C15_HUMAN | Keratin, type I cytoskeletal 15 | KRT15 | 456 |
| | | (Cytokeratin-15) (CK-15) (Keratin- | KRTB | |
| | | 15) (K15) | | |
| P08779 | K1C16_HUMAN | Keratin, type I cytoskeletal 16 | KRT16 | 473 |
| | | (Cytokeratin-16) (CK-16) (Keratin- | KRT16A | |
| | | 16) (K16) | | |
| Q04695 | K1C17_HUMAN | Keratin, type I cytoskeletal 17 (39.1) | KRT17 | 432 |
| | | (Cytokeratin-17) (CK-17) (Keratin- | | |
| | | 17) (K17) | | |
| P05783 | K1C18_HUMAN | Keratin, type I cytoskeletal 18 (Cell | KRT18 | 430 |
| | | proliferation-inducing gene 46 | CYK18 | |
| | | protein) (Cytokeratin-18) (CK-18) | PIG46 | |
| | | (Keratin-18) (K18) | | |
| P08727 | K1C19_HUMAN | Keratin, type I cytoskeletal 19 | KRT19 | 400 |
| | | (Cytokeratin-19) (CK-19) (Keratin- | | |
| | | 19) (K19) | | |
| P35900 | K1C20_HUMAN | Keratin, type I cytoskeletal 20 | KRT20 | 424 |
| | | (Cytokeratin-20) (CK-20) (Keratin- | | |
| | | 20) (K20) (Protein IT) | | |
| Q9C075 | K1C23_HUMAN | Keratin, type I cytoskeletal 23 | KRT23 | 422 |
| | | (Cytokeratin-23) (CK-23) (Keratin- | | |
| | | 23) (K23) | | |
| Q2M2I5 | K1C24_HUMAN | Keratin, type I cytoskeletal 24 | KRT24 | 525 |
| | | (Cytokeratin-24) (CK-24) (Keratin- | KA24 | |
| | | 24) (K24) (Type I keratin-24) | | |
| Q7Z3Z0 | K1C25_HUMAN | Keratin, type I cytoskeletal 25 | KRT25 | 450 |
| | | (Cytokeratin-25) (CK-25) (Keratin- | KRT25A | |
| | | 25) (K25) (Keratin-25A) (K25A) | | |
| | | (Type I inner root sheath-specific | | |
| | | keratin-K25irs1) | | |
| Q7Z3Y9 | K1C26_HUMAN | Keratin, type I cytoskeletal 26 | KRT26 | 468 |
| | | (Cytokeratin-26) (CK-26) (Keratin- | KRT25B | |
| | | 25B) (K25B) (Keratin-26) (K26) | | |
| | | (Type I inner root sheath-specific | | |
| | | keratin-K25irs2) | | |
| Q7Z3Y8 | K1C27_HUMAN | Keratin, type I cytoskeletal 27 | KRT27 | 459 |
| | | (Cytokeratin-27) (CK-27) (Keratin- | KRT25C | |
| | | 25C) (K25C) (Keratin-27) (K27) | | |
| | | (Type I inner root sheath-specific | | |
| | | keratin-K25irs3) | | |
| Q7Z3Y7 | K1C28_HUMAN | Keratin, type I cytoskeletal 28 | KRT28 | 464 |
| | | (Cytokeratin-28) (CK-28) (Keratin- | KRT25D | |
| | | 25D) (K25D) (Keratin-28) (K28) | | |
| | | (Type I inner root sheath-specific | | |
| | | keratin-K25irs4) | | |
| Q15323 | K1H1_HUMAN | Keratin, type I cuticular Hal (Hair | KRT31 | 416 |
| | | keratin, type I Hal) (Keratin-31) | HHA1 | |
| | | (K31) | HKA1 | |
| | | | KRTHA1 | |
| Q14532 | K1H2_HUMAN | Keratin, type I cuticular Ha2 (Hair | KRT32 | 448 |
| | | keratin, type I Ha2) (Keratin-32) | HHA2 | |
| | | (K32) | HKA2 | |
| | | | KRTHA2 | |
| O76009 | KT33A_HUMAN | Keratin, type I cuticular Ha3-I (Hair | KRT33A | 404 |
| | | keratin, type I Ha3-I) (Keratin-33A) | HHA3-I | |
| | | (K33A) | HKA3A | |
| | | | KRTHA3A | |
| Q14525 | KT33B_HUMAN | Keratin, type I cuticular Ha3-II (Hair | KRT33B | 404 |
| | | keratin, type I Ha3-II) (Keratin-33B) | HHA3-II | |
| | | (K33B) | HKA3B | |
| | | | KRTHA3B | |
| O76011 | KRT34_HUMAN | Keratin, type I cuticular Ha4 (Hair | KRT34 | 436 |
| | | keratin, type I Ha4) (Keratin-34) | HHA4 | |
| | | (K34) | HKA4 | |
| | | | KRTHA4 | |
| Q92764 | KRT35_HUMAN | Keratin, type I cuticular Ha5 (Hair | KRT35 | 455 |
| | | keratin, type I Ha5) (Keratin-35) | HHA5 | |
| | | (K35) | HKA5 | |
| | | | KRTHA5 | |
| O76013 | KRT36_HUMAN | Keratin, type I cuticular Ha6 (Hair | KRT36 | 467 |
| | | keratin, type I Ha6) (Keratin-36) | HHA6 | |
| | | (K36) | HKA6 | |
| | | | KRTHA6 | |
| O76014 | KRT37_HUMAN | Keratin, type I cuticular Ha7 (Hair | KRT37 | 449 |
| | | keratin, type I Ha7) (Keratin-37) | HHA7 | |
| | | (K37) | HKA7 | |
| | | | KRTHA7 | |
| O76015 | KRT38_HUMAN | Keratin, type I cuticular Ha8 (Hair | KRT38 | 456 |
| | | keratin, type I Ha8) (Keratin-38) | HHA8 | |
| | | (K38) | HKA8 | |
| | | | KRTHA8 | |
| Q6A163 | K1C39_HUMAN | Keratin, type I cytoskeletal 39 | KRT39 | 491 |
| | | (Cytokeratin-39) (CK-39) (Keratin- | KA35 | |
| | | 39) (K39) (Type I hair keratin Ka35) | | |
| Q6A162 | K1C40_HUMAN | Keratin, type I cytoskeletal 40 | KRT40 | 431 |
| | | (Cytokeratin-40) (CK-40) (Keratin- | KA36 | |
| | | 40) (K40) (Type I hair keratin Ka36) | | |
| P04264 | K2C1_HUMAN | Keratin, type II cytoskeletal 1 (67 | KRT1 KRTA | 644 |
| | | kDa cytokeratin) (Cytokeratin-1) | | |
| | | (CK-1) (Hair alpha protein) (Keratin- | | |
| | | 1) (K1) (Type-II keratin Kb1) | | |
| P35908 | K22E_HUMAN | Keratin, type II cytoskeletal 2 | KRT2 | 639 |
| | | epidermal (Cytokeratin-2e) (CK-2e) | KRT2A | |
| | | (Epithelial keratin-2e) (Keratin-2 | KRT2E | |
| | | epidermis) (Keratin-2e) (K2e) (Type- | | |
| | | II keratin Kb2) | | |
| P12035 | K2C3_HUMAN | Keratin, type II cytoskeletal 3 (65 | KRT3 | 628 |
| | | kDa cytokeratin) (Cytokeratin-3) | | |
| | | (CK-3) (Keratin-3) (K3) (Type-II | | |
| | | keratin Kb3) | | |
| P19013 | K2C4_HUMAN | Keratin, type II cytoskeletal 4 | KRT4 CYK4 | 520 |
| | | (Cytokeratin-4) (CK-4) (Keratin-4) | | |
| | | (K4) (Type-II keratin Kb4) | | |
| P13647 | K2C5_HUMAN | Keratin, type II cytoskeletal 5 (58 | KRT5 | 590 |
| | | kDa cytokeratin) (Cytokeratin-5) | | |
| | | (CK-5) (Keratin-5) (K5) (Type-II | | |
| | | keratin Kb5) | | |
| P02538 | K2C6A_HUMAN | Keratin, type II cytoskeletal 6A | KRT6A K6A | 564 |
| | | (Cytokeratin-6A) (CK-6A) | KRT6D | |
| | | (Cytokeratin-6D) (CK-6D) (Keratin- | | |
| | | 6A) (K6A) (Type-II keratin Kb6) | | |
| | | (allergen Hom s 5) | | |
| P04259 | K2C6B_HUMAN | Keratin, type II cytoskeletal 6B | KRT6B K6B | 564 |
| | | (Cytokeratin-6B) (CK-6B) (Keratin- | KRTL1 | |
| | | 6B) (K6B) (Type-II keratin Kb10) | | |
| P48668 | K2C6C_HUMAN | Keratin, type II cytoskeletal 6C | KRT6C | 564 |
| | | (Cytokeratin-6C) (CK-6C) | KRT6E | |
| | | (Cytokeratin-6E) (CK-6E) (Keratin | | |
| | | K6h) (Keratin-6C) (K6C) (Type-II | | |
| | | keratin Kb12) | | |
| P08729 | K2C7_HUMAN | Keratin, type II cytoskeletal 7 | KRT7 SCL | 469 |
| | | (Cytokeratin-7) (CK-7) (Keratin-7) | | |
| | | (K7) (Sarcolectin) (Type-II keratin | | |
| | | Kb7) | | |
| P05787 | K2C8_HUMAN | Keratin, type II cytoskeletal 8 | KRT8 CYK8 | 483 |
| | | (Cytokeratin-8) (CK-8) (Keratin-8) | | |
| | | (K8) (Type-II keratin Kb8) | | |
| Q01546 | K220_HUMAN | Keratin, type II cytoskeletal 2 oral | KRT76 | 638 |
| | | (Cytokeratin-2P) (CK-2P) (K2P) | KRT2B | |
| | | (Keratin-76) (K76) (Type-II keratin | KRT2P | |
| | | Kb9) | | |
| Q7Z794 | K2C1B_HUMAN | Keratin, type II cytoskeletal 1b | KRT77 | 578 |
| | | (Cytokeratin-1B) (CK-1B) (Keratin- | KRT1B | |
| | | 77) (K77) (Type-II keratin Kb39) | | |
| Q8N1N4 | K2C78_HUMAN | Keratin, type II cytoskeletal 78 | KRT78 K5B | 520 |
| | | (Cytokeratin-78) (CK-78) (Keratin- | KB40 | |
| | | 5b) (Keratin-78) (K78) (Type-II | | |
| | | keratin Kb40) | | |
| Q5XKE5 | K2C79_HUMAN | Keratin, type II cytoskeletal 79 | KRT79 K6L | 535 |
| | | (Cytokeratin-79) (CK-79) (Keratin-6- | KB38 | |
| | | like) (Keratin-6L) (Keratin-79) (K79) | KRT6L | |
| | | (Type-II keratin Kb38) | | |
| Q6KB66 | K2C80_HUMAN | Keratin, type II cytoskeletal 80 | KRT80 | 452 |
| | | (Cytokeratin-80) (CK-80) (Keratin- | KB20 | |
| | | 80) (K80) (Type-II keratin Kb20) | | |
| Q3SY84 | K2C71_HUMAN | Keratin, type II cytoskeletal 71 | KRT71 | 523 |
| | | (Cytokeratin-71) (CK-71) (Keratin- | K6IRS1 | |
| | | 71) (K71) (Type II inner root sheath- | KB34 | |
| | | specific keratin-K6irs1) (Keratin 6 | KRT6IRS1 | |
| | | irs) (hK6irs) (hK6irs1) (Type-II | | |
| | | keratin Kb34) | | |
| Q14CN4 | K2C72_HUMAN | Keratin, type II cytoskeletal 72 | KRT72 | 511 |
| | | (Cytokeratin-72) (CK-72) (Keratin- | K6IRS2 | |
| | | 72) (K72) (Type II inner root sheath- | KB35 KRT6 | |
| | | specific keratin-K6irs2) (Type-II | KRT6IRS2 | |
| | | keratin Kb35) | | |
| Q86Y46 | K2C73_HUMAN | Keratin, type II cytoskeletal 73 | KRT73 | 540 |
| | | (Cytokeratin-73) (CK-73) (Keratin- | K6IRS3 | |
| | | 73) (K73) (Type II inner root sheath- | KB36 | |
| | | specific keratin-K6irs3) (Type-II | KRT6IRS3 | |
| | | keratin Kb36) | | |
| Q7RTS7 | K2C74_HUMAN | Keratin, type II cytoskeletal 74 | KRT74 | 529 |
| | | (Cytokeratin-74) (CK-74) (Keratin- | K6IRS4 | |
| | | 5c) (K5C) (Keratin-74) (K74) (Type | KB37 | |
| | | II inner root sheath-specific keratin- | KRT5C | |
| | | K6irs4) (Type-II keratin Kb37) | KRT6IRS4 | |
| 095678 | K2C75_HUMAN | Keratin, type II cytoskeletal 75 | KRT75 | 551 |
| | | (Cytokeratin-75) (CK-75) (Keratin-6 | K6HF KB18 | |
| | | hair follicle) (hK6hf) (Keratin-75) | | |
| | | (K75) (Type II keratin-K6hf) (Type-II | | |
| | | keratin Kb18) | | |
| Q14533 | KRT81_HUMAN | Keratin, type II cuticular Hb1 (Hair | KRT81 | 505 |
| | | keratin K2.9) (Keratin, hair, basic, 1) | KRTHB1 | |
| | | (Keratin-81) (K81) (Metastatic lymph | MLN137 | |
| | | node 137 gene protein) (MLN 137) | | |
| | | (Type II hair keratin Hb1) (Type-II | | |
| | | keratin Kb21) (ghHKb1) (ghHb1) | | |
| Q9NSB4 | KRT82_HUMAN | Keratin, type II cuticular Hb2 | KRT82 | 513 |
| | | (Keratin-82) (K82) (Type II hair | KRTHB2 | |
| | | keratin Hb2) (Type-II keratin Kb22) | | |
| P78385 | KRT83_HUMAN | Keratin, type II cuticular Hb3 (Hair | KRT83 | 493 |
| | | keratin K2.10) (Keratin-83) (K83) | KRTHB3 | |
| | | (Type II hair keratin Hb3) (Type-II | | |
| | | keratin Kb23) | | |
| Q9NSB2 | KRT84 HUMAN | Keratin, type II cuticular Hb4 | KRT84 | 600 |
| | | (Keratin-84) (K84) (Type II hair | KRTHB4 | |
| | | keratin Hb4) (Type-II keratin Kb24) | | |
| P78386 | KRT85_HUMAN | Keratin, type II cuticular Hb5 (Hair | KRT85 | 507 |
| | | keratin K2.12) (Keratin-85) (K85) | KRTHB5 | |
| | | (Type II hair keratin Hb5) (Type-II | | |
| | | keratin Kb25) | | |
| 043790 | KRT86_HUMAN | Keratin, type II cuticular Hb6 (Hair | KRT86 | 486 |
| | | keratin K2.11) (Keratin-86) (K86) | KRTHB6 | |
| | | (Type II hair keratin Hb6) (Type-II | | |
| | | keratin Kb26) | | |

The keratin can be a full-length keratin, or a truncated keratin. The keratin amino acid sequence can be derived from a human keratin sequence, or it can be from another organism, for example another mammal. In preferred embodiments, the keratin is derived from a human keratin, such as K12, K31, K33a or K33b. The full-length keratin protein sequences of such human keratins are listed below in Table 3. The keratin may be a homologue or a fragment of any thereof, and/or may comprise an amino acid sequence having at least about 70% sequence identity to any of the keratin sequences listed in Table 3 or a fragment of any thereof.

TABLE 3
Exemplary full-length human keratin sequences

| Name | Amino Acid Sequence |
|---|---|
| K12 (Human) | MDLSNNTMSLSVRTPGLSRRLSSQSVIGRPRGMSASSVGSGYGGSAFGF |
| | GASCGGGFSAASMFGSSSGFGGGSGSSMAGGLGAGYGRALGGGSFGGLG |
| | MGFGGSPGGGSLGILSGNDGGLLSGSEKETMQNLNDRLASYLDKVRALE |
| | EANTELENKIREWYETRGTGTADASQSDYSKYYPLIEDLRNKIISASIG |
| | NAQLLLQIDNARLAAEDFRMKYENELALRQGVEADINGLRRVLDELTLT |
| | RTDLEMQIESLNEELAYMKKNHEDELQSFRVGGPGEVSVEMDAAPGVDL |
| | TRLLNDMRAQYETIAEQNRKDAEAWFIEKSGELRKEISTNTEQLQSSKS |
| | EVTDLRRAFQNLEIELQSQLAMKKSLEDSLAEAEGDYCAQLSQVQQLIS |
| | NLEAQLLQVRADAERQNVDHQRLLNVKARLELEIETYRRLLDGEAQGDG |
| | LEESLFVTDSKSQAQSTDSSKDPTKTRKIKTVVQEMVNGEVVSSQVQEI |
| | EELM (SEQ ID NO: 2) |
| K31 (Human) | MPYNFCLPSLSCRTSCSSRPCVPPSCHSCTLPGACNIPANVSNCNWFCE |
| | GSFNGSEKETMQFLNDRLASYLEKVRQLERDNAELENLIRERSQQQEPL |
| | LCPSYQSYFKTIEELQQKILCTKSENARLVVQIDNAKLAADDFRTKYQT |
| | ELSLRQLVESDINGLRRILDELTLCKSDLEAQVESLKEELLCLKSNHEQ |
| | EVNTLRCQLGDRLNVEVDAAPTVDLNRVLNETRSQYEALVETNRREVEQ |
| | WFTTQTEELNKQVVSSSEQLQSYQAEIIELRRTVNALEIELQAQHNLRD |
| | SLENTLTESEARYSSQLSQVQSLITNVESQLAEIRSDLERQNQEYQVLL |
| | DVRARLECEINTYRSLLESEDCNLPSNPCATTNACSKPIGPCLSNPCTS |
| | CVPPAPCTPCAPRPRCGPCNSFVR (SEQ ID NO: 3) |
| K33a (Human) | MSYSCGLPSLSCRTSCSSRPCVPPSCHGCTLPGACNIPANVSNCNWFCE |
| | GSFNGSEKETMQFLNDRLASYLEKVRQLERDNAELENLIRERSQQQEPL |
| | VCASYQSYFKTIEELQQKILCSKSENARLVVQIDNAKLASDDFRTKYET |
| | ELSLRQLVESDINGLRRILDELTLCRSDLEAQVESLKEELLCLKQNHEQ |
| | EVNTLRCQLGDRLNVEVDAAPTVDLNQVLNETRSQYEALVETNRREVEQ |
| | WFATQTEELNKQVVSSSEQLQSYQAEIIELRRTVNALEIELQAQHNLRD |
| | SLENTLTESEARYSSQLSQVQRLITNVESQLAEIRSDLERQNQEYQVLL |
| | DVRARLECEINTYRSLLESEDCKLPSNPCATTNACDKSTGPCISNPCGL |
| | RARCGPCNTFGY (SEQ ID NO: 4) |
| K33b (Human) | MPYNFCLPSLSCRTSCSSRPCVPPSCHGYTLPGACNIPANVSNCNWFCE |
| | GSFNGSEKETMQFLNDRLASYLEKVRQLERDNAELENLIRERSQQQEPL |
| | LCPSYQSYFKTIEELQQKILCSKSENARLVVQIDNAKLAADDFRTKYQT |
| | EQSLRQLVESDINSLRRILDELTLCRSDLEAQMESLKEELLSLKQNHEQ |
| | EVNTLRCQLGDRLNVEVDAAPAVDLNQVLNETRNQYEALVETNRREVEQ |
| | WFATQTEELNKQVVSSSEQLQSYQAEIIELRRTVNALEIELQAQHNLRY |
| | SLENTLTESEARYSSQLSQVQSLITNVESQLAEIRSDLERQNQEYQVLL |
| | DVRARLECEINTYRSLLESEDCKLPSNPCATTNACEKPIGSCVTNPCGP |
| | RSRCGPCNTFGY (SEQ ID NO: 5) |

In other embodiments, the keratin may be full-length or truncated keratin polypeptides comparable to human keratins. Suitable comparable human keratin sequences include, for example, the following protein NCBI accession numbers: AAB30058.2, AAB59562.1, CAA73943.1, BAA09320.1, CAA31695.1, P13647.3, P04264.6, P35908.2, Q14533.3, P05787.7, P48668.3, P02538.3, 043790.1, P78385.2, P78386.1, NP_002272.2, P12035.3, Q3SY84.3, 095678.2, and NP_002274.1. In alternative embodiments, the keratin may be derived from an animal keratin, such as other mammals.

In other embodiments, the keratin may be full-length or truncated keratin polypeptides comparable to chicken keratins, or other poultry keratins, such as from domestic fowls, including chickens, turkeys, geese, and ducks. Suitable comparable keratin (or truncate thereof) sequences from Gallus gallus (chicken) include NCBI accession numbers P02450.2, P20308.2, P20307.2, P04458.3, P04459.3, P25692.2, AA085139.1, NP_001264675.1, AAA48932.1, AAA48931.1, XP_040510675.1, NP_001075171.2, and NP_990263.2.

In other embodiments, the keratin may be full-length or truncated keratin polypeptides comparable to keratins or keratin truncates from Bos taurus (cattle), including, for example, the following NCBI protein accession numbers: Q148H8.1, AOJND2.1, A7YWK3.1, Q5XQN5.1, A4FUZO.1, Q148H4.1, Q148H7.1, Q29S21.1, P05786.3, Q148H5.1, Q08D91.1, A3KN27.1, and A6QNX5.1

In certain preferred embodiments, the keratin segment of the non-naturally occurring polypeptides provided herein has at least about 70%, at least 70%, at least about 75%, at least 75%, at least about 80%, at least 80%, at least about 85%, at least 85%, at least about 90%, at least 90%, at least about 91%, at least 91%, at least about 92%, at least 92%, at least about 93%, at least 93%, at least about 94%, at least 94%, at least about 95%, at least 95%, at least about 96%, at least 96%, at least about 97%, at least 97%, at least about 98%, at least 98%, at least about 99%, or at least 99% sequence identity to the corresponding region of a full-length keratin, or a truncate or a fragment thereof. In some instances, a portion or portions of a natural amino acid sequence is deleted, but the remainder of the sequence is substantially similar or identical to the natural amino acid sequence. In certain preferred embodiments, the keratin segment of the non-naturally occurring polypeptide is a portion of the full-length keratin from which it was derived, and the % sequence identity is relative to the corresponding region of the full-length keratin.

In some embodiments, the keratin segment is a keratin polypeptide that is smaller than a full-length (e.g., natural) keratin wherein one or more portions (e.g., internal and/or terminal portion(s)) of the full-length (e.g., natural) keratin is not present. In various instances, the non-naturally occurring polypeptides provided herein comprise a keratin that is truncated at the C-terminal end, the N-terminal end, truncated by removal of internal portion(s) of the full-length keratin polypeptide (e.g., internal truncation), truncated at both the C-terminal end and the N-terminal end, or comprises one or both of a C-terminal truncation and an N-terminal truncation as well as an internal truncation. In some instances, the term “truncated keratin” is interchangeably used with the term “keratin fragment”. In some instances, the truncated keratin includes any contiguous keratin amino acid residues that are at least about 10%, at least 10%, at least about 15%, at least 15%, at least about 20%, at least 20%, at least about 25%, at least 25%, at least about 30%, at least 30%, at least about 35%, at least 35%, at least about 40%, at least 40%, at least about 45%, at least 45%, at least about 50%, at least 50%, at least about 55%, at least 55%, at least about 60%, at least 60%, at least about 65%, at least 65%, at least about 70%, at least 70%, at least about 75%, at least 75%, at least about 80%, at least 80%, at least about 85%, at least 85%, at least about 90%, at least 90%, at least about 95%, or at least 95% of full-length natural or naturally present corresponding keratin. In some instances, the truncated keratin (e.g., K31) includes any contiguous keratin amino acid residues that are less than about 15%, less than 15%, less than about 20%, less than 20%, less than about 25%, less than 25%, less than about 30%, less than 30%, less than about 35%, less than 35%, less than about 40%, less than 40%, less than about 45%, less than 45%, less than about 50%, less than 50%, less than about 55%, less than 55%, less than about 60%, less than 60%, less than about 65%, less than 65%, less than about 70%, less than 70%, less than about 75%, or less than 75%, of full-length natural or naturally present corresponding keratin (e.g., K31). A truncated keratin includes a polypeptide that has at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 110 amino acids, at least 120 amino acids, at least 130 amino acids, at least 140 amino acids, at least 150 amino acids, at least 160 amino acids, at least 170 amino acids, at least 180 amino acids, at least 190 amino acids, at least 200 amino acids, at least 210 amino acids, at least 220 amino acids, at least 230 amino acids, at least 240 amino acids, at least 250 amino acids, at least 260 amino acids, at least 270 amino acids, at least 280 amino acids, at least 290 amino acids, or at least 300 amino acids of a keratin polypeptide.

In some embodiments, the keratin polypeptide is prepared as a composition comprising a recombinant polypeptide, wherein the recombinant polypeptide comprises an amino acid sequence having at least at least about 70%, at least 70%, at least about 75%, at least 75%, at least about 80%, at least 80%, at least about 85%, at least 85%, at least about 90%, at least 90%, at least about 91%, at least 91%, at least about 92%, at least 92%, at least about 93%, at least 93%, at least about 94%, at least 94%, at least about 95%, at least 95%, at least about 96%, at least 96%, at least about 97%, at least 97%, at least about 98%, at least 98%, at least about 99%, or at least 99% sequence identity to the corresponding region of a full-length keratin having a truncation (i.e., a truncated keratin) of at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids. In preferred embodiments, the recombinant polypeptide has a purity (e.g., in the composition) of at least 60%, at least 65%, at least 70%, at least 75%, at least 80%, at least 85%, at least 90%, at least 95%, at least 96%, at least 97%, at least 98%, or at least 99% when measured by high-performance liquid chromatography or mass spectrometry.

A truncated keratin provided herein may be truncated (e.g., a total truncation) by 50 amino acids to 600 amino acids, 50 amino acids to 550 amino acids, 50 amino acids to 500 amino acids, 50 amino acids to 450 amino acids, 50 amino acids to 400 amino acids, 50 amino acids to 350 amino acids, 50 amino acids to 300 amino acids, 50 amino acids to 250 amino acids, 50 amino acids to 200 amino acids, 50 amino acids to 150 amino acids, or 50 amino acids to 100 amino acids (e.g., relative to a full-length keratin). In another embodiment, a truncated keratin is truncated by at least 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids (e.g., relative to a full-length keratin).

In some embodiments, the truncated keratin may be truncated at the C-terminal end (relative to a full-length keratin) by any suitable number of amino acid residues, such as 10 to 600, 10 to 550, 10 to 500, 10 to 450, 10 to 400, 10 to 350, 10 to 300, 10 to 250, 10 to 200, 10 to 150, 10 to 100, 50 to 600, 50 to 550, 50 to 500, 50 to 450, 50 to 400, 50 to 350, 50 to 300, 50 to 250, 50 to 200, 50 to 150, 50 to 100, and the like. In some cases, the truncated keratin provided herein may be truncated at the C-terminal end (relative to a full-length keratin) by at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids.

In some embodiments, the truncated keratin may be truncated at the N-terminal end (relative to a full-length keratin) by any suitable number of amino acid residues, such as 10 to 600, 10 to 550, 10 to 500, 10 to 450, 10 to 400, 10 to 350, 10 to 300, 10 to 250, 10 to 200, 10 to 150, 10 to 100, 50 to 600, 50 to 550, 50 to 500, 50 to 450, 50 to 400, 50 to 350, 50 to 300, 50 to 250, 50 to 200, 50 to 150, 50 to 100, and the like. In some cases, the truncated keratin provided herein may be truncated at the N-terminal end (relative to a full-length keratin) by at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids.

In some embodiments, the truncated keratin may be truncated at both the N-terminal end and the C-terminal end (relative to a full-length keratin) by any suitable number of combined amino acid residues, such as 10 to 600, 10 to 550, 10 to 500, 10 to 450, 10 to 400, 10 to 350, 10 to 300, 10 to 250, 10 to 200, 10 to 150, 10 to 100, 50 to 600, 50 to 550, 50 to 500, 50 to 450, 50 to 400, 50 to 350, 50 to 300, 50 to 250, 50 to 200, 50 to 150, 50 to 100, and the like. In some cases, the truncated keratin provided herein can have a combined N-terminal and C-terminal truncation (relative to a full-length keratin) of at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids.

In some embodiments, the truncated keratin provided herein may have a N-terminal truncation of 50 to 100 amino acids and a C-terminal truncation of 50 to 100 amino acids, a N-terminal truncation of 50 to 100 amino acids and a C-terminal truncation of 50 to 150 amino acids, a N-terminal truncation of 50 to 100 amino acids and a C-terminal truncation of 50 to 200 amino acids, a N-terminal truncation of 50 to 100 amino acids and a C-terminal truncation of 50 to 250 amino acids, a N-terminal truncation of 50 to 100 amino acids and a C-terminal truncation of 50 to 300 amino acids, a N-terminal truncation of 50 to 100 amino acids and a C-terminal truncation of 50 to 350 amino acids, or a N-terminal truncation of 50 to 100 amino acids and a C-terminal truncation of 50 to 400 amino acids.

In some embodiments, the truncated keratin provided herein may have a C-terminal truncation of 50 to 100 amino acids and a N-terminal truncation of 50 to 150 amino acids, a C-terminal truncation of 50 to 100 amino acids and a N-terminal truncation of 50 to 200 amino acids, a C-terminal truncation of 50 to 100 amino acids and a N-terminal truncation of 50 to 250 amino acids, a C-terminal truncation of 50 to 100 amino acids and a N-terminal truncation of 50 to 300 amino acids, a C-terminal truncation of 50 to 100 amino acids and a N-terminal truncation of 50 to 350 amino acids, or a C-terminal truncation of 50 to 100 amino acids and a N-terminal truncation of 50 to 400 amino acids.

In some embodiments, the truncated keratin provided herein may have a N-terminal truncation of 50 to 250 amino acids and a C-terminal truncation of 50 to 100 amino acids, a N-terminal truncation of 50 to 250 amino acids and a C-terminal truncation of 50 to 150 amino acids, a N-terminal truncation of 50 to 250 amino acids and a C-terminal truncation of 50 to 200 amino acids, a N-terminal truncation of 50 to 250 amino acids and a C-terminal truncation of 50 to 250 amino acids, a N-terminal truncation of 50 to 250 amino acids and a C-terminal truncation of 50 to 300 amino acids, a N-terminal truncation of 50 to 250 amino acids and a C-terminal truncation of 50 to 350 amino acids, or a N-terminal truncation of 50 to 250 amino acids and a C-terminal truncation of 50 to 400 amino acids.

In some embodiments, the truncated keratin provided herein may have a C-terminal truncation of 50 to 250 amino acids and a N-terminal truncation of 50 to 100 amino acids, a C-terminal truncation of 50 to 250 amino acids and a N-terminal truncation of 50 to 150 amino acids, a C-terminal truncation of 50 to 250 amino acids and a N-terminal truncation of 50 to 200 amino acids, a C-terminal truncation of 50 to 250 amino acids and a N-terminal truncation of 50 to 250 amino acids, a C-terminal truncation of 50 to 250 amino acids and a N-terminal truncation of 50 to 300 amino acids, a C-terminal truncation of 50 to 250 amino acids and a N-terminal truncation of 50 to 350 amino acids, or a C-terminal truncation of 50 to 250 amino acids and a N-terminal truncation of 50 to 400 amino acids.

In some embodiments, the truncated keratin may be internally truncated (relative to a full-length keratin) by any suitable number of amino acid residues, 10 to 600, 10 to 550, 10 to 500, 10 to 450, 10 to 400, 10 to 350, 10 to 300, 10 to 250, 10 to 200, 10 to 150, 10 to 100, 50 to 600, 50 to 550, 50 to 500, 50 to 450, 50 to 400, 50 to 350, 50 to 300, 50 to 250, 50 to 200, 50 to 150, 50 to 100, and the like. In some cases, the truncated keratin may be internally truncated (relative to a full-length keratin) by at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids.

In some embodiments, the keratin polypeptide may be a truncated K12, K31, K33a or K33b polypeptide. In certain embodiments, the keratin polypeptide may be a sequence provided below, or a fragment thereof.

Truncated human keratin type 12, having 179 amino acids.

(SEQ ID NO: 6)
LQIDNARLAAEDFRMKYENELALRQGVEADINGLRRVLDELTLTRTDLEM
QIESLNEELAYMKKNHEDELQSFRVGGPGEVSVEMDAAPGVDLTRLLNDM
RAQYETIAEQNRKDAEAWFIEKSGELRKEISTNTEQLQSSKSEVTDLRRA
FQNLEIELQSQLAMKKSLEDSLAEAEGDY

Truncated human keratin type 31 (also known as type 1, cuticular Ha1) having 147 amino acids.

(SEQ ID NO: 7)
QLGDRLNVEVDAAPTVDLNRVLNETRSQYEALVETNRREVEQWFTTQTEE
LNKQVVSSSEQLQSYQAEIIELRRTVNALEIELQAQHNLRDSLENTLTES
EARYSSQLSQVQSLITNVESQLAEIRSDLERQNQEYQVLLDVRARLE

Truncated human keratin type 31 (also known as type 1, cuticular Ha1) having 125 amino acids.

(SEQ ID NO: 8)
QLGDRLNVEVDAAPTVDLNRVLNETRSQYEALVETNRREVEQWFTTQTEE
LNKQVVSSSEQLQSYQAEIIELRRTVNALEIELQAQHNLRDSLENTLTES
EARYSSQLSQVQSLITNVESQLAEI.

Truncated human keratin type 31 (also known as type 1, cuticular Ha1) having 100 amino acids.

(SEQ ID NO: 9)
QLGDRLNVEVDAAPTVDLNRVLNETRSQYEALVETNRREVEQWFT
TQTEELNKQVVSSSEQLQSYQAEIIELRRTVNALEIELQAQHNLR
DSLENTLTES

Truncated human keratin type 31 (also known as type 1, cuticular Ha1) having 75 amino acids.

(SEQ ID NO: 10)
QLGDRLNVEVDAAPTVDLNRVLNETRSQYEALVETNRREVEQWFT
TQTEELNKQVVSSSEQLQSYQAEIIELRRT

Truncated human keratin type 31 (also known as type 1, cuticular Ha1) having 50 amino acids.

(SEQ ID NO: 11)
QLGDRLNVEVDAAPTVDLNRVLNETRSQYEALVETNRREVEQWFT
TQTEE

Truncated human keratin type 33a (also known as type 1, cuticular Ha31), having 147 amino acids.

(SEQ ID NO: 12)
QLGDRLNVEVDAAPTVDLNQVLNETRSQYEALVETNRREVEQWFA
TQTEELNKQVVSSSEQLQSYQAEIIELRRTVNALEIELQAQHNLR
DSLENTLTESEARYSSQLSQVQRLITNVESQLAEIRSDLERQNQE
YQVLLDVRARLE

### Solubility-Enhancing Amino Acid Sequences

Keratin polypeptides are difficult to express in recombinant systems, such as through traditional microbial systems such as Escherichia coli. Attempts at expression of recombinant keratins typically result in insoluble keratin polypeptides, and expression of keratin polypeptides in inclusion bodies. Inclusion bodies hinder the production of soluble and functional recombinant proteins, particularly at commercial scale. The methods of the present disclosure provide solutions to overcome keratin expression problems in recombinant systems, in particular microbial expression systems. Nevertheless, the methods of the present disclosure can also be readily be adaptable in eukaryotic expression systems, such as in mammalian cells In one embodiment, the present disclosure provides solubility-enhancing amino acid sequences that may be used together with the keratin polypeptide sequences to produce recombinant non-naturally occurring polypeptides.

Solubility-enhancing amino acid sequences (also referred to as “solubility-enhancing polypeptides” or “solubility-enhancing tags”) as provided herein can be expressed with many types of recombinantly produced keratins to improve protein expression, solubility and/or purification. As shown herein, a variety of solubility-enhancing sequences can be designed and used, and a variety of target polypeptide sequences can be expressed using the methods disclosed.

In some embodiments, a solubility-enhancing amino acid sequence can be used as a method of improving solubility of a polypeptide of interest, the method comprising expressing the polypeptide of interest in the presence of a solubility-enhancing amino acid sequence, wherein the solubility-enhancing amino acid sequence comprises at least 24 amino acids in length and at least 25% Gly amino acid residues, wherein the solubility of the polypeptide of interest is improved when in the presence of the solubility-enhancing amino acid sequence as compared to when in the absence of the solubility-enhancing amino acid sequence. In some embodiments, the solubility-enhancing amino acid sequence has at least 15% amino acid residues selected from the group consisting of: Asp, Glu, Arg, Ser, and combinations thereof. In some embodiments, the solubility-enhancing amino acid sequence has at least one of Asp, Glu, Arg, and Ser. In some embodiments, the solubility-enhancing amino acid sequence has less than 15% amino acid residues selected from the group consisting of: Phe, Ile, Leu, Met, Val, Trp, Tyr, and combinations thereof. In some embodiments, the solubility-enhancing amino acid sequence has less than 15% of Phe, Ile, Leu, Met, Val, Trp, and Tyr. In some embodiments, the solubility-enhancing amino acid sequence has none of Phe, Ile, Leu, Met, Val, Trp, and Tyr. In some embodiments, the solubility-enhancing amino acid sequence consists of a triplet amino acid pattern of (Gly-X—Y)ₙ, wherein X and Y are any amino acid residue and n=8-300. In some embodiments, at least one (Gly-X—Y) triplet, X and Y are the same amino acid residue. In some embodiments, at least one (Gly-X—Y) triplet, X and Y are different amino acid residues. In some embodiments, at least one (Gly-X—Y) triplet is different from another (Gly-X—Y) triplet. In some embodiments, at least one (Gly-X—Y) triplet is the same as another (Gly-X—Y) triplet. In some embodiments, the polypeptide of interest is expressed as a fusion polypeptide with the solubility-enhancing amino acid sequence. In some embodiments, the solubility-enhancing amino acid sequence is fused to the N-terminus of the polypeptide of interest. In some embodiments, the solubility-enhancing amino acid sequence is fused to the C-terminus of the polypeptide of interest. In some embodiments, the solubility-enhancing amino acid sequence comprises a first solubility-enhancing amino acid sequence fused to the N-terminus of the polypeptide of interest, and a second solubility-enhancing amino acid sequence fused to the C-terminus of the polypeptide of interest. In some embodiments, the solubility-enhancing amino acid sequence and the polypeptide of interest are directly linked by a peptide bond. In some embodiments, the solubility-enhancing amino acid sequence and the polypeptide of interest are linked via a linker sequence. In some embodiments, the linker sequence is selected from the group consisting of a flexible linker, a rigid linker, and a cleavable linker. In some embodiments, the cleavable linker comprises a protease cleavage site. In some embodiments, the polypeptide of interest and the solubility-enhancing amino acid sequence are expressed as separate polypeptides. In some embodiments, only the polypeptide of interest is expressed. In some embodiments, the solubility-enhancing amino acid sequence has at least 80% sequence identity to a fragment of a collagen polypeptide. In some embodiments, the collagen polypeptide is a human collagen polypeptide. In some embodiments, the solubility-enhancing amino acid sequence has at least 80% sequence identity to the corresponding region of a full-length human type I, alpha 1 collagen polypeptide and is at least 50 amino acids in length. In some embodiments, the solubility-enhancing amino acid sequence is from 50-300 amino acids in length. In some embodiments, the collagen polypeptide is a non-human animal collagen polypeptide. In some embodiments, the method may further comprise culturing a host cell in a culture medium, wherein the host cell comprises one or more exogenous polynucleotides encoding: (i) the solubility-enhancing amino acid sequence; (ii) the polypeptide of interest; or (iii) both. In some embodiments, the host cell expresses the solubility-enhancing amino acid sequence, the polypeptide of interest, or both. In some embodiments, the host cell expresses the solubility-enhancing amino acid sequence and the polypeptide of interest as a fusion polypeptide. In some embodiments, the host cell expresses the solubility-enhancing amino acid sequence and the polypeptide of interest as separate polypeptides. In some embodiments, the host cell does not express the solubility-enhancing amino acid sequence. In some embodiments, the method may further comprise separating the solubility-enhancing amino acid sequence from the polypeptide of interest, thereby producing a target polypeptide of interest. In some embodiments, the polypeptide of interest and the solubility-enhancing amino acid sequence are expressed as a fusion polypeptide, the fusion polypeptide comprises a cleavable linker linking the polypeptide of interest to the solubility-enhancing amino acid sequence, and the separating comprises cleaving the cleavable linker to separate the polypeptide of interest from the solubility-enhancing amino acid sequence. In some embodiments, the method may further comprise recovering the polypeptide of interest from the culture medium. In some embodiments, the method may further comprise comprising purifying the polypeptide of interest. In some embodiments, the host cell is a microbial host cell. In some embodiments, the microbial host cell is a bacterial cell. In some embodiments, the bacterial cell is Escherichia coli. In some embodiments, the polypeptide of interest is not expressed or is not present in an inclusion body. In some embodiments, the host cell is a eukaryotic host cell. In some embodiments, the eukaryotic host cell is a mammalian cell. In some embodiments, the polypeptide of interest is secreted from the host cell into the culture medium. In some embodiments, the polypeptide of interest is present in the culture medium as a soluble polypeptide.

In some embodiments, a solubility-enhancing amino acid sequence can be fused to a keratin polypeptide e.g., to increase or enhance the expression, solubility, and/or purification of the keratin polypeptide. Solubility-enhancing polypeptide fusions can be particularly helpful for improving the purification of polypeptides or proteins that are otherwise difficult to purify on their own using traditional methods. In one embodiment, keratin amino acid sequences expressed together with solubility-enhancing tags provided herein provide increased solubility at a low pH. When a keratin polypeptide is expressed and secreted into the culture media according to methods herein, the culture media can then be acidified to a pH level that causes contaminating proteins to “crash out” or precipitate, while leaving the keratin polypeptide in solution. This provides for a simple purification step to purify the keratin polypeptide. In another embodiment, keratin sequences expressed together with solubility-enhancing tags provided herein exhibit differential precipitation at different ranges of salts, such as ammonium sulfate. This provides for a different and simple purification method. In another embodiment, the keratin polypeptide is expressed and secreted into the culture media according to methods herein, the culture media can then be subjected to a temperature gradient that causes contaminating proteins to precipitate, while leaving the keratin polypeptide in solution.

In preferred embodiments, the solubility-enhancing amino acid sequence consists of a triplet amino acid pattern of (Gly-X—Y)ₙ, where Gly represents the amino acid glycine, and X and Y each represent any amino acid residue. In some cases, X and Y may be the same amino acid residue. In other cases, X and Y may be different amino acid residues. In some cases, at least one (Gly-X—Y) triplet, X and Y are the same amino acid residue. In some cases, at least one (Gly-X—Y) triplet, X and Y are different amino acid residues. In some cases, at least one (Gly-X—Y) triplet is different from another (Gly-X—Y) triplet. In some cases, at least one (Gly-X—Y) triplet is the same as another (Gly-X—Y) triplet. The amino acid residues present in the Gly-X—Y triplet may be any of the standard amino acid residues that naturally occur in proteins, and/or any non-standard amino acid residues that do not naturally occur in proteins, and/or any modified amino acid residues.

The triplet pattern (Gly-X—Y) is repeated “n” times with any triplet matching the Gly-X—Y pattern, where n is at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300. In other preferred embodiments the triplet pattern is repeated “n” times, where n is 3-10, 3-15, 3-20, 5-10, 5-15, 5-20, 10-15, 10-20, 15-20, 10-100, 15-100, 20-100, 25-100, 30-100, 35-100, 40-100, 45-100, 50-100, 55-100, 60-100, 65-100, 70-100, 75-100, 80-100, 85-100, 90-100, 95-100, 10-200, 15-200, 20-200, 25-200, 30-200, 35-200, 40-200, 45-200, 50-200, 55-200, 60-200, 65-200, 70-200, 75-200, 80-200, 85-200, 90-200, 95-200, 100-200, 110-200, 120-200, 130-200, 140-200, 150-200, 160-200, 170-200, 180-200, 190-200, 20-80, 25-80, 30-80, 35-80, 40-80, 45-80, 50-80, 55-80, 60-80, 65-80, 70-80, 75-80, 20-60, 25-60, 30-60, 35-60, 40-60, 45-60, 50-60, 55-60, 8-300, 10-300, 20-300, 30-300, 40-300, 50-300, 60-300, 70-300, 80-300, 90-300, 100-300, 110-300, 120-300, 130-300, 140-300, 150-300, 160-300, 170-300, 180-300, 190-300, or 200-300. It is understood that “n” may be varied to maximize the solubility-enhancing function, and “n” may be any integer between 3 and 300, or any range having a lower number of 3 to a higher number of 300.

In certain embodiments, the solubility-enhancing function may provide an increased level of soluble polypeptide in the expression stages, for example, by decreasing the presence of polypeptide in inclusion bodies, by increasing the secretion of polypeptide into the media, by altering the structural properties or folding of the polypeptide, and the like. In other embodiments, the solubility-enhancing function may provide an increased level of soluble polypeptide in the purification stages, for example, by increasing the presence of polypeptide in soluble supernatant fractions as opposed to cell pellet fractions upon separating cells from media, increasing the presence of polypeptide in the soluble fractions upon purifications steps with various filtration steps, various chemical separation steps, various physical separation steps, various treatments used to separate polypeptide fractions, for example treatment with acids or bases to alter pH, or various salts to alter ionic compositions, and the like, or by decreasing the precipitation (e.g., as a result of insolubility) of the polypeptide during any such treatments. Accordingly, the solubility-enhancing function may be improved by testing various solubility-enhancing amino acid sequences in combination with the target protein to be expressed in the context of typical expression and purifications used for the target protein according to methods provided herein.

In certain embodiments, the solubility-enhancing amino acid sequence may be entirely designed de novo in accordance with the triplet pattern provided herein. In other embodiments, the solubility-enhancing amino acid sequence may be derived from a naturally occurring sequence, such as a collagen polypeptide or a fragment thereof, wherein the resulting solubility-enhancing amino acid sequence triplet pattern is maintained. In certain embodiments the solubility-enhancing amino acid sequence is a sub-sequence of the naturally occurring collagen polypeptide having at least about 40%, at least 40%, at least about 50%, at least 50%, at least about 60%, at least 60%, at least about 70%, at least 70%, at least about 75%, at least 75%, at least about 80%, at least 80%, at least about 85%, at least 85%, at least about 90%, at least 90%, at least about 91%, at least 91%, at least about 92%, at least 92%, at least about 93%, at least 93%, at least about 94%, at least 94%, at least about 95%, at least 95%, at least about 96%, at least 96%, at least about 97%, at least 97%, at least about 98%, at least 98%, at least about 99%, or at least 99% sequence identity to the corresponding region of a full-length collagen polypeptide.

In certain embodiments the solubility-enhancing amino acid has a length of from 60-300 amino acids, from 60-291 amino acids from 60-282 amino acids, from 60-273 amino acids, from 60-264 amino acids, from 60-255 amino acids, from 60-246 amino acids, from 60-237 amino acids, from 60-228 amino acids, from 60-219 amino acids, from 60-210 amino acids, from 60-201 amino acids, from 60-192 amino acids, from 60-183 amino acids, from 60-174 amino acids, from 60-165 amino acids, from 60-156 amino acids, from 60-147 amino acids, from 60-138 amino acids, from 60-129 amino acids, from 60-120 amino acids, from 60-111 amino acids, from 60-102 amino acids, from 60-93 amino acids, from 60-84 amino acids, or from 60-75 amino acids, In certain embodiments, this length of solubility-enhancing amino acid residues corresponds to a sub-segment of a naturally occurring collagen polypeptide.

In certain embodiments, the solubility-enhancing amino acid has a length of from 9-99 amino acids, 9-96 amino acids, 9-93 amino acids, 9-90 amino acids, 9-87 amino acids, 9-84 amino acids, 9-81 amino acids, 9-78 amino acids, 9-75 amino acids, 9-72 amino acids, 9-69 amino acids, 9-66 amino acids, 9-63 amino acids, 9-60 amino acids, 9-57 amino acids, 9-54 amino acids, 9-51 amino acids, 9-48 amino acids, 9-45 amino acids, 9-42 amino acids, 9-39 amino acids, 9-36 amino acids, 9-33 amino acids, 9-30 amino acids, 9-27 amino acids, 9-24 amino acids, 9-21 amino acids, 9-18 amino acids, 9-15 amino acids, or 9-12 amino acids. Such lengths may be particularly useful for enhancing solubility of shorter target polypeptides or peptides.

In certain preferred embodiments the solubility-enhancing amino acid sequence has a length of at least 9 amino acids, at least 12 amino acids, at least 15 amino acids, at least 18 amino acids, at least 21 amino acids, at least 24 amino acids, at least 27 amino acids, at least 30 amino acids, at least 33 amino acids, at least 36 amino acids, at least 39 amino acids, at least 42 amino acids, at least 45 amino acids, at least 48 amino acids, at least 51 amino acids, at least 54 amino acids, at least 57 amino acids, at least 60 amino acids, at least 63 amino acids, at least 66, at least 69 amino acids, at least 72 amino acids, at least 75 amino acids, at least 78 amino acids, at least 81 amino acids, at least 84 amino acids at least 87 amino acids, at least 90 amino acids, at least 93 amino acids, at least 96 amino acids, at least 99 amino acids, at least 102 amino acids, at least 105 amino acids, at least 108 amino acids, at least 111 amino acids, at least 114 amino acids, at least 117 amino acids, at least 120 amino acids, at least 123 amino acids, at least 126 amino acids, at least 129 amino acids, at least 132 amino acids, at least 135 amino acids, at least 138 amino acids, at least 141 amino acids, at least 144 amino acids, at least 147 amino acids, at least 150 amino acids, at least 153 amino acids, at least 156 amino acids, at least 159 amino acids, at least 162 amino acids, at least 165 amino acids, at least 168 amino acids, at least 171 amino acids, at least 174 amino acids, at least 177 amino acids, at least 180 amino acids, at least 183, at least 186 amino acids, at least 189 amino acids, at least 192 amino acids, at least 195 amino acids, at least 198 amino acids, at least 201 amino acids, at least 204 amino acids, at least 207 amino acids, at least 210 amino acids, at least 213 amino acids, at least 216 amino acids, at least 219 amino acids, at least 222 amino acids, at least 225 amino acids, at least 228 amino acids, at least 231 amino acids, at least 234 amino acids, at least 237 amino acids, at least 240 amino acids, at least 243 amino acids, at least 246 amino acids, at least 249 amino acids, at least 252 amino acids, at least 255 amino acids, at least 258 amino acids, at least 261 amino acids, at least 264 amino acids, at least 267 amino acids, at least 270 amino acids, at least 273 amino acids, at least 276 amino acids, at least 279 amino acids, at least 282 amino acids, at least 285 amino acids, at least 288 amino acids, at least 291 amino acids, at least 294 amino acids, at least 297 amino acids, or at least 300 amino acids. In certain embodiments this length of solubility-enhancing amino acid residues corresponds to a sub-segment of a naturally occurring collagen polypeptide.

Collagen is an abundant protein present in various connective tissues in the body including tendons, ligaments, skin, and hair. Collagens or collagen supplements are popular in medical, cosmetic, and/or health purposes (e.g., stimulating skin growth, promoting wound healing, strengthening nails or joints, etc.). The structure of natural collagen can be a triple helix in which three polypeptide strands together form a helical coil. The individual polypeptide strands are composed of repeating triplet amino acid sequences designated as GLY—X—Y, where X and Y can be any amino acid, and the first amino acid is glycine. The amino acids proline and hydroxyproline are found in high concentrations in collagen. The most common triplet in naturally occurring collagen is glycine-proline-hydroxyproline (Gly-Pro-Hyp) accounting for approximately 10.5% of the triplets in collagen.

In certain embodiments, the solubility-enhancing amino acid residues corresponds to a sub-segment of a naturally occurring collagen polypeptide. In certain preferred embodiments the collagen is a human collagen. In other embodiments the collagen is an animal collagen, such as a chicken collagen. Exemplary collagens are described in Table 4.

TABLE 4
Exemplary Collagen Types

| Collagen Classification | | Collagen Types |
|---|---|---|
| Fibrillar | | I, II, III, V, XI |
| Non-fibrillar | Fibril associated collagens with | IX, XII, XIV, XIX, XXI |
| | interrupted triple helices (FACIT) | |
| Non-fibrillar | Short chain | VIII, X |
| Non-fibrillar | Basement membrane | IV |
| Non-fibrillar | Multiple triple helix domains with | XV, XVIII |
| | interruptions - multiplexin | |
| Non-fibrillar | Microfibril forming | VI |
| Non-fibrillar | Anchoring fibrils | VII |

Exemplary sequences and descriptions of members of various human collagen types can be found by the UniProt Knowledgebase (KB) identifier provided in Table 5 below. UniProtKB available at uniprot.org/uniprot/. Other suitable collagen sequences can be readily identified in the art.

TABLE 5
Exemplary Human Collagens

| UniProtKB | | | | |
|---|---|---|---|---|
| Identifier | Entry name | Protein names | Gene names | Length |
| P02452 | CO1A1_HUMAN | Collagen alpha-1(I) chain | COL1A1 | 1464 |
| | | (Alpha-1 type I collagen) | | |
| P08123 | CO1A2_HUMAN | Collagen alpha-2(I) chain | COL1A2 | 1366 |
| | | (Alpha-2 type I collagen) | | |
| P02458 | CO2A1_HUMAN | Collagen alpha-1(II) chain | COL2A1 | 1487 |
| | | (Alpha-1 type II collagen) | | |
| | | [Cleaved into: Collagen alpha- | | |
| | | 1(II) chain; Chondrocalcin] | | |
| P02461 | CO3A1_HUMAN | Collagen alpha-1(III) chain | COL3A1 | 1466 |
| P02462 | CO4A1_HUMAN | Collagen alpha-1(IV) chain | COL4A1 | 1669 |
| | | [Cleaved into: Arresten] | | |
| P08572 | CO4A2_HUMAN | Collagen alpha-2(IV) chain | COL4A2 | 1712 |
| | | [Cleaved into: Canstatin] | | |
| Q01955 | CO4A3_HUMAN | Collagen alpha-3(IV) chain | COL4A3 | 1670 |
| | | (Goodpasture antigen) [Cleaved | | |
| | | into: Tumstatin] | | |
| P53420 | CO4A4_HUMAN | Collagen alpha-4(IV) chain | COL4A4 | 1690 |
| P29400 | CO4A5_HUMAN | Collagen alpha-5(IV) chain | COL4A5 | 1685 |
| Q14031 | CO4A6_HUMAN | Collagen alpha-6(IV) chain | COL4A6 | 1691 |
| P20908 | CO5A1_HUMAN | Collagen alpha-1(V) chain | COL5A1 | 1838 |
| P05997 | CO5A2_HUMAN | Collagen alpha-2(V) chain | COL5A2 | 1499 |
| P25940 | CO5A3_HUMAN | Collagen alpha-3(V) chain | COL5A3 | 1745 |
| P12109 | CO6A1_HUMAN | Collagen alpha-1(VI) chain | COL6A1 | 1028 |
| P12110 | CO6A2_HUMAN | Collagen alpha-2(VI) chain | COL6A2 | 1019 |
| P12111 | CO6A3_HUMAN | Collagen alpha-3(VI) chain | COL6A3 | 3177 |
| A8TX70 | CO6A5_HUMAN | Collagen alpha-5(VI) chain | COL6A5 | 2615 |
| | | (Collagen alpha-1(XXIX) chain) | COL29A1 | |
| | | (von Willebrand factor A | VWA4 | |
| | | domain-containing protein 4) | | |
| A6NMZ7 | CO6A6_HUMAN | Collagen alpha-6(VI) chain | COL6A6 | 2263 |
| Q02388 | CO7A1_HUMAN | Collagen alpha-1(VII) chain | COL7A1 | 2944 |
| | | (Long-chain collagen) (LC | | |
| | | collagen) | | |
| P27658 | CO8A1_HUMAN | Collagen alpha-1 (VIII) chain | COL8A1 | 744 |
| | | (Endothelial collagen) [Cleaved | C3orf7 | |
| | | into: Vastatin] | | |
| P25067 | CO8A2_HUMAN | Collagen alpha-2(VIII) chain | COL8A2 | 703 |
| | | (Endothelial collagen) | | |
| P20849 | CO9A1_HUMAN | Collagen alpha-1(IX) chain | COL9A1 | 921 |
| Q14055 | CO9A2_HUMAN | Collagen alpha-2(IX) chain | COL9A2 | 689 |
| Q14050 | CO9A3_HUMAN | Collagen alpha-3(IX) chain | COL9A3 | 684 |
| Q03692 | COAA1_HUMAN | Collagen alpha-1(X) chain | COL10A1 | 680 |
| P12107 | COBA1_HUMAN | Collagen alpha-1(XI) chain | COL11A1 | 1806 |
| | | | COLL6 | |
| P13942 | COBA2_HUMAN | Collagen alpha-2(XI) chain | COL11A2 | 1736 |
| Q99715 | COCA1_HUMAN | Collagen alpha-1(XII) chain | COL12A1 | 3063 |
| | | | COL12A1L | |
| Q5TAT6 | CODA1_HUMAN | Collagen alpha-1(XIII) chain | COL13A1 | 717 |
| | | (COLXIIIA1) | | |
| Q05707 | COEA1_HUMAN | Collagen alpha-1(XIV) chain | COL14A1 | 1796 |
| | | (Undulin) | UND | |
| P39059 | COFA1_HUMAN | Collagen alpha-1(XV) chain | COL15A1 | 1388 |
| | | [Cleaved into: Restin | | |
| | | (Endostatin-XV) (Related to | | |
| | | endostatin) (Restin-I); Restin-2 | | |
| | | (Restin-II); Restin-3 (Restin-III); | | |
| | | Restin-4 (Restin-IV)] | | |
| Q07092 | COGA1_HUMAN | Collagen alpha-1(XVI) chain | COL16A1 | 1604 |
| | | | FP1572 | |
| Q9UMD9 | COHA1_HUMAN | Collagen alpha-1(XVII) chain | COL17A1 | 1497 |
| | | (180 kDa bullous pemphigoid | BP180 | |
| | | antigen 2) (Bullous pemphigoid | BPAG2 | |
| | | antigen 2) [Cleaved into: 120 | | |
| | | kDa linear IgA disease antigen | | |
| | | (120 kDa linear IgA dermatosis | | |
| | | antigen) (Linear IgA disease | | |
| | | antigen 1) (LAD-1); 97 kDa | | |
| | | linear IgA disease antigen (97 | | |
| | | kDa linear IgA bullous | | |
| | | dermatosis antigen) (97 kDa | | |
| | | LAD antigen) (97-LAD) (Linear | | |
| | | IgA bullous disease antigen of | | |
| | | 97 kDa) (LABD97)] | | |
| P39060 | COIA1_HUMAN | Collagen alpha-1(XVIII) chain | COL18A1 | 1754 |
| | | [Cleaved into: Endostatin; Non- | | |
| | | collagenous domain 1 (NC1)] | | |
| Q14993 | COJA1_HUMAN | Collagen alpha-1(XIX) chain | COL19A1 | 1142 |
| | | (Collagen alpha-1(Y) chain) | | |
| Q9P218 | COKA1_HUMAN | Collagen alpha-1(XX) chain | COL20A1 | 1284 |
| | | | KIAA1510 | |
| Q96P44 | COLA1_HUMAN | Collagen alpha-1(XXI) chain | COL21A1 | 957 |
| | | | COL1AL | |
| | | | FP633 | |
| Q8NFW1 | COMA1_HUMAN | Collagen alpha-1(XXII) chain | COL22A1 | 1626 |
| Q86Y22 | CONA1_HUMAN | Collagen alpha-1(XXIII) chain | COL23A1 | 540 |
| Q17RW2 | COOA1_HUMAN | Collagen alpha-1(XXIV) chain | COL24A1 | 1714 |
| Q9BXSO | COPA1_HUMAN | Collagen alpha-1(XXV) chain | COL25A1 | 654 |
| | | (Alzheimer disease amyloid- | | |
| | | associated protein) (AMY) | | |
| | | (CLAC-P) [Cleaved into: | | |
| | | Collagen-like Alzheimer | | |
| | | amyloid plaque component | | |
| | | (CLAC)] | | |
| Q96A83 | COQA1_HUMAN | Collagen alpha-1(XXVI) chain | COL26A1 | 441 |
| | | (Alpha-1 type XXVI collagen) | EMID2 EMU2 | |
| | | (EMI domain-containing protein | | |
| | | 2) (Emilin and multimerin | | |
| | | domain-containing protein 2) | | |
| | | (Emu2) | | |
| Q8IZC6 | CORA1_HUMAN | Collagen alpha-1(XXVII) chain | COL27A1 | 1860 |
| | | | KIAA1870 | |
| Q2UY09 | COSA1_HUMAN | Collagen alpha-1(XXVIII) chain | COL28A1 | 1125 |
| | | | COL28 | |

In some embodiments, the solubility-enhancing amino acid sequence may be derived from a human type I, alpha 1 collagen polypeptide, a human type I, alpha 2 collagen polypeptide, a human type 21, alpha 1 collagen polypeptide, a chicken type 21 collagen polypeptide, or a human type III, alpha 1 collagen polypeptide, or a truncate or fragment of any thereof, or a similar sequence based on % identity to a region of the full-length of any thereof.

Human full-length collagen Type 21,
alpha 1 (957 aa) (NCBI AAI43866.1):
(SEQ ID NO: 13)
MAHYITFLCMVLVLLLQNSVLAEDGEVRSSCRTAPTDLVFILDGS
YSVGPENFEIVKKWLVNITKNFDIGPKFIQVGVVQYSDYPVLEIP
LGSYDSGEHLTAAVESILYLGGNTKTGKAIQFALDYLFAKSSRFL
TKIAVVLTDGKSQDDVKDAAQAARDSKITLFAIGVGSETEDAELR
AIANKPSSTYVFYVEDYIAISKIREVMKQKLCEESVCPTRIPVAA
RDERGFDILLGLDVNKKVKKRIQLSPKKIKGYEVTSKVDLSELTS
NVFPEGLPPSYVFVSTQRFKVKKIWDLWRILTIDGRPQIAVTLNG
VDKILLFTTTSVINGSQVVTFANPQVKTLFDEGWHQIRLLVTEQD
VTLYIDDQQIENKPLHPVLGILINGQTQIGKYSGKEETVQFDVQK
LRIYCDPEQNNRETACEIPGFNGECLNGPSDVGSTPAPCICPPGK
PGLQGPKGDPGLPGNPGYPGQPGQDGKPGYQGIAGTPGVPGSPGI
QGARGLPGYKGEPGRDGDKGDRGLPGFPGLHGMPGSKGEMGAKGD
KGSPGFYGKKGAKGEKGNAGFPGLPGPAGEPGRHGKDGLMGSPGF
KGEAGSPGAPGQDGTRGEPGIPGFPGNRGLMGQKGEIGPPGQQGK
KGAPGMPGLMGSNGSPGQPGTPGSKGSKGEPGIQGMPGASGLKGE
PGATGSPGEPGYMGLPGIQGKKGDKGNQGEKGIQGQKGENGRQGI
PGQQGIQGHHGAKGERGEKGEPGVRGAIGSKGESGVDGLMGPAGP
KGQPGDPGPQGPPGLDGKPGREFSEQFIRQVCTDVIRAQLPVLLQ
SGRIRNCDHCLSQHGSPGIPGPPGPIGPEGPRGLPGLPGRDGVPG
LVGVPGRPGVRGLKGLPGRNGEKGSQGFGYPGEQGPPGPPGPEGP
PGISKEGPPGDPGLPGKDGDHGKPGIQGQPGPPGICDPSLCFSVI
ARRDPFRKGPNY
Human full-length collagen Type I,
alpha 1 (1464 aa) (NCBI AAH36531.1):
(SEQ ID NO: 14)
MFSFVDLRLLLLLAATALLTHGQEEGQVEGQDEDIPPITCVQNGL
RYHDRDVWKPEPCRICVCDNGKVLCDDVICDETKNCPGAEVPEGE
CCPVCPDGSESPTDQETTGVEGPKGDTGPRGPRGPAGPPGRDGIP
GQPGLPGPPGPPGPPGPPGLGGNFAPQLSYGYDEKSTGGISVPGP
MGPSGPRGLPGPPGAPGPQGFQGPPGEPGEPGASGPMGPRGPPGP
PGKNGDDGEAGKPGRPGERGPPGPQGARGLPGTAGLPGMKGHRGF
SGLDGAKGDAGPAGPKGEPGSPGENGAPGQMGPRGLPGERGRPGA
PGPAGARGNDGATGAAGPPGPTGPAGPPGFPGAVGAKGEAGPQGP
RGSEGPQGVRGEPGPPGPAGAAGPAGNPGADGQPGAKGANGAPGI
AGAPGFPGARGPSGPQGPGGPPGPKGNSGEPGAPGSKGDTGAKGE
PGPVGVQGPPGPAGEEGKRGARGEPGPTGLPGPPGERGGPGSRGF
PGADGVAGPKGPAGERGSPGPAGPKGSPGEAGRPGEAGLPGAKGL
TGSPGSPGPDGKTGPPGPAGQDGRPGPPGPPGARGQAGVMGFPGP
KGAAGEPGKAGERGVPGPPGAVGPAGKDGEAGAQGPPGPAGPAGE
RGEQGPAGSPGFQGLPGPAGPPGEAGKPGEQGVPGDLGAPGPSGA
RGERGFPGERGVQGPPGPAGPRGANGAPGNDGAKGDAGAPGAPGS
QGAPGLQGMPGERGAAGLPGPKGDRGDAGPKGADGSPGKDGVRGL
TGPIGPPGPAGAPGDKGESGPSGPAGPTGARGAPGDRGEPGPPGP
AGFAGPPGADGQPGAKGEPGDAGAKGDAGPPGPAGPAGPPGPIGN
VGAPGAKGARGSAGPPGATGFPGAAGRVGPPGPSGNAGPPGPPGP
AGKEGGKGPRGETGPAGRPGEVGPPGPPGPAGEKGSPGADGPAGA
PGTPGPQGIAGQRGVVGLPGQRGERGFPGLPGPSGEPGKQGPSGA
SGERGPPGPMGPPGLAGPPGESGREGAPGAEGSPGRDGSPGAKGD
RGETGPAGPPGAPGAPGAPGPVGPAGKSGDRGETGPAGPTGPVGP
VGARGPAGPQGPRGDKGETGEQGDRGIKGHRGFSGLQGPPGPPGS
PGEQGPSGASGPAGPRGPPGSAGAPGKDGLNGLPGPIGPPGPRGR
TGDAGPVGPPGPPGPPGPPGPPSAGFDFSFLPQPPQEKAHDGGRY
YRADDANVVRDRDLEVDTTLKSLSQQIENIRSPEGSRKNPARTCR
DLKMCHSDWKSGEYWIDPNQGCNLDAIKVFCNMETGETCVYPTQP
SVAQKNWYISKNPKDKRHVWFGESMTDGFQFEYGGQGSDPADVAI
QLTFLRLMSTEASQNITYHCKNSVAYMDQQTGNLKKALLLQGSNE
IEIRAEGNSRFTYSVTVDGCTSHTGAWGKTVIEYKTTKTSRLPII
DVAPLDVGAPDQEFGFDVGPVCFL
Human full-length collagen Type I,
alpha 2 (1366 aa) (NCBI AAH42586.1)
(SEQ ID NO: 15)
MLSFVDTRTLLLLAVTLCLATCQSLQEETVRKGPAGDRGPRGERG
PPGPPGRDGEDGPTGPPGPPGPPGPPGLGGNFAAQYDGKGVGLGP
GPMGLMGPRGPPGAAGAPGPQGFQGPAGEPGEPGQTGPAGARGPA
GPPGKAGEDGHPGKPGRPGERGVVGPQGARGFPGTPGLPGFKGIR
GHNGLDGLKGQPGAPGVKGEPGAPGENGTPGQTGARGLPGERGRV
GAPGPAGARGSDGSVGPVGPAGPIGSAGPPGFPGAPGPKGEIGAV
GNAGPAGPAGPRGEVGLPGLSGPVGPPGNPGANGLTGAKGAAGLP
GVAGAPGLPGPRGIPGPVGAAGATGARGLVGEPGPAGSKGESGNK
GEPGSAGPQGPPGPSGEEGKRGPNGEAGSAGPPGPPGLRGSPGSR
GLPGADGRAGVMGPPGSRGASGPAGVRGPNGDAGRPGEPGLMGPR
GLPGSPGNIGPAGKEGPVGLPGIDGRPGPIGPAGARGEPGNIGFP
GPKGPTGDPGKNGDKGHAGLAGARGAPGPDGNNGAQGPPGPQGVQ
GGKGEQGPPGPPGFQGLPGPSGPAGEVGKPGERGLHGEFGLPGPA
GPRGERGPPGESGAAGPTGPIGSRGPSGPPGPDGNKGEPGVVGAV
GTAGPSGPSGLPGERGAAGIPGGKGEKGEPGLRGEIGNPGRDGAR
GAPGAVGAPGPAGATGDRGEAGAAGPAGPAGPRGSPGERGEVGPA
GPNGFAGPAGAAGQPGAKGERGAKGPKGENGVVGPTGPVGAAGPA
GPNGPPGPAGSRGDGGPPGMTGFPGAAGRTGPPGPSGISGPPGPP
GPAGKEGLRGPRGDQGPVGRTGEVGAVGPPGFAGEKGPSGEAGTA
GPPGTPGPQGLLGAPGILGLPGSRGERGLPGVAGAVGEPGPLGIA
GPPGARGPPGAVGSPGVNGAPGEAGRDGNPGNDGPPGRDGQPGHK
GERGYPGNIGPVGAAGAPGPHGPVGPAGKHGNRGETGPSGPVGPA
GAVGPRGPSGPQGIRGDKGEPGEKGPRGLPGLKGHNGLQGLPGIA
GHHGDQGAPGSVGPAGPRGPAGPSGPAGKDGRTGHPGTVGPAGIR
GPQGHQGPAGPPGPPGPPGPPGVSGGGYDFGYDGDFYRADQPRSA
PSLRPKDYEVDATLKSLNNQIETLLTPEGSRKNPARTCRDLRLSH
PEWSSGYYWIDPNQGCTMDAIKVYCDFSTGETCIRAQPENIPAKN
WYRSSKDKKHVWLGETINAGSQFEYNVEGVTSKEMATQLAFMRLL
ANYASQNITYHCKNSIAYMDEETGNLKKAVILQGSNDVELVAEGN
SRFTYTVLVDGCSKKTNEWGKTIIEYKTNKPSRLPFLDIAPLDIG
GADQEFFVDIGPVCFK
Human full-length collagen Type III,
alpha 1 (1466 aa) (NCBI AGL34959.1)
(SEQ ID NO: 16)
MMSFVQKGSWLLLALLHPTIILAQQEAVEGGCSHLGQSYADRDVW
KPEPCQICVCDSGSVLCDDIICDDQELDCPNPEIPFGECCAVCPQ
PPTAPTRPPNGQGPQGPKGDPGPPGIPGRNGDPGIPGQPGSPGSP
GPPGICESCPTGPQNYSPQYDSYDVKSGVAVGGLAGYPGPAGPPG
PPGPPGTSGHPGSPGSPGYQGPPGEPGQAGPSGPPGPPGAIGPSG
PAGKDGESGRPGRPGERGLPGPPGIKGPAGIPGFPGMKGHRGFDG
RNGEKGETGAPGLKGENGLPGENGAPGPMGPRGAPGERGRPGLPG
AAGARGNDGARGSDGQPGPPGPPGTAGFPGSPGAKGEVGPAGSPG
SNGAPGQRGEPGPQGHAGAQGPPGPPGINGSPGGKGEMGPAGIPG
APGLMGARGPPGPAGANGAPGLRGGAGEPGKNGAKGEPGPRGERG
EAGIPGVPGAKGEDGKDGSPGEPGANGLPGAAGERGAPGFRGPAG
PNGIPGEKGPAGERGAPGPAGPRGAAGEPGRDGVPGGPGMRGMPG
SPGGPGSDGKPGPPGSQGESGRPGPPGPSGPRGQPGVMGFPGPKG
NDGAPGKNGERGGPGGPGPQGPPGKNGETGPQGPPGPTGPGGDKG
DTGPPGPQGLQGLPGTGGPPGENGKPGEPGPKGDAGAPGAPGGKG
DAGAPGERGPPGLAGAPGLRGGAGPPGPEGGKGAAGPPGPPGAAG
TPGLQGMPGERGGLGSPGPKGDKGEPGGPGADGVPGKDGPRGPTG
PIGPPGPAGQPGDKGEGGAPGLPGIAGPRGSPGERGETGPPGPAG
FPGAPGQNGEPGGKGERGAPGEKGEGGPPGVAGPPGGSGPAGPPG
PQGVKGERGSPGGPGAAGFPGARGLPGPPGSNGNPGPPGPSGSPG
KDGPPGPAGNTGAPGSPGVSGPKGDAGQPGEKGSPGAQGPPGAPG
PLGIAGITGARGLAGPPGMPGPRGSPGPQGVKGESGKPGANGLSG
ERGPPGPQGLPGLAGTAGEPGRDGNPGSDGLPGRDGSPGGKGDRG
ENGSPGAPGAPGHPGPPGPVGPAGKSGDRGESGPAGPAGAPGPAG
SRGAPGPQGPRGDKGETGERGAAGIKGHRGFPGNPGAPGSPGPAG
QQGAIGSPGPAGPRGPVGPSGPPGKDGTSGHPGPIGPPGPRGNRG
ERGSEGSPGHPGQPGPPGPPGAPGPCCGGVGAAAIAGIGGEKAGG
FAPYYGDEPMDFKINTDEIMTSLKSVNGQIESLISPDGSRKNPAR
NCRDLKFCHPELKSGEYWVDPNQGCKLDAIKVFCNMETGETCISA
NPLNVPRKHWWTDSSAEKKHVWFGESMDGGFQFSYGNPELPEDVL
DVHLAFLRLLSSRASQNITYHCKNSIAYMDQASGNVKKALKLMGS
NEGEFKAEGNSKFTYTVLEDGCTKHTGEWSKTVFEYRTRKAVRLP
IVDIAPYDIGGPDQEFGVDVGPVCFL
Gallus gallus (chicken) type 21, alpha 1
full-length collagen (957 aa)
(XP_004940520.2)
(SEQ ID NO: 17)
MAQLLRLFQTLLILLLRDYISAEDGETRASCRTAPADLVFILDGS
YSVGPENFEIIKSWLVNITRNFDIGPKFIQVGVVQYSDYPVLEIP
LGTHESTENLIKEMESIHYLGGNTKTGRAIQFAYDHLFAKSSRFL
TKIAVVLTDGKSQDEVKDVAAEARKNKITLFAIGVGSEIEEDELK
AIANKPSSTYVFYVEDYIAISRIKEVIKQKLCEESVCPTRIPVAA
RDEKGFDILVGLGVKKRVKKRIQIPTTNAKAYEVTSRVDLSELTR
NVFPEGLPPSYVFVSTQRFKVKKTWDLWRVLSLDKRPQIAVTING
EEKTLSFTTTSLINGTQVITFAAPRVKTLFDEGWHQIRLLVTEDF
VTLYIDDQEIETKPLHPVLGIYISGLTQIGKYSGKEETVQFDIQK
LRIYCDPEQNNRETVCEIPGFNGECMNGPSDVGSTPAPCICPPGK
QGPPGPKGDPGQPGNHGYPGQPGPDGKPGYQGSAGTPGIPGTPGV
QGPRGLPGIKGEPGKDGTKGDRGLPGFPGLHGMPAPKGERGPKGD
QGVPGIYGKKGSKGEKGDTGFPGMPGRSGDPGRSGKDGLPGSPGF
KGEVGQPGSPGLEGHRGEPGIPGIPGNQGAKGQKGEIGPPGLPGA
KGSPGETGLMGPEGSFGLPGAPGPKGDKGEPGLQGKPGSSGAKGE
PGGPGAPGEPGYPGIPGTQGIKGDKGSQGESGIQGRKGEKGRQGN
PGLQGTEGLRGEQGEKGEKGDPGIRGINGQKGESGIQGLVGPPGV
RGQPGDRGPPGPPGSDGKPAREFSEEFIRQVCSDVLRTQLPVILQ
SGRLQNCNHCQSQSASPGLPGPPGPRGPEGPRGFPGLPGNDGVPG
LTGIPGRPGARGTRGLPGKNGAKGNQGIGAPGIQGPPGPPGPEGP
PGMSKEGRPGERGQPGKDGDRGSPGMPGPVGPPGICDPSLCFSVI
VGRDPFRKGPNY

In certain embodiments, the solubility-enhancing amino acid residues correspond to a sub-segment of a naturally occurring collagen polypeptide. The amino acid sequences provided below, or fragments thereof, may be useful as exemplary solubility-enhancing amino acid sequences:

Truncated collagen type 21, alpha 1 polypeptide from Gallus gallus (chicken), having 211 amino acids.

(SEQ ID NO: 18)
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKGDKGSQGESGIQGRKGEKGRQGNPGLQGTEGLRGEQGEKG
EKGDPGIRGINGQKGESGIQGLVGPPGVRGQ

Truncated collagen type 21, alpha 1 polypeptide from Gallus gallus (chicken), having 188 amino acids. (GL21)

(SEQ ID NO: 19)
DTGFPGMPGRSGDPGRSGKDGLPGSPGFKGEVGQPGSPGLEGHRGE
PGIPGIPGNQGAKGQKGEIGPPGLPGAKGSPGETGLMGPEGSFGL
PGAPGPKGDKGEPGLQGKPGSSGAKGEPGGPGAPGEPGYPGIPGT
QGIKGDKGSQGESGIQGRKGEKGRQGNPGLQGTEGLRGEQGEKGE
KGDPGIR

Truncated collagen type 21, alpha 1 polypeptide from Gallus gallus (chicken), having 141 amino acids. (GL21)

(SEQ ID NO: 20
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKG.

Truncated collagen type 21, alpha 1 polypeptide from Gallus gallus (chicken), having 93 amino acids. (GL21)

(SEQ ID NO: 21)
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPG

Truncated collagen type 21, alpha 1 polypeptide from Gallus gallus (chicken), having 47 amino acids. (GL21)

(SEQ ID NO: 22)
DTGFPGMPGRSGDPGRSGKDGLPGSPGFKGEVGQPGSPGLEGHRG
EP.

Truncated collagen type II, alpha 1 polypeptide from Acipenser schrenckii (Japanese sturgeon), having 199 amino acids.

(SEQ ID NO: 23)
GLQGMPGERGASGIAGAKGDRGDVGEKGPEGASGKDGSRGLTGPI
GPPGPAGPNGEKGESGPSGPPGAAGTRGAPGDRGENGPPGPAGFA
GPPGADGQPGAKGEQGEGGQKGDAGAPGPQGPSGAPGPQGPTGVS
GPKGARGAQGPPGATGFPGAAGRVGPPGPNGNPGPSGPAGSAGKD
GPKGVRGDAGPPGRAGDAG

Truncated human collagen type 21, alpha 1, having 187 amino acids.

(SEQ ID NO: 24)
AGFPGLPGPAGEPGRHGKDGLMGSPGFKGEAGSPGAPGQDGTRGE
PGIPGFPGNRGLMGQKGEIGPPGQQGKKGAPGMPGLMGSNGSPGQ
PGTPGSKGSKGEPGIQGMPGASGLKGEPGATGSPGEPGYMGLPGI
QGKKGDKGNQGEKGIQGQKGENGRQGIPGQQGIQGHHGAKGERGE
KGEPGVR

Truncated human collagen type III, alpha 1, having 200 amino acids.

(SEQ ID NO: 25)
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKGDKGSQGESGIQGRKGEKGRQGNPGLQGTEGLRGEQGEKG
EKGDPGIR

Truncated human collagen type I, alpha 1, having 262 amino acids. (HsCol1a1-27)

(SEQ ID NO: 26)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVG

Truncated human collagen type I, alpha 1, having 225 amino acids. (Cde115)

(SEQ ID NO: 27)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP

Truncated human collagen type I, alpha 1, having 219 amino acids:

(SEQ ID NO: 119)
NDGAKGDAGAPGAPGSQGAPGLQGMPGERGAAGLPGPKGDRGDAGPKGAD
GSPGKDGVRGLTGPIGPPGPAGAPGDKGESGPSGPAGPTGARGAPGDRGE
PGPPGPAGFAGPPGADGQPGAKGEPGDAGAKGDAGPPGPAGPAGPPGPIG
NVGAPGAKGARGSAGPPGATGFPGAAGRVGPPGPSGNAGPPGPPGPAGKE
GGKGPRGETGPAGRPGEVG

Truncated human collagen type I, alpha 1, having 196 amino acids. (Cde125)

(SEQ ID NO: 28)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVG.

Truncated human collagen type I, alpha 1, having 130 amino acids. (Cde150)

(SEQ ID NO: 29)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAG.

Truncated human collagen type I, alpha 1, having 64 amino acids. (Cde175)

(SEQ ID NO: 30)
GVPGDLGAPGPSGARGERGFPGERGVQGPPGPAGPRGANGAPGNDGAKGD
AGAPGAPGSQGAPG.

Truncated human collagen type I, alpha 1, having 25 amino acids.

(SEQ ID NO: 31)
GVPGDLGAPGPSGARGERGFPGERG.

In some aspects, the solubility-enhancing segment of the non-naturally occurring polypeptides provided herein may or may not contain one or more domains from natural collagen (e.g., Von Willebrand factor type A (vWA) domain, laminin G domain, fibrillar collagen C-terminal domain). In some aspects, the solubility-enhancing segment of a non-naturally occurring polypeptide provided herein may contain one or more collagen triple helix repeat domains.

In some embodiments, the solubility-enhancing amino acid sequence is present as an amino acid fusion to a keratin polypeptide. In other embodiments, the solubility-enhancing amino acid sequence can be expressed in the same cell, but as a separate protein from the keratin polypeptide, for example, from different nucleotide sequences and/or different expression vectors. Thus, although they are not fused into one protein, the presence of the solubility-enhancing sequence in the post-fermentation culture broth along with the keratin polypeptide can assist in the solubility of the keratin polypeptide, for example, through intramolecular associations.

In other embodiments, the solubility-enhancing amino acid sequence is prepared separately and is then added to a solution containing a keratin polypeptide in order to increase its solubility characteristics. The addition of a solubility-enhancing polypeptide can be used to increase the stability of keratin polypeptides during storage.

In another embodiment, the solubility-enhancing amino acid sequence and the keratin polypeptide are prepared separately, but are fused at some point during the isolation, purification or formulation process. Any suitable fusion method can be used. The fusion can occur between amino acid side chains of both polypeptides, or can be from, for example, intramolecular peptide bonds, or amide bonds between the N-terminal and C-terminal amino acid residues. Chemical cross-linking methods can be used. The solubility-enhancing amino acid sequence can thus aid in the solubility of a keratin polypeptide at multiple stages in its preparation.

In another embodiment, the solubility-enhancing amino acid sequence can have additional, secondary functions in addition to its ability to increase the solubility of the keratin polypeptide. The secondary function can be, for example, the ability to bind the protein of interest to another moiety, such as another protein, a substrate, a surface, a metal, a paper, a filter, an in vivo or ex vivo scaffolding, a plastic, a container surface, a purification tag, a fluorescent tag, a chemical compound, an organic substrate, and the like. The secondary function can be reversible, or it can be irreversible. The secondary function can be induced, for example, by specific changes, such as salt, pH, temperature, or the presence of other compounds.

In some embodiments, a fusion protein of the solubility-enhancing amino acid sequence and the keratin polypeptide can have useful characteristics that are derived from both the solubility-enhancing amino acid sequence and from the keratin polypeptide. Such functions may be complementary or synergistic, for example a combination of collagen and keratin in composition for use as a personal care product.

### Polypeptides Comprising a Solubility-Enhancing Amino Acid Sequence and a Keratin-Derived Target Amino Acid Sequence

In some embodiments of the disclosure, non-naturally occurring polypeptides comprise both a solubility-enhancing amino acid sequence and a target amino acid sequence derived from a keratin polypeptide. In certain embodiments the solubility-enhancing amino acid and the target amino acid sequence are directly linked by a peptide bond, for example as a fusion protein. As provided herein, the combination of the solubility-enhancing amino acid sequence with the target amino acid sequence results in an increased ease in expressing, isolating and purifying the target protein, in comparison to that of the target protein by itself.

In some embodiments, the solubility-enhancing amino acid sequence comprises at least 9 amino acids, at least 12 amino acids, at least 15 amino acids, at least 18 amino acids, at least 21 amino acids, at least 24 amino acids, at least 27 amino acids, at least 30 amino acids, at least 33 amino acids, at least 36 amino acids, at least 39 amino acids, at least 42 amino acids, at least 45 amino acids, at least 48 amino acids, at least 51 amino acids, at least 54 amino acids, at least 57 amino acids, at least 60 amino acids, at least 63 amino acids, at least 66, at least 69 amino acids, at least 72 amino acids, at least 75 amino acids, at least 78 amino acids, at least 81 amino acids, at least 84 amino acids at least 87 amino acids, at least 90 amino acids, at least 93 amino acids, at least 96 amino acids, at least 99 amino acids, at least 102 amino acids, at least 105 amino acids, at least 108 amino acids, at least 111 amino acids, at least 114 amino acids, at least 117 amino acids, at least 120 amino acids, at least 123 amino acids, at least 126 amino acids, at least 129 amino acids, at least 132 amino acids, at least 135 amino acids, at least 138 amino acids, at least 141 amino acids, at least 144 amino acids, at least 147 amino acids, at least 150 amino acids, at least 153 amino acids, at least 156 amino acids, at least 159 amino acids, at least 162 amino acids, at least 165 amino acids, at least 168 amino acids, at least 171 amino acids, at least 174 amino acids, at least 177 amino acids, at least 180 amino acids, at least 183, at least 186 amino acids, at least 189 amino acids, at least 192 amino acids, at least 195 amino acids, at least 198 amino acids, at least 201 amino acids, at least 204 amino acids, at least 207 amino acids, at least 210 amino acids, at least 213 amino acids, at least 216 amino acids, at least 219 amino acids, at least 222 amino acids, at least 225 amino acids, at least 228 amino acids, at least 231 amino acids, at least 234 amino acids, at least 237 amino acids, at least 240 amino acids, at least 243 amino acids, at least 246 amino acids, at least 249 amino acids, at least 252 amino acids, at least 255 amino acids, at least 258 amino acids, at least 261 amino acids, at least 264 amino acids, at least 267 amino acids, at least 270 amino acids, at least 273 amino acids, at least 276 amino acids, at least 279 amino acids, at least 282 amino acids, at least 285 amino acids, at least 288 amino acids, at least 291 amino acids, at least 294 amino acids, at least 297 amino acids, or at least 300 amino acids, and the keratin polypeptide comprises at least 25 aa at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 110 amino acids, at least 120 amino acids, at least 130 amino acids, at least 140 amino acids, at least 150 amino acids, at least 160 amino acids, at least 170 amino acids, at least 180 amino acids, at least 190 amino acids, at least 200 amino acids, at least 210 amino acids, at least 220 amino acids, at least 230 amino acids, at least 240 amino acids, at least 250 amino acids, at least 260 amino acids, at least 270 amino acids, at least 280 amino acids, at least 290 amino acids, or at least 300 amino acids of a keratin polypeptide.

In certain embodiments, the solubility-enhancing amino acid sequence and the target amino acid sequence are linked via a linker sequence. This linker sequence may be a flexible linker, a rigid linker, or a cleavable linker, as are known in the art. An example of a rigid linker is the Pro₍₆₎ or “polyproline” linker system. This type of linker allows polypeptide segments to be physically separated from one another. Flexible linkers can also be used. Two examples of a suitable flexible linker are the di-peptide sequence Gly-Ser, as well as (GGGGS)₃ (SEQ ID NO: 32). The linker between the two polypeptide segments may also be cleavable, for example, containing a protease cleavage sequence.

In certain embodiments, the non-naturally occurring polypeptide may further comprise additional amino acid sequences providing additional functionalities, such as a purification tag (e.g., a histidine tag), a labelling or detection tag (e.g., a fluorescent tag or binding site therefor), a binding tag (e.g., an antibody binding site), a secretion tag, and the like. Any suitable secretion signal sequence (e.g., hydrophobic signaling peptides, Sec signal peptides, Tat signal peptides, etc.) that can induce the non-naturally occurring to be secreted to the periplasmic and/or extracellular space (e.g., when produced in a recombinant host cell). Exemplary secretion signal sequences include a peptide having an amino acid sequence of any one of SEQ ID NOs: 37, 39, 41, 43, 45, 47, 49, 51, 53, and 55. The secretion signal sequence is preferably located at the N-terminus of the non-naturally occurring polypeptide. Yet, it is contemplated that the secretion signal sequence can be located at other than N-terminus where the secretion signal sequence remains functional.

In some embodiments, the non-naturally occurring polypeptide comprises a protease cleavage site. The protease cleavage site may be useful, e.g., to separate or remove the solubility-enhancing amino acid sequence from the target amino acid sequence. This may be performed by contacting the polypeptide with a protease corresponding to the protease cleavage site in the polypeptide, e.g., after the polypeptide is recovered from the culture medium. The protease cleavage site can also be used to remove certain added amino acid sequences as provided herein, such as those that either aid in the production of the protein, aid in the purification process, or other sequences. The proteases may comprise endoproteases, exoproteases, serine proteases, cysteine proteases, threonine proteases, aspartic proteases, glutamic proteases, and metalloproteases. Exemplary protease cleavage sites include amino acids that are cleaved by thrombin, TEV (Tobacco etch virus) protease, Factor Xa, enteropeptidase, rhinovirus 3C protease, and the like.

An exemplary protease cleavage system is the TEV protease system. The TEV protease recognizes the specific amino acid sequence ENLYFQS (SEQ ID NO: 33) or ENLYFQG (SEQ ID NO: 34). When treated with the TEV protease, a protein containing this specific amino acid sequence is cleaved between the Q and G/S residues of the above recognition site. Another exemplary protease cleavage system is the enterokinase cleavage system. Enterokinase is a highly specific serine protease, which recognizes the specific amino acid sequence DDDDKX (SEQ ID NO: 35) and cleaves a protein containing such a sequence between the K and X residues (where X is any amino acid). Similar commercially prepared protease systems are well known in the art, such as the FLAG® protein expression system (Sigma, St. Louis, Missouri).

An exemplary purification amino acid sequence is a histidine tag, which is an amino acid affinity tag system that can aid in the purification of recombinant proteins. The histidine tag is typically composed of 6-10 consecutive histidine residues at either terminus of the protein of interest, often separated by a protease-cleavage site. The histidine residues allow an inexpensive yet fast purification of the protein, by readily binding to certain metal cations (such as Ni²⁺ or Co²⁺) that are immobilized on beads or a resin for purification. The unwanted proteins and other impurities can be rapidly washed out of the system, and then the bound protein containing the histidine tag can be eluted. The histidine tag sequence can then be removed, if desired (such as by a protease cleavage site), or it can remain on the final protein.

In some embodiments, the secretion tag is a secretion signal sequence that causes the recombinant host cell to secrete the polypeptide into a culture media. In some embodiments, the secretion tag is a secretion signal sequence that causes the recombinant host cell to secrete the polypeptide into the periplasm. In some embodiments, the secretion tag is a secretion signal sequence that causes the recombinant host cell to secrete the polypeptide to the extracellular space. Exemplary secretion tags that may be used include, e.g., DsbA, PelB, OmpA, ToIB, MalE, lpp, TorA, or HylA, DegP, Skp(Ec), PhoE, NucB, DegP(Rn), DsbA(Ec), LamB, eco, OmpF, PhoA, TolB signal sequences and the like. Comparable secretion tags can be readily found in the art and paired with the respective host, e.g., a yeast secretion tag as is known in the art for expression and secretion in a yeast host cell system.

The nucleotide sequence encoding a Skp(Ec) secretion sequence is disclosed below:

(SEQ ID NO: 36)
ATGAAAGTGATGCGTACAACAGTCGCGACGGTTGTTGCGGCCACGTTATC
CATGTCCGCCTTTAGTGTATTTGCA

The amino acid sequence corresponding to the above nucleotide sequence is:

(SEQ ID NO: 37)
MKKWLLAAGLGLALATSAQA

The nucleotide sequence encoding a PhoE secretion sequence is disclosed below:

(SEQ ID NO: 38)
ATGAAAAAATGGCTGCTGGCTGCTGGTCTGGGTCTGGCTCTGGCTACCTC
TGCTCAGGCT

The amino acid sequence corresponding to the above nucleotide sequence is:

(SEQ ID NO: 39)
MKKSTLALVVMGIVASASVQA

The nucleotide sequence encoding a NucB secretion sequence is disclosed below:

(SEQ ID NO: 40)
ATGAAAAAGTGGATGGCGGGTTTGTTCCTGGCTGCAGCGGTTTTATTGTG
TCTCATGGTGCCGCAGCAAATACAAGGCGCAAGTTCT

The amino acid sequence corresponding to the above nucleotide sequence is:

(SEQ ID NO: 41)
MKKWMAGLFLAAAVLLCLMVPQQIQGASS

The nucleotide sequence encoding a DegP(Rn) secretion sequence is disclosed below:

(SEQ ID NO: 42)
ATGAAAAAAAACATCCTGTCTCTGTCTATGGTTGCTCTGTCTCTGTCTCT
GGCTCTGGGTTCTGTTTCTGTTACCGCT

The amino acid sequence corresponding to the above nucleotide sequence is:

(SEQ ID NO: 43)
MKKNILSLSMVALSLSLALGSVSVTA

The nucleotide sequence encoding a DsbA(Ec) secretion sequence is disclosed below:

(SEQ ID NO: 44)
ATGAAAAAAATCTGGCTGGCTCTGGCTGGTCTGGTTCTGGCTTTCTCTGC
TTCTGCT

The amino acid sequence corresponding to the above nucleotide sequence is:

(SEQ ID NO: 45)
MKKNILSLSMVALSLSLALGSVSVTA

The nucleotide sequence encoding a LamB secretion sequence is disclosed below:

(SEQ ID NO: 46)
ATGATGATCACCCTGCGTAAACTGCCGCTGGCTGTTGCTGTTGCT
GCTGGTGTTATGTCTGCTCAGGCTATGGCT

The amino acid sequence corresponding to the above nucleotide sequence is:

(SEQ ID NO: 47)
MMITLRKLPLAVAVAAGVMSAQAMA

The nucleotide sequence encoding an eco secretion sequence is disclosed below:

(SEQ ID NO: 48)
ATGAAAACCATCCTGCCGGCTGTTCTGTTCGCTGCTTTCGCTACC
ACCTCTGCTTGGGCT

The amino acid sequence corresponding to the above nucleotide sequence is:

(SEQ ID NO: 49)
MKTILPAVLFAAFATTSAWA

The nucleotide sequence encoding an OmpF secretion sequence is disclosed below:

(SEQ ID NO: 50)
ATGATGAAACGTAACATCCTGGCTGTTATCGTTCCGGCTCTGCTG
GTTGCTGGTACCGCTAACGCT

The amino acid sequence corresponding to the above nucleotide sequence is:

(SEQ ID NO: 51)
MMKRNILAVIVPALLVAGTANA

The nucleotide sequence encoding a PhoA secretion sequence is disclosed below:

(SEQ ID NO: 52)
ATGAAACAGTCTACCATCGCTCTGGCTCTGCTGCCGCTGCTGTTC
ACCCCGGTTACCAAAGCT

The amino acid sequence corresponding to the above nucleotide sequence is:

(SEQ ID NO: 53)
MKQSTIALALLPLLFTPVTKA

The nucleotide sequence encoding a TolB secretion sequence is disclosed below:

(SEQ ID NO: 54)
ATGAAACAGGCTCTGCGTGTAGCGTTCGGTTTCCTGATACTGTGG
GCTTCTGTTCTGCACGCT

The amino acid sequence corresponding to the above nucleotide sequence is:

(SEQ ID NO: 55)
MKQALRVAFGFLILWASVLHA

In certain embodiments, the non-naturally occurring polypeptide comprises a solubility-enhancing amino acid sequence that is a truncated collagen polypeptide and a keratin-derived target amino acid sequence. Exemplary sequences of this combination are provided below.

A truncated human collagen type I, alpha 1 polypeptide (262 amino acids) and a truncated keratin type 12 polypeptide (K12, 179 amino acids):

(SEQ ID NO: 56)
GVPGDLGAPGPSGARGERGFPGERGVQGPPGPAGPRGANGAPGND
GAKGDAGAPGAPGSQGAPGLQGMPGERGAAGLPGPKGDRGDAGP
KGADGSPGKDGVRGLTGPIGPPGPAGAPGDKGESGPSGPAGPTGA
RGAPGDRGEPGPPGPAGFAGPPGADGQPGAKGEPGDAGAKGDAGP
PGPAGPAGPPGPIGNVGAPGAKGARGSAGPPGATGFPGAAGRVGP
PGPSGNAGPPGPPGPAGKEGGKGPRGETGPAGRPGEVGLQIDNAR
LAAEDFRMKYENELALRQGVEADINGLRRVLDELTLTRTDLEMQI
ESLNEELAYMKKNHEDELQSFRVGGPGEVSVEMDAAPGVDLTRLL
NDMRAQYETIAEQNRKDAEAWFIEKSGELRKEISTNTEQLQSSKS
EVTDLRRAFQNLEIELQSQLAMKKSLEDSLAEAEGDY

A truncated human collagen type I, alpha 1 polypeptide (262 amino acids) and a truncated keratin type 31 polypeptide (K31, 147 amino acids):

(SEQ ID NO: 57)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEAL VETNRREVEQWFTTQTEELN
KQVVSSSEQLQSYQAEIIELRRTVNALEIELQAQHNLRDSLENTL
TESEARYSSQLSQVQSLITNVESQLAEIRSDLERQNQEYQVLLDV
RARLE

A truncated human collagen type I, alpha 1 polypeptide (262 amino acids) and a truncated keratin type 33a polypeptide (K33, 147 amino acids):

(SEQ ID NO: 58)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNQVLNETRSQYEALVETNRREVEQWFATQTEELNK
QVVSSSEQLQSYQAEIIELRRTVNALEIELQAQHNLRDSLENTLT
ESEARYSSQLSQVQRLITNVESQLAEIRSDLERQNQEYQVLLDVR
ARLE

A truncated human collagen type III, alpha 1 polypeptide (200 amino acids) and a truncated keratin type 31 polypeptide (K31, 147 amino acids):

(SEQ ID NO: 59)
GEPGANGLPGAAGERGAPGFRGPAGPNGIPGEKGPAGERGAPGPA
GPRGAAGEPGRDGVPGGPGMRGMPGSPGGPGSDGKPGPPGSQGES
GRPGPPGPSGPRGQPGVMGFPGPKGNDGAPGKNGERGGPGGPGPQ
GPPGKNGETGPQGPPGPTGPGGDKGDTGPPGPQGLQGLPGTGGPP
GENGKPGEPGPKGDAGAPGAQLGDRLNVEVDAAPTVDLNRVLNET
RSQYEALVETNRREVEQWFTTQTEELNKQVVSSSEQLQSYQAEII
ELRRTVNALEIELQAQHNLRDSLENTLTESEARYSSQLSQVQSLI
TNVESQLAEIRSDLERQNQEYQVLLDVRARLE.

A truncated human collagen type I, alpha 1 polypeptide (225 amino acids) and a truncated keratin type 31 polypeptide (K31, 147 amino acids):

(SEQ ID NO: 60)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
QLGDRLNVEVDAAPTVDLNRVLNETRSQYEALVETNRREVEQWFT
TQTEELNKQVVSSSEQLQSYQAEIIELRRTVNALEIELQAQHNLR
DSLENTLTESEARYSSQLSQVQSLITNVESQLAEIRSDLERQNQE
YQVLLDVRARLE.

A truncated human collagen type I, alpha 1 polypeptide (196 amino acids) and a truncated keratin type 31 polypeptide (K31, 147 amino acids):

(SEQ ID NO: 61)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGQLGD
RLNVEVDAAPTVDLNRVLNETRSQYEALVETNRREVEQWFTTQTEELNKQ
VVSSSEQLQSYQAEIIELRRTVNALEIELQAQHNLRDSLENTLTESEARY
SSQLSQVQSLITNVESQLAEIRSDLERQNQEYQVLLDVRARLE.

A truncated human collagen type I, alpha 1 polypeptide (130 amino acids) and a truncated keratin type 31 polypeptide (K31, 147 amino acids):

(SEQ ID NO: 62)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGQLGDRLNVEVDAAPTVDLNR
VLNETRSQYEALVETNRREVEQWFTTQTEELNKQVVSSSEQLQSYQAEII
ELRRTVNALEIELQAQHNLRDSLENTLTESEARYSSQLSQVQSLITNVES
QLAEIRSDLERQNQEYQVLLDVRARLE.

A truncated chicken collagen type 21 polypeptide (188 amino acids) and a truncated keratin type 31 polypeptide (K31, 147 amino acids):

(SEQ ID NO: 63)
DTGFPGMPGRSGDPGRSGKDGLPGSPGFKGEVGQPGSPGLEGHRGEPGIP
GIPGNQGAKGQKGEIGPPGLPGAKGSPGETGLMGPEGSFGLPGAPGPKGD
KGEPGLQGKPGSSGAKGEPGGPGAPGEPGYPGIPGTQGIKGDKGSQGESG
IQGRKGEKGRQGNPGLQGTEGLRGEQGEKGEKGDPGIRQLGDRLNVEVDA
APTVDLNRVLNETRSQYEALVETNRREVEQWFTTQTEELNKQVVSSSEQL
QSYQAEIIELRRTVNALEIELQAQHNLRDSLENTLTESEARYSSQLSQVQ
SLITNVESQLAEIRSDLERQNQEYQVLLDVRARLE.

A truncated chicken collagen type 21 polypeptide (141 amino acids) and a truncated keratin type 31 polypeptide (K31, 147 amino acids):

(SEQ ID NO: 64)
DTGFPGMPGRSGDPGRSGKDGLPGSPGFKGEVGQPGSPGLEGHRGEPGIP
GIPGNQGAKGQKGEIGPPGLPGAKGSPGETGLMGPEGSFGLPGAPGPKGD
KGEPGLQGKPGSSGAKGEPGGPGAPGEPGYPGIPGTQGIKGQLGDRLNVE
VDAAPTVDLNRVLNETRSQYEALVETNRREVEQWFTTQTEELNKQVVSSS
EQLQSYQAEIIELRRTVNALEIELQAQHNLRDSLENTLTESEARYSSQLS
QVQSLITNVESQLAEIRSDLERQNQEYQVLLDVRARLE.

A truncated chicken collagen type 21 polypeptide (93 amino acids) and a truncated keratin type 31 polypeptide (K31, 147 amino acids):

(SEQ ID NO: 65)
DTGFPGMPGRSGDPGRSGKDGLPGSPGFKGEVGQPGSPGLEGHRGEPGIP
GIPGNQGAKGQKGEIGPPGLPGAKGSPGETGLMGPEGSFGLPGQLGDRLN
VEVDAAPTVDLNRVLNETRSQYEALVETNRREVEQWFTTQTEELNKQVVS
SSEQLQSYQAEIIELRRTVNALEIELQAQHNLRDSLENTLTESEARYSSQ
LSQVQSLITNVESQLAEIRSDLERQNQEYQVLLDVRARLE.

A truncated chicken collagen type 21 polypeptide (47 amino acids) and a truncated keratin type 31 polypeptide (K31, 147 amino acids):

(SEQ ID NO: 66)
DTGFPGMPGRSGDPGRSGKDGLPGSPGFKGEVGQPGSPGLEGHRGEPQLG
DRLNVEVDAAPTVDLNRVLNETRSQYEALVETNRREVEQWFTTQTEELNK
QVVSSSEQLQSYQAEIIELRRTVNALEIELQAQHNLRDSLENTLTESEAR
YSSQLSQVQSLITNVESQLAEIRSDLERQNQEYQVLLDVRARLE.

A truncated human collagen type I, alpha 1 polypeptide (262 amino acids) and a truncated keratin type 31 polypeptide (K31, 100 amino acids):

(SEQ ID NO: 67)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGAPGA
KGARGSAGPPGATGFPGAAGRVGPPGPSGNAGPPGPPGPAGKEGGKGPRG
ETGPAGRPGEVGQLGDRLNVEVDAAPTVDLNRVLNETRSQYEALVETNRR
EVEQWFTTQTEELNKQVVSSSEQLQSYQAEIIELRRTVNALEIELQAQHN
LRDSLENTLTES.

A truncated human collagen type I, alpha 1 polypeptide (262 amino acids) and a truncated keratin type 31 polypeptide (K31, 75 amino acids):

(SEQ ID NO: 68)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGAPGA
KGARGSAGPPGATGFPGAAGRVGPPGPSGNAGPPGPPGPAGKEGGKGPRG
ETGPAGRPGEVGQLGDRLNVEVDAAPTVDLNRVLNETRSQYEALVETNRR
EVEQWFTTQTEELNKQVVSSSEQLQSYQAEIIELRRT.

A truncated human collagen type I, alpha 1 polypeptide (262 amino acids) and a truncated keratin type 31 polypeptide (K31, 50 amino acids):

(SEQ ID NO: 69)
GVPGDLGAPGPSGARGERGFPGERGVQGPPGPAGPRGANGAPGNDGAKGD
AGAPGAPGSQGAPGLOGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGAPGA
KGARGSAGPPGATGFPGAAGRVGPPGPSGNAGPPGPPGPAGKEGGKGPRG
ETGPAGRPGEVGQLGDRLNVEVDAAPTVDLNRVLNETRSQYEALVETNRR
EVEQWFTTQTEE.

A truncated human collagen type I, alpha 1 polypeptide (196 amino acids) and a truncated keratin type 31 polypeptide (K31, 100 amino acids):

(SEQ ID NO: 70)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGQLGD
RLNVEVDAAPTVDLNRVLNETRSQYEALVETNRREVEQWFTTQTEELNKQ
VVSSSEQLQSYQAEIIELRRTVNALEIELQAQHNLRDSLENTLTES.

A truncated human collagen type I, alpha 1 polypeptide (196 amino acids) and a truncated keratin type 31 polypeptide (K31, 50 amino acids):

(SEQ ID NO: 71)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGQLGD
RLNVEVDAAPTVDLNRVLNETRSQYEALVETNRREVEQWFTTQTEE.

A truncated human collagen type I, alpha 1 polypeptide (64 amino acids) and a truncated keratin type 31 polypeptide (K31, 100 amino acids):

(SEQ ID NO: 72)
GVPGDLGAPGPSGARGERGFPGERGVQGPPGPAGPRGANGAPGNDGAKGD
AGAPGAPGSQGAPGQLGDRLNVEVDAAPTVDLNRVLNETRSQYEALVETN
RREVEQWFTTQTEELNKQVVSSSEQLQSYQAEIIELRRTVNALEIELQAQ
HNLRDSLENTLTES.

A truncated human collagen type I, alpha 1 polypeptide (64 amino acids) and a truncated keratin type 31 polypeptide (K31, 50 amino acids):

(SEQ ID NO: 73)
GVPGDLGAPGPSGARGERGFPGERGVQGPPGPAGPRGANGAPGNDGAKGD
AGAPGAPGSQGAPGQLGDRLNVEVDAAPTVDLNRVLNETRSQYEALVETN
RREVEQWFTTQTEE.

A truncated human collagen type I, alpha 1 polypeptide (25 amino acids) and a truncated keratin type 31 polypeptide (K31, 147 amino acids):

(SEQ ID NO: 74)
GVPGDLGAPGPSGARGERGFPGERGQLGDRLNVEVDAAPTVDLNRVLNET
RSQYEALVETNRREVEQWFTTQTEELNKQVVSSSEQLQSYQAEIIELRRT
VNALEIELQAQHNLRDSLENTLTESEARYSSQLSQVQSLITNVESQLAEI
RSDLERQNQEYQVLLDVRARLE.

In certain embodiments, the ratio of the solubility-enhancing amino acid sequence length to the target amino acid sequence length (as measured by relative amino acid length) can be varied. For example, the ratio of the solubility-enhancing amino acid sequence to the target amino acid sequence can be at least 15:1, at least 14:1, at least 13:1, at least 12:1, at least 11:1, at least 10:1, at least 9:1, at least 8:1, at least 7:1, at least 6:1, at least 5:1, at least 4:1, at least 3:1, at least 2:1, at least 1.5:1, at least 1:1, at least 1:1.5, at least 1:2, at least 1:3, or at least 1:4. The ratio of the solubility-enhancing amino acid sequence to the target amino acid sequence can be, for example, about 4:1, 3.5:1, 3:1, 2.5:1, 2:1, 1.5:1, 1.25:1, 1:1, 1:1.25, 1:1.5, or 1:2. In certain embodiments the ratio of the solubility-enhancing amino acid sequence relative to the target amino acid sequence is from 1:1 to 1.25:1, from 1:1 to 1.5:1, from 1:1 to 1.75, from 1:1 to 2:1, from 1:1 to 2.25:1, from 1:1 to 2.5:1, from 1:1 to 2.75:1, from 1:1 to 3:1, from 1:1, to 3.25:1, from 1:1 to 3.5:1, from 1:1 to 3.75:1, from 1:1 to 4:1, from 1:1 to 4.25:1, from 1:1 to 4.5:1, from 1:1 to 4.75:1, from 1:1 to 5:1, from 1.5:1 to 1.75:1, 1.5:1 to 2:1, 1.5:1 to 2.25:1, 1.5:1 to 2.5:1, 1.5:1 to 2.75:1, 1.5:1 to 3:1, 1.5:1 to 3.25:1, 1.5:1 to 3.5:1, 1.5:1 to 3.75:1, 1.5:1 to 4:1, 1.5:1 to 4.25:1, 1.5:1 to 4.5:1, 1.5:1 to 4.75:1, 1.5:1 to 5:1, 2:1 to 2.25:1, 2:1 to 2.5:1, 2:1 to 2.75:1, 2:1 to 3:1, 2:1 to 3.25:1, 2:1 to 3.5:1, 2:1 to 3.75:1, 2:1 to 4:1, 2:1 to 4.25:1, 2:1 to 4.5:1, 2:1 to 4.75:1, 2:1 to 5:1, from 2.5:1 to 3:1, from 2.5: to 3.25:1, from 2.5: to 3.5:1, from 2.5: to 3.75:1, from 2.5: to 4:1, from 2.5: to 4.25:1, from 2.5: to 4.5:1, from 2.5: to 5:1, from 3:1 to 3.25:1, 3:1 to 3.5:1, 3:1 to 3.75:1, 3:1 to 4:1, 3:1 to 4.25:1, 3:1 to 4.5:1, 3:1 to 4.75:1, 3:1 to 5:1, from 3.5:1 to 3.75:1, 3.5:1 to 4:1, 3.5:1 to 4.25:1, 3.5:1 to 4.5:1, 3.5:1 to 4.75:1, 3.5:1 to 5:1, from 4:1 to 4.25:1, from 4:1 to 4.5:1, from 4:1 to 4.75:1, from 4:1 to 5:1, from 4.5:1 to 4.75:1, from 4.5:1 to 5:1. In certain embodiments, the higher the ratio of the solubility-enhancing amino acid sequence to the target amino acid sequence, the more likely a given fusion protein is to be soluble at a low pH. This can vary, for example, depending on such factors as the type and solubility characteristics of the target amino acid sequence, variations in the amino acid content of the solubility-enhancing amino acid sequence, the presence of post-translational modifications to the amino acids in the target sequence, the pH of the separation mixture, the presence (and amount) of contaminants in the mixture, the salinity of the mixture, the presence of additional solubilizing agents, and the temperature of the mixture.

In some embodiments, the non-naturally occurring polypeptide may include one or more solubility-enhancing amino acid sequence (S) together with one or more keratin-derived amino acid sequence (K) in any order. For example, S—K, K—S, S—K—S, K—S—K, etc. In certain embodiments, the non-naturally occurring polypeptide may include one or more solubility-enhancing amino acid sequence that is a truncated collagen polypeptide (C) together with one or more keratin-derived target amino acid sequence (K) in any order. For example, C—K, K—C, C—K—C, K—C—K, etc. These sequences may be directly linked or combined via a linker sequence as provided herein.

### Expression and Purification of Polypeptides Comprising a Solubility-Enhancing Amino Acid Sequence and a Keratin-Derived Target Amino Acid Sequence

Vectors. The non-naturally occurring polypeptides as described herein can be expressed or generated via a nucleic acid sequence encoding the non-naturally occurring polypeptides. Thus, another aspect of the disclosure includes a vector, e.g., an expression vector, comprising a nucleic acid sequence encoding the non-naturally occurring polypeptide. In some embodiments, the expression vector is a bacterial expression vector. In some embodiments, the expression vector is a yeast expression vector. In some embodiments, the expression vector is an insect cell expression vector. In other embodiments the expression vector is a mammalian expression vector, or a viral transfection vector. Any suitable expression vector that can induce the protein expression from the inserted nucleic acid encoding the non-naturally occurring polypeptide may be used in the corresponding host cell. Exemplary bacterial expression vectors may include pGEX vectors where glutathione S-transferase is used as a fusion partner and gene expression is under the control of the tac promoter, or pET vectors (e.g., pET28 vector, etc.) which uses a T7 promoter. Exemplary yeast expression vectors may include pPIC vectors, which uses the AOX1 promoter inducible with methanol. Exemplary mammalian cell expression vectors may include pcDNA™3.1(+) vectors, which use cytomegalovirus (CMV) enhancer-promoter elements, pcDNA™5/TO vectors, which use CMV enhancer-promoter elements coupled with tetracycline resistance operons for tetracycline-regulated expression, or lentiviral or retroviral expression vectors, which may rely on the strong constitutive EF-1 alpha promoter. In some embodiments, the expression vector is in a plasmid form (e.g., including bacterial artificial chromosome form, etc.) that are independently present in the host cell (e.g., cells expressing the recombinant polypeptide). In some embodiments, the expression vector is stably integrated into the chromosome of the host cell via random or targeted integration.

In some embodiments, the expression vector may include one or more selection agent. The selection agents include certain sugars including galactose containing sugars or antibiotics including ampicillin, hygromycin, G418 and others. Enzymes that are used to confer resistance to the selection agent include chloramphenicol acetyl-transferase (CAT) or a β-lactamase. Alternatively, and/or additionally, the expression vector includes an inducible promoter or a constitutive promoter (e.g., CMV promoter, etc.) such that the nucleic acid encoding the recombinant protein is operatively linked to the inducible promoter or the constitutive promoter. For example, the expression vector may include tetracycline-inducible promoter pTET, araC-ParaBAD inducible promoter, or IPTG inducible lac promoter. As used herein, “operatively linked” promoter and nucleic acid means that the expression of the nucleic acid (e.g., transcription, translation, etc.) is at least under partial control of the promoter.

Nucleotide sequences encoding the non-naturally occurring polypeptide or portions thereof may be cloned into expression vectors by any appropriate means known in the art. Such prepared expression vectors can be used to generate genetically engineered or modified organisms, or a recombinant cell to produce the non-naturally occurring polypeptides described herein. Preferably, the recombinant cells contain at least one copy of a plasmid or a stably integrated heterologous nucleic acid sequence encoding the non-naturally occurring polypeptide. In some embodiments, the recombinant cell is a microbial cell. For example, where the expression vector is bacterial expression vector, the expression vector can be inserted into (e.g., via any suitable transformation method) the bacterial cells for protein expression (e.g., Escherichia coli including BL-21 cells, etc.) to be independently present in the cytoplasm of the bacteria (e.g., as a plasmid form) or to be at least temporarily and/or stably integrated into the bacterial chromosome. Introduction of the vectors into the host cells may be performed using any appropriate means known in the art.

Host cells. In some embodiments, the nucleic acid sequence encoding the non-naturally occurring polypeptide is codon-optimized to be expressed in a heterologous host cell, preferably in microbial cells, e.g., bacterial cells. As used herein, “codon-optimized” means that the codon composition is improved for expression in the heterologous host cells (e.g., microbial cells, bacterial cells, etc.) by altering the nucleotide sequence to more closely reflect the codon usage of the host without altering the encoded amino acid sequences.

Recombinant polypeptides may be produced from non-animal sources, such as from a variety of host cell types. In some embodiments, the host cell can be, for example, an animal cell, an avian cell, a mammalian cell, an insect cell, a cell from a marine species, a plant cell, a blue-green algal cell, a microbial cell, a bacterial cell, an algal cell, a yeast cell, or a fungal cell. Respective conditions for transformation and growth of various host cell types are well known in the art.

Suitable bacterial host cells include, but are not limited to, cells of Escherichia, Proteus, Bacillus, Ralstonia, Lactobacillus, Lactococcus, Pseudomonas, Staphylococcus, and Streptomyces. Suitable cells of bacterial species include, but are not limited to, cells of Escherichia coli, Bacillus subtilis, Bacillus licheniformis, Bacillus megaterium, Lactobacillus brevis, Pseudomonas aeruginosa, Pseudomonas fluorescens, Pseudomonas stutzerei, Staphylococcus carnosus, Lactococcus lactis, Ralstonia eutropha, Proteus mirabilis, and Streptomyces lividans.

Suitable yeast host cells include, but are not limited to, cells of Saccharomyces (such as S. pombe and S. cerevisiae), Schizosaccharomyces, Candida, Hansenula, Pichia, Kluyveromyces, Yarrowia and Phaffia.

Suitable host cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

An example of a suitable avian host cell type that can be used includes, but is not limited to, an EB66 cell.

Examples of mammalian cells include, but are not limited to, HEK 293 cells, A549 cells, Vero cells, CHO cells, BHK cells, HeLa cells, COS cells, MRC 5 cells, FS4 cells, and MDCK cells.

Examples of plant cells include, but are not limited to, plant cell cultures and plant cell lines derived from e.g., carrot, tomato, rice, arabidopsis, and tobacco.

Examples of insect cells include, but are not limited to, Sf21, Sf9, and BTI-TN-5B1-4 (High Five) for use the AcNPV baculovirus systems, and Drosophila melanogaster Schneider 2(S2).

Exemplary growth conditions. In certain preferred embodiments, the host cell is Escherichia coli. In some embodiments, transformed cells can then be transferred to a larger volume of growth media (e.g., minimal media) and grown for at least 4 hours, at least 5 hours, at least 6 hours, at least 7 hours, at least 8 hours, from 5 to 10 hours, from 5 to 9 hours, from 6 to 9 hours, and/or alternatively until the cell density in the media reaches the desired optical density (OD).

Additionally, fermentation process can be performed at various temperature ranging from 22° C. to 37° C. In some embodiments, the temperature of the fermentation can be maintained at a constant temperature and immediately upon completion of fermentation the non-naturally occurring polypeptide can be purified. Alternatively, the temperature of the fermentations can be maintained for a desired period of time and when cell densities of OD600 of 10-20 are reached, then the temperature can be reduced to induce protein production. In such embodiments, typically, the temperature is reduced from 28° C. to 25° C. During the fermentation, protein expression in the bacteria can be induced by adding induction reagent. For example, where the expression vector contains lac promoter and the nucleic acid encoding the non-naturally occurring polypeptide is under the control of the lac promoter, the expression of the nucleic acid can be induced by adding isopropyl β-d-1-thiogalactopyranoside (IPTG) at a concentration ranging from 0.1-1.5 mM, from 0.1-1.0 mM, or from 0.1-0.5 mM. Fermentation can be continued for 20-24 hours, or in some embodiments, for 40-60 hours.

It is contemplated that such generated recombinant cells (e.g., recombinant bacteria transformed with the expression vector) intracellularly express the non-naturally occurring polypeptides encoded by the nucleic acids in the expression vector. Such intracellularly expressed polypeptides can then be secreted (via a secretion signal sequence) to the extracellular space (e.g., into a culture media). Thus, in some embodiments, the culture media can contain secreted recombinant protein encoded by the nucleic acids.

Recovery of recombinant polypeptides. In some embodiments, the recombinant polypeptides are purified from the culture medium where the recombinant host cells grow and secrete the recombinant polypeptides thereto. In some embodiments, the recombinant polypeptides are purified from cell lysates or homogenates, e.g., using a purification tag. In some embodiments, the recombinant polypeptide is coupled with a tag (e.g., histidine tag) such that the recombinant polypeptide can be purified using affinity purification is known as immobilized metal affinity chromatography (IMAC). Alternatively, the recombinant polypeptide can be purified via column chromatography. In some embodiments, the recombinant polypeptide can be purified by acid treatment of homogenized growth media in an acidification step. The recombinant cells are then separated using centrifugation, providing a pellet and a supernatant fraction. The recombinant polypeptides disclosed herein are particularly advantaged by the addition of the solubility-enhancing amino acid sequence in this purification step. As is disclosed herein, polypeptides comprising the solubility-enhancing amino acid sequence are fractionated into the supernatant following the acidification steps to reduce pH than comparable polypeptides lacking the solubility-enhancing amino acid sequence, which are often susceptible to precipitation or “crashing out” during these purification steps. Accordingly, the polypeptides are secreted into the extracellular media and remain soluble at lower acidic ranges, e.g., pH 3, in contrast to other contaminating proteins which are mainly insoluble at such pH ranges. pH levels can be altered to optimize the recovery of the recombinant polypeptide, for example, by lowering the pH to 6, 5.5, 5, 4.5, 4, 3.5, 3, or 2.5, and monitoring recovery of the recombinant polypeptide in soluble fractions versus pellet or precipitated fractions. This allows for rapid and cost-effective purification of the recombinant polypeptides. Supernatant of the acidified broth can be tested on a polyacrylamide gel and determined whether it contains the recombinant polypeptide in relatively high abundance compared to starting pellet.

The acidification step can occur, for example, at a pH of from about 5.0, 4.5, 4.0, 3.5, 3.0, 2.5, to about 2.0. The acidification step can occur at any pH between each of these numbers. The mixture can be pH treated with or without stirring, and at room temperature, or at a temperature range of from about 4° C. to about 40° C. Any suitable acid can be used to lower the pH. The low pH treatment can be from about 5 seconds, 30 seconds, 2 minutes, 5 minutes, 10 minutes, 15 minutes, 20 minutes, 30 minutes, 45 minutes, 60 minutes, or more. In preferred embodiments, the pH of the growth media (e.g., fermentation broth) can be decreased to from 3 to 3.5 using 5-50% sulfuric acid.

In some cases, this acidification step is the final purification. In other cases, the pH isolated protein is then purified by one or more additional means. Once the protein of interest has been produced, it may can isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins. The purified recombinant polypeptide can then be analyzed on an SDS-PAGE gel. Quantification of titers and purity can be further conducted using reverse phase and size exclusion HPLC chromatography. It is preferred that the purity of the purified recombinant polypeptides is at least at least about 80%, at least 80%, at least about 85%, at least 85%, at least about 90%, at least 90%, at least about 95%, at least 95%, at least about 99%, or at least 99%. The purified proteins so produced (i.e., the recombinant polypeptides) are animal-free and can be utilized either in the fused form (e.g., having characteristics of both collagens and keratins if so designed), or can be subsequently cleaved into the component polypeptides if designed with a cleavage linker, and further purified, if desired.

### Function of Recombinant Polypeptides

The non-naturally occurring polypeptides provided herein are not normally found in nature. Generally, the non-naturally occurring polypeptides described herein exhibit one or more differences from naturally occurring keratins. In some cases, the non-naturally occurring polypeptides may have a different structure from a naturally occurring keratin. Similarly, if a collagen polypeptide is used as a solubility-enhancing amino acid sequence, it may exhibit different structure or function from naturally occurring collagen. The quaternary structure of natural collagen is a triple helix, typically composed of three polypeptides. In some aspects, the non-naturally occurring polypeptides described herein may not form the same structure of either collagen, keratin, or both, as would occur in the native organism. In some aspects, the non-naturally occurring polypeptides described herein are monomeric and/or do not form multimeric structures under normal biological circumstances. In other aspects, the non-naturally occurring polypeptides described herein may, in some instances, form multimeric structures with identical monomers (e.g., homodimers, homotrimers, etc.). The non-naturally occurring polypeptides disclosed herein often have advantageous properties related to their monomeric structure and/or lack of amino acids capable of cross-linking with other polypeptide strands, e.g., the lack of hydroxyproline residues. In addition, hydrolysates of the non-naturally occurring polypeptides disclosed herein often maintain increased solubility as compared to full-length or natural proteins or polypeptides.

Generally, the non-naturally occurring polypeptides provided herein may have a certain function and/or provide a benefit (e.g., as provided herein) similar or substantially similar to that of a natural or a full-length polypeptide. In some cases, the non-naturally occurring polypeptides provided herein may have improved or increased function and/or benefit (e.g., as provided herein) as compared to a natural or a full-length polypeptide. In some embodiments, the non-naturally occurring polypeptides provided herein may have one or more different functions as compared to a natural or a full-length polypeptide. Target sequences may be bio-designed according to methods provided herein to achieve the desired functional properties.

In some embodiments, the non-naturally occurring polypeptides provided herein are capable of providing both collagen-like characteristics and/or functions and keratin-like characteristics and/or functions, all in one fusion protein. For example, topical application of such a polypeptide can provide skin benefits typically associated with collagen, as well as skin benefits typically associated with keratin. Additionally, application of a formulation containing the fusion protein to hair can have benefits that are typically associated with collagen, in addition to benefits typically associated with keratin.

In some embodiments, the non-naturally occurring polypeptides may provide functions such as, for example, decreasing skin damage, promoting the repair of damaged skin, protecting the skin against UV damage, protecting skin cells against the effects of exposure to urban dust, increasing viability of skin cells, increasing the viability of fibroblast cells, increasing the viability of keratinocyte cells, increasing procollagen synthesis, decreasing the production of inflammatory cytokines, repairing or strengthening the nail plate, repairing dry or damaged nails, preventing damage to nails, improving strength or growth of nails, or any combination thereof.

In some embodiments, the non-naturally occurring polypeptides may provide functions such as, for example, in improving hair strength, improving hair shininess, smoothness, suppleness, sheen or feel, improving hair combability or manageability, improving hair flexibility, increasing hair strand diameter, strengthening hair, repairing split ends, repairing hair damaged by atmospheric agents, repairing hair damaged or weakened by mechanical or chemical treatments, protecting hair from damage by atmospheric agents, mechanical or chemical treatments, or any combination thereof.

### Compositions and Formulations of Recombinant Polypeptides

In various aspects, provided herein are compositions and formulations comprising a polypeptide of the disclosure and one or more additional ingredients. The compositions and formulations of the present disclosure can include or be incorporated into all types of vehicles and carriers. The vehicle or carrier can be a cosmetically or dermatologically acceptable vehicle or carrier. Compositions may be formulated for topical applications, e.g., personal care products for application to skin, hair, scalp, or nails. Topical application includes application on skin and/or keratinous tissue. Keratinous tissue includes keratin-containing layers disposed as the outermost protective covering of mammals and includes, but is not limited to, lips, skin, hair, and nails. Non-limiting examples of vehicles or carriers include water, glycerin, alcohol, oil, a silicon containing compound, a silicone compound, and wax. Formulations and variations and other appropriate vehicles will be apparent to the skilled artisan and are appropriate for use in compositions and formulations of the present disclosure. In some embodiments, the formulation or personal care product may be mask, a skin cleaner, a cleansing cream, a cleansing lotion, a facial cleanser, a cleansing milk, a cleansing pad, a facial wash, a facial cream, a facial lotion, a body cream, a body lotion, a facial moisturizer, a body moisturizer, a facial serum, a facial mask, a body mask, a facial toner, a facial mist, an eye cream, an exfoliator formula, a lip balm, a lipstick, a nail treatment, a hair shampoo, a hair conditioner, a body shampoo, a hair serum, a scalp serum, a hair mist, a hair spray, an eye shadow, a concealer, a mascara, and the like. Examples of hair care products include scalp or hair medications, scalp or hair growth modifiers or stimulants, scalp or hair cleansers, scalp or hair conditioners, serums, combing cream, a styling mousse, lotions, leave-in rinses, sprays, creams, gels, powders, and the like.

In certain aspects, the concentrations and combinations of the compounds, ingredients, and agents can be selected in such a way that the combinations are chemically compatible and do not form complexes which precipitate from the finished product.

Provided in certain embodiments herein are (e.g., topical) compositions, formulations, and/or personal care products comprising one or more non-naturally occurring polypeptide provided herein (e.g., for cosmetic use). In some embodiments, the compositions, formulations, and/or personal care products provide any suitable amount of polypeptide provided herein, such as in any suitable amount (e.g., an amount suitable to provide a benefit when given or applied to an individual or a cell). In some specific embodiments, the compositions, formulations, and/or personal care products comprise an amount suitable to provide a beneficial effect to the skin, hair, scalp and/or nails of an individual when (e.g., topically) applied to the skin of the individual. In specific embodiments, the compositions, formulations, and/or personal care products comprise about 0.001% to about 30% w/w of a polypeptide (or non-naturally occurring recombinant polypeptide) such as provided herein. In more specific embodiments, the compositions, formulations, and/or personal care products comprise about 0.001% to about 20% w/w of a polypeptide (or non-naturally occurring recombinant polypeptide) such as provided herein, about 0.001% to about 10% w/w of a polypeptide (or non-naturally occurring recombinant polypeptide) such as provided herein, about 0.001% to about 5% w/w of a polypeptide (or non-naturally occurring recombinant polypeptide) such as provided herein, about 0.001% to about 4% w/w of a polypeptide (or non-naturally occurring recombinant polypeptide) such as provided herein, about 0.001% to about 3% w/w of a polypeptide (or non-naturally occurring recombinant polypeptide) such as provided herein, about 0.001% to about 2% w/w of a polypeptide (or non-naturally occurring recombinant polypeptide) such as provided herein, about 0.001% to about 1% w/w of a polypeptide (or non-naturally occurring recombinant polypeptide) such as provided herein, about 0.001% to about 0.5% w/w of a polypeptide (or non-naturally occurring recombinant polypeptide) such as provided herein, and about 0.001% to about 0.2% w/w of a polypeptide (or non-naturally occurring recombinant polypeptide) such as provided herein.

In various embodiments, the concentration or amount of a non-naturally occurring polypeptide provided herein is in a composition, formulation, and/or personal care product provided herein in any suitable amount and may, e.g., vary depending on the use or formulation (e.g., gel, capsule, liquid, powder, etc.). Exemplary concentrations of the non-naturally occurring polypeptides (e.g., recombinant proteins) in the compositions, formulations, and/or personal care products can be at least about 0.01%, at least about 0.05%, at least about 0.1%, at least about 0.2%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% (w/v or w/w). Alternatively and/or additionally, the exemplary concentration of the non-naturally occurring polypeptides in the compositions, formulations, and/or personal care products can be about 0.01%, about 0.05%, about 0.1%, about 0.2%, about 0.5%, about 1%, about 2%, about 3%, at about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98% (w/v or w/w). Alternatively and/or additionally, the exemplary concentration of the non-naturally occurring polypeptides in the compositions, formulations, and/or personal care products can range from about 0.01% to about 99%, from about 0.05% to about 99%, from about 0.1% to about 99%, from about 0.1% to about 99%, from about 0.5% to about 99%, from about 0.1% to about 10%, from about 1% to about 99%, from about 5% to about 99%, from about 10% to about 99%, from about 15% to about 99%, from about 20% to about 99%, from about 25% to about 99%, from about 30% to about 99%, from about 35% to about 99%, from about 40% to about 99%, from about 45% to about 99%, from about 50% to about 99%, from about 55% to about 99%, from about 60% to about 99%, from about 65% to about 99%, from about 70% to about 99%, from about 75% to about 99%, from about 80% to about 99%, from about 85% to about 99%, from about 90% to about 99%, from about 95% to about 99%, from about 0.1% to about 90%, from about 1% to about 90%, from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, from about 80% to about 90%, from about 85% to about 90%, from about 20% to about 80%, from about 25% to about 80%, from about 30% to about 80%, from about 35% to about 80%, from about 40% to about 80%, from about 45% to about 80%, from about 50% to about 80%, from about 55% to about 80%, from about 60% to about 80%, from about 65% to about 80%, from about 70% to about 80%, from about 75% to about 80%, from about 70% to about 99%, from about 75% to about 99%, from about 80% to about 99%, etc. (w/w or w/v). Alternatively, and/or additionally, the exemplary concentration of the non-naturally occurring polypeptides (e.g., recombinant polypeptides) in the compositions, formulations, and/or personal care products can be less than about 95%, about 90%, about 85%, about 80%, about 75%, about 70%, about 65%, about 60%, about 55%, about 50%, about 45%, about 40%, etc. (w/w or w/v).

In some embodiments, the schedule of application varies depending on the purpose, gender, age, or health condition of the subject. For example, in some embodiments, the compositions, formulations, and/or personal care products are applied (e.g., topically) once a day, twice a day, three times a day, up to 6 times a day, every 2 days, every 3 days, every 4 days, every 5 days, every 6 days, etc. Alternatively, and/or additionally, in some embodiments, the compositions, formulations, and/or personal care products are applied (e.g., topically) a plurality of times in an irregular interval, or increased interval, or decreased interval. In certain embodiments, the compositions, formulations, and/or personal care products are topically applied in a dose and/or schedule sufficient or effective to achieve results as provided herein.

The topical application can be for cosmetic purpose. The topical formulation can be any type of topical formulation, including, but not limited to, a powder, a cream, a gel, a gel cream, a liquid, a lotion, an oil, and the like. In such embodiments, the composition may further include at least one of a carrier molecule (e.g., vehicle), a preservative, and/or additional ingredients. Any suitable carrier molecules are contemplated, and the exemplary carrier molecule may include water, oil, alcohol, propylene glycol, or emulsifiers, liposome, biodegradable microcapsule, lotion, spray, aerosol, dusting powder, biodegradable polymer, mineral oil, triglyceride oil, silicone oil, glycerin, glycerin monostearate, alcohols, emulsifying agents, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, and cyclopentasiloxane.

In addition, any suitable preservatives are contemplated, and the exemplary preservatives include zinc oxide, parabens, formaldehyde releasers, isothiazolinones, phenoxyethanol, or organic acids such as benzoic acid, sodium benzoate, or butylene glycol, hexanediol, potassium sorbate, tocopherol, diiodomethyl-p-tolylsulfone, 2-bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-ethylbicyclooxazolidine, phenoxyethanol, butylene glycol, 1,2 Hexanediol, methyl paraben, sorbic acid, Germaben® II, rosemary extract, and EDTA.

Such compositions are typically dermatologically acceptable in that they do not have undue toxicity, incompatibility, instability, allergic response, and the like, when applied to skin and/or keratinous tissue. Topical skin care compositions of the present disclosure can have a selected viscosity to avoid significant dripping or pooling after application to skin and/or keratinous tissue.

The personal care products may be useful for increasing the firmness, elasticity, brightness, hydration, tactile texture, or visual texture of skin and/or stimulate collagen production. The personal care products may be useful for reducing deep lines and wrinkles, reducing fine lines and wrinkles, evening uneven skin tone, increasing skin radiance, reducing photodamage, reducing sagging skin, reducing loss of facial volume, increasing skin barrier function, reducing redness of the skin, reducing skin dryness, reducing peeling or flaking, or increasing expression and/or production of collagen, elastin, fibronectin or laminin.

Hair care products and methods of use. In some embodiments, the recombinant polypeptide is useful as an ingredient in a hair care product, for example in shampoo and conditioning compositions. The recombinant polypeptide can be useful for treating damaged hair or scalp. The recombinant polypeptide can also be useful for protecting hair from future damage. Examples of factors that can cause hair damage over time include environmental influences, nutrition, heat treatments, and chemical treatments. Damaged hair can become weak, creating broken hair strands (“split ends”). The damaged hair can also have a dull appearance. The recombinant polypeptide can be useful addition to hair or scalp conditioners.

The recombinant polypeptide can be incorporated into or included in a rinse-off or rinse out skin or hair composition. This refers to a composition that is applied to the skin, hair, and/or scalp and is subsequently rinsed, optionally after a period of time, such as after about 10 seconds, after about 20 seconds, after about 30 seconds, after about 1 minute, after about 5 minutes, after about 10 minutes, after about 20 minutes, after about 30 minutes, or after about an hour. For example, the rinse-off composition may be a skin-cleansing composition or a shampoo composition, such as a conditioning shampoo composition. The recombinant polypeptide compositions can be applied to the skin, hair, and/or scalp and subsequently rinsed off. For example, the skin, hair, and/or scalp may be washed or cleansed in a first step of applying the composition of the disclosure onto the skin, hair, and/or scalp, with an optional leave-on time, followed by a second step of rinsing the hair with water, for example after an optional leave-on time, such as from 1 minute, 5 minutes, 10 minutes, 15 minutes or about 20 minutes or more.

The methods of treating and/or caring for the skin, hair, and/or scalp according to the disclosure may, in various embodiments, impart moisture benefits to the skin or scalp, or conditioning and manageability benefits to the hair, even after the composition is rinsed off. In addition, hair treated with the recombinant polypeptide conditioning systems and/or rinse-off hair compositions according to the disclosure can result in greater ease of detangling, greater smoothness, greater shininess, greater discipline without a greasy coating or weighed-down feeling, moisturized feel, split-end seal, and/or reduced static, may be more sleek, and/or may have greater frizz control, relative to hair not having been treated with a solubility-enhancing amino acid sequence—keratin fusion protein—containing composition according to the disclosure.

In some embodiments, the recombinant polypeptide is useful as an ingredient in a hair care product that is a “leave-in” product. Such products can be applied to the hair after shampooing, or during the hair styling process. Further, the recombinant polypeptide compositions can be useful in taming curly or unruly hair. Curly hair tends to significantly expand in volume and lose its curl definition when exposed to high humidity conditions or changes in humidity environment.

Additional ingredients. The compositions can contain other cosmetic ingredients suitable for human use. In some aspects, the compositions and formulations of the present disclosure can further include a surfactant, a silicone containing compound, a UV agent, an oil, and/or other ingredients identified in this specification or those known in the art. The composition can be a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, gel serums, gel emulsions, etc. The composition can be formulated for topical skin, scalp, or hair application at least 1, at least 2, at least 3, at least 4, at least 5, at least 6, at least 7, or more times a day during use.

The compositions and formulations of the present disclosure can include a triglyceride. Non-limiting examples include small, medium, and large chain triglycerides. The compositions and formulations of the present disclosure can also include preservatives. Non-limiting examples of preservatives include phenoxyethanol, methylparaben, propylparaben, iodopropynyl butylcarbamate, potassium sorbate, sodium benzoate, or any mixture thereof. In some embodiments, the compositions and formulations of the disclosure are paraben-free.

Compositions and formulations of the present disclosure can have UVA and UVB absorption properties. The compositions and formulations of the present disclosure can have a sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions and formulations of the present disclosure can be sunscreen lotions, sprays, or creams.

The compositions and formulations of the present disclosure can also include any one of, any combination of, or all of the following additional ingredients: a conditioning agent, a moisturizing agent, a coloring agent, a pH adjuster, a structuring agent, inorganic salts, a preservative, a thickening agent, a silicone containing compound, an essential oil, a fragrance, a vitamin, a pharmaceutical ingredient, or an antioxidant, or any combination of such ingredients or mixtures of such ingredients. The amounts of such ingredients can range from 0.0001% to 99.9% by weight or volume of the composition, or any integer or range in between.

The CTFA International Cosmetic Ingredient Dictionary and Handbook (2004 and 2008) describes a wide variety of non-limiting cosmetic ingredients that can be used in the context of the present disclosure. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, gluconolactone, calcium gluconate, cyclohexasiloxane, and dipotassium glycyrrhizate).

In some embodiments, the compositions, formulations, and/or personal care products of the present disclosure include one or more additional ingredients selected from the group consisting of: levulinic acid, polyglyceryl-3 methylglucose distearate, glyceryl undecylenate, Simmondsia chinensis (Jojoba) seed oil, polyacrylate cross-polymer, squalane, sodium hyaluronate, acrylic acid polymers (carbomers), pentylene glycol, sodium lauryl sulfoacetate, sodium oleoyl sarcosinate, sodium oleate, Ricinus communis (castor) seed oil, Copernicia cerifera (Carnauba) wax, Candelilla wax, Theobroma cacao (Cocoa) Seed Butter, isononyl isononanoate, ozokerite, isopropyl titanium triisostearate, polyhydroxystearic acid, iron oxide, titanium dioxide, sodium levulinate, and hydroxypropyl guar.

Characteristic properties. In various aspects, the compositions, formulations, and/or personal care products provided herein are animal-free. For example, the compositions, formulations, and/or personal care products provided herein do not include any ingredients obtained from an animal. In some cases, the compositions, formulations, and/or personal care products provided herein comprise and/or are made from materials obtained from plants or materials with a plant origin. In some cases, the compositions, formulations, and personal care products provided herein comprise materials obtained synthetically or materials with a synthetic origin (e.g., produced in a microbial cell, e.g., a bacterial cell, a yeast cell, a fungal cell). In some cases, the compositions, formulations, and/or personal care products provide herein do not contain Animal Derived Ingredients (ADIs). Thus, the compositions, formulations, and/or personal care products provided herein are free of Bovine Spongiform Encephalopathy (BSE) and/or Transmissible Spongiform Encephalopathies (TSE). In some embodiments, the compositions, formulations, and/or personal care products provided herein are not tested on animals.

In various aspects, the compositions, formulations, and/or personal care products provided herein do not comprise any detectable genetically modified organisms or any detectable genetically modified organism genetic material. In some cases, the compositions, formulations, and/or personal care products provided herein are characterized by the absence of live microflora, as determined by a colony forming unit (CFU) assay. In some cases, the compositions, formulations, and/or personal care products provided herein are characterized by the absence of live microflora DNA, as determined by polymerase chain reaction (PCR).

In various aspects, the compositions, formulations, and/or personal care products provided herein do not contain any naturally occurring and/or synthetic chemicals that are known to cause cancer or birth defects or other reproductive harm. A non-limiting list of such ingredients may be found at oehha.ca.gov/proposition-65/proposition-65-list. In various aspects, the compositions, formulations, and/or personal care products provided herein do not contain any carcinogenic, mutagenic, or toxic to reproduction (CMR) substances. In various aspects, the compositions, formulations, and/or personal care products provided herein do not contain a substance of very high concern (SVHC). In various aspects, the compositions, formulations, and/or personal care products do not contain any Convention on International Trade in Endangered Species of Wild Fauna and Flora (CITES) ingredients (e.g., any ingredients derived from, obtained from, or originating from any species protected by CITES). In various aspects, the compositions, formulations, and/or personal care products do not contain any conflict minerals or conflict resources.

In various aspects, the compositions, formulations, and/or personal care products are fragrance-free. In various aspects, the compositions, formulations, and/or personal care products are compliant with the International Fragrance Association (IFRA).

In various aspects, the compositions, formulations, and/or personal care do not contain any known allergens. In some cases, the compositions, formulations, and/or personal care products are free of any source of tree nut or peanut-based materials. In some cases, the compositions, formulations, and/or personal care products are not processed using equipment that has been in contact with tree nut or peanut-based materials. In some cases, the compositions, formulations, and/or personal care products are free of any source of coconut-based materials. In some cases, the compositions, formulations, and/or personal care products are not processed using equipment that has been in contact with coconut-based materials. In some cases, the compositions, formulations, and/or personal care products are free of any source of wheat-based materials. In some cases, the compositions, formulations, and/or personal care products are not processed using equipment that has been in contact with wheat-based materials. In some cases, the compositions, formulations, and/or personal care products are free of any source of gluten (e.g., are gluten-free). In some cases, the compositions, formulations, and/or personal care products are free of any source of lactose or lactose derivatives (e.g., are lactose-free). In some cases, the compositions, formulations, and/or personal care products are free of any source of latex or latex derivatives (e.g., are latex-free).

In various aspects, the compositions, formulations, and/or personal care products are free of one or more ingredient selected from the group consisting of: phthalates, parabens, triclosan, urea, butylated hydroxytoluene (BHT), butylated hydroxyanisole (BHA), formaldehyde, a mixture of methylchloroisothiazolinone and methylisothiazolinone (e.g., Kathon®), mineral oil, phenoxyethanol, petrolatum, monoethanolamine (MEA), diethanolamine (DEA), triethanolamine (TEA), ethylenediaminetetraacetic acid (EDTA), ethylene glycol, sulfates (e.g., sodium lauryl sulfate (SLS), sodium lauryl ether sulfate (SLES)), retinyl palmitate, ethylene oxide, 1,4-dioxane, and any combination thereof. In various aspects, the compositions, formulations, and/or personal care products are free of pesticides. In various aspects, the compositions, formulations, and/or personal care products are free of nanoparticles (“nano-free”). In various aspects, the compositions, formulations, and/or personal care products are free of aflatoxins. In various aspects, the compositions, formulations, and/or personal care products are free of mycotoxins. In various aspects, the compositions, formulations, and/or personal care products are free of poly aromatic hydrocarbons (PAH). In various aspects, the compositions, formulations, and/or personal care products are free of silicones (e.g., cyclosiloxanes). In various aspects, the compositions, formulations, and/or personal care products are not manufactured using any solvents listed in USP <467> or ICH Q3C (R6). In various aspects, the compositions, formulations, and/or personal care products do not contain any volatile organic compounds as defined by the Swiss Ordinance 814.018.

In various aspects, the compositions, formulations, and/or personal care products contain less than 0.5 ppm arsenic. In various aspects, the compositions, formulations, and/or personal care products contain less than 0.1 ppm mercury. In various aspects, the compositions, formulations, and/or personal care products contain less than 0.1 ppm cadmium. In various aspects, the compositions, formulations, and/or personal care products contain less than 2 ppm lead.

In various aspects, the compositions, formulations, and/or personal care products are certified as Vegan. In various aspects, the compositions, formulations, and/or personal care products are certified as Cruelty-Free. In various aspects, the compositions, formulations, and/or personal care products are certified as Halal.

### Uses of Recombinant Polypeptides as Skin Compositions and Formulations

In certain embodiments, provided herein are methods of promoting, maintaining, and/or improving youthful skin (e.g., appearance of skin, texture of skin, etc.) of an individual, comprising applying a composition, a formulation, and/or a personal care product (e.g., containing a non-naturally occurring polypeptide of the disclosure) to the skin of an individual. Promoting and/or maintaining youthful skin may comprise promoting, maintaining, and/or improving the appearance of the skin of an individual. In some cases, the appearance of the skin of the individual, after application of the compositions, formulations, and/or personal care products provided herein (e.g., containing a non-naturally occurring polypeptide of the disclosure) more closely resembles the appearance of the skin of a young individual (e.g., less than 30 years old, less than 25 years old, less than 20 years old, less than 15 years old, etc.). Promoting and/or maintaining youthful skin may comprise promoting, maintaining, and/or improving the texture of the skin of an individual. In some cases, the texture of the skin of the individual, after application of the compositions, formulations, and/or personal care products provided herein (e.g., containing a non-naturally occurring polypeptide of the disclosure) more closely resembles the texture of the skin of a young individual (e.g., less than 30 years old, less than 25 years old, less than 20 years old, less than 15 years old, etc.).

In various aspects, the methods provided herein comprise applying the compositions, formulations, and/or personal care products (e.g., containing a non-naturally occurring polypeptide of the disclosure) to the skin an individual to promote, maintain, and/or improve the firmness of the skin. In some cases, the firmness of the skin of the individual, after application of the compositions, formulations, and/or personal care products provided herein (e.g., containing a non-naturally occurring polypeptide of the disclosure) more closely resembles the firmness of the skin of a young individual (e.g., less than 30 years old, less than 25 years old, less than 20 years old, less than 15 years old, etc.). In some cases, promoting, maintaining, and/or improving the firmness of the skin involves increasing the firmness of the skin (e.g., relative to the skin prior to application of the compositions, formulations, and/or personal care products provided herein (e.g., containing a non-naturally occurring polypeptide of the disclosure). In some cases, the firmness of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by measuring the resistance of the skin to negative pressure. In some cases, the resistance of the skin to negative pressure is measured by using a Cutometer®.

In various aspects, the methods provided herein comprise applying the compositions, formulations, and/or personal care products (e.g., containing a non-naturally occurring polypeptide of the disclosure) to the skin of an individual to promote, maintain, and/or improve the elasticity of the skin. In some cases, the elasticity of the skin of the individual, after application of the compositions, formulations, and/or personal care products provided herein (e.g., containing a non-naturally occurring polypeptide of the disclosure) more closely resembles the elasticity of the skin of a young individual (e.g., less than 30 years old, less than 25 years old, less than 20 years old, less than 15 years old, etc.). In some cases, promoting, maintaining, and/or improving the elasticity of the skin involves increasing the elasticity of the skin (e.g., relative to the skin prior to application of the compositions, formulations, and/or personal care products provided herein (e.g., containing a non-naturally occurring polypeptide of the disclosure). In some cases, the elasticity of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by measuring the ability of the skin to return to its original position after deformation. In some cases, the ability of the skin to return to its original position after deformation is measured by using a Cutometer®.

In various aspects, the methods provided herein comprise applying the compositions, formulations, and/or personal care products (e.g., containing a non-naturally occurring polypeptide of the disclosure) to the skin of an individual to promote, maintain, and/or improve the brightness of the skin. In some cases, the brightness of the skin of the individual, after application of the compositions, formulations, and/or personal care products provided herein (e.g., containing a non-naturally occurring polypeptide of the disclosure) more closely resembles the brightness of the skin of a young individual (e.g., less than 30 years old, less than 25 years old, less than 20 years old, less than 15 years old, etc.). In some cases, promoting, maintaining, and/or improving the brightness of the skin involves increasing the brightness of the skin (e.g., relative to the skin prior to application of the compositions, formulations, and/or personal care products provided herein (e.g., containing a non-naturally occurring polypeptide of the disclosure). In some cases, the brightness of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by an expert clinical grader.

In various aspects, the methods provided herein comprise applying the compositions, formulations, and/or personal care products (e.g., containing a non-naturally occurring polypeptide of the disclosure) to the skin or hair of an individual to promote, maintain, and/or improve the hydration of the skin or hair. In some cases, the hydration of the skin or hair of the individual, after application of the compositions, formulations, and/or personal care products provided herein (e.g., containing a non-naturally occurring polypeptide of the disclosure) more closely resembles the hydration of the skin or hair of a young individual (e.g., less than 30 years old, less than 25 years old, less than 20 years old, less than 15 years old, etc.). In some cases, promoting, maintaining, and/or improving the hydration of the skin involves increasing the hydration of the skin (e.g., relative to the skin prior to application of the compositions, formulations, and/or personal care products provided herein (e.g., containing a non-naturally occurring polypeptide of the disclosure). In some cases, the hydration of the skin or hair, after application of the compositions, formulations, and/or personal care products provided herein (e.g., containing a non-naturally occurring polypeptide of the disclosure) is increased (e.g., relative to the skin or hair prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by measuring capacitance of the skin or hair. In some cases, capacitance of the skin or hair is measured by a Corneometer®.

In various aspects, the methods provided herein comprise applying the compositions, formulations, and/or personal care products (e.g., containing a non-naturally occurring polypeptide of the disclosure) to the skin or hair of an individual to promote, maintain, and/or improve tactile texture of the skin or hair. In some cases, tactile texture of the skin or hair of the individual, after application of the compositions, formulations, and/or personal care products provided herein (e.g., containing a non-naturally occurring polypeptide of the disclosure) more closely resembles tactile texture of the skin or hair of a young individual (e.g., less than 30 years old, less than 25 years old, less than 20 years old, less than 15 years old, etc.).

In various aspects, the methods provided herein comprise applying the compositions, formulations, and/or personal care products (e.g., containing a non-naturally occurring polypeptide of the disclosure) to the skin or hair of an individual to promote, maintain, and/or improve visual texture of the skin or hair. In some cases, visual texture of the skin or hair of the individual, after application of the compositions, formulations, and/or personal care products provided herein (e.g., containing a non-naturally occurring polypeptide of the disclosure) more closely resembles visual texture of the skin or hair of a young individual (e.g., less than 30 years old, less than 25 years old, less than 20 years old, less than 15 years old, etc.).

In various aspects, the methods provided herein comprise applying the compositions, formulations, and/or personal care products (e.g., containing a non-naturally occurring polypeptide of the disclosure) to the skin of an individual to promote, maintain, and/or improve the collagen content of the skin. In some cases, the collagen content of the skin of the individual, after application of the compositions, formulations, and/or personal care products provided herein (e.g., containing a non-naturally occurring polypeptide of the disclosure) more closely resembles the collagen content of the skin of a young individual (e.g., less than 30 years old, less than 25 years old, less than 20 years old, less than 15 years old, etc.). In some cases, promoting, maintaining, and/or improving the collagen content of the skin involves increasing the collagen content of the skin (e.g., relative to the skin prior to application of the compositions, formulations, and/or personal care products provided herein (e.g., containing a non-naturally occurring polypeptide of the disclosure). In some cases, the collagen content of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%.

In various aspects, the methods provided herein comprise applying the compositions, formulations, and/or personal care products (e.g., containing a non-naturally occurring polypeptide of the disclosure) to the skin of an individual to promote, maintain, and/or improve the elastin content of the skin. In some cases, the elastin content of the skin of the individual, after application of the compositions, formulations, and/or personal care products provided herein (e.g., containing a non-naturally occurring polypeptide of the disclosure) more closely resembles the elastin content of the skin of a young individual (e.g., less than 30 years old, less than 25 years old, less than 20 years old, less than 15 years old, etc.). In some cases, promoting, maintaining, and/or improving the elastin content of the skin involves increasing the elastin content of the skin (e.g., relative to the skin prior to application of the compositions, formulations, and/or personal care products provided herein (e.g., containing a non-naturally occurring polypeptide of the disclosure). In some cases, the elastin content of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%.

In various aspects, the methods provided herein comprise applying the compositions, formulations, and/or personal care products (e.g., containing a non-naturally occurring polypeptide of the disclosure) to the skin of an individual to improve the redness of the skin. In some cases, the redness of the skin of the individual, after application of the compositions, formulations, and/or personal care products provided herein (e.g., containing a non-naturally occurring polypeptide of the disclosure) more closely resembles the redness of the skin of a young individual (e.g., less than 30 years old, less than 25 years old, less than 20 years old, less than 15 years old, etc.). In some cases, improving the redness of the skin involves decreasing the redness of the skin (e.g., relative to the skin prior to application of the compositions, formulations, and/or personal care products provided herein (e.g., containing a non-naturally occurring polypeptide of the disclosure). In some cases, the redness of the skin, after application of the compositions, formulations, and/or personal care products provided herein (e.g., containing a non-naturally occurring polypeptide of the disclosure) is decreased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by an expert clinical grader.

In various aspects, the methods provided herein comprise applying the compositions, formulations, and/or personal care products (e.g., containing a non-naturally occurring polypeptide of the disclosure) to the skin of an individual to improve fine lines and/or wrinkles of the skin. In some cases, the fine lines and/or wrinkles of the skin of the individual, after application of the compositions, formulations, and/or personal care products provided herein (e.g., containing a non-naturally occurring polypeptide of the disclosure) more closely resembles the fine lines and/or wrinkles skin of a young individual (e.g., less than 30 years old, less than 25 years old, less than 20 years old, less than 15 years old, etc.). In some cases, improving the fine lines and/or wrinkles of the skin involves decreasing fine lines and/or wrinkles (e.g., decreasing the amount, decreasing the size, etc.) of the skin (e.g., relative to the skin prior to application of the compositions, formulations, and/or personal care products provided herein (e.g., containing a non-naturally occurring polypeptide of the disclosure). In some cases, the fine lines and/or wrinkles of the skin, after application of the compositions, formulations, and/or personal care products provided herein (e.g., containing a non-naturally occurring polypeptide of the disclosure) is decreased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by an expert clinical grader.

In various aspects, the methods provided herein comprise applying the compositions, formulations, and/or personal care products (e.g., containing a non-naturally occurring polypeptide of the disclosure) to the skin of an individual to promote, maintain, and/or improve epidermal thickness of the skin. In some cases, the epidermal thickness of the skin of the individual, after application of the compositions, formulations, and/or personal care products provided herein (e.g., containing a non-naturally occurring polypeptide of the disclosure) more closely resembles the epidermal thickness of the skin of a young individual (e.g., less than 30 years old, less than 25 years old, less than 20 years old, less than 15 years old, etc.). In some cases, promoting, maintaining, and/or improving the epidermal thickness of the skin involves increasing the epidermal thickness of the skin (e.g., relative to the skin prior to application of the compositions, formulations, and/or personal care products provided herein (e.g., containing a non-naturally occurring polypeptide of the disclosure). In some cases, the epidermal thickness of the skin, after application of the compositions, formulations, and/or personal care products provided herein (e.g., containing a non-naturally occurring polypeptide of the disclosure) is increased (e.g., relative to the skin prior to the application) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, as determined by reflectance confocal microscopy. In some cases, reflectance confocal microscopy is performed by a Vivascope®.

In various aspects, the compositions, formulations, and/or personal care products provided herein (e.g., containing a non-naturally occurring polypeptide of the disclosure) increase keratinocyte growth (e.g., proliferation) after application to the skin of an individual. In some cases, keratinocyte growth (e.g., proliferation) is increased by at least about 10%, at least about 20%, at least about 30%, at least about 40%, at least about 50%, at least about 60%, at least about 70%, at least about 80%, at least about 90%, at least about 100%, or greater, after the composition, formulation, and/or personal care product (e.g., as disclosed herein; e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase keratinocyte regeneration after application to the skin of an individual. In some cases, keratinocyte regeneration is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase keratinocyte regeneration after application to the skin of an individual. In some cases, keratinocyte regeneration is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase collagen production by fibroblasts after application to the skin of an individual. In some cases, collagen production by fibroblasts is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase fibroblast migration after application to the skin of an individual. In some cases, fibroblast migration is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase fibroblast proliferation after application to the skin of an individual. In some cases, fibroblast proliferation is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase fibroblast adhesion after application to the skin of an individual. In some cases, fibroblast adhesion is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In various aspects, the compositions, formulations, and/or personal care products provided herein (e.g., containing a non-naturally occurring polypeptide of the disclosure) increase keratinocyte viability after exposure to urban dust (e.g., when the compositions, formulations, and/or personal care products are applied to the skin prior to urban dust exposure). In some cases, keratinocyte viability after exposure to urban dust is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In some embodiments, the compositions, formulations, and/or personal care products disclosed herein (e.g., containing a non-naturally occurring polypeptide of the disclosure) have anti-oxidative capacity. In some embodiments, the anti-oxidative capacity may be measured using an oxygen radical absorbance capacity (ORAC) assay. In some embodiments, the antioxidative capacity may be measured in Trolox equivalent units. In some embodiments, the antioxidative capacity of the composition may be at least about 50 μM, 100 μM, 150 μM, 200 μM, 250 μM, or more than 250 μM Trolox equivalent units.

In various aspects, the compositions, formulations, and/or personal care products provided herein (e.g., containing a non-naturally occurring polypeptide of the disclosure) increase expression of one or more genes (e.g., one or more genes involved in cell proliferation, cell migration, cell adhesion, etc.) (by a cell present in the skin, e.g., keratinocytes, fibroblasts) after application to the skin of an individual. In some cases, expression of one or more genes (e.g., by a cell present in the skin, e.g., fibroblast, keratinocyte) is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product (e.g., as disclosed herein, e.g., containing a non-naturally occurring polypeptide of the disclosure) is applied to the skin of an individual (e.g., as compared to the skin prior to application of the composition, formulation, and/or personal care product).

In some embodiments, the one or more genes are involved in a signaling pathway (e.g., involved in cell proliferation, cell migration, cell adhesion). In some cases, the one or more genes are involved in a VEGFA/VEGFR2 signaling pathway. In some cases, the one or more genes involved in a VEGFA/VEGFR2 signaling pathway is selected from the group consisting of: MYOC1, FLII, ROCK1, ROCK2, CLTC, LIMK 1, EGR1, and any combination thereof.

In some cases, the one or more genes are involved in a focal adhesion signaling pathway. In some cases, the one or more genes involved in a focal adhesion signaling pathway is selected from the group consisting of: ITGA3, TNC, LAMC1, FLNA, TLN1, ZYX, DIAPH1, and any combination thereof.

In some cases, the one or more genes are involved in an endothelin signaling pathway. In some cases, the one or more genes involved in an endothelin signaling pathway is selected from the group consisting of: TRIOBP, WNK1, MMP2, VCAN, ACTA2, GNA12, EGR1, and any combination thereof.

In some cases, the one or more genes are involved in an EGF/EGFR signaling pathway. In some cases, the one or more genes involved in an EGF/EGFR signaling pathway is selected from the group consisting of: ATXN2, JAK1, RPS6KA2, ROCK1, SHC1, IQGAP1, PLCG1, and any combination thereof.

In some cases, the one or more genes are involved in a transforming growth factor-beta (TGF-beta) signaling pathway. In some cases, the one or more genes involved in a TGF-beta signaling pathway is selected from the group consisting of: SMURF1, SPTBN1, PAK2, ROCK1, SHC1, TGFBR3, TGFBR1, and any combination thereof.

### Uses of Recombinant Polypeptides as Hair and Scalp Compositions and Formulations

In various aspects, the methods provided herein comprise applying the compositions, formulations, and/or personal care products (e.g., containing a non-naturally occurring polypeptide of the disclosure) to the scalp or hair of an individual. The application can result in an increase in the shininess of the hair, increased hair strength, increased combability, increased hydration upon a heat treatment (such as hair curling irons or straightening irons, blow dryers, etc.), increased hair strength, and increased hair diameter.

Hair fiber is composed by three main structures: cuticle, cortex and medulla. Physical properties of hair that can be measured include elasticity, smoothness, volume, shine, and softness due to both the significant adherence of the cuticle scales and the movement control (malleability), as well as the easiness of combing (“combability”), since they reduce the fibers static electricity.

In some embodiments, compositions containing the non-naturally occurring polypeptides of the disclosure are useful as personal care products that are hair care products. Several assays can be used to determine whether such compositions are beneficial to the hair. Exemplary hair characteristics that can be measured after treatment with such compositions include, for example, the health, shininess, combability, detangling, straightening ability, and the diameter of an individual hair.

In various aspects, the compositions, formulations, and/or personal care products provided herein (e.g., containing recombinant polypeptides of the disclosure) can increase the combability of hair. In some cases, the combability of the hair is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product is applied to the hair of an individual (e.g., as compared to prior to application of the composition, formulation, and/or personal care product).

In various aspects, the compositions, formulations, and/or personal care products provided herein (e.g., containing recombinant polypeptides of the disclosure) can increase the shininess of hair. In some embodiments, the shininess of the hair, as measured by a glossmeter, is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product is applied to the hair of an individual (e.g., as compared to prior to application of the composition, formulation, and/or personal care product).

In various aspects, the compositions, formulations, and/or personal care products provided herein (e.g., containing recombinant polypeptides of the disclosure) can increase the elasticity of hair. In some embodiments, the elasticity of the hair is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product is applied to the hair of an individual (e.g., as compared to prior to application of the composition, formulation, and/or personal care product).

In various aspects, the compositions, formulations, and/or personal care products provided herein (e.g., containing recombinant polypeptides of the disclosure) can increase the strength of hair. In some embodiments, the strength of the hair is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%, after the composition, formulation, and/or personal care product is applied to the hair of an individual (e.g., as compared to prior to application of the composition, formulation, and/or personal care product).

In various aspects, the compositions, formulations, and/or personal care products provided herein (e.g., containing recombinant polypeptides of the disclosure) can penetrate the hair cortex by the first application. In some embodiments, increasing application frequency or duration can further increase penetration of the compositions, formulations, and/or personal care products provided herein (e.g., containing recombinant polypeptides of the disclosure). In various aspects, the compositions, formulations, and/or personal care products provided herein (e.g., containing recombinant polypeptides of the disclosure) can resist multiple shampoo cycles after application to hair. In various aspects, the compositions, formulations, and/or personal care products provided herein (e.g., containing recombinant polypeptides of the disclosure) can confer thermal protection to hair (e.g., enhanced hair thermal protection in response to heat damage). In various aspects, the compositions, formulations, and/or personal care products provided herein (e.g., containing recombinant polypeptides of the disclosure) can modulate hair suppleness after application to hair. In various aspects, the compositions, formulations, and/or personal care products provided herein (e.g., containing recombinant polypeptides of the disclosure) can improve hair style retention after application to hair. In various aspects, the compositions, formulations, and/or personal care products provided herein (e.g., containing recombinant polypeptides of the disclosure) can improve frizz control after application to hair. In various aspects, the compositions, formulations, and/or personal care products provided herein (e.g., containing recombinant polypeptides of the disclosure) can modulate hair combing performance (i.e., the force required to run a comb through hair) after application to hair. In various aspects, the compositions, formulations, and/or personal care products provided herein (e.g., containing recombinant polypeptides of the disclosure) can provide antioxidant benefits. In some instances, the antioxidant benefits can decrease keratin denaturation by protecting the structural proteins of hair against oxidative stress. In various aspects, the compositions, formulations, and/or personal care products provided herein (e.g., containing recombinant polypeptides of the disclosure) can improve the viability of cell populations contributing to hair and scalp health (e.g., fibroblasts and/or keratinocytes). In various aspects, the compositions, formulations, and/or personal care products provided herein (e.g., containing recombinant polypeptides of the disclosure) can modulate hair suppleness after application to hair. In various aspects, the compositions, formulations, and/or personal care products provided herein (e.g., containing recombinant polypeptides of the disclosure) can provide wound healing benefits that can be beneficial for applications in beauty, personal care, and biomedicine.

The evaluation of the effects of an application to hair can be carried out by several methods. Among these are optical and electron microscopy, confocal microscopy, mechanical resistance measuring, shine evaluation and optical coherence tomography (OCT). Other microscopy methods for measuring characteristics of treated hair include SEM photomicrography, which is useful for evaluating hair surface morphology, and atomic force microscopy (AFM), which uses a probe to generate an image that can be quantitated. Optical coherence tomography can also be used to examine hair. Mechanical assays that can be used to measure the effect of a treatment on hair strength include, for example, a dynamometer to measure the rupture load of a hair thread. This system is also used to evaluate elasticity, combability, and detangling. Subjective tests can also be performed. These tests can be performed by trained volunteers (to simulate the end user), or by specialized technicians.

A Method of Purifying Recombinant Polypeptides from Host Cells

A method of purifying a recombinant polypeptide from a plurality of host cells is described herein. The method of purifying a recombinant polypeptide from a plurality of host cells comprises (a) providing or obtaining a mixture comprising the recombinant polypeptide and host cells or host cell lysate; (b) separating the mixture into a first soluble fraction comprising the recombinant polypeptide and a first insoluble fraction; (c) adjusting the pH of the first soluble fraction to a pH of 5 or less to generate a pH-adjusted mixture; and (d) separating the pH-adjusted mixture into a second soluble fraction comprising the recombinant polypeptide and a second insoluble fraction, wherein the recombinant polypeptide comprises at least 50% of protein in the second soluble fraction.

In some aspects, the host cell can be a microbial cell. In some aspects, the microbial cell can be a bacterial cell. In some aspects, the bacterial cell can be a gram-negative bacterium. In some aspects, the gram-negative bacterium can be E. coli. In some aspects, the separating of the mixture in step (c) can comprise centrifugation, ultracentrifugation, filtration, ultrafiltration, or a combination thereof. Alternative methods that can separate mixtures into soluble and insoluble fractions can also be adopted.

In some aspects, the recombinant polypeptide can comprise a solubility-enhancing amino acid sequence. In some aspects, the recombinant polypeptide does not comprise a solubility-enhancing amino acid sequence. In some aspects, the recombinant polypeptide can be a pH-stable protein. In some aspects, the recombinant polypeptide can be expressed intracellularly. In some aspects, the recombinant polypeptide can be expressed and directed to the periplasm. In some aspects, the recombinant polypeptide can be expressed extracellularly.

In some aspects, the adjusting of the pH in step (c) can comprise adding an acid to the first soluble fraction. In some aspects, the acid can be a weak acid or a strong acid. In some aspects, the acid can be H₂SO₄. In some aspects, the adjusting of the pH in step (c) can comprise adjusting the pH of the first soluble fraction to a pH of 4 or less. In some aspects, the adjusting of the pH in step (c) can comprise adjusting the pH of the first soluble fraction to a pH of 3 or less.

In some aspects, the method of purifying a recombinant polypeptide from a plurality of host cells can further comprise a step (e) for adjusting the pH of the second soluble fraction to a pH that is lower than the pH of (c) to generate a second pH-adjusted mixture; and (f) for separating the second pH-adjusted mixture into a third soluble fraction comprising the recombinant polypeptide and a third insoluble fraction. In some aspects, the method can further comprise a step (g) for adjusting the pH of the third soluble fraction to a pH that is lower than the pH of (e) to generate a third pH-adjusted mixture; and (h) for separating the third pH-adjusted mixture into a fourth soluble fraction comprising the recombinant polypeptide and a fourth insoluble fraction.

In some aspects, a purity of the recombinant polypeptide is higher in the third soluble fraction than in the second soluble fraction; and wherein a purity of the recombinant polypeptide is higher in the fourth soluble fraction than in the third soluble fraction. In some aspects, the recombinant polypeptide, or portions thereof, is at least 55% of protein present in the second soluble fraction. In some aspects, the recombinant polypeptide, or portions thereof, is at least 60% of protein present in the second soluble fraction. In some aspects, the recombinant polypeptide, or portions thereof, is at least 65% of protein present in the second soluble fraction. In some aspects, the recombinant polypeptide, or portions thereof, is at least 70% of protein present in the second soluble fraction. In some aspects, the recombinant polypeptide, or portions thereof, is at least 75% of protein present in the second soluble fraction. In some aspects, the recombinant polypeptide, or portions thereof, is at least 80% of protein present in the second soluble fraction. In some aspects, the recombinant polypeptide, or portions thereof, is at least 85% of protein present in the second soluble fraction. In some aspects, the recombinant polypeptide, or portions thereof, is at least 90% of protein present in the second soluble fraction. In some aspects, the recombinant polypeptide, or portions thereof, is at least 94% of protein present in the second soluble fraction. In some aspects, the recombinant polypeptide, or portions thereof, is at least 95% of protein present in the second soluble fraction. In some aspects, the recombinant polypeptide, or portions thereof, is at least 96% of protein present in the second soluble fraction. In some aspects, the recombinant polypeptide, or portions thereof, is at least 97% of protein present in the second soluble fraction. In some aspects, the recombinant polypeptide, or portions thereof, is at least 98% of protein present in the second soluble fraction. In some aspects, the recombinant polypeptide, or portions thereof, is at least 99% of protein present in the second soluble fraction. In some aspects, the final product comprising the recombinant polypeptide is devoid of host cell debris. In some aspects, the final product comprising the recombinant polypeptide comprises at most 50% endogenous host cell protein relative to total protein. In some aspects, the final product comprising the recombinant polypeptide comprises at most 45% endogenous host cell protein relative to total protein. In some aspects, the final product comprising the recombinant polypeptide comprises at most 40% endogenous host cell protein relative to total protein. In some aspects, the final product comprising the recombinant polypeptide comprises at most 35% endogenous host cell protein relative to total protein. In some aspects, the final product comprising the recombinant polypeptide comprises at most 30% endogenous host cell protein relative to total protein. In some aspects, the final product comprising the recombinant polypeptide comprises at most 25% endogenous host cell protein relative to total protein. In some aspects, the final product comprising the recombinant polypeptide comprises at most 20% endogenous host cell protein relative to total protein. In some aspects, the final product comprising the recombinant polypeptide comprises at most 15% endogenous host cell protein relative to total protein. In some aspects, the final product comprising the recombinant polypeptide comprises at most 10% endogenous host cell protein relative to total protein. In some aspects, the final product comprising the recombinant polypeptide comprises at most 6% endogenous host cell protein relative to total protein. In some aspects, the final product comprising the recombinant polypeptide comprises at most 5% endogenous host cell protein relative to total protein. In some aspects, the final product comprising the recombinant polypeptide comprises at most 4% endogenous host cell protein relative to total protein. In some aspects, the final product comprising the recombinant polypeptide comprises at most 3% endogenous host cell protein relative to total protein. In some aspects, the final product comprising the recombinant polypeptide comprises at most 2% endogenous host cell protein relative to total protein. In some aspects, the final product comprising the recombinant polypeptide comprises at most 1% endogenous host cell protein relative to total protein. In some aspects, the final product comprising the recombinant polypeptide is devoid of endogenous host cell protein.

### EXAMPLES

### Example 1. Production of a Variety of Non-Naturally Occurring Keratin Polypeptides

This example shows the recombinant expression and purification of various recombinant keratin polypeptides. The desired polynucleotide sequences of human collagen type I, alpha 1, keratin 12, keratin 31, and keratin 33A were synthesized by IDT. Overlaps between a pET vector and the insert polynucleotide sequence encoding the recombinant keratin polypeptides were designed to be between 20 and 30 bp long and added using PCR with the enzyme PrimeStar® GXL polymerase (available at takarabio.com/products/pcr/gc-rich-pcr/primestar-gxl-dna-polymerase). The opened pETno vector and insert DNA were assembled together into the final plasmid using In-Fusion Cloning (available at takarabio.com/products/cloning/in-fusion-cloning) according to manufacturer's instructions. In all cases, coding sequences were preceded by a secretion signal sequence, the nucleotide sequence encoding a DegP secretion sequence from Rosenbergiella nectarea below:

(SEQ ID NO: 42)
ATGAAAAAAAACATCCTGTCTCTGTCTATGGTTGCTCTGTCTCTGTCTCT
GGCTCTGGGTTCTGTTTCTGTTACCGCT

Plasmid sequences were verified through Sanger sequencing. E. coli host cells were transformed with final plasmids and subsequently cultivated in vegan Luria Bertani (LB) broth and frozen in 1.5 milliliter aliquots with a solution of 50% vegetable glycerin diluted in water at a ratio of 50:50 of liquid culture to glycerin solution. One vial of this frozen culture was revived in 50 ml of minimal media overnight at 37° C., 200 rpm. Cells were then transferred into 300 ml of minimal media and grown for 6-9 hours to reach an OD600 of 3-10.

### Minimal Media Formulation:

1) Autoclave 5 L of 550 g/kg Glucose syrup at concentration in DI water (VWR, product #97061-170).
2) Autoclave in 3946 mL of DI water:
20 g (NH₄)₂HPO₄ (VWR, product #97061-932).
66.5 g KH₂PO₄ (VWR, product #97062-348).
22.5 g H₃C₆H₅O₇(VWR, product #BDH9228-2.5KG).
8.85 g MgSO4·7H2O (VWR, product #97062-134).
10 mL of 1000× Trace metals formulation (formulated by Geltor).

After autoclaving, add
118 g of (1) to (2)
5 mL of 25 mg/mL Kanamycin Sulfate (VWR-V0408)
Use 28% NH₄OH (VWR, product #BDH3022) to adjust pH to 6.1.

### Trace Metals Formulation:

Ferrous Sulfate Heptahydrate, 27.8 g/L (Spectrum, 7782-63-0)
Zinc Sulfate heptahydrate, 2.88 g/L (Spectrum, 7446-20-0)
Calcium chloride dihydrate, 2.94 g/L (Spectrum, 2971347)
Sodium molybdate dihydrate, 0.48 g/L (Spectrum, 10102-40-6)
Manganese chloride tetrahydrate, 1.26 g/L (Spectrum, 13446-34-9)
Sodium selenite, 0.35 g/L (Spectrum, 10102-18-8)
Boric acid, 0.12 g/L (Spectrum, 10043-35-3)

The minimal medium for culturing the transformed host cells was prepared according to the following method. 5 L of 550 g/kg glucose syrup was autoclaved. Separately, the following ingredients were mixed and autoclaved: 3946 mL of DI water, 20 g (NH₄)₂HPO₄; 66.5 g KH₂PO₄; 22.5 g H₃C₆H₅O₇; and 8.85 g MgSO4·7H2O. After autoclaving, 118 g of the glucose syrup was added to the autoclaved nutrient mixture. 10 ml of a 1000× Trace metals formulation was added. Next, 5 mL of 25 mg/mL kanamycin sulfate was added. The medium was then adjusted to pH to 6.1 using 28% NH₄OH.

The fermentations were performed at a temperature of 28° C. Induction was carried out by adding IPTG to the media at concentrations ranging from 0.1-0.5 mM. Fermentations were continued for 24-60 hours. The recombinant polypeptides were purified as follows; the pH of the fermentation broth was decreased to between 3-3.5 using 5-50% sulfuric acid. The cells were then separated using centrifugation. Supernatant of the acidified broth was run on an SDS-PAGE gel and found to contain the recombinant proteins in relatively high abundance compared to starting pellet.

Several recombinant polypeptides containing both the solubility-enhancing amino acid sequence together with the target keratin polypeptide sequence, as well as keratin polypeptides alone (as controls) were designed, transformed to a bacterial strain, produced, and purified.

The nucleotide sequence encoding a solubility-enhancing amino acid sequence derived from truncated human collagen type I, alpha 1 sequence fused to a truncated human keratin type 12 sequence (strain 8737) is shown below:

(SEQ ID NO: 75)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCTGCAGATTGATAATGCACGTCTG
GCAGCCGAAGATTTTCGTATGAAATATGAAAATGAACTGGCACTG
CGTCAGGGTGTTGAAGCAGATATTAATGGTCTGCGTCGTGTTCTG
GATGAACTGACCCTGACACGTACAGATCTGGAAATGCAGATCGAA
AGCCTGAATGAAGAACTGGCCTACATGAAAAAAAACCACGAAGAT
GAACTGCAGAGCTTTCGTGTTGGTGGTCCGGGTGAAGTTAGCGTT
GAAATGGATGCAGCACCGGGTGTTGATCTGACCCGTCTGCTGAAT
GATATGCGTGCACAGTATGAAACAATTGCCGAACAGAATCGTAAA
GATGCAGAAGCCTGGTTTATTGAAAAAAGCGGTGAACTGCGTAAA
GAGATTAGCACCAATACAGAACAGCTGCAGAGCAGCAAAAGCGAA
GTTACCGATCTGCGTCGCGCATTTCAGAACCTGGAAATCGAACTG
CAGTCACAGCTGGCAATGAAAAAGAGCCTGGAAGATAGCCTGGCC
GAAGCCGAAGGTGATTAT.

The amino acid sequence corresponding to the above nucleic acid sequence is:

(SEQ ID NO: 76)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGLQIDNARL
AAEDFRMKYENELALRQGVEADINGLRRVLDELTLTRTDLEMQIE
SLNEELAYMKKNHEDELQSFRVGGPGEVSVEMDAAPGVDLTRLLN
DMRAQYETIAEQNRKDAEAWFIEKSGELRKEISTNTEQLQSSKSE
VTDLRRAFQNLEIELQSQLAMKKSLEDSLAEAEGDY.

The nucleotide sequence encoding a solubility-enhancing sequence amino acid derived from truncated human collagen type I, alpha sequence fused to a truncated human keratin type 33A sequence (strain 8739) is shown below:

(SEQ ID NO: 77)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCAGCTGGGTGATCGCCTGAATGTT
GAAGTTGATGCAGCACCGACCGTTGATCTGAATCAGGTGCTGAAT
GAAACCCGTAGCCAGTATGAAGCCCTGGTGGAAACCAATCGTCGT
GAAGTTGAACAGTGGTTTGCAACCCAGACCGAAGAACTGAATAAA
CAGGTTGTTAGCAGCAGCGAACAGCTGCAGAGCTATCAGGCAGAA
ATCATTGAACTGCGTCGCACCGTTAATGCACTGGAAATCGAACTG
CAGGCACAGCATAATCTGCGTGATAGCCTGGAAAATACCCTGACC
GAAAGCGAAGCACGTTATAGCAGCCAGCTGAGCCAGGTTCAGCGT
CTGATTACCAATGTTGAAAGTCAGCTGGCAGAAATTCGTTCTGAT
CTGGAACGTCAGAATCAAGAATATCAGGTTCTGCTGGATGTTCGT
GCCCGTCTGGAA.

The amino acid sequence corresponding to the above nucleic acid sequence is:

(SEQ ID NO: 78)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNQVLNETRSQYEALVETNRREVEQWFATQTEELNK
QVVSSSEQLQSYQAEIIELRRTVNALEIELQAQHNLRDSLENTLT
ESEARYSSQLSQVQRLITNVESQLAEIRSDLERQNQEYQVLLDVR
ARLE.

The nucleotide sequence encoding a solubility-enhancing amino acid sequence derived from truncated human collagen type 1, alpha sequence fused to a truncated human keratin type 31 sequence (strain 8738) is shown below:

(SEQ ID NO: 79)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCAGCTGGGTGATCGCCTGAATGTT
GAAGTTGATGCAGCACCGACCGTTGATCTGAATCGTGTGCTGAAT
GAAACCCGTAGCCAGTATGAAGCCCTGGTGGAAACCAATCGTCGT
GAAGTTGAACAGTGGTTTACCACACAGACCGAAGAACTGAATAAA
CAGGTTGTTAGCAGCAGCGAACAGCTGCAGAGTTATCAGGCAGAA
ATCATTGAACTGCGTCGCACCGTTAATGCACTGGAAATCGAACTG
CAGGCACAGCATAATCTGCGTGATAGCCTGGAAAATACCCTGACC
GAAAGCGAAGCACGTTATAGCAGCCAGCTGAGCCAGGTTCAGAGC
CTGATTACCAATGTTGAAAGTCAGCTGGCAGAAATTCGTAGTGAT
CTGGAACGTCAGAATCAAGAATATCAGGTTCTGCTGGATGTTCGT
GCCCGTCTGGAA.

The amino acid sequence corresponding to the above nucleic acid sequence is:

(SEQ ID NO: 80)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEELNK
QVVSSSEQLQSYQAEIIELRRTVNALEIELQAQHNLRDSLENTLT
ESEARYSSQLSQVQSLITNVESQLAEIRSDLERQNQEYQVLLDVR
ARLE.

Additional solubility-enhancing amino acid sequence-keratin polypeptide sequences are indicated in the examples below.

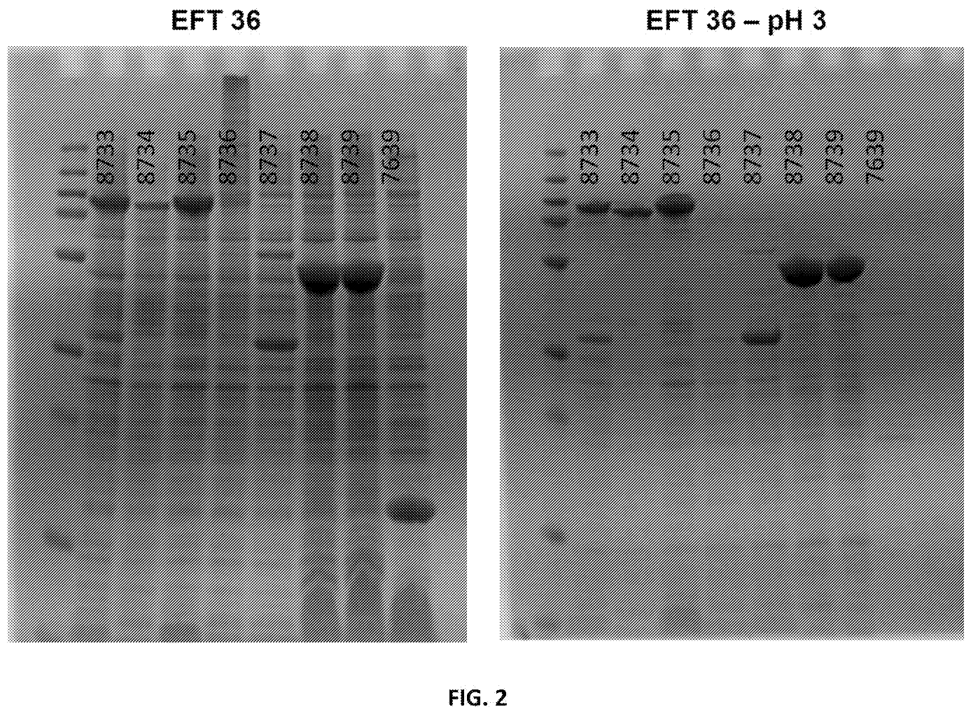

The presence of each of the polypeptides in the culture medium was confirmed using SDS-PAGE followed by protein staining with Coomassie blue, showing a band at the predicted size range for each of the expressed constructs. After the pH treatment, the samples were centrifuged. The soluble fractions were loaded onto the SDS-PAGE gel. Samples were taken at either the end of fermentation (“EFT 36”), or after pH adjustment to pH 3 (“EFT 36-pH 3”). A thick and clear band was observed at the expected sizes for each of the respective polypeptides (as shown in FIG. 2). The left gel shows the proteins present at the end of fermentation, while the right gel shows the proteins present in the supernatant at pH 3.0. As seen in FIG. 2, the addition of the solubility-enhancing amino acid sequence to various human keratin polypeptides improved solubility and purification at pH 3 as compared to expression and purification of the human keratin polypeptide alone (7639); human keratin type 12 polypeptide (8737), human keratin type 31 polypeptide (8738), and human keratin type 33a polypeptide (8739).

In addition, the addition of two solubility-enhancing amino acid sequences on both sides of various human keratin polypeptides (e.g., as a “sandwich” construct; or “C—K—C”) also improved solubility and purification at pH 3; human keratin type 12 polypeptide sandwich (8733), and human keratin type 33a polypeptide sandwich (8734).

The amino acid sequence of the C—K—C sandwich (strain 8733) (human collagen type I, alpha 1 truncate (262 aa)-human keratin type 12 truncate-(179 aa)-human collagen type I, alpha 1 truncate (219 aa)) is shown below:

(SEQ ID NO: 81)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGLQIDNARL
AAEDFRMKYENELALRQGVEADINGLRRVLDELTLTRTDLEMQIE
SLNEELAYMKKNHEDELQSFRVGGPGEVSVEMDAAPGVDLTRLLN
DMRAQYETIAEQNRKDAEAWFIEKSGELRKEISTNTEQLQSSKSE
VTDLRRAFQNLEIELQSQLAMKKSLEDSLAEAEGDYGVPGDLGAP
GPSGARGERGFPGERGVQGPPGPAGPRGANGAPGNDGAKGDAGAP
GAPGSQGAPGLQGMPGERGAAGLPGPKGDRGDAGPKGADGSPGKD
GVRGLTGPIGPPGPAGAPGDKGESGPSGPAGPTGARGAPGDRGEP
GPPGPAGFAGPPGADGQPGAKGEPGDAGAKGDAGPPGPAGPAGPP
GPIGNVGAPGAKGARGSAGPPGATGFPGAAGRVGPPGPSGNAGPP
GPPGPAGKEGGKGPRGETGPAGRPGEVG.

The amino acid sequence of the C—K—C sandwich using a different keratin truncate (strain 8734) (human collagen type I, alpha 1 truncate (262 aa)-human keratin type 33a truncate-(147 aa)-human collagen type 1, alpha 1 truncate (219 aa)) is shown below:

(SEQ ID NO: 82)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNQVLNETRSQYEALVETNRREVEQWFATQTEELNK
QVVSSSEQLQSYQAEIIELRRTVNALEIELQAQHNLRDSLENTLT
ESEARYSSQLSQVQRLITNVESQLAEIRSDLERQNQEYQVLLDVR
ARLEGVPGDLGAPGPSGARGERGFPGERGVQGPPGPAGPRGANGA
PGNDGAKGDAGAPGAPGSQGAPGLQGMPGERGAAGLPGPKGDRGD
AGPKGADGSPGKDGVRGLTGPIGPPGPAGAPGDKGESGPSGPAGP
TGARGAPGDRGEPGPPGPAGFAGPPGADGQPGAKGEPGDAGAKGD
AGPPGPAGPAGPPGPIGNVGAPGAKGARGSAGPPGATGFPGAAGR
VGPPGPSGNAGPPGPPGPAGKEGGKGPRGETGPAGRPGEVG.

TABLE 6
Production of solubility-enhancing amino acid sequences fused to various keratins

| | | Protein |
|---|---|---|
| | | SEQ ID |
| Strain | Description | NO: |
| 8733 | solubility-enhancing amino acid sequence derived from human | |
| | collagen type I, alpha 1 truncate (262 aa)-human keratin type 12 | |
| | truncate-(179 aa) -human collagen type I, alpha 1 truncate (219 aa) | |
| 8734 | solubility-enhancing amino acid sequence derived from human | 82 |
| | collagen type I, alpha 1 truncate (262 aa)-human keratin type 33a | |
| | truncate-(147 aa)-human collagen type I, alpha 1 truncate (219 aa) | |
| 8737 | solubility-enhancing amino acid sequence derived from human | 76 |
| | collagen type I, alpha 1 truncate (262 aa)-human keratin type 12 | |
| | truncate-(179 aa) | |
| 8738 | solubility-enhancing amino acid sequence derived from human | 80 |
| | collagen type I, alpha 1 truncate (262 aa)-human keratin type 31 | |
| | truncate-(147 aa) | |
| 8739 | solubility-enhancing amino acid sequence derived from human | 78 |
| | collagen type I, alpha 1 truncate (262 aa)-human keratin type 33a | |
| | truncate-(147 aa) | |
| 7639 | Human keratin type 33a truncate (147 aa) control | 12 |

### Example 2. Solubility of Keratin Polypeptides is Enhanced Through Use of the Solubility-Enhancing Amino Acid Sequences of the Present Disclosure

To demonstrate that the use of the solubility-enhancing amino acid sequences of the present disclosure can increase the solubility of keratin polypeptides, experiments were performed with either a keratin polypeptide alone, or combined with a collagen-derived solubility-enhancing amino acid sequence as fusion proteins. Previous work showed that a variety of recombinant collagen polypeptides were soluble at a low pH, and could be separated from other polypeptides and contaminants in culture media by lowering the pH of the culture medium to about 3.0, then centrifuging the medium. The non-recombinant polypeptides and other contaminants precipitated and became part of the pellet fraction, while the recombinant collagen remained soluble in the supernatant fraction. This property was then used to impart solubility characteristics to a variety of keratin polypeptides that were otherwise not readily expressed or purified recombinantly.

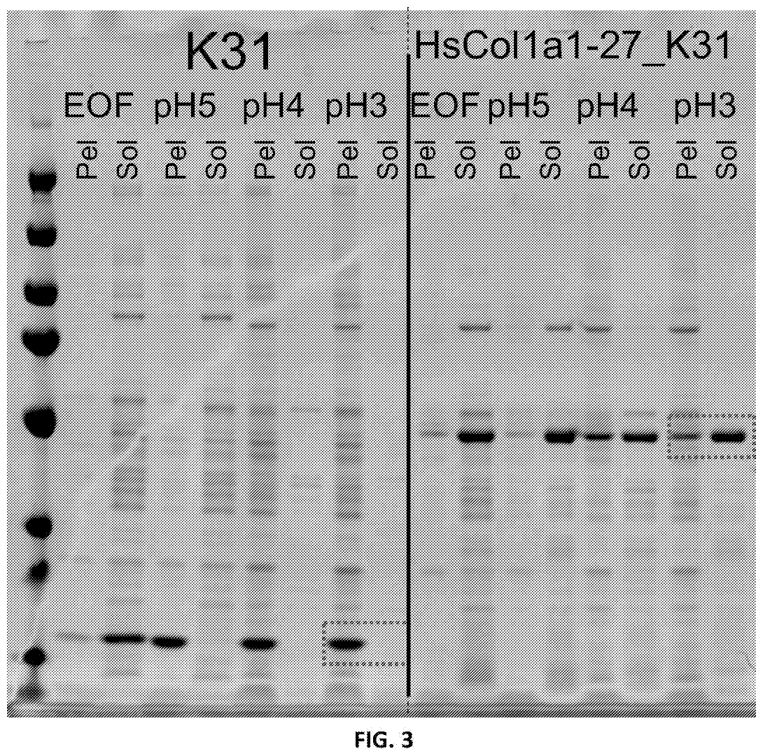

To determine whether combining a solubility-enhancing polypeptide with a keratin polypeptide would allow it to remain more soluble at a low pH (to allow for rapid purification procedures), a human keratin type 31 polypeptide (“K31”) (SEQ ID NO: 7) was expressed by itself (left panel of FIG. 3) or was recombinantly fused to a type I, alpha 1 human collagen fragment of 262 amino acids (“HsCol1a1-27”; SEQ ID NO: 26) to result in a fusion protein having the sequence of SEQ ID NO: 80 (right panel of FIG. 3).

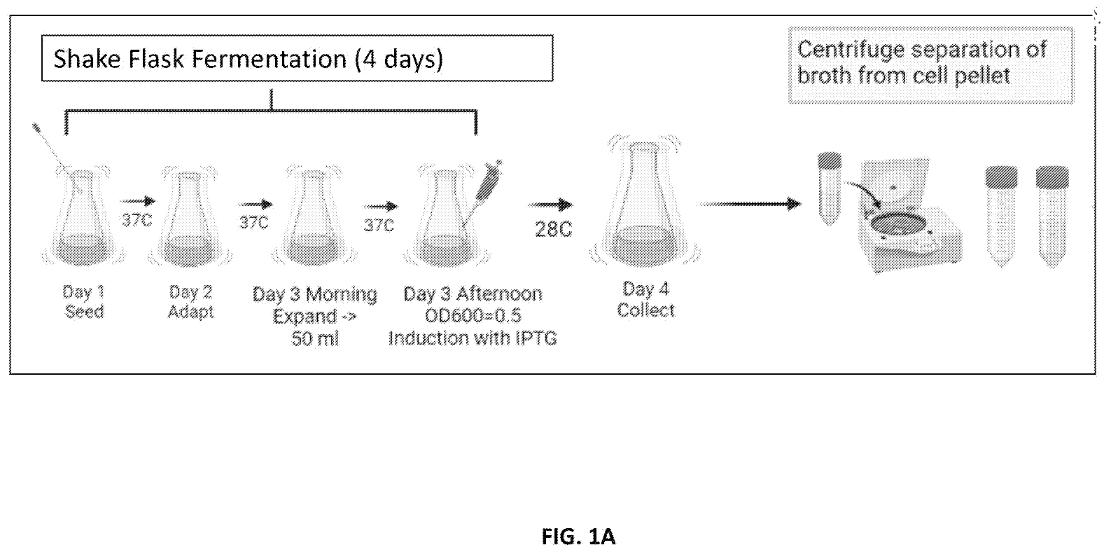

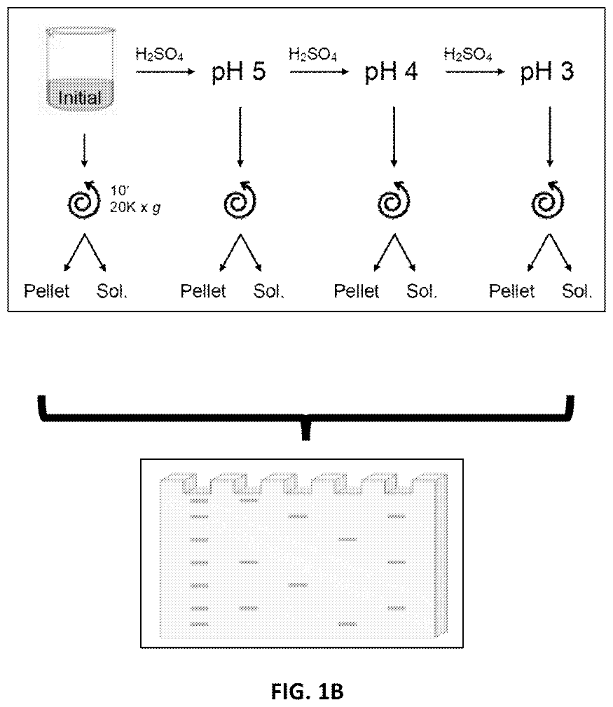

A 147 amino acid truncated human keratin polypeptide sequence (human keratin type 31; SEQ ID NO: 7) was produced in E. coli following the method of Example 1. The culture extract was subjected to either pH 5, pH 4, or pH 3 by the addition of H₂SO₄, following the method diagrammed in FIG. 1A and FIG. 1B. The extract was then centrifuged for 10 minutes at 20,000×g. Both the pellet (“Pel” insoluble) and the supernatant (“Sol” soluble) were examined by SDS-PAGE (FIG. 3).

When the keratin polypeptide was expressed by itself (left panel of FIG. 3) the majority of the target keratin polypeptide was present in the supernatant fraction (indicated as Sol) at the end of the fermentation but was subsequently found in the pellet fraction after acidification (indicated as Pel). In contrast, when the keratin fragment was fused to the solubility-enhancing amino acid sequence (right panel of FIG. 3), the fusion protein largely remained in the supernatant fraction while most contaminating proteins were located in the pellet fraction. The bands in the boxes show a comparison of the two conditions for the pH 3 samples. The increased solubility at a low pH allowed the keratin polypeptides to be easily separated from the contaminating polypeptides. This was also seen with combinations of the collagen-derived solubility-enhancing amino acid sequence with human keratin type 12 and human keratin type 33A as shown in Example 1 above.

This indicates that solubility-enhancing amino acid sequences of the present disclosure are able to endow a variety of recombinant keratin polypeptides with solubility, enabling simple and inexpensive purification of desired keratin polypeptides.

### Example 3. Various Types of Solubility-Enhancing Amino Acid Sequences Enhance Solubility of Keratin Polypeptides

In addition to the polypeptides shown in the above Examples, other types of solubility-enhancing amino acids derived from other collagen polypeptides were also found to confer improved solubility of the keratin polypeptides.

A 188 amino acid collagen type 21 fragment from Gallus (chicken) (GL21) (SEQ ID NO: 19) was fused to a 147 amino acid human keratin type 31 polypeptide (SEQ ID NO: 7) as described herein.

The nucleotide sequence encoding a truncated chicken collagen type 21 sequence fused to a truncated human keratin type 31 sequence was:

(SEQ ID NO: 83)
GATACTGGTTTCCCGGGGATGCCTGGGCGCTCAGGTGATCCGGGG
CGTAGTGGAAAAGACGGTCTGCCGGGGTCCCCGGGCTTTAAGGGT
GAGGTGGGTCAGCCCGGTAGTCCAGGTTTAGAAGGTCACCGCGGA
GAGCCCGGGATTCCAGGCATTCCTGGCAACCAGGGTGCCAAGGGA
CAGAAAGGCGAAATTGGTCCGCCCGGCCTACCGGGCGCGAAAGGT
TCTCCTGGTGAAACCGGTCTCATGGGTCCGGAAGGTAGCTTCGGC
CTGCCCGGCGCACCTGGTCCGAAGGGCGATAAGGGGGAGCCTGGG
CTGCAAGGTAAACCGGGTAGTTCTGGCGCCAAAGGTGAACCCGGC
GGTCCCGGTGCGCCAGGGGAACCAGGTTATCCTGGTATTCCTGGA
ACCCAAGGAATTAAAGGTGACAAAGGCTCACAGGGCGAAAGTGGT
ATACAGGGTCGCAAGGGCGAAAAAGGACGTCAGGGCAATCCAGGC
CTGCAGGGTACTGAAGGCCTGCGTGGAGAACAGGGTGAGAAAGGT
GAAAAAGGAGATCCTGGTATTCGCCAGCTGGGTGATCGCCTGAAT
GTTGAAGTTGATGCAGCACCGACCGTTGATCTGAATCGTGTGCTG
AATGAAACCCGTAGCCAGTATGAAGCCCTGGTGGAAACCAATCGT
CGTGAAGTTGAACAGTGGTTTACCACACAGACCGAAGAACTGAAT
AAACAGGTTGTTAGCAGCAGCGAACAGCTGCAGAGTTATCAGGCA
GAAATCATTGAACTGCGTCGCACCGTTAATGCACTGGAAATCGAA
CTGCAGGCACAGCATAATCTGCGTGATAGCCTGGAAAATACCCTG
ACCGAAAGCGAAGCACGTTATAGCAGCCAGCTGAGCCAGGTTCAG
AGCCTGATTACCAATGTTGAAAGTCAGCTGGCAGAAATTCGTAGT
GATCTGGAACGTCAGAATCAAGAATATCAGGTTCTGCTGGATGTT
CGTGCCCGTCTGGAA.

The amino acid sequence corresponding to the above nucleic acid sequence was:

(SEQ ID NO: 84)
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKGDKGSQGESGIQGRKGEKGRQGNPGLQGTEGLRGEQGEKG
EKGDPGIRQLGDRLNVEVDAAPTVDLNRVLNETRSQYEALVETNR
REVEQWFTTQTEELNKQVVSSSEQLQSYQAEIIELRRTVNALEIE
LQAQHNLRDSLENTLTESEARYSSQLSQVQSLITNVESQLAEIRS
DLERQNQEYQVLLDVRARLE.

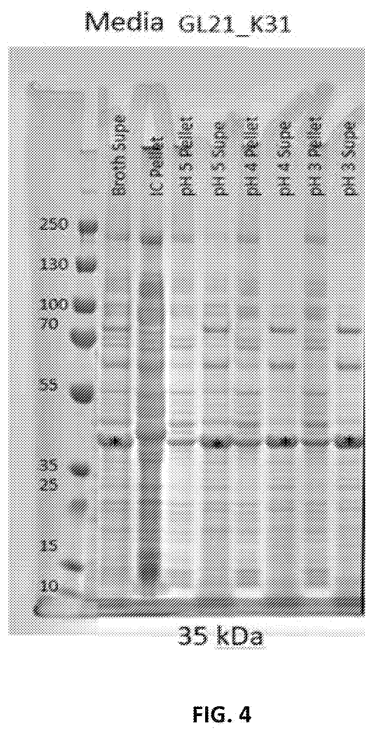

As seen with the human type I, alpha 1 collagen fragment evaluated previously, when the solubility-enhancing amino acid sequence derived from a chicken type 21 collagen fragment (FIG. 4) was expressed together with the keratin polypeptide (“GL21_K31”), the resultant keratin polypeptide remained largely in the supernatant fraction upon acidification, in comparison to the keratin polypeptide alone, which precipitated out of solution and was found in the pellet fraction (left panel of FIG. 3).

As another example, the 147 aa human keratin type 31 polypeptide truncate (SEQ ID NO: 7) was also fused to a 200 amino acid human collagen type III, alpha 1 fragment (SEQ ID NO: 25) as described herein.

The nucleotide sequence encoding a human collagen type III, alpha 1 fragment fused to a truncated human keratin type 31 sequence was:

(SEQ ID NO: 85)
GGAGAACCTGGAGCTAACGGATTACCTGGTGCAGCCGGCGAACGT
GGTGCGCCTGGTTTTCGTGGCCCGGCAGGCCCCAATGGTATCCCA
GGCGAAAAGGGACCGGCAGGTGAGCGTGGAGCACCTGGACCTGCT
GGACCTCGTGGTGCTGCAGGTGAACCCGGTCGTGATGGTGTGCCT
GGTGGGCCCGGTATGCGTGGCATGCCAGGGAGCCCAGGTGGCCCG
GGCTCAGATGGAAAACCGGGCCCACCTGGATCGCAAGGTGAATCT
GGACGTCCAGGTCCGCCCGGTCCTTCGGGTCCCCGTGGCCAACCT
GGTGTAATGGGGTTTCCCGGTCCGAAAGGTAATGATGGCGCGCCT
GGTAAGAATGGAGAACGTGGTGGTCCTGGTGGTCCGGGTCCACAA
GGTCCGCCCGGTAAGAATGGCGAAACAGGTCCACAGGGTCCACCG
GGACCTACAGGCCCGGGTGGTGATAAAGGGGATACAGGCCCGCCT
GGACCTCAGGGGTTGCAAGGACTCCCCGGCACAGGTGGACCGCCA
GGTGAAAATGGCAAACCTGGAGAGCCGGGTCCGAAAGGTGATGCT
GGCGCTCCCGGAGCACAGCTGGGTGATCGCCTGAATGTTGAAGTT
GATGCAGCACCGACCGTTGATCTGAATCGTGTGCTGAATGAAACC
CGTAGCCAGTATGAAGCCCTGGTGGAAACCAATCGTCGTGAAGTT
GAACAGTGGTTTACCACACAGACCGAAGAACTGAATAAACAGGTT
GTTAGCAGCAGCGAACAGCTGCAGAGTTATCAGGCAGAAATCATT
GAACTGCGTCGCACCGTTAATGCACTGGAAATCGAACTGCAGGCA
CAGCATAATCTGCGTGATAGCCTGGAAAATACCCTGACCGAAAGC
GAAGCACGTTATAGCAGCCAGCTGAGCCAGGTTCAGAGCCTGATT
ACCAATGTTGAAAGTCAGCTGGCAGAAATTCGTAGTGATCTGGAA
CGTCAGAATCAAGAATATCAGGTTCTGCTGGATGTTCGTGCCCGT
CTGGAA.

The amino acid sequence corresponding to the above nucleic acid sequence was:

(SEQ ID NO: 86)
GEPGANGLPGAAGERGAPGFRGPAGPNGIPGEKGPAGERGAPGPA
GPRGAAGEPGRDGVPGGPGMRGMPGSPGGPGSDGKPGPPGSQGES
GRPGPPGPSGPRGQPGVMGFPGPKGNDGAPGKNGERGGPGGPGPQ
GPPGKNGETGPQGPPGPTGPGGDKGDTGPPGPQGLQGLPGTGGPP
GENGKPGEPGPKGDAGAPGAQLGDRLNVEVDAAPTVDLNRVLNET
RSQYEALVETNRREVEQWFTTQTEELNKQVVSSSEQLQSYQAEII
ELRRTVNALEIELQAQHNLRDSLENTLTESEARYSSQLSQVQSLI
TNVESQLAEIRSDLERQNQEYQVLLDVRARLE.

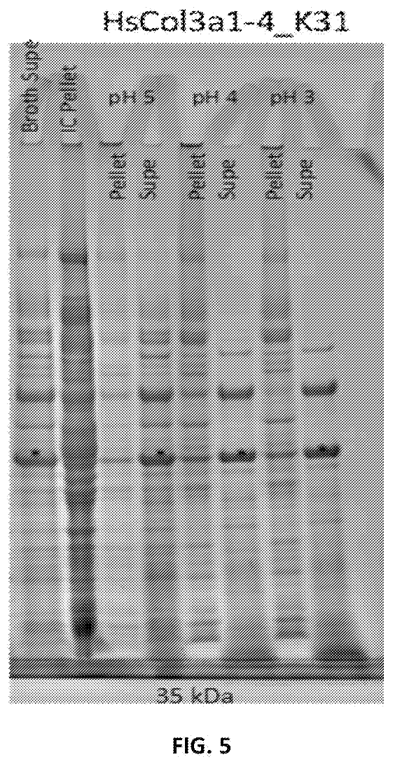

As seen with collagen-derived solubility-enhancing amino acid sequences evaluated previously, when the solubility-enhancing amino acid sequence derived from a human type III, alpha 1 collagen fragment was expressed together with the keratin polypeptide (“HsCol13a1-4_K31”), the resultant keratin polypeptide remained largely in the supernatant fraction (“Supe”) upon acidification (FIG. 5).

This indicates that multiple types of solubility-enhancing amino acid sequences derived from a variety of collagen fragments are able to endow the recombinant keratin polypeptides of the present disclosure with solubility, enabling simple and inexpensive purification of desired keratin polypeptides.

### Example 4. Various Lengths of Solubility-Enhancing Amino Acid Sequences Enhance Solubility of Keratin Polypeptides

To determine whether the length of the solubility-enhancing amino acid sequence was important in imparting solubility to the keratin polypeptide, fragments of various types and lengths of solubility-enhancing amino acid sequences were fused to a human keratin type 31 polypeptide fragment (SEQ ID NO: 7) (herein labeled “K147”). In addition to the various keratin polypeptides using various solubility-enhancing amino acid sequences shown in the above examples, some of the collagen-derived solubility-enhancing tags were also able to increase the solubility of the keratin polypeptides.

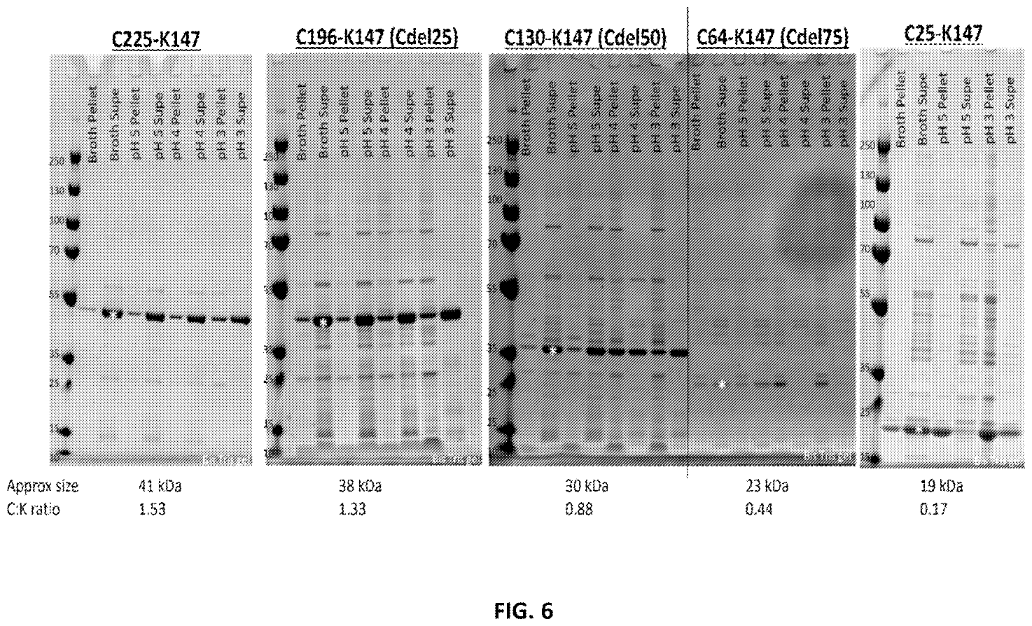

Solubility-enhancing amino acid sequences of 225 amino acids (C225) (SEQ ID NO: 27), 196 amino acids (C196) (SEQ ID NO: 28), 130 amino acids (C130) (SEQ ID NO: 29), 64 amino acids (C64) (SEQ ID NO: 30) or 25 amino acids (C25) (SEQ ID NO: 31), all derived from human collagen type I, alpha 1 polypeptides, were all fused to a 147 amino acid long human keratin type 31 fragment (K147); (SEQ ID NO: 7), and proteins were expressed and secreted into the fermentation broth as described herein (FIG. 6). The table below lists several truncated variations of length that were used to test the solubility of the keratin polypeptide.

TABLE 7
Various lengths of solubility-enhancing amino
acid sequences used in keratin fusions

| | Collagen- | | | | Improved | | |
|---|---|---|---|---|---|---|---|
| | derived | | | | solubility | DNA | Protein |
| | solubility | Keratin | | | of keratin | SEQ | SEQ |
| Polypeptide | enhancing | type 31 | Approx. | C:K | at low | ID | ID |
| name | sequence | fragment | size | ratio | pH? | NO: | NO: |
| C225-K147 | 225 aa | 147 aa | 41 kDa | 1.53 | Yes | 87 | 88 |
| C196-K147 | 196 aa | 147 aa | 38 kDa | 1.33 | Yes | 89 | 90 |
| C130-K147 | 130 aa | 147 aa | 30 kDa | 0.88 | Yes | 91 | 92 |
| C64-K147 | 64 aa | 147 aa | 23 kDa | 0.44 | Some | 93 | 94 |
| C25-K147 | 25 aa | 147 aa | 19 kDa | 0.17 | Not | 95 | 96 |
| | | | | | detectable | | |
| CO-K147 | none | 147 aa | ** | (control) | (control) | — | 7 |

The nucleic acid sequences and the corresponding amino acid sequences of the above-described fusion proteins are shown below.

The nucleotide sequence encoding C225-K147 was:

(SEQ ID NO: 87)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
CAGCTGGGTGATCGCCTGAATGTTGAAGTTGATGCAGCACCGACC
GTTGATCTGAATCGTGTGCTGAATGAAACCCGTAGCCAGTATGAA
GCCCTGGTGGAAACCAATCGTCGTGAAGTTGAACAGTGGTTTACC
ACACAGACCGAAGAACTGAATAAACAGGTTGTTAGCAGCAGCGAA
CAGCTGCAGAGTTATCAGGCAGAAATCATTGAACTGCGTCGCACC
GTTAATGCACTGGAAATCGAACTGCAGGCACAGCATAATCTGCGT
GATAGCCTGGAAAATACCCTGACCGAAAGCGAAGCACGTTATAGC
AGCCAGCTGAGCCAGGTTCAGAGCCTGATTACCAATGTTGAAAGT
CAGCTGGCAGAAATTCGTAGTGATCTGGAACGTCAGAATCAAGAA
TATCAGGTTCTGCTGGATGTTCGTGCCCGTCTGGAA.

The amino acid sequence corresponding to the above nucleic acid sequence was:

(SEQ ID NO: 88)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
QLGDRLNVEVDAAPTVDLNRVLNETRSQYEALVETNRREVEQWFT
TQTEELNKQVVSSSEQLQSYQAEIIELRRTVNALEIELQAQHNLR
DSLENTLTESEARYSSQLSQVQSLITNVESQLAEIRSDLERQNQE
YQVLLDVRARLE.

The nucleotide sequence encoding C196-K147 was:

(SEQ ID NO: 89)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCCAGCTGGGTGATCGCCTGAATGTTGAAGTTGATGCAGCACCG
ACCGTTGATCTGAATCGTGTGCTGAATGAAACCCGTAGCCAGTAT
GAAGCCCTGGTGGAAACCAATCGTCGTGAAGTTGAACAGTGGTTT
ACCACACAGACCGAAGAACTGAATAAACAGGTTGTTAGCAGCAGC
GAACAGCTGCAGAGTTATCAGGCAGAAATCATTGAACTGCGTCGC
ACCGTTAATGCACTGGAAATCGAACTGCAGGCACAGCATAATCTG
CGTGATAGCCTGGAAAATACCCTGACCGAAAGCGAAGCACGTTAT
AGCAGCCAGCTGAGCCAGGTTCAGAGCCTGATTACCAATGTTGAA
AGTCAGCTGGCAGAAATTCGTAGTGATCTGGAACGTCAGAATCAA
GAATATCAGGTTCTGCTGGATGTTCGTGCCCGTCTGGAA.

The amino acid sequence corresponding to the above nucleic acid sequence was:

(SEQ ID NO: 90)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGPTGARGAPGDRGEPGPPGPA
GFAGPPGADGQPGAKGEPGDAGAKGDAGPPGPAGPAGPPGPIGNVGQLGD
RLNVEVDAAPTVDLNRVLNETRSQYEALVETNRREVEQWFTTQTEELNKQ
VVSSSEQLQSYQAEIIELRRTVNALEIELQAQHNLRDSLENTLTESEARY
SSQLSQVQSLITNVESQLAEIRSDLERQNQEYQVLLDVRARLE.

The nucleotide sequence encoding C130-K147 was:

(SEQ ID NO: 91)
GGTGTACCTGGAGATTTGGGAGCTCCTGGACCATCAGGCGCTCGCGGCGA
ACGCGGATTTCCCGGTGAACGTGGGGTTCAAGGTCCACCCGGCCCTGCTG
GCCCTCGTGGTGCGAACGGTGCGCCAGGAAATGATGGTGCCAAAGGCGAT
GCTGGCGCGCCTGGCGCTCCTGGATCTCAAGGAGCCCCAGGCTTACAAGG
TATGCCCGGAGAACGTGGCGCCGCAGGCTTACCTGGACCTAAAGGTGACC
GTGGAGATGCAGGACCTAAAGGAGCAGATGGCTCTCCTGGAAAGGATGGA
GTACGCGGTTTAACTGGTCCTATTGGTCCACCCGGCCCTGCAGGCGCTCC
CGGGGACAAAGGTGAGTCAGGACCTTCAGGTCCTGCTGGTCAGCTGGGTG
ATCGCCTGAATGTTGAAGTTGATGCAGCACCGACCGTTGATCTGAATCGT
GTGCTGAATGAAACCCGTAGCCAGTATGAAGCCCTGGTGGAAACCAATCG
TCGTGAAGTTGAACAGTGGTTTACCACACAGACCGAAGAACTGAATAAAC
AGGTTGTTAGCAGCAGCGAACAGCTGCAGAGTTATCAGGCAGAAATCATT
GAACTGCGTCGCACCGTTAATGCACTGGAAATCGAACTGCAGGCACAGCA
TAATCTGCGTGATAGCCTGGAAAATACCCTGACCGAAAGCGAAGCACGTT
ATAGCAGCCAGCTGAGCCAGGTTCAGAGCCTGATTACCAATGTTGAAAGT
CAGCTGGCAGAAATTCGTAGTGATCTGGAACGTCAGAATCAAGAATATCA
GGTTCTGCTGGATGTTCGTGCCCGTCTGGAA.

The amino acid sequence corresponding to the above nucleic acid sequence was:

(SEQ ID NO: 92)
GVPGDLGAPGPSGARGERGFPGERGVQGPPGPAGPRGANGAPGNDGAKGD
AGAPGAPGSQGAPGLQGMPGERGAAGLPGPKGDRGDAGPKGADGSPGKDG
VRGLTGPIGPPGPAGAPGDKGESGPSGPAGQLGDRLNVEVDAAPTVDLNR
VLNETRSQYEALVETNRREVEQWFTTQTEELNKQVVSSSEQLQSYQAEII
ELRRTVNALEIELQAQHNLRDSLENTLTESEARYSSQLSQVQSLITNVES
QLAEIRSDLERQNQEYQVLLDVRARLE.

The nucleotide sequence encoding C64-K147 was:

(SEQ ID NO: 93)
GGTGTACCTGGAGATTTGGGAGCTCCTGGACCATCAGGCGCTCGCGGCGA
ACGCGGATTTCCCGGTGAACGTGGGGTTCAAGGTCCACCCGGCCCTGCTG
GCCCTCGTGGTGCGAACGGTGCGCCAGGAAATGATGGTGCCAAAGGCGAT
GCTGGCGCGCCTGGCGCTCCTGGATCTCAAGGAGCCCCAGGCCAGCTGGG
TGATCGCCTGAATGTTGAAGTTGATGCAGCACCGACCGTTGATCTGAATC
GTGTGCTGAATGAAACCCGTAGCCAGTATGAAGCCCTGGTGGAAACCAAT
CGTCGTGAAGTTGAACAGTGGTTTACCACACAGACCGAAGAACTGAATAA
ACAGGTTGTTAGCAGCAGCGAACAGCTGCAGAGTTATCAGGCAGAAATCA
TTGAACTGCGTCGCACCGTTAATGCACTGGAAATCGAACTGCAGGCACAG
CATAATCTGCGTGATAGCCTGGAAAATACCCTGACCGAAAGCGAAGCACG
TTATAGCAGCCAGCTGAGCCAGGTTCAGAGCCTGATTACCAATGTTGAAA
GTCAGCTGGCAGAAATTCGTAGTGATCTGGAACGTCAGAATCAAGAATAT
CAGGTTCTGCTGGATGTTCGTGCCCGTCTGGAA.

The amino acid sequence corresponding to the above nucleic acid sequence was:

(SEQ ID NO: 94)
GVPGDLGAPGPSGARGERGFPGERGVQGPPGPAGPRGANGAPGNDGAKGD
AGAPGAPGSQGAPGQLGDRLNVEVDAAPTVDLNRVLNETRSQYEALVETN
RREVEQWFTTQTEELNKQVVSSSEQLQSYQAEIIELRRTVNALEIELQAQ
HNLRDSLENTLTESEARYSSQLSQVQSLITNVESQLAEIRSDLERQNQEY
QVLLDVRARLE.

The nucleotide sequence encoding C25-K147 was:

(SEQ ID NO: 95)
GGTGTACCTGGAGATTTGGGAGCTCCTGGACCATCAGGCGCTCGCGGCGA
ACGCGGATTTCCCGGTGAACGTGGGCAGCTGGGTGATCGCCTGAATGTTG
AAGTTGATGCAGCACCGACCGTTGATCTGAATCGTGTGCTGAATGAAACC
CGTAGCCAGTATGAAGCCCTGGTGGAAACCAATCGTCGTGAAGTTGAACA
GTGGTTTACCACACAGACCGAAGAACTGAATAAACAGGTTGTTAGCAGCA
GCGAACAGCTGCAGAGTTATCAGGCAGAAATCATTGAACTGCGTCGCACC
GTTAATGCACTGGAAATCGAACTGCAGGCACAGCATAATCTGCGTGATAG
CCTGGAAAATACCCTGACCGAAAGCGAAGCACGTTATAGCAGCCAGCTGA
GCCAGGTTCAGAGCCTGATTACCAATGTTGAAAGTCAGCTGGCAGAAATT
CGTAGTGATCTGGAACGTCAGAATCAAGAATATCAGGTTCTGCTGGATGT
TCGTGCCCGTCTGGAA.

The amino acid sequence corresponding to the above nucleic acid sequence was:

(SEQ ID NO: 96)
GVPGDLGAPGPSGARGERGFPGERGQLGDRLNVEVDAAPTVDLNRVLNET
RSQYEALVETNRREVEQWFTTQTEELNKQVVSSSEQLQSYQAEIIELRRT
VNALEIELQAQHNLRDSLENTLTESEARYSSQLSQVQSLITNVESQLAEI
RSDLERQNQEYQVLLDVRARLE.

As the size of the solubility-enhancing amino acid sequence fragment attached to the keratin polypeptide was reduced, the proportion of the keratin polypeptide which remained in the supernatant fraction (“Supe”) upon acidification was also reduced (FIG. 6).

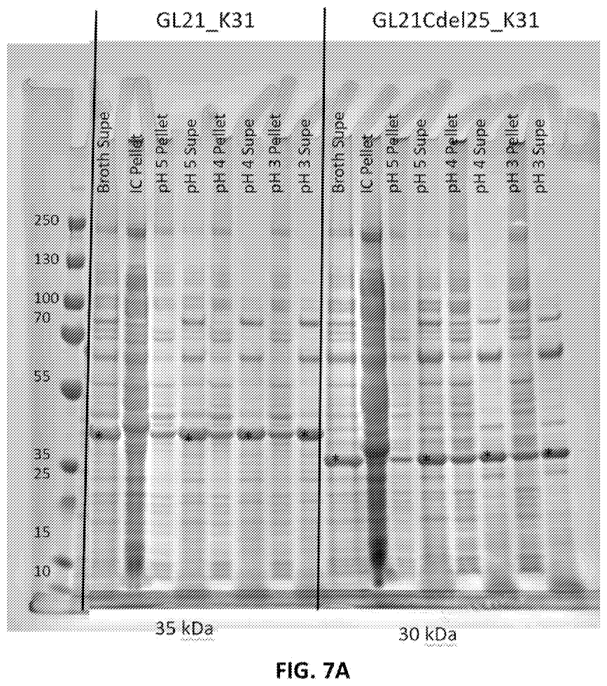

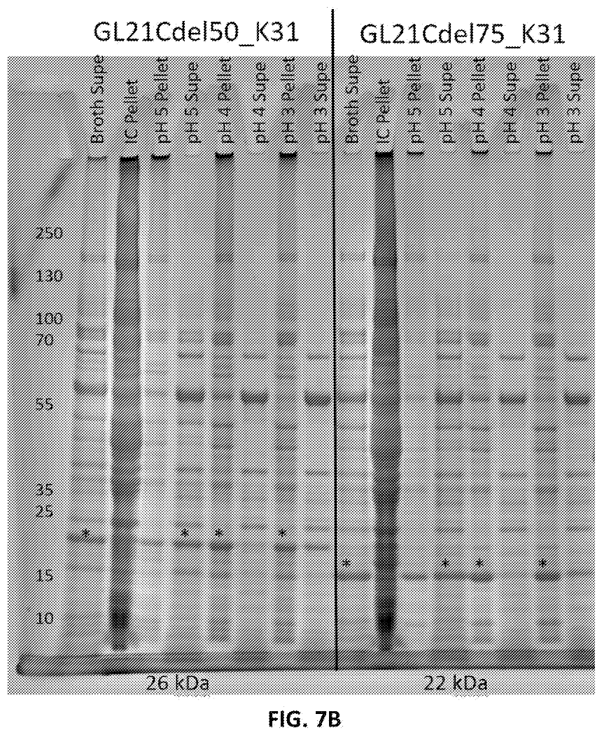

To determine if the effect of length could be seen with a collagen-derived solubility-enhancing amino acid sequence from a different source, the experiment was attempted with solubility-enhancing amino acid sequences of 188 amino acids (SEQ ID NO: 19), 141 amino acids (SEQ ID NO: 20), 93 amino acids (SEQ ID NO: 21), or 47 amino acids (SEQ ID NO: 22), all derived from chicken collagen, type 21 polypeptides, all fused to a 147 amino acid long human keratin type 31 fragment (K147); (SEQ ID NO: 7). and proteins were expressed and secreted into the fermentation broth as described herein (FIGS. 7A and 7B). The table below lists several truncated variations of length that were used to test the solubility of the keratin polypeptide.

TABLE 8
Various lengths of solubility-enhancing amino
acid sequences used in keratin fusions

| | Collagen- | | | | | | |
|---|---|---|---|---|---|---|---|
| | derived | | | | Improved | DNA | Protein |
| | solubility | Keratin | | | solubility of | SEQ | SEQ |
| Polypeptide | enhancing | type 31 | Approx. | C:K | keratin at | ID | ID |
| name | sequence | fragment | size | ratio | low pH? | NO: | NO: |
| GL21_K31 | 188 aa | 147 aa | 35 kDa | 1.28 | Yes | 97 | 63 |
| GL21Cdel25_{—} | 141 aa | 147 aa | 30 kDa | 0.96 | Yes | 98 | 64 |
| K31 | | | | | | | |
| GL21Cdel50_{—} | 93 aa | 147 aa | 26 kDa | 0.63 | Some | 99 | 65 |
| K31 | | | | | | | |
| GL21Cdel75_{—} | 48 aa | 147 aa | 22 kDa | 0.32 | Some | 100 | 66 |
| K31 | | | | | | | |

The nucleic acid sequences and the corresponding amino acid sequences of the above-described fusion proteins are shown below.

The nucleic acid sequence encoding GL21_K31 was:

(SEQ ID NO: 97)
GATACTGGTTTCCCGGGGATGCCTGGGCGCTCAGGTGATCCGGGGCGTAG
TGGAAAAGACGGTCTGCCGGGGTCCCCGGGCTTTAAGGGTGAGGTGGGTC
AGCCCGGTAGTCCAGGTTTAGAAGGTCACCGCGGAGAGCCCGGGATTCCA
GGCATTCCTGGCAACCAGGGTGCCAAGGGACAGAAAGGCGAAATTGGTCC
GCCCGGCCTACCGGGCGCGAAAGGTTCTCCTGGTGAAACCGGTCTCATGG
GTCCGGAAGGTAGCTTCGGCCTGCCCGGCGCACCTGGTCCGAAGGGCGAT
AAGGGGGAGCCTGGGCTGCAAGGTAAACCGGGTAGTTCTGGCGCCAAAGG
TGAACCCGGCGGTCCCGGTGCGCCAGGGGAACCAGGTTATCCTGGTATTC
CTGGAACCCAAGGAATTAAAGGTGACAAAGGCTCACAGGGCGAAAGTGGT
ATACAGGGTCGCAAGGGCGAAAAAGGACGTCAGGGCAATCCAGGCCTGCA
GGGTACTGAAGGCCTGCGTGGAGAACAGGGTGAGAAAGGTGAAAAAGGAG
ATCCTGGTATTCGCCAGCTGGGTGATCGCCTGAATGTTGAAGTTGATGCA
GCACCGACCGTTGATCTGAATCGTGTGCTGAATGAAACCCGTAGCCAGTA
TGAAGCCCTGGTGGAAACCAATCGTCGTGAAGTTGAACAGTGGTTTACCA
CACAGACCGAAGAACTGAATAAACAGGTTGTTAGCAGCAGCGAACAGCTG
CAGAGTTATCAGGCAGAAATCATTGAACTGCGTCGCACCGTTAATGCACT
GGAAATCGAACTGCAGGCACAGCATAATCTGCGTGATAGCCTGGAAAATA
CCCTGACCGAAAGCGAAGCACGTTATAGCAGCCAGCTGAGCCAGGTTCAG
AGCCTGATTACCAATGTTGAAAGTCAGCTGGCAGAAATTCGTAGTGATCT
GGAACGTCAGAATCAAGAATATCAGGTTCTGCTGGATGTTCGTGCCCGTC
TGGAA.

The amino acid sequence corresponding to the above nucleic acid sequence was:

(SEQ ID NO: 63)
DTGFPGMPGRSGDPGRSGKDGLPGSPGFKGEVGQPGSPGLEGHRGEPGIP
GIPGNQGAKGQKGEIGPPGLPGAKGSPGETGLMGPEGSFGLPGAPGPKGD
KGEPGLQGKPGSSGAKGEPGGPGAPGEPGYPGIPGTQGIKGDKGSQGESG
IQGRKGEKGRQGNPGLQGTEGLRGEQGEKGEKGDPGIRQLGDRLNVEVDA
APTVDLNRVLNETRSQYEALVETNRREVEQWFTTQTEELNKQVVSSSEQL
QSYQAEIIELRRTVNALEIELQAQHNLRDSLENTLTESEARYSSQLSQVQ
SLITNVESQLAEIRSDLERQNQEYQVLLDVRARLE.

The nucleic acid sequence encoding GL21del25_K31 was:

(SEQ ID NO: 98)
GATACTGGTTTCCCGGGGATGCCTGGGCGCTCAGGTGATCCGGGGCGTAG
TGGAAAAGACGGTCTGCCGGGGTCCCCGGGCTTTAAGGGTGAGGTGGGTC
AGCCCGGTAGTCCAGGTTTAGAAGGTCACCGCGGAGAGCCCGGGATTCCA
GGCATTCCTGGCAACCAGGGTGCCAAGGGACAGAAAGGCGAAATTGGTCC
GCCCGGCCTACCGGGCGCGAAAGGTTCTCCTGGTGAAACCGGTCTCATGG
GTCCGGAAGGTAGCTTCGGCCTGCCCGGCGCACCTGGTCCGAAGGGCGAT
AAGGGGGAGCCTGGGCTGCAAGGTAAACCGGGTAGTTCTGGCGCCAAAGG
TGAACCCGGCGGTCCCGGTGCGCCAGGGGAACCAGGTTATCCTGGTATTC
CTGGAACCCAAGGAATTAAAGGTCAGCTGGGTGATCGCCTGAATGTTGAA
GTTGATGCAGCACCGACCGTTGATCTGAATCGTGTGCTGAATGAAACCCG
TAGCCAGTATGAAGCCCTGGTGGAAACCAATCGTCGTGAAGTTGAACAGT
GGTTTACCACACAGACCGAAGAACTGAATAAACAGGTTGTTAGCAGCAGC
GAACAGCTGCAGAGTTATCAGGCAGAAATCATTGAACTGCGTCGCACCGT
TAATGCACTGGAAATCGAACTGCAGGCACAGCATAATCTGCGTGATAGCC
TGGAAAATACCCTGACCGAAAGCGAAGCACGTTATAGCAGCCAGCTGAGC
CAGGTTCAGAGCCTGATTACCAATGTTGAAAGTCAGCTGGCAGAAATTCG
TAGTGATCTGGAACGTCAGAATCAAGAATATCAGGTTCTGCTGGATGTTC
GTGCCCGTCTGGAA.

The amino acid sequence corresponding to the above nucleic acid sequence was:

(SEQ ID NO: 64)
DTGFPGMPGRSGDPGRSGKDGLPGSPGFKGEVGQPGSPGLEGHRGEPGIP
GIPGNQGAKGQKGEIGPPGLPGAKGSPGETGLMGPEGSFGLPGAPGPKGD
KGEPGLQGKPGSSGAKGEPGGPGAPGEPGYPGIPGTQGIKGQLGDRLNVE
VDAAPTVDLNRVLNETRSQYEALVETNRREVEQWFTTQTEELNKQVVSSS
EQLQSYQAEIIELRRTVNALEIELQAQHNLRDSLENTLTESEARYSSQLS
QVQSLITNVESQLAEIRSDLERQNQEYQVLLDVRARLE.

The nucleic acid sequence encoding GL21del50_K31 was:

(SEQ ID NO: 99)
GATACTGGTTTCCCGGGGATGCCTGGGCGCTCAGGTGATCCGGGGCGTAG
TGGAAAAGACGGTCTGCCGGGGTCCCCGGGCTTTAAGGGTGAGGTGGGTC
AGCCCGGTAGTCCAGGTTTAGAAGGTCACCGCGGAGAGCCCGGGATTCCA
GGCATTCCTGGCAACCAGGGTGCCAAGGGACAGAAAGGCGAAATTGGTCC
GCCCGGCCTACCGGGCGCGAAAGGTTCTCCTGGTGAAACCGGTCTCATGG
GTCCGGAAGGTAGCTTCGGCCTGCCCGGCCAGCTGGGTGATCGCCTGAAT
GTTGAAGTTGATGCAGCACCGACCGTTGATCTGAATCGTGTGCTGAATGA
AACCCGTAGCCAGTATGAAGCCCTGGTGGAAACCAATCGTCGTGAAGTTG
AACAGTGGTTTACCACACAGACCGAAGAACTGAATAAACAGGTTGTTAGC
AGCAGCGAACAGCTGCAGAGTTATCAGGCAGAAATCATTGAACTGCGTCG
CACCGTTAATGCACTGGAAATCGAACTGCAGGCACAGCATAATCTGCGTG
ATAGCCTGGAAAATACCCTGACCGAAAGCGAAGCACGTTATAGCAGCCAG
CTGAGCCAGGTTCAGAGCCTGATTACCAATGTTGAAAGTCAGCTGGCAGA
AATTCGTAGTGATCTGGAACGTCAGAATCAAGAATATCAGGTTCTGCTGG
ATGTTCGTGCCCGTCTGGAA.

The amino acid sequence corresponding to the above nucleic acid sequence was:

(SEQ ID NO: 65)
DTGFPGMPGRSGDPGRSGKDGLPGSPGFKGEVGQPGSPGLEGHRGEPGIP
GIPGNQGAKGQKGEIGPPGLPGAKGSPGETGLMGPEGSFGLPGQLGDRLN
VEVDAAPTVDLNRVLNETRSQYEALVETNRREVEQWFTTQTEELNKQVVS
SSEQLQSYQAEIIELRRTVNALEIELQAQHNLRDSLENTLTESEARYSSQ
LSQVQSLITNVESQLAEIRSDLERQNQEYQVLLDVRARLE.

The nucleic acid sequence encoding GL21del75_K31 was:

(SEQ ID NO: 100)
GATACTGGTTTCCCGGGGATGCCTGGGCGCTCAGGTGATCCGGGGCGTAG
TGGAAAAGACGGTCTGCCGGGGTCCCCGGGCTTTAAGGGTGAGGTGGGTC
AGCCCGGTAGTCCAGGTTTAGAAGGTCACCGCGGAGAGCCCGGGCAGCTG
GGTGATCGCCTGAATGTTGAAGTTGATGCAGCACCGACCGTTGATCTGAA
TCGTGTGCTGAATGAAACCCGTAGCCAGTATGAAGCCCTGGTGGAAACCA
ATCGTCGTGAAGTTGAACAGTGGTTTACCACACAGACCGAAGAACTGAAT
AAACAGGTTGTTAGCAGCAGCGAACAGCTGCAGAGTTATCAGGCAGAAAT
CATTGAACTGCGTCGCACCGTTAATGCACTGGAAATCGAACTGCAGGCAC
AGCATAATCTGCGTGATAGCCTGGAAAATACCCTGACCGAAAGCGAAGCA
CGTTATAGCAGCCAGCTGAGCCAGGTTCAGAGCCTGATTACCAATGTTGA
AAGTCAGCTGGCAGAAATTCGTAGTGATCTGGAACGTCAGAATCAAGAAT
ATCAGGTTCTGCTGGATGTTCGTGCCCGTCTGGAA.

The amino acid sequence corresponding to the above nucleic acid sequence was:

(SEQ ID NO: 66)
DTGFPGMPGRSGDPGRSGKDGLPGSPGFKGEVGQPGSPGLEGHRGEPQLG
DRLNVEVDAAPTVDLNRVLNETRSQYEALVETNRREVEQWFTTQTEELNK
QVVSSSEQLQSYQAEIIELRRTVNALEIELQAQHNLRDSLENTLTESEAR
YSSQLSQVQSLITNVESQLAEIRSDLERQNQEYQVLLDVRARLE.

As seen with the human collagen-derived solubility-enhancing amino acid sequence construct, as the size of the chicken-derived solubility-enhancing amino acid sequence fragment attached to the keratin polypeptide was reduced, the proportion of the keratin polypeptide which remained in the supernatant fraction (“Supe”) upon acidification was also reduced (FIGS. 7A and 7B).

This indicates that multiple lengths of solubility-enhancing amino acid sequences derived from a variety of collagen fragments are able to endow the recombinant keratin polypeptides of the present disclosure with solubility, enabling simple and inexpensive purification of desired keratin polypeptides.

### Example 5. Various Lengths of Keratin Polypeptides were Expressed and Purified Using Solubility-Enhancing Amino Acid Sequences

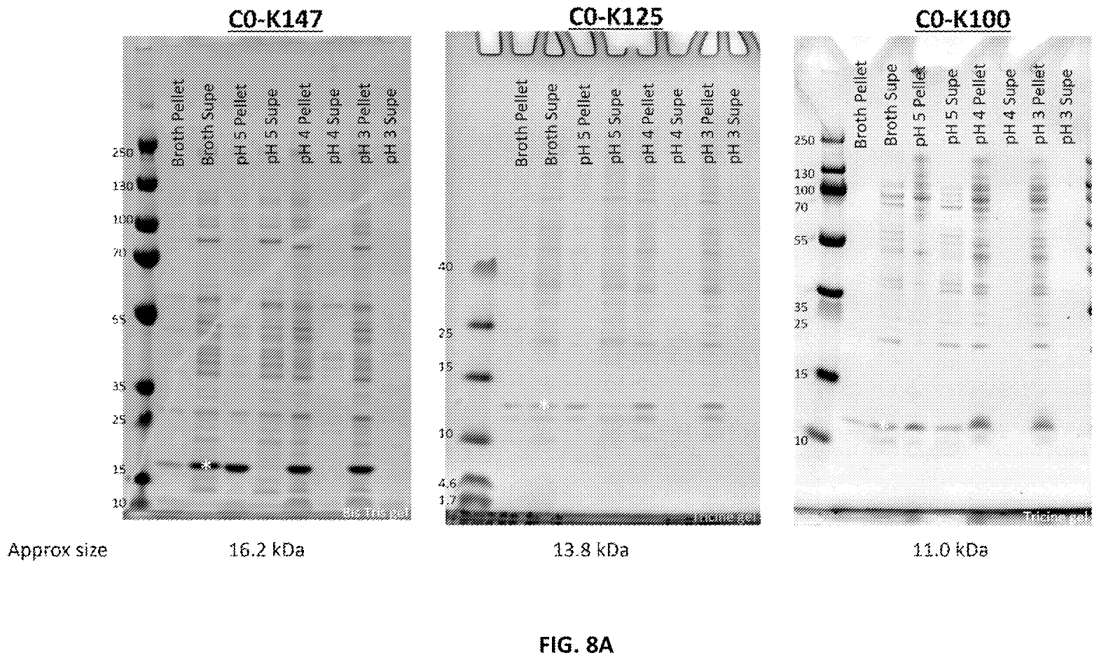

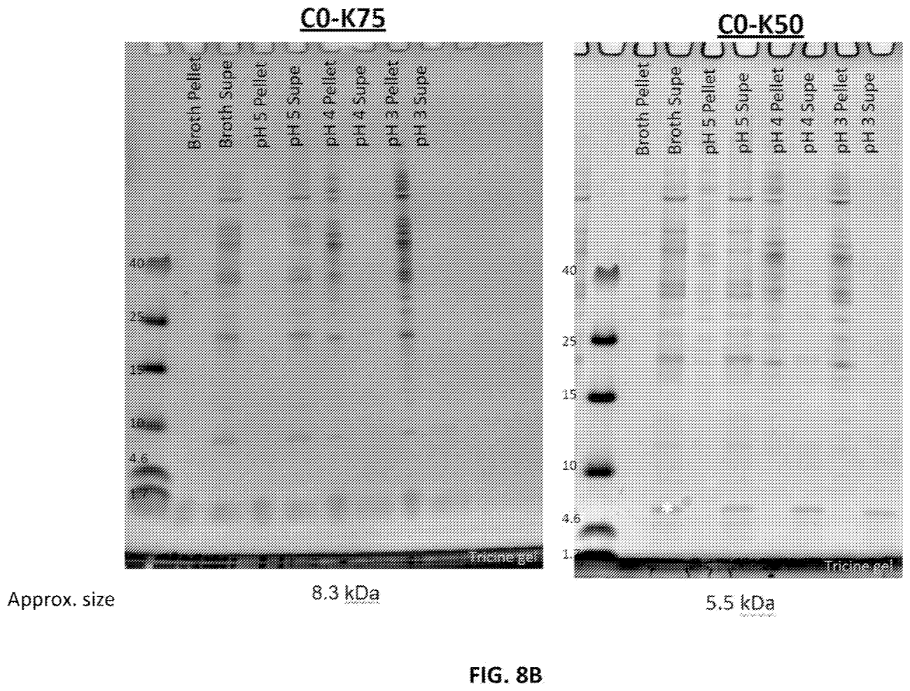

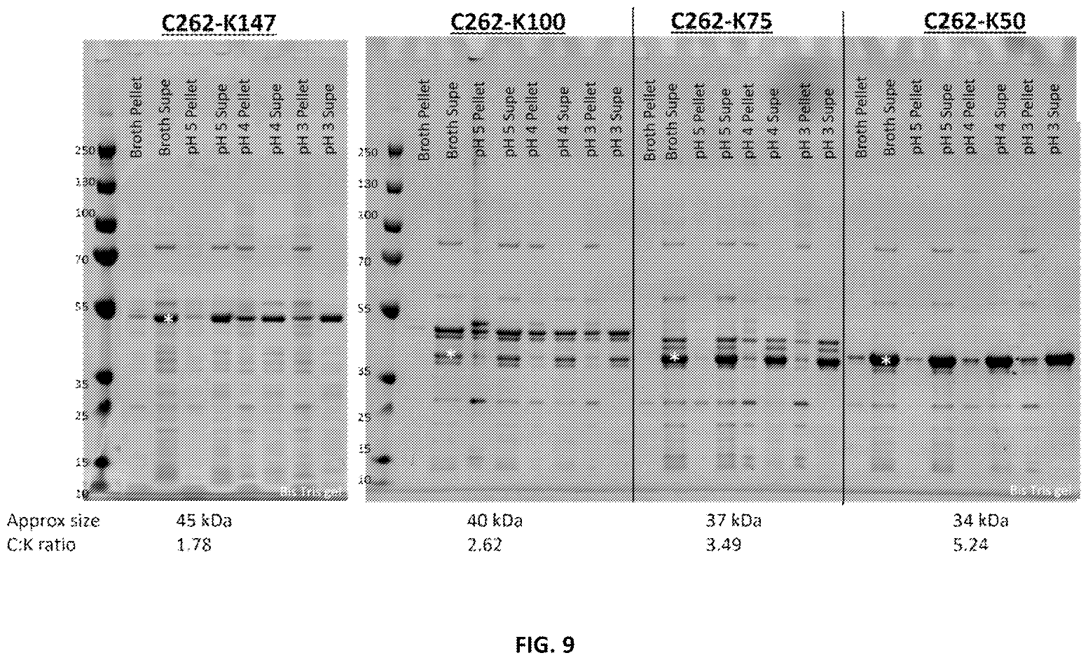

To determine whether various lengths of keratin polypeptides could be expressed and purified using the solubility-enhancing amino acid sequences of the present invention, the following experiment was performed. Various lengths of human keratin type 31 polypeptide fragment were tested both without (FIGS. 8A and 8B) and with (FIG. 9) a solubility-enhancing amino acid sequence derived from a human collagen type I, alpha 1 polypeptide fragment (C262) (SEQ ID NO: 26). Human keratin type 31 polypeptide lengths tested were 147 amino acids (K147) (SEQ ID NO: 7), 100 amino acids (K100) (SEQ ID NO: 9), 75 amino acids (K75) (SEQ ID NO: 10) and 50 amino acids (K50) (SEQ ID NO: 11), and proteins were expressed and secreted into the fermentation broth as described herein.

TABLE 9
Effect of solubility-enhancing amino acid sequences
on various length keratin polypeptides

| | Collagen- | | | | Improved | |
|---|---|---|---|---|---|---|
| | derived | Keratin | | | solubility | |
| Poly- | solubility | type 31 | | | of keratin | Protein |
| peptide | enhancing | frag- | Approx | C:K | at low | SEQ ID |
| name | sequence | ment | size | ratio | pH? | NO: |
| C262-K147 | 262 aa | 147 aa | 45 kDa | 1.78 | Yes | 102 |
| C262-K100 | 262 aa | 100 aa | 40 kDa | 2.62 | Yes | 104 |
| C262-K75 | 262 aa | 75 aa | 37 kDa | 3.49 | Yes | 106 |
| C262-K50 | 262 aa | 50 aa | 34 kDa | 5.24 | Yes | 108 |

Keratin only:

| | | | | | | |
|---|---|---|---|---|---|---|
| C0-K147 | — | 147 aa | 16 kDa | — | — | 7 |
| C0-K125 | — | 125 aa | 13 kDa | — | — | 8 |
| C0-K100 | — | 100 aa | 11 kDa | — | — | 9 |
| C0-K75 | — | 75 aa | 8 kDa | — | — | 10 |
| C0-K50 | — | 50 aa | 6 kDa | — | — | 11 |

The nucleic acid sequences and the corresponding amino acid sequences of the above-described fusion proteins are shown below.

The nucleotide sequence encoding C262-K147 was:

(SEQ ID NO: 101)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCAGCTGGGTGATCGCCTGAATGTT
GAAGTTGATGCAGCACCGACCGTTGATCTGAATCGTGTGCTGAAT
GAAACCCGTAGCCAGTATGAAGCCCTGGTGGAAACCAATCGTCGT
GAAGTTGAACAGTGGTTTACCACACAGACCGAAGAACTGAATAAA
CAGGTTGTTAGCAGCAGCGAACAGCTGCAGAGTTATCAGGCAGAA
ATCATTGAACTGCGTCGCACCGTTAATGCACTGGAAATCGAACTG
CAGGCACAGCATAATCTGCGTGATAGCCTGGAAAATACCCTGACC
GAAAGCGAAGCACGTTATAGCAGCCAGCTGAGCCAGGTTCAGAGC
CTGATTACCAATGTTGAAAGTCAGCTGGCAGAAATTCGTAGTGAT
CTGGAACGTCAGAATCAAGAATATCAGGTTCTGCTGGATGTTCGT
GCCCGTCTGGAA.

The amino acid sequence corresponding to the above nucleic acid sequence was:

(SEQ ID NO: 102)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEELNK
QVVSSSEQLQSYQAEIIELRRTVNALEIELQAQHNLRDSLENTLT
ESEARYSSQLSQVQSLITNVESQLAEIRSDLERQNQEYQVLLDVR
ARLE.

The nucleotide sequence encoding C262-K100 was:

(SEQ ID NO: 103)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCAGCTGGGTGATCGCCTGAATGTT
GAAGTTGATGCAGCACCGACCGTTGATCTGAATCGTGTGCTGAAT
GAAACCCGTAGCCAGTATGAAGCCCTGGTGGAAACCAATCGTCGT
GAAGTTGAACAGTGGTTTACCACACAGACCGAAGAACTGAATAAA
CAGGTTGTTAGCAGCAGCGAACAGCTGCAGAGTTATCAGGCAGAA
ATCATTGAACTGCGTCGCACCGTTAATGCACTGGAAATCGAACTG
CAGGCACAGCATAATCTGCGTGATAGCCTGGAAAATACCCTGACC
GAAAGC.

The amino acid sequence corresponding to the above nucleic acid sequence was:

(SEQ ID NO: 104)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEELNK
QVVSSSEQLQSYQAEIIELRRTVNALEIELQAQHNLRDSLENTLT
ES.

The nucleotide sequence encoding C262-K75 was:

(SEQ ID NO: 105)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCAGCTGGGTGATCGCCTGAATGTT
GAAGTTGATGCAGCACCGACCGTTGATCTGAATCGTGTGCTGAAT
GAAACCCGTAGCCAGTATGAAGCCCTGGTGGAAACCAATCGTCGT
GAAGTTGAACAGTGGTTTACCACACAGACCGAAGAACTGAATAAA
CAGGTTGTTAGCAGCAGCGAACAGCTGCAGAGTTATCAGGCAGAA
ATCATTGAACTGCGTCGCACC.

The amino acid sequence corresponding to the above nucleic acid sequence was:

(SEQ ID NO: 106)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEELNK
QVVSSSEQLQSYQAEIIELRRT.

The nucleotide sequence encoding C262-K50 was:

(SEQ ID NO: 107)
GGTGTACCTGGAGATTTGGGAGCTCCTGGACCATCAGGCGCTCGC
GGCGAACGCGGATTTCCCGGTGAACGTGGGGTTCAAGGTCCACCC
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGCAGCTGGGTGATCGCCTGAATGTT
GAAGTTGATGCAGCACCGACCGTTGATCTGAATCGTGTGCTGAAT
GAAACCCGTAGCCAGTATGAAGCCCTGGTGGAAACCAATCGTCGT
GAAGTTGAACAGTGGTTTACCACACAGACCGAAGAA.

The amino acid sequence corresponding to the above nucleic acid sequence was:

(SEQ ID NO: 108)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEE.

As shown in FIGS. 8A and 8B, the keratin polypeptides alone remained insoluble throughout the acidification process. The solubility-enhancing amino acid sequence was remarkably effective in producing soluble keratin polypeptides, particularly shorter keratin polypeptides (FIG. 9). As the size of the keratin portion of the fusion protein was reduced, the proportion of the fusion protein which remained in the supernatant fraction upon acidification was increased. FIG. 9 shows this difference, especially by a comparison of the amount of protein present in the pH 3 pellet lane to the amount of protein present in the pH 3 supernatant lane. Also, as the size of the keratin fragment was reduced, the ratio of collagen to keratin (“C:K”) increased. The proportion of the fusion protein remaining in the supernatant fraction versus the pellet fraction at acidified pH also increased.

The failure to purify keratin polypeptides lacking the solubility-enhancing amino acid sequence, e.g., the unfused 147 amino acid, 125 amino acid and 100 amino acid keratin 31 constructs in acidified media is shown in FIGS. 8A and 8B. None of the keratin fragments lacking a solubility-enhancing segment that were examined were soluble in acidified media when expressed alone.

This indicates that solubility-enhancing amino acid sequences of the present disclosure are able to endow various lengths of recombinant keratin polypeptides with solubility, enabling simple and inexpensive purification of desired keratin polypeptides.

### Example 6. Variations of the Length Ratio of Solubility-Enhancing Amino Acid Sequences to the Keratin Polypeptide Fragments

To examine the effects of the ratio (as determined by amino acid length) of the solubility-enhancing amino acid sequence to the keratin polypeptide, the following experiment was performed. Various lengths of collagen-derived solubility-enhancing amino acid sequences were combined with various lengths of human keratin type 31 polypeptides.

TABLE 10
Collagen and Keratin amino acid length ratios

| Collagen- | | | | | |
|---|---|---|---|---|---|
| derived | | | | | |
| solubility | Keratin | | | | |
| enhancing | type 31 | Total | Approx | C:K | |
| sequence | fragment | Residues | size (kDa) | ratio | Notes |
| 262 aa | 147 aa | 409 aa | 44.99 | 1.78 | Original HsCol1a1-27_K31 |
| | | | | | construct |
| 262 aa | 100 aa | 362 aa | 39.82 | 2.62 | |
| 262 aa | 75 aa | 337 aa | 37.07 | 3.49 | |
| 262 aa | 50 aa | 312 aa | 34.32 | 5.24 | Longest collagen, shortest |
| | | | | | keratin |
| 225 aa | 147 aa | 372 aa | 40.92 | 1.53 | |
| 196 aa | 147 aa | 343 aa | 37.73 | 1.33 | “_Cdel25” |
| 130 aa | 147 aa | 277 aa | 30.47 | 0.88 | “_Cdel50” |
| 64 aa | 147 aa | 211aa | 23.21 | 0.44 | “_Cdel75” |
| 25 aa | 147 aa | 172 aa | 18.92 | 0.17 | Shortest collagen, longest |
| | | | | | keratin |
| 196 aa | 100 aa | 296 aa | 32.56 | 1.96 | C:K ratio spread; different |
| | | | | | total size |
| 196 aa | 50 aa | 246 aa | 27.06 | 3.92 | C:K ratio spread; different |
| | | | | | total size |
| 64 aa | 100 aa | 164 aa | 18.04 | 0.64 | C:K ratio spread; different |
| | | | | | total size |
| 64 aa | 50 aa | 114 aa | 12.54 | 1.28 | C:K ratio spread; different |
| | | | | | total size |
| 0 | 147aa | 147 aa | 16.17 | 0 | Keratin only control |
| 0 | 125 aa | 125 aa | 13.75 | 0 | Keratin only control |
| 0 | 100 aa | 100 aa | 11.0 | 0 | Keratin only control |
| 0 | 75 aa | 75 aa | 8.25 | 0 | Keratin only control |
| 0 | 50 aa | 50 aa | 5.5 | 0 | Keratin only control |

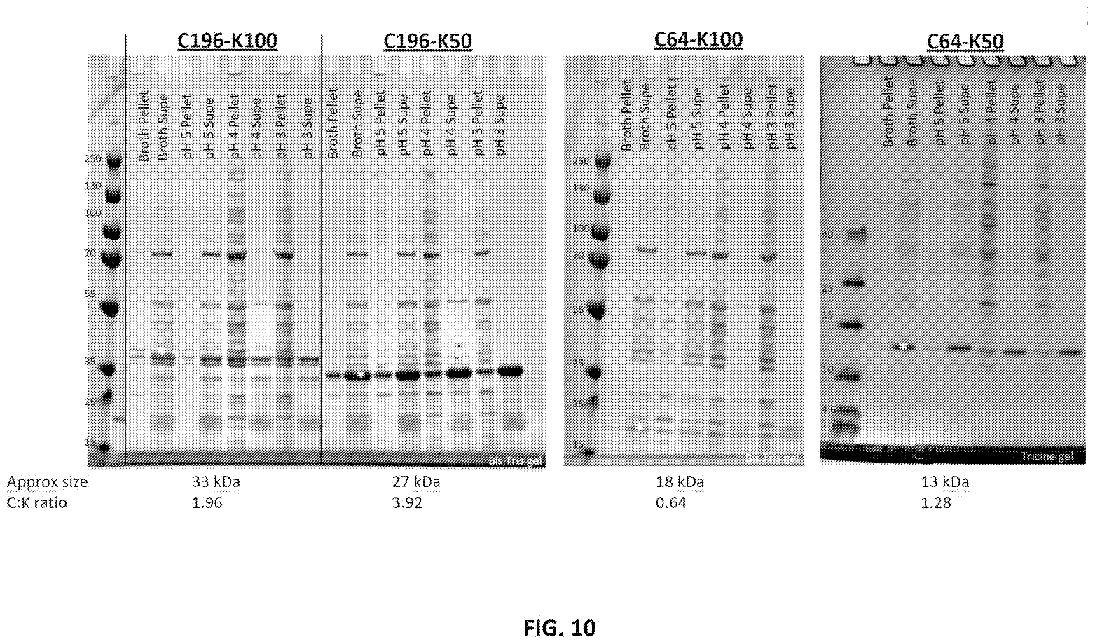

Human collagen type I, alpha 1 polypeptide fragment of 196 aa (SEQ ID NO: 28) with human keratin type 31 polypeptide of 100 aa (SEQ ID NO: 9) to result in “C196-K100” (SEQ ID NO: 70); human collagen type I, alpha 1 polypeptide fragment of 196 aa (SEQ ID NO: 28) with human keratin type 31 polypeptide of 50 aa (SEQ ID NO: 11) to result in “C196-K50” (SEQ ID NO: 71); human collagen type I, alpha 1 polypeptide fragment of 64 aa (SEQ ID NO 30) with human keratin type 31 polypeptide of 100 aa (SEQ ID NO: 9) to result in “C64-K100” (SEQ ID NO: 72), and human collagen type I, alpha 1 polypeptide fragment of 64 aa (SEQ ID NO: 30) with human keratin type 31 polypeptide of 50 aa (SEQ ID NO: 11) to result in “C64-K50” (SEQ ID NO: 73) (FIG. 10).

TABLE 11
Effect of ratio of solubility-enhancing amino acid
sequences to keratin polypeptides sequences

| | Collagen- | | | | Improved | |
|---|---|---|---|---|---|---|
| | derived | Keratin | | | solubility | |
| Poly- | solubility | type 31 | | | of keratin | Protein |
| peptide | enhancing | frag- | Approx | C:K | at low | SEQ ID |
| name | sequence | ment | size | ratio | pH? | NO: |
| C196-K100 | 196 aa | 100 aa | 33 kDa | 1.96 | Yes | 70 |
| C196-K50 | 196 aa | 50 aa | 27 kDa | 3.92 | Yes | 71 |
| C64-K100 | 64 aa | 100 aa | 18 kDa | 0.64 | Some | 72 |
| C64-K50 | 64 aa | 50 aa | 12 kDa | 1.28 | Yes | 73 |

As seen with the earlier Examples, the solubility-enhancing amino acid sequences of various lengths attached to the keratin polypeptides of various lengths increased the proportion of the keratin polypeptide which remained in the supernatant fraction (“Supe”) upon acidification (FIG. 10). The ratios of C:K are indicated below each gel section. The keratin polypeptides having a higher C:K ratio had a larger amount of polypeptide present in the soluble fraction, particularly at pH 3. The C64:K100 protein, having a low C:K ratio (0.64) had very little protein present in the supernatant. This demonstrates that as the ratio of the length of the solubility-enhancing amino acid sequence to the keratin sequence increases, the solubility increases.

### Example 7. Variations of the Position and Number of Solubility-Enhancing Amino Acid Sequences with Respect to the Keratin Polypeptide

This example was performed to determine whether adding flanking regions of solubility-enhancing amino acid sequences to both the N-terminal and C-terminal regions of keratin polypeptides would improve its solubility, thereby facilitating the downstream process.

Constructs were prepared according to the table below. Upon confirming the accuracy of the nucleic acid sequences encoding the fusion proteins, and expression of the fusion proteins in microbial cells, the proteins were analyzed by SDS-PAGE to confirm the correct size of the resulting protein. Solubility-enhancing amino acid sequences were derived from a human collagen type I, alpha 1 polypeptide fragments and combined with various types of keratin polypeptides.

TABLE 12
Collagen-based Stability Sandwiches

| | | Collagen- | | Additional | | Improved |
|---|---|---|---|---|---|---|
| Type of | | derived | | collagen | | solubility |
| fusion | | solubility | | truncates | SEQ | of keratin |
| (C = collagen; | | enhancing | Keratin | (name and | ID | at low |
| K = keratin) | Polypeptide Name | sequence | Fragment | size) | NO: | pH? |
| C-K-C | HsCol1A- | Human | Human Keratin | Human | 81 | Yes |
| | 027_K12_HsCol1A- | collagen | 12 (179 aa) | collagen 27 | | |
| | 028 | type 1 alpha | | alpha 1 (219 | | |
| | | 1 (262 aa) | | aa) | | |
| C-K | HsCol1A-027_K31 | Human | Human Keratin | NA | 109 | Yes |
| | | collagen | 31 (147 aa) | | | |
| | | type 1 alpha | | | | |
| | | 1 (262 aa) | | | | |
| C-K-C | HsCol1A- | Human | Human Keratin | Human | 82 | Yes |
| | 027_Keratin-33A- | collagen | 33A (147 aa) | collagen 27 | | |
| | lowC_HsCol1A- | type 1 alpha | | alpha 1 (219 | | |
| | 028 | 1 (262 aa) | | aa) | | |
| C-K-C | “K82- | Human | Human Keratin | Human | 110 | Yes |
| | 2” (HsCol1A- | collagen | “K82-2” | collagen 27 | | |
| | 027_K82- | type 1 alpha | | alpha 1 (219 | | |
| | 2_HsCol1A-028) | 1 (262 aa) | | aa) | | |
| CC-K | | Human | human keratin | Human | 111 | Not |
| | | collagen | 31 (147 aa) | collagen type | | tested |
| | | type 1 alpha | | 1 alpha 1 | | |
| | | 1 (262 aa) | | (262 aa) | | |
| C-K-CC | | Human | human keratin | Human | 112 | Not |
| | | collagen | 31 (147 aa) | collagen type | | tested |
| | | type 1 alpha | | 1 alpha 1 | | |
| | | 1 (262 aa) | | (262 aa) | | |
| K-C | | Human | human keratin | Human | 113 | Not |
| | | collagen | 31 (147 aa) | collagen type | | tested |
| | | type 1 alpha | | 1 alpha 1 | | |
| | | 1 (262 aa) | | (262 aa) | | |

The amino acid sequence corresponding to the “C—K” sequence in Table 12 is shown below:

(SEQ ID NO: 109)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEEQLG
DRLNVEVDAAPTVDLNRVLNETRSQYEALVETNRREVEQWFTTQT
EELNKQVVSSSEQLQSYQAEIIELRRTVNALEIELQAQHNLRDSL
ENTLTESEARYSSQLSQVQSLITNVESQLAEIRSDLERQNQEYQV
LLDVRARLE.

The amino acid sequence corresponding to the “C—K—C” sequence in Table 12 is shown below:

(SEQ ID NO: 110)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGGYISALRR
QLDCVSGDRVRLESELCSLQAALEGYKKKYEEELSLRPCVENEFV
ALKKDVDTAFLMKADLETNAEALVQEIDFLKSLYEEEICLLQSQI
SETSVIVKMDNSRELDVDGIIAEIKAQYDDIASRSKAEAEAWYQC
RYEELRVTAGNHCDNLRNRKNEILEMNKLIQNDGAKGDAGAPGAP
GSQGAPGLQGMPGERGAAGLPGPKGDRGDAGPKGADGSPGKDGVR
GLTGPIGPPGPAGAPGDKGESGPSGPAGPTGARGAPGDRGEPGPP
GPAGFAGPPGADGQPGAKGEPGDAGAKGDAGPPGPAGPAGPPGPI
GNVGAPGAKGARGSAGPPGATGFPGAAGRVGPPGPSGNAGPPGPP
GPAGKEGGKGPRGETGPAGRPGEVG.

The amino acid sequence corresponding to the “C—C—K” sequence in Table 12 is shown below:

(SEQ ID NO: 111)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGGVPGDLGA
PGPSGARGERGFPGERGVQGPPGPAGPRGANGAPGNDGAKGDAGA
PGAPGSQGAPGLQGMPGERGAAGLPGPKGDRGDAGPKGADGSPGK
DGVRGLTGPIGPPGPAGAPGDKGESGPSGPAGPTGARGAPGDRGE
PGPPGPAGFAGPPGADGQPGAKGEPGDAGAKGDAGPPGPAGPAGP
PGPIGNVGAPGAKGARGSAGPPGATGFPGAAGRVGPPGPSGNAGP
PGPPGPAGKEGGKGPRGETGPAGRPGEVGQLGDRLNVEVDAAPTV
DLNRVLNETRSQYEALVETNRREVEQWFTTQTEELNKQVVSSSEQ
LQSYQAEIIELRRTVNALEIELQAQHNLRDSLENTLTESEARYSS
QLSQVQSLITNVESQLAEIRSDLERQNQEYQVLLDVRARLE.

The amino acid sequence corresponding to the “C—K—C—C” sequence in Table 12 is shown below:

(SEQ ID NO: 112)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGQLGDRLNV
EVDAAPTVDLNRVLNETRSQYEALVETNRREVEQWFTTQTEELN
KQVVSSSEQLQSYQAEIIELRRTVNALEIELQAQHNLRDSLENTL
TESEARYSSQLSQVQSLITNVESQLAEIRSDLERQNQEYQVLLDV
RARLEGVPGDLGAPGPSGARGERGFPGERGVQGPPGPAGPRGANG
APGNDGAKGDAGAPGAPGSQGAPGLQGMPGERGAAGLPGPKGDRG
DAGPKGADGSPGKDGVRGLTGPIGPPGPAGAPGDKGESGPSGPAG
PTGARGAPGDRGEPGPPGPAGFAGPPGADGQPGAKGEPGDAGAKG
DAGPPGPAGPAGPPGPIGNVGAPGAKGARGSAGPPGATGFPGAAG
RVGPPGPSGNAGPPGPPGPAGKEGGKGPRGETGPAGRPGEVGGVP
GDLGAPGPSGARGERGFPGERGVQGPPGPAGPRGANGAPGNDGAK
GDAGAPGAPGSQGAPGLQGMPGERGAAGLPGPKGDRGDAGPKGAD
GSPGKDGVRGLTGPIGPPGPAGAPGDKGESGPSGPAGPTGARGAP
GDRGEPGPPGPAGFAGPPGADGQPGAKGEPGDAGAKGDAGPPGPA
GPAGPPGPIGNVGAPGAKGARGSAGPPGATGFPGAAGRVGPPGPS
GNAGPPGPPGPAGKEGGKGPRGETGPAGRPGEVG.

The amino acid sequence corresponding to the “K—C” sequence in Table 12 is shown below:

(SEQ ID NO: 113)
QLGDRLNVEVDAAPTVDLNRVLNETRSQYEALVETNRREVEQWFT
TQTEELNKQVVSSSEQLQSYQAEIIELRRTVNALEIELQAQHNLR
DSLENTLTESEARYSSQLSQVQSLITNVESQLAEIRSDLERQNQE
YQVLLDVRARLEGVPGDLGAPGPSGARGERGFPGERGVQGPPGPA
GPRGANGAPGNDGAKGDAGAPGAPGSQGAPGLQGMPGERGAAGLP
GPKGDRGDAGPKGADGSPGKDGVRGLTGPIGPPGPAGAPGDKGES
GPSGPAGPTGARGAPGDRGEPGPPGPAGFAGPPGADGQPGAKGEP
GDAGAKGDAGPPGPAGPAGPPGPIGNVGAPGAKGARGSAGPPGAT
GFPGAAGRVGPPGPSGNAGPPGPPGPAGKEGGKGPRGETGPAGRP
GEVG.

Keratin polypeptides that had a collagen-derived solubility-enhancing sequence fused to both the N-terminal and C-terminal locations (“stability sandwiches”) were found to have an increased solubility at low pH, in comparison to keratin polypeptides without the collagen-derived solubility-enhancing sequence (data not shown). Additionally, the polypeptide sequences can also be designed, if desired, to have repeated collagen sequences at one or both ends of the keratin sequence. By use of this method, the keratin polypeptide in combination with collagen-derived solubility-enhancing sequence is more soluble at a low pH than the keratin polypeptide alone.

### Example 8. Use of a Cleavable Linker Between the Solubility-Enhancing Amino Acid Sequence and the Keratin Polypeptide Fragment

In some instances, when the keratin polypeptide is produced according to methods of the present disclosure, it results in a fusion protein with the solubility-enhancing amino acid sequence. If desired, the fusion protein can be readily separated to its separate polypeptides by using an enzymatically cleavable linker region in the initial design. After cleaving the linker with the corresponding protease, further purification of one or both polypeptides can be performed.

The following example demonstrated successful cleavage of the keratin polypeptide. A nucleic acid sequence encoding the protease cleavage site “ENLYFQG” (SEQ ID NO: 34) or alternatively “ENLYFQ” (SEQ ID NO: 114) was inserted between the C-terminus of the solubility-enhancing amino acid sequence derived from a human collagen type I, alpha 1 polypeptide (HSCol1a-27) encoding nucleic acid sequence and the N-terminus of the human keratin type 31 polypeptide fragment (K31) encoding nucleic acid sequence. The TEV protease cleaves between the Q and G residues of the canonical TEV protease cleavage site ENLYFQG (“tev”) (SEQ ID NO: 34). The TEV cleavage system would therefore normally produce a glycine amino acid “scar” on the cleaved keratin polypeptide. To produce a keratin polypeptide without the scar, the fusion nucleic acid sequence was modified so that the keratin sequence is truncated by 3 amino acids on its N-terminal end; instead of (tev)-QLGDRL . . . (SEQ ID NO: 115), the sequence (tev)-DRL . . . was used. Because the post-cleavage glycine “scar” is part of the native keratin sequence, the resultant fragment (GDRL . . . ) (SEQ ID NO: 116) can be considered scarless.

The nucleotide sequence encoding the above-described fusion protein was:

(SEQ ID NO: 117)
GGTGTACCTGGAGATTTGGGAGCTCCTGGACCATCAGGCGCTCGC
GGCGAACGCGGATTTCCCGGTGAACGTGGGGTTCAAGGTCCACCc
GGCCCTGCTGGCCCTCGTGGTGCGAACGGTGCGCCAGGAAATGAT
GGTGCCAAAGGCGATGCTGGCGCGCCTGGCGCTCCTGGATCTCAA
GGAGCCCCAGGCTTACAAGGTATGCCCGGAGAACGTGGCGCCGCA
GGCTTACCTGGACCTAAAGGTGACCGTGGAGATGCAGGACCTAAA
GGAGCAGATGGCTCTCCTGGAAAGGATGGAGTACGCGGTTTAACT
GGTCCTATTGGTCCACCCGGCCCTGCAGGCGCTCCCGGGGACAAA
GGTGAGTCAGGACCTTCAGGTCCTGCTGGTCCAACAGGTGCTCGC
GGAGCACCAGGAGATCGTGGAGAGCCAGGACCCCCAGGACCCGCG
GGCTTCGCAGGTCCGCCTGGTGCCGACGGACAACCTGGTGCGAAA
GGTGAACCAGGAGATGCTGGCGCTAAAGGAGATGCCGGACCACCT
GGACCAGCGGGACCTGCGGGTCCACCCGGCCCAATCGGAAATGTT
GGCGCACCAGGTGCCAAAGGCGCACGCGGATCCGCCGGTCCCCCA
GGAGCCACGGGTTTCCCTGGTGCGGCTGGTCGCGTAGGGCCACCA
GGTCCCTCAGGCAACGCGGGTCCACCTGGTCCGCCAGGACCAGCA
GGCAAGGAAGGTGGTAAGGGACCACGTGGTGAGACGGGCCCAGCG
GGCCGTCCAGGCGAAGTAGGGGAAAACCTGTATTTTCAGGGTGAT
CGCCTGAATGTTGAAGTTGATGCAGCACCGACCGTTGATCTGAAT
CGTGTGCTGAATGAAACCCGTAGCCAGTATGAAGCCCTGGTGGAA
ACCAATCGTCGTGAAGTTGAACAGTGGTTTACCACACAGACCGAA
GAACTGAATAAACAGGTTGTTAGCAGCAGCGAACAGCTGCAGAGT
TATCAGGCAGAAATCATTGAACTGCGTCGCACCGTTAATGCACTG
GAAATCGAACTGCAGGCACAGCATAATCTGCGTGATAGCCTGGAA
AATACCCTGACCGAAAGCGAAGCACGTTATAGCAGCCAGCTGAGC
CAGGTTCAGAGCCTGATTACCAATGTTGAAAGTCAGCTGGCAGAA
ATTCGTAGTGATCTGGAACGTCAGAATCAAGAATATCAGGTTCTG
CTGGATGTTCGTGCCCGTCTGGAA

The amino acid sequence corresponding to the above nucleic acid sequence was:

(SEQ ID NO: 118)
GVPGDLGAPGPSGARGERGFPGERGVQGPPGPAGPRGANGAPGND
GAKGDAGAPGAPGSQGAPGLQGMPGERGAAGLPGPKGDRGDAGPK
GADGSPGKDGVRGLTGPIGPPGPAGAPGDKGESGPSGPAGPTGAR
GAPGDRGEPGPPGPAGFAGPPGADGQPGAKGEPGDAGAKGDAGPP
GPAGPAGPPGPIGNVGAPGAKGARGSAGPPGATGFPGAAGRVGPP
GPSGNAGPPGPPGPAGKEGGKGPRGETGPAGRPGEVGENLYFQGD
RLNVEVDAAPTVDLNRVLNETRSQYEALVETNRREVEQWFTTQTE
ELNKQVVSSSEQLQSYQAEIIELRRTVNALEIELQAQHNLRDSLE
NTLTESEARYSSQLSQVQSLITNVESQLAEIRSDLERQNQEYQVL
LDVRARLE.

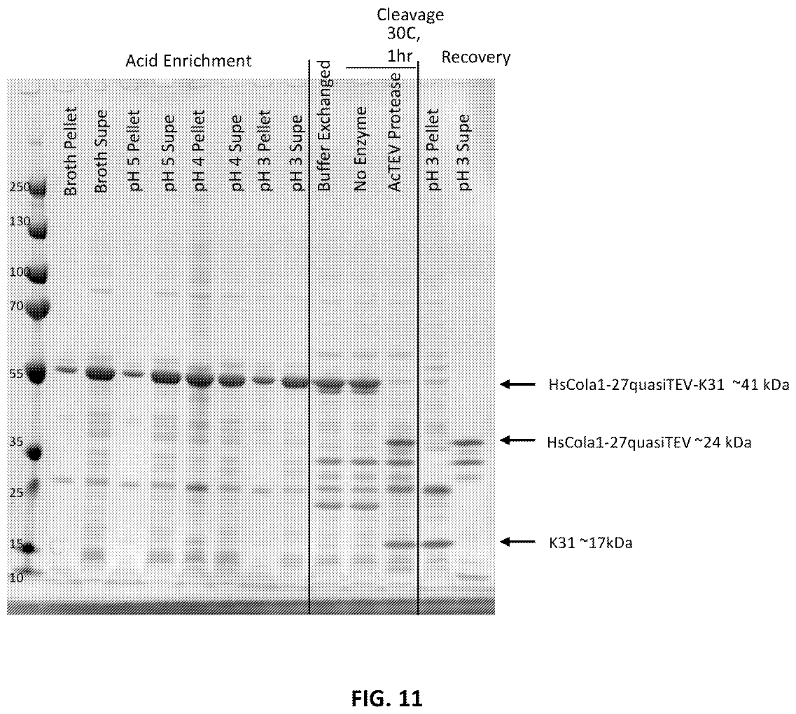

The fusion protein was prepared as described herein, and acid-enriched purification of the fusion protein was performed as described herein. After centrifugation, a 280 μl sample from the acidified (pH 3.0) supernatant was buffer exchanged into 50 mM Tris-HCl pH 8.0+0.5 mM ETDA. To the buffer-exchanged fusion protein, 70 μl AcTEV protease was added, in a total volume of 2100 μl. The mixture was incubated 30° C. for 1 hour, followed by centrifugation. 10 μl of either the pellet or the supernatant fraction of the cleaved fusion protein was separated on an SDS-PAGE gel and stained with Coomassie Blue to confirm that the cleavage was successful (FIG. 11). Further, as expected, the resulting cleaved keratin polypeptide was located in the pellet fraction, while the cleaved collagen-derived solubility-enhancing amino acid sequence was located in the supernatant fraction. This demonstrates that the method can be used to isolate difficult to purify proteins, such as keratin polypeptides, through the use of fusion constructs incorporating a cleavable linker, and then separating the components to generate the desired keratin polypeptide alone.

### Example 9. Scaled Fermentation and Purification of a Keratin Polypeptide

Following fermentation that achieves a suitable cell density and protein expression level, the fermentation broth is chilled until <15° C. is reached. At this point, a concentrated sulfuric acid solution (98.5% wt) is titrated into the fermentor to reduce the broth pH to 3.0-3.2. Centrifugation is used as the primary broth clarification step to remove the cell biomass and larger cell debris. Microfiltration is then used to remove any residual cells and cell debris in the product-containing stream from centrifugation. Ultrafiltration is used to remove residual salts, sugars, soluble fermentation byproducts and water from the microfiltration permeate stream. Additional steps may include treatment with activated carbon to de-color and de-odorize protein concentrate, preservative addition, pH adjustment (to 4-5) and sterile filtration. The formulated protein concentrate then is spray dried to remove most of the water and generate the final polypeptide powder. Alternatively, polypeptide can be formulated to a solution form. The polypeptide can then be used in the manufacture of compositions and formulations as provided herein.

It may also be desirable to produce hydrolysates of the recombinant polypeptides. Recombinant polypeptides can be dissolved in a suitable solvent (such as an aqueous buffer solution) and treated with either heat or acid, while mixing, to create a hydrolysate having a similar amino acid composition, but with smaller fragments than the recombinant protein. The hydrolysate is then spray-dried or lyophilized. The hydrolysate is useful for personal care products, the food industry, cosmetics industry, or the hair care industry. Further, the hydrolysate peptides may be particularly useful, for example, in strengthening normal or damaged hair, due to the ability of the smaller peptides to enter into the inside of the individual hair strands, strengthening the hair from the inside. The recombinant polypeptide may be dissolved in an aqueous solution, and either heated, treated with an acidic solution, or treated with an enzyme in order to break a portion of the peptide bonds and create smaller peptides. The hydrolysate material may then be spray-dried or lyophilized.

### Example 10. Preparation of Various Personal Care Products from a Powder of the Keratin Polypeptides

Powders comprising keratin polypeptides of the present disclosure can be used to prepare a variety of products for beauty and personal care as described herein. This example demonstrates generally applicable formulations with the use of keratin polypeptides produced by the methods described herein.

Combined powder: Each formulation is started by mixing ingredients prior to adding the powder. With mixing in progress, the powder is then gradually added. Faster hydration of the powder is achieved with the use of a disperser disk or a high-shear mixer. Alternatively, the powder is hydrated in a concentrated premix prior to its incorporation in the formulation. To facilitate hydration and prevent the formation of lumps, the powder can be premixed into a slurry with a liquid ingredient (e.g., glycerin, propanediol) before hydration. The powder is incorporated after any neutralization steps of acidic or alkaline components. The powder can also be dispersed in anhydrous systems using a high-shear mixer. The formulation is mixed prior to adding the powder. With mixing in progress, the powder is gradually added until homogenously dispersed. If finer particles are required, a milling step may need to be applied.

An aqueous solution: The powdered polypeptide may be readily converted to a liquid solution for further use as an ingredient. The powder is slowly dissolved in a compatible solution, typically at a concentration or 1-2%.

Combined cream or gel formulation: A 1-2% solution of a powdered polypeptide is also readily incorporated into the aqueous phase of a formulation with standard processing tools. Best results are achieved when the solutions were incorporated into the formulation below 40° C. and after any neutralization steps of acidic or alkaline components.

TABLE 13
An exemplary gel cream formulation 0.1% (w/w) of a keratin polypeptide.

| Phase | Ingredient | Amount (% w/w) |
|---|---|---|
| Water phase | Water | 77.65 |
| | Levulinic Acid, Water, Glycerin, | 4.00 |
| | Sodium Levulinate blend | |
| | Polyglyceryl-3 Methylglucose | 2.00 |
| | Distearate | |
| | Glyceryl Undecylenate | 2.00 |
| Oil phase | Simmondsia Chinensis (Jojoba) Seed | 2.00 |
| | Oil | |
| | Polyacrylate Crosspolymer | 1.25 |
| | Squalane | 1.00 |
| Active ingredient | Water | 10.00 |
| phase | Keratin Polypeptide | 0.10 |

TABLE 14
An exemplary aqueous gel formulation containing
0.1% (w/w) of keratin polypeptide.

| Ingredient | % wt. | Phase |
|---|---|---|
| Water | 45.0 | A |
| Sodium Hyaluronate | 0.1 | |
| Carbomer (Carbopol ® Ultrez 30 Polymer) | 0.4 | B |
| Glycerin | 1.0 | C |
| Phenoxyethanol | 1.0 | |
| Pentylene Glycol (Hydrolite ® 5 Green) | 0.5 | |
| Water | 45.0 | D |
| Keratin Polypeptide | 0.1 | |
| Water (and) Sodium | 0.5 | E |
| Lauryl Sulfoacetate (and) Pentylene Glycol (and) | | |
| Sodium Oleoyl Sarcosinate (and) Sodium Chloride (and) | | |
| Sodium Oleate (SymSol ® PF-3) | | |
| Water (And) Sodium Hydroxide (10N) | PH | F |
| Water | QS | G |
| | 100 | |

Hair care formulation: Formulation A and Formulation B, below, are prepared and mixed well. The formulation is tested on freshly washed hair and dried hair as either a rinsed treatment or a leave-on treatment. Shininess, combability, and hair strength are measured from 30 minutes to 2 hours after application.

TABLE 15
Exemplary hair care formulations containing the keratin polypeptide.

| | Hair Care Gel Formulation | Hair Care Gel Formulation |
|---|---|---|
| Component | A (approximate %) | B (approximate %) |
| Keratin polypeptide | 2% | 0.5% |
| Glycol distearate | 5% | 8% |
| Polyquaternium-10 | 0.1% | 0.2% |
| Polyquaternium-67 | 0.2% | 0.1% |
| Sodium chloride | 0.5% | 0.8% |
| Coco-glucoside | 10% | 8% |
| Sodium cocyl isethionate | 2% | 4% |
| Cocamidopropyl betaine | 5% | 3% |
| Coco-betaine | 0.1% | 0.5% |
| PEG-55 Propylene glycol | 0.7% | 0.5% |
| oleate | | |
| PEG-150 Distearate | 0.4% | 0.8% |
| Coco-caprylate | 0.5% | 0.8% |
| Glycerin | 6.5% | 0.75% |
| Dimethicone | 1% | 0.2% |
| Water | QS | QS |

### Example 11. Toxicology Analyses of a Formulation Comprising a Keratin Polypeptide

A variety of toxicology assays are performed in vitro to screen for any potential negative impact of formulations containing a keratin polypeptide.

Bacterial Reverse Mutation Assay: The polypeptide is evaluated for the ability to induce a mutagenic response in four different strains of Salmonella typhimurium and an E. coli strain. Samples are screened at different dose levels by plating them with the tester strains both with and without Arocolor™ 1254 induced rat liver microsomes (S9). Samples are considered mutagenic if they cause an increase in revertant colonies above the spontaneous background level. The assay is known in the art and is performed compliant with OECD 4714 Guideline for Testing of Chemicals: Bacterial Reverse Mutation Assay.

EpiDerm™ Skin Model in vitro Toxicity Testing: the polypeptide is evaluated for irritancy potential utilizing the MatTek Corporation EpiDerm™ in vitro toxicity testing system as is known in the art. Briefly, normal, human-derived epidermal keratinocytes (NHEK) which have been cultured to form a multilayered, highly differentiated model of the human epidermis are tested with substances and evaluated for damage to mitochondrial enzyme succinate dehydrogenase, as monitored by a color reaction. The enzyme converts a water-soluble, yellow MTT to a purple, insoluble product, and the amount of MTT converted is proportional to the number of viable cells. Triton X-100 (1%) is used as a positive control.

TABLE 16
Standard ranges for EpiDerm ™ Skin
Model in vitro Toxicity Testing System

| ET-50 (hrs) | Expected In vivo Irritancy | Example |
|---|---|---|
| <0.5 | Severe, probably corrosive | Conc. Nitric Acid |
| 0.5-4 | Moderate | 1% Sodium Dodecyl Sulfate |
| 4-12 | Moderate to Mild | 1% Triton X-100 |
| 12-24 | Very Mild | Baby Shampoo |
| 24 | Non-irritating | 10% Tween 20 |

EpiOcular™ Tissue Model in vitro toxicity testing system: The polypeptide is evaluated for irritancy potential utilizing the MatTek Corporation EpiOcular™ in vitro toxicity testing system as is known in the art. Briefly, normal, human-derived epidermal keratinocytes which have been cultured to form a stratified, squamous epithelium similar to that found in the cornea are tested with substances and evaluated for damage to mitochondrial enzyme succinate dehydrogenase, as monitored by a color reaction. The enzyme converts a water-soluble, yellow MTT to a purple, insoluble product, and the amount of MTT converted is proportional to the number of viable cells. Triton X-100 (0.3%) is used as a positive control.

TABLE 17
Standard ranges for EpiOcular ™ Tissue Model in vitro toxicity testing

| Draize Score | Irritancy Classification | Example | EpiOcular ET-50 (min) |
|---|---|---|---|
| 0-15 | Non-irritating, Minimal | PEG-75 Lanolin, Tween 20 | >256-26.5 |
| 15.1-25 | Mild | 3% Sodium Dodecyl Sulfate | <26.5-11.7 |
| 25.1-50 | Moderate | 5% Triton X-100 | <11.7-3.45 |
| 50.1-110 | Severe, Extreme | 5% Benzalkonium Chloride | <3.45 |

### Example 12. In Vitro Studies of a Keratin Polypeptide of the Present Disclosure on Skin Cells

Healthy skin is primarily composed of collagen types I and III, hyaluronans, fibronectin and elastin, and a basal lamina that includes other proteins such as laminins and collagen IV. Fibroblasts are the major cell type that produces these structural proteins, including collagen. Collectively the proteins are known as extra cellular matrix (ECM), and they support the skin's structure. Fibroblast output of collagen decreases with age, so fibroblasts are a primary target for the activity of cosmetics to try to rescue skin aging.

Keratinocytes are the major cell type forming the epidermis, or outer layers of the skin. HaCaT cells are an immortal keratinocyte cell line derived from adult skin. Both cell types are used to demonstrate the benefits of formulations on skin. These cells have a high turn-over and receive the brunt of everyday pollution and radiation. They are negatively affected by the environments we subject them to, which leads to increased inflammation and damage to our natural skin barrier. Hallmarks used to assess keratinocyte health include inflammatory markers, cell turnover, and DNA integrity.

Toxicity. Human primary fibroblasts, HaCaT cells, and human primary keratinocytes are treated with keratin polypeptides and viability is determined using a standard MTT assay.

### Protocol:

1. The cells are seeded at confluency in 96-well plate.
2. 24 hours later the media is changed to low serum media (to avoid any effects due to serum).
3. The cells are treated with polypeptide in the same low serum media for 24 hrs.
4. Post treatment with polypeptide, the supernatants were saved, and cells are incubated with MTT dye for 60 mins at 37° C.
5. MTT is metabolized to formazan salts by viable cells.
6. These salts are dissolved using isopropanol and the color produced is quantified using a cell plate reader.

Effect on keratinocyte growth and regeneration. Keratinocytes or HaCaT cells are treated with keratin polypeptides and growth and regeneration is measured.

Effect on fibroblast production of collagen type I. Fibroblasts are cultured with keratin polypeptide and culture supernatants are analyzed by Enzyme Linked Immunosorbent Assay (ELISA) for pro-collagen type I C-peptide, which is a readout for total secreted collagen type I protein.

### ELISA Protocol:

1. Primary human fibroblasts are cultured in standard media DMEM/F12+10% FBS.
2. Supernatants are used to determine the level of collagen type I present.
3. The kit used here is Takara Procollagen type I C-peptide detection ELISA kit.
4. Manufacturer's protocol is followed to measure the quantity of Collagen type I in the supernatants.

Effect on fibroblast production of genes for extracellular matrix proteins. Fibroblasts are incubated with keratin polypeptide for approximately 48 hours and then RNA sequencing is then performed to analyze global gene expression in the cells, including several extracellular matrix genes such as collagen type I gene (COL1A), the elastin gene (ELN), and the fibronectin gene (FN1), and genes in several pathways responsible for cell proliferation, migration, adhesion.

### Microarray RNA Analysis Protocol:

1. The cells are seeded at confluency in 6-well plates.
2. 24 hrs later the media is changed to low serum media.
3. The cells are treated with 0.05% polypeptide and control.
4. The QIAGEN RNeasy kit is used to extract the RNA and the extracted RNA for analysis.

Effect on wound healing. Wound healing is a dynamic process that includes a sequence of events, including cell proliferation and migration. Fibroblast migration and proliferation plays a crucial role in wound closure by secreting various chemicals, including collagen and other matrix proteins. A gap is induced by scratching a confluent layer of fibroblasts, and treatment with keratin polypeptides is monitored by cell proliferation and closing of the gap.

### Protocol:

1. The cells are seeded at confluency in 24 well plate.
2. 24 hours later the media is changed to low serum media and the cells are starved for 6-8 hrs.
3. Post starvation, the wells containing cells are scratched and treated. Images are taken at this time (i.e., time 0 h) and after 24 h.
4. Images were analyzed using Image J software.

Effect on cell viability of keratinocytes exposed to urban dust pollution. HaCaT cells are pre-treated with keratin polypeptide and then exposed to a government-certified urban dust sample at 2 mg/ml.

### Protocol:

1. The cells are seeded at confluency in 96-well plate.
2. The cells are treated with polypeptide for 24 hrs (that is pre-treating the cells before they are expose to Urban dust)
3. The desired urban dust concentration is prepared, and the cells are exposed to it for 24 h.
4. Post urban dust exposure, the supernatants are stored to run different inflammatory cytokines and the cells are incubated with MTT dye for 60 mins at 37° C.
5. MTT is metabolized to formazan salts by viable cells.
6. These salts are dissolved using isopropanol and the color produced is quantified using a cell plate reader.

Antioxidative capacity. The oxygen radical absorbance capacity (ORAC) assay (a cell-free assay that uses a fluorescent readout) is used to show the antioxidant capacity of the keratin polypeptide. Data is reported in Trolox (Vitamin E) equivalents.

Effect on inflammation of keratinocytes irradiated with UVB light. Human primary keratinocytes are irradiated with 40 mJ/cm² UVB light, and then treated with 0.15% of a collagen-keratin fusion polypeptide for 24 hours. Levels of the pro-inflammatory cytokines, such as IL-1a are then determined by ELISA.

### Example 13. Human Clinical Studies of a Topical Skincare Product Containing a Keratin Polypeptide

Anti-aging study to assess the anti-wrinkle efficacy of keratin polypeptide formulations in comparison with placebo product. Female subjects with healthy skin in the face and visible wrinkles in the periorbital regions are enrolled. Skin hydration effects are measured by Corneometer, skin elasticity and firmness effects by Cutometer and epidermal thickness by Vivascope. Additionally, objective evaluation of fine lines and wrinkles, brightness and redness is performed, and images are taken (Colorface) for image analysis. Assessments are performed before, directly after the first product application as well as after 4 and 12 weeks of product application. Furthermore, subjects will fill in a questionnaire concerning product traits.

Parameters—P1: Visual Assessment: appearance of fine lines and wrinkles, brightness, redness [trained grader], P2: Skin hydration [Corneometer], P3: Skin elasticity and firmness [Cutometer], P4: Dermal thickness [Vivascope], P5: Photographic Imaging, Full Face and Profiles (Left and Right), CP, Std60 [ColorFace], P6: Image Analysis (e.g. fine lines and wrinkles, dark spots) [Newtone], P7: Questionnaire (up to 10 questions) [Subject].

Test Area—The measurement of the anti-wrinkle properties (skin roughness) is performed periorbitally, i.e., in the region of crowfeet. Skin moisturizing and firming effects of the cosmetic product are evaluated on the bones of the cheeks. The study is performed in split-face design. Test Procedure Day 1: Baseline measurement after acclimatization P1-P5; Dispensing of test products to the subjects; Application of test product by subjects under supervision, P1-P5, P7; Day 2-85: Application of test product by the subjects at home, twice daily; Day 29: Measurement after acclimatization: P1-P5; Day 57: Compliance check via telephone; Day 86: Measurement after acclimatization: P1-P5, P7, return of products.

### After Day 86: P6.

Panel 100 female subjects (approx. 33 per product) aged between 35 and 70 years with visible eye wrinkles according to proDERM score 3 to 6 with no further specific inclusion. 28 subjects finish per product. Exploratory, randomized, blind for subjects, intra-individual comparison, split-face, placebo-controlled; comparison between test product and placebo per subgroup, comparison between assessment times (baseline day 1 before product application, day one after product application, after 4 and 12 weeks of product application).

Analysis Comparison of times per test product P1-P4: Descriptive statistics for P6, P7; Comparison of each test product vs reference product are performed separately.

Climate Conditions: All investigations are performed in rooms that are completely air-conditioned, especially equipped for the above-described tests after a defined period of acclimation of the panelists. The last application usually takes place the evening before the measurements.

Cutometer—skin firmness by measuring total elasticity and elastic recovery. Skin elasticity is measured with a Cutometer. The measuring principle is based on a suction method. In the measuring head, a vacuum is induced that is set to 300 mbar. The skin on the measured area is sucked into the opening of the measuring head for 5 seconds with a subsequent measuring period of another 5 seconds after release. Using an optical measuring system, it is detected contactlessly how far the skin is sucked into the measuring head; this value gives a measure for skin elasticity. From the resultant measuring curves, 2 parameters are calculated: Total elasticity Uf and quotient of elastic relaxation to total elasticity Ur/Uf.

Corneometer—skin hydration. Measurement of stratum corneum hydration is performed by the electrical capacitance method with the CorneometerCM 825 (Courage & Khazaka, Cologne, Germany). The measuring principle is based on changes in the capacitance of the measuring head, functioning as a condensator. Between the conductors consisting of gold, an electrical field is built. By these means, the dielectricity of the upper skin layer is measured. Because the dielectricity varies as a function of the skin's water content, the stratum corneum hydration can be measured. An increase in Corneometer values shows a skin-moisturizing effect.

Questionnaire—self assessment. Subjects will judge the test product at the end of the study in a questionnaire with up to 10 questions. The questionnaire consists of closed questions with predefined identical options to tick. If the questionnaire strongly deviates from the given structure, additional costs are charged.

Objective evaluation by trained grader, appearance of fine lines and wrinkles, brightness, and redness.

VivaScope—epidermal thickness. The VivaScope®1500 is a device for in vivo confocal scanning laser microscopy. Confocal microscopy is a technique that allows optical sectioning of turbid objects (e.g., skin). With this technique skin can be imaged in vivo in its native state without further preparation. This method enables an in vivo mapping of the skin up to a depth of 350 μm depending on the skin type since the different microstructures within the skin cause naturally variations of the refraction index and therefore provides images with contrast. For example, cytoplasm with a refraction index coming close to that of water (reflectance index 1.33) is depicted with a very low contrast. Melanin and keratin (refrectance index 1.7), however, have a relatively high refraction index and thus act as natural contrasting agents. The VivaScope® 1500 can produce in vivo section of the skin with an optical section thickness less than 5 micrometers and therefore is comparable with histological skin section. The VivaCam® macrocamera allows to capture a macroimages of the test area and to correlate the confocal images with the macroimage.

### Image Analysis by Newtone:

Pigmented spot. Parameters: Colorimetric visibility; color parameters in and outside of the spots (based on baseline spot detection); calculation of contrast Morphological visibility—contrast to the skin: conspicuous area (apparent surface detection in contrast to complexion, based on spot detection at each timepoint), Modalities: CP, View: Profile.

Multi Pigmented spot. Parameters: Area, L*, a*, b*, in pigmented spots and outside, dE76 contrast, Modalities: CP, View: Profile.

Crow's feet wrinkles. Parameters: Conspicuous length, conspicuous surface, conspicuous depth and conspicuous volume, Modalities: Standard60, View: Profile.

Nasolabial folds. Parameters: Conspicuous length, conspicuous surface, conspicuous depth and conspicuous volume, Modalities: Standard60, View: Front face.

Index which can be correlated to radiance. Parameters: Saturation, luminosity, parameter correlated to radiance, Modalities: CP and Std60, View: Profile.

Under eyes wrinkles. Parameters: Conspicuous length, conspicuous surface, conspicuous depth and conspicuous volume, Modalities: Standard60, View: Front face.

Forehead wrinkles. Parameters: Conspicuous length, conspicuous surface, conspicuous depth and conspicuous volume, Modalities: Standard60, View: Front face

Wrinkle Analysis by Colorface (Crow's feet wrinkles, nasolabial folds, under eye wrinkles and forehead wrinkles; Color/Tone Analysis by Colorface (pigmented spots, multi pigmented spots, index correlated to radiance, pores).

### Example 14. Human Clinical Studies of a Haircare Formulation Containing a Keratin Polypeptide

Hair straightening. The formulation is tested for its ability to straighten hair. An amount of partially curly hair is chosen for the test procedure. Each treatment sample included 12 hairs, each about 50 mm in length. To simulate damaged hair, some of the samples are treated with 10% bleach for 10 minutes, followed by rinsing with water. An aqueous solution of the collagen-keratin fusion protein is applied to both the natural hair and the bleach treated hair.

Hair shine. The formulation is tested for its ability to add shine to hair. Samples of freshly washed and cut hair test strands of approximately 100 hairs each (8 inches long) are prepared. An aqueous solution of the collagen-keratin fusion protein is applied to the hair. In some instances, the hair strands are allowed to dry at room temperature, and the application is repeated 2× or 3×. One sample of strands is left untreated, as the negative control. A commercially available shine agent is applied to a sample of the hair strands, as a positive control. After 1 hour of drying at room temperature, the strands are measured for shininess using a glossmeter. A glossmeter is a piece of equipment designed to measure the hair shine. The regularity of the hair surface helps to determine the light reflection. When the light follows a uniform surface, as in a mirror, the incidence angle is exactly equals to the reflection angle. However, the hair is not totally uniform and at some points the light beam is reflected forming different angles. The glossmeter can easily quantify the ability of a hair treatment to increase the shine of the hair strands.

Hair shine/luster. Three bleached type II hair tresses of 4 grams and 20 cm in length are prepared for each treatment. The formulation, in the form of a conditioning wash-out product, is applied to wet hair after a shampoo treatment. The product is left in the hair for 10 minutes. Excess product is then rinsed from the hair. The hair is air dried for 1 hour. The tresses are then photographed using a shine chamber. Two images for each tress are taken in order to obtain the diffuse (orthogonal polarization) and specular (parallel polarization) reflection. The reflections are computed by an internally developed software and stored for Luster calculation. The results are statistically analyzed in comparison to controls.

Protective effects on hair. The loss of water content from treated and untreated hair strands, before and after heat treatment by application of a hair treatment iron, is examined by thermal gravimetric analysis (TGA) using aa TGA 4000 system (Perkin Elmer, Waltham, MA, US). The measurements are performed from 30° C. to 250° C. at 15° C. per minute. To evaluate thermal protection, one bleached type II hair fiber of 1 gram and 15 cm in length is prepared for each treatment. The formulation, in the form of a conditioning wash-out product, is applied to wet hair after a shampoo treatment. The product is left in the hair for 10 minutes. Excess product is then rinsed from the hair. The hair is air dried for 1 hour, then flat ironed at 450° F. for 1 minute. The heat-treated tresses are then analyzed by differential scanning calorimetry. Five crucibles containing 10 mg of hair are analyzed, and the enthalpy of keratin denaturation is computed and statistically analyzed. The higher the enthalpy the higher the thermal protection.

Hair smoothness. The formulation is tested for its ability to smooth the surface of a single hair, using standard SEM photomicrography methods. Several locks of freshly washed and cut hair test strands of approximately 100 hairs each (8 inches long) are prepared. An aqueous solution of polypeptide is applied to the hair. Control hair locks, having an application of the above-described base formulation, but without the polypeptide, are also included in the test. The locks of hair are allowed to dry at room temperature for two hours. Individual hairs of each treated lock are chosen and prepared for SEM photomicrography using standard methods available in the art. The photomicrograph images are examined for differences between the controls and the collagen-keratin treated hairs. The hair is examined visually for sleekness, and microscopically for visible cracks, gaps, and/or fissures.

Hair smoothness on bleached hair. Six bleached type II hair tresses of 5 grams and 20 cm long are prepared for each treatment. The formulation, in the form of a conditioning wash-out product, is applied to wet hair after a shampoo treatment. The product is left in the hair for 10 minutes. Excess product is then rinsed from the hair. The hair is air dried for 1 hour. The hair is then analyzed using an accessory system attached to Instron model 5565. The tresses are passed through a Teflon ring of 12 mm in diameter and 20 mm in width and the resistance force along with the displacement recorded by Instron. The average energy (area under the curve Force vs displacement) is statistically compared where the lower the energy, the higher the hair smoothness.

Hair thickness. Locks of approximately 50 hairs each are either untreated or chemically treated with 8% bleach for 1 hour, then rinsed and dried, in order to simulate the types of hair damage that occurs during certain hairdressing treatments. A solution of polypeptide in a base formulation or the base formulation alone, is applied to the various locks of hair. The hairs are then rinsed three times with water and air-dried for two hours. Diameter of the hairs before and after treatment with the collagen-keratin fusion protein is measured using an inverted microscope.

Hair thickness and health using a hydrolyzed polypeptide formulation. A 1-gram amount of keratin polypeptide is produced as described herein. This is hydrolyzed into shorter polypeptides by boiling in 10% hydrochloric acid for 4 hours. The mixture is cooled and neutralized to pH 6.0 with NaOH. The mixture is filtered, and spray dried to a powder. Locks of hair (about 70 hairs per lock, about 15 cm in length) are taken from several volunteers (with differing hair types). A 1% solution of the hydrolysate powder in water is prepared. The solution is applied to either normal hair, or bleach damaged hair. To approximate a typical use of a hair conditioner by an individual, the locks are washed with shampoo, rinsed, and then incubated for 10 minutes in the collagen-keratin hydrolysate solution in a petri dish. The locks are rinsed with water for 1 minute, allowed to dry, and the process is repeated 2×, 24 hours apart (again, to approximate use by an individual). The hair is then examined using confocal microscopy to determine changes in diameter, health of hair strand, and cracks and crevices in the hair surface (i.e., smoothness of the hair strand). Digital images of the hair cross-sections are taken, and differences in the hair diameter between the untreated and treated hair are quantified.

Extended treatment, evaluation and self-reporting. A standard hair conditioning formula is chosen. To this conditioner, a keratin polypeptide (powder form) or a hydrolysate of keratin polypeptide (powder form) is added and mixed thoroughly until dissolved. In a double-blind study, volunteers are given either the conditioner alone, or the conditioner plus polypeptide, in order to add to their daily haircare routine. After 3 weeks, the volunteer's hair is sampled, and tested for the following: combability, shininess, strength, hydration, and protection from heat damage (such as hair irons). Volunteers report on hair characteristics, e.g., strength, hydration, shine, feel, combability, and protection from heat damage.

Hair breakage. Four bleached type II hair tresses (approximately 3 grams and 20 cm long) are chosen for each treatment. A hair treatment product containing 1% keratin polypeptide is applied to the hair samples and allowed to dry at room temperature for 1 hour. The tresses are then inserted in an automatic brushing machine (BLPA 101) where they are brushed 8000 times in cycles of 1000 times. After each cycle, the number of broken fibers is counted, and statistical comparison of the mean broken fiber value is performed.

Hair strength/tensile strength. 50 bleached hair fibers of 60 mm in length are prepared for each treatment. A hair treatment product containing 1% keratin polypeptide is applied to the hair fibers and allowed to dry at room temperature for 1 hour. The hair strands are then analyzed using an Instron model 5565 tensile testing system, where the mechanical behavior for each fiber is recorded. Three main parameters of the average tensile curve are evaluated for three factors: maximum stress (the stress at the breaking point, which is an indicator of fiber strength), Young modulus (the elasticity modulus of the Hookean region, which is an indicator of fiber stiffness), and maximum strain (the strain at the breaking point). The results are analyzed statistically.

Repair of split ends. Six bleached type II hair tresses (but otherwise untreated) are prepared for each treatment. The tresses are then inserted in an automatic brushing machine (BLPA 101) where they are brushed 2000 times. The split ends are then counted. The formulation is applied to the hair and allowed to dry for 3 hours at room temperature. The split ends are re-counted, and the results are statistically analyzed.

Hair frizz and volume. Six bleached type III hair tresses are prepared for each treatment. The formulation, in the form of a leave-on hair rinse product is applied to the hair tresses, and the hair tresses are placed in a humidity and temperature control chamber where they will remain for 24 hours (55±5% Relative Humidity (RH); 22±2° C.). The tresses are then photographed, and the images are analyzed using digital imaging software to determine the level of frizz and volume (T0). The tresses are then reinserted into the humidity and temperature control chamber where they will remain for further 24 hours (85±5% RH; 22±2° C.). The tresses are photographed and analyzed once again (T24), and the values of frizz and volume are statistically compared between the treatments for both time-points.

Hair curl retention. Six virgin type VI hair tresses (curly) are prepared for each treatment. The formulation, in the form of a conditioning wash-out product, is applied to wet hair after a shampoo treatment. The product is left in the hair for 10 minutes. Excess product is then rinsed from the hair, and the hair is air dried for 1 hour. The hair is photographed and analyzed using digital imaging software to measure the frizz and volume, number of curls and length (TO). Then, the tresses are inserted into the humidity and temperature control chamber where they will remain for 24 hours (85±5% RH; 22±2° C.). The tresses are photographed and analyzed once again (T24) and the values of frizz and volume, number of curls and length is statistically compared between the treatments for both time-points.

Microscopic analysis of hair fibers. Samples of bleached type II hair fiber of 1 gram and 15 cm in length are prepared for each treatment. The formulation, in the form of a conditioning wash-out product, is applied to wet hair after a shampoo treatment. The product is left in the hair for 10 minutes. Excess product is then rinsed from the hair. The hair is air dried for 1 hour. The hair is then examined by SEM microscopy, at each of the following treatment steps: just after application, after the heat damage with mechanical breakage, and after damaging with a flat iron. The images are statistically analyzed using digital imaging software.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 15. Purifying Recombinant Proteins with pH Modulation

Protein purification is a crucial step in recombinant protein production that can be time and resource consuming. Described in this example is a method of purifying recombinant proteins from microbial host cells to achieve recombinant protein products of high purity. This purification method can be used to purify recombinant proteins that are retained intracellularly or secreted. This purification method can also be used to purify recombinant proteins from cell lysate or supernatant. A schematic workflow of the purification process is illustrated in FIG. 1B.

Cell lysate of microbial host cells expressing a recombinant protein of interest can be used as the input material for this purification process. The cell lysate can be separated into a soluble fraction and an insoluble fraction (e.g., using centrifugation). Supernatant from cell culture media comprising microbial host cells that express a recombinant protein of interest can also be used as the input material for this purification process. The supernatant can similarly be separated into a soluble fraction and an insoluble fraction (e.g., using centrifugation).

First, the separated soluble fraction can be treated with an acid (e.g., H₂SO₄) to a pH of about 5 to generate an acid-treated mixture followed by separation of the acid-treated mixture into a soluble and insoluble fraction. The acid treatment and separation steps can be iterated to adjust the soluble fraction to a pH of about 4, then to a pH of about 3. This method greatly reduces contaminating proteins, such as endogenous host cell proteins, yielding a highly pure recombinant protein product (FIG. 2, Example 1; FIG. 6—Example 4). This purification method can yield purified recombinant protein products of high purity when analyzed by mass spectrometry, where purity is defined as the percentage of proteins containing a matching sequence to the target protein relative to all proteins present.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 16. A Recombinant Keratin Fusion Polypeptide of the Disclosure is Deposited on the Hair Surface

This example was performed to determine the level of hair surface deposition of an exemplary recombinant keratin fusion polypeptide of the disclosure (SEQ ID NO: 71), a property that can impact the durability of benefits potentiated by application of the fusion polypeptide. A solution comprising 1% of the recombinant keratin fusion polypeptide formulated at a pH of 4 was used to evaluate hair surface deposition on the surface of type I hair that was bleached three times (3× bleached type I hair) with various application regimens. The recombinant polypeptide having an amino acid sequence of SEQ ID NO: 71 used in this experiment was labeled with a Cy5-conjugated NHS ester, enabling visualization of the recombinant keratin fusion polypeptide on hair strands using fluorescence microscopy (EVOS M5000, ThermoFisher Scientific).

Hair strands were soaked in 1% of solution comprising the recombinant keratin fusion polypeptide, with both ends kept outside of the solution, for 1, 3, or 5 applications (5 minutes per application) or for 1 hour. Hair strands were washed with deionized water for 30 seconds after each application and were gently pressed between two layers of laboratory paper wiper to dry.

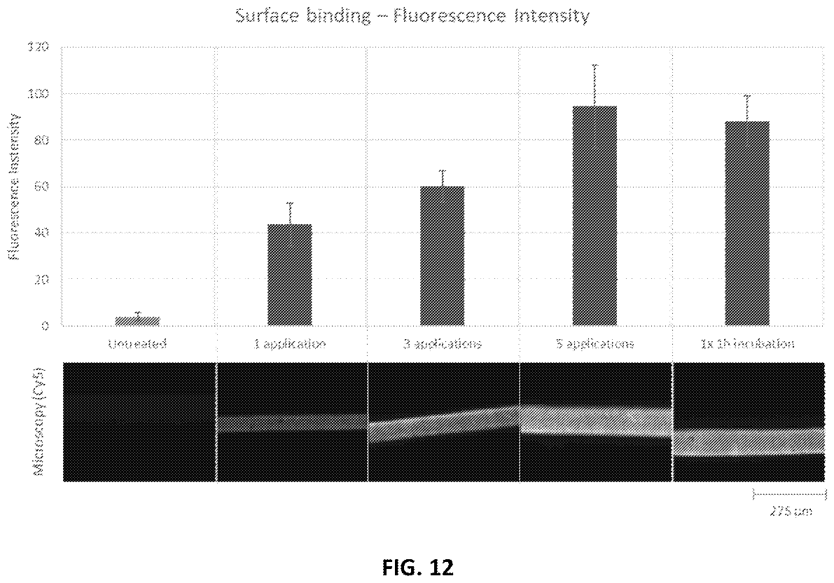

Recombinant keratin fusion polypeptide hair surface deposition was evaluated using fluorescence microscopy in which hair surface deposition was quantified using ImageJ (FIG. 12). The fluorescence intensity described here was defined as the mean gray value of the hair (8-bit) minus the mean gray value of the background. The data show that the recombinant keratin fusion polypeptide can be found on the hair surface just after 1 application and wash cycle (FIG. 12). Furthermore, the data demonstrate that the deposition of the recombinant keratin fusion polypeptide on the hair surface increases with repeated application as suggested by the increase in fluorescence intensity (FIG. 12). Moreover, it can be seen that the extent of recombinant keratin fusion polypeptide hair surface deposition achieved through 5 short applications can be recapitulated with a single 1-hour incubation period (FIG. 12). Collectively, the data show that the recombinant keratin fusion polypeptide is deposited on the hair surface after one short application, and that more recombinant keratin fusion polypeptide can be deposited onto the hair surface by increasing the frequency or duration of application.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 17. A Recombinant Keratin Fusion Polypeptide of the Disclosure Penetrates Hair Strands

This example was performed to evaluate the ability of an exemplary recombinant keratin fusion polypeptide of the disclosure (SEQ ID NO: 71) to penetrate hair strands, a property that can impact the potency and durability of the beneficial effects of the recombinant keratin fusion polypeptide. A solution comprising 1% of the recombinant keratin fusion polypeptide formulated at a pH of 4 was used to evaluate penetration into type I hair that was bleached three times (3× bleached type I hair) with various application regimens. The recombinant keratin fusion polypeptide used in this experiment was labeled with a Cy5-conjugated NHS ester, enabling visualization of the recombinant keratin fusion polypeptide on hair strands using fluorescence microscopy.

Hair strands were soaked in solution comprising 1% recombinant keratin fusion polypeptide, with both ends kept outside of the solution, for 1, 3, or 5 applications (5 minutes per application) or for 1 hour. Hair strands were washed with deionized water for 30 seconds after each application and were gently pressed between two layers of laboratory paper wiper to dry. Cross sections of treated hair were prepared for subsequent imaging by fluorescence microscopy (EVOS M5000, ThermoFisher Scientific).

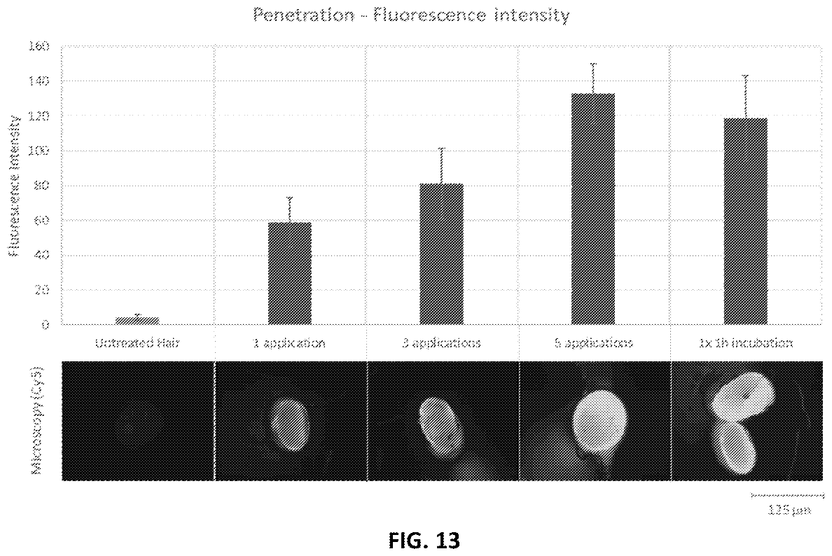

Recombinant keratin fusion polypeptide hair penetration was evaluated using fluorescence microscopy in which penetration was quantified using ImageJ (FIG. 13). The fluorescence intensity described here was defined as the mean gray value of the hair (8-bit) minus the mean gray value of the background. The data show that the recombinant keratin fusion polypeptide can be found in the cortex of hair just after 1 application and wash cycle (FIG. 13). Similar to hair surface deposition findings, recombinant keratin fusion polypeptide hair penetration increased with repeated application, and the effect of 5 short repeated applications can be recapitulated with a single 1-hour incubation (FIG. 13). Taken together, the data show that the recombinant keratin fusion polypeptide can effectively penetrate the hair cortex just after 1 short application, and that increasing the frequency or duration of the recombinant keratin fusion polypeptide application can substantially enhance recombinant keratin fusion polypeptide penetration.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 18. A Recombinant Keratin Fusion Polypeptide of the Disclosure Resists Multiple Shampoo Cycles

This example was performed to evaluate the ability of an exemplary recombinant keratin fusion polypeptide of the disclosure (SEQ ID NO: 71) to resist multiple shampoo cycles, a property that can impact the durability of benefits potentiated by recombinant keratin fusion polypeptide application. A solution comprising 1% of the recombinant keratin fusion polypeptide formulated at a pH of 4 was used for application to type I hair that was bleached three times (3× bleached type I hair). The recombinant keratin fusion polypeptide used in this experiment was labeled with a Cy5-conjugated NHS ester, enabling visualization of the recombinant keratin fusion polypeptide on hair strands using fluorescence microscopy (EVOS M5000, ThermoFisher Scientific).

The recombinant keratin fusion polypeptide was applied to hair strands by soaking the hair strands in 1% solution comprising the recombinant keratin fusion polypeptide for 1 hour followed by a 30 second wash with deionized water. Hair strands were dried by gently pressing the hair strands between two layers of laboratory paper wiper. Next, hair strands underwent shampoo cycles consisting of 1) soaking in 2% sodium lauryl sulfate (SLS) solution for 1 minute, 2) rinsing with deionized water for 30 seconds, and 3) drying by gently pressing hair strands between two layers of laboratory paper wiper. Treated hair strands underwent 0, 1, 3, or 5 consecutive shampoo cycles. Hair strands and hair cross sections were prepared for subsequent imaging by fluorescence microscopy.

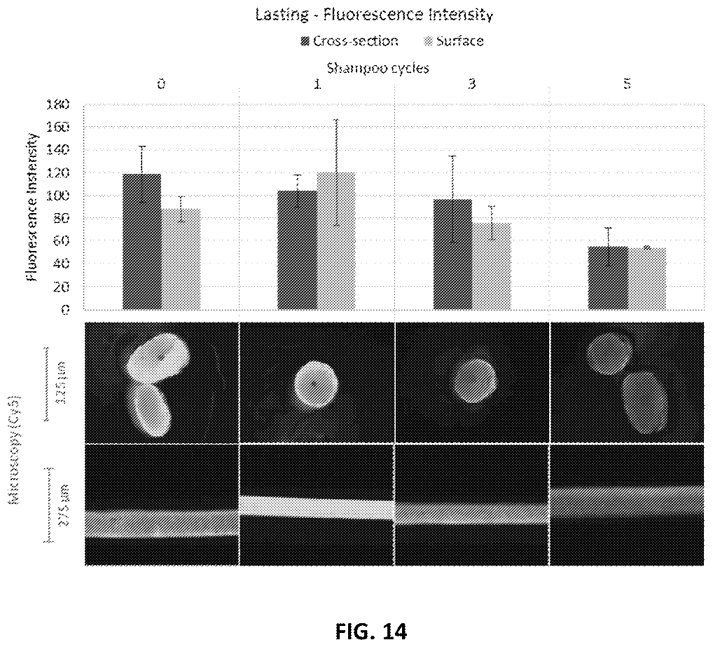

The recombinant keratin fusion polypeptide resisted multiple shampoo cycles as evidenced by the presence of remaining recombinant keratin fusion polypeptide on the surface and within the cortex (FIG. 14). Notably, hair strands that had undergone 5 consecutive shampoo cycles still retained ˜50% of the recombinant keratin fusion polypeptide in the cortex when compared to recombinant keratin fusion polypeptide-treated hair strands that were not shampooed (FIG. 14). This data highlights the robustness of the recombinant keratin fusion polypeptide and a potential for long lasting performance for hair care.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 19. A Recombinant Keratin Fusion Polypeptide of the Disclosure Promotes Thermal Protection to Hair

This example was performed to evaluate the ability of an exemplary recombinant keratin fusion polypeptide of the disclosure (SEQ ID NO: 71) to confer thermal protection to hair. A solution comprising 1% of the recombinant keratin fusion polypeptide formulated at a pH of 4 using sodium hydroxide, was used for application to European medium brown hair tresses.

Tresses were first cleaned with 15% sodium lauryl ethyl sulfate (SLES) solution (0.1 mL/g_{dry hair}) and subsequently dried on a towel, a cleaning cycle that was repeated 3 times. 1% recombinant keratin fusion polypeptide solution was applied to cleaned tresses (0.2 mL/g_{dry hair}) as a leave-on apart from control tresses, which were treated with 1 mL of deionized water. Then, tresses were subjected to a heat damage protocol consisting of 10 strokes of flat iron 450° F. (232° C.) with the exception of one control tress that did not undergo heat treatment. After heat treatment, tresses were cleansed with 15% SLES solution, incubated in deionized water overnight, and then dried overnight. Fiber snippets from the middle section of the hair were collected for analysis using differential scanning calorimetry.

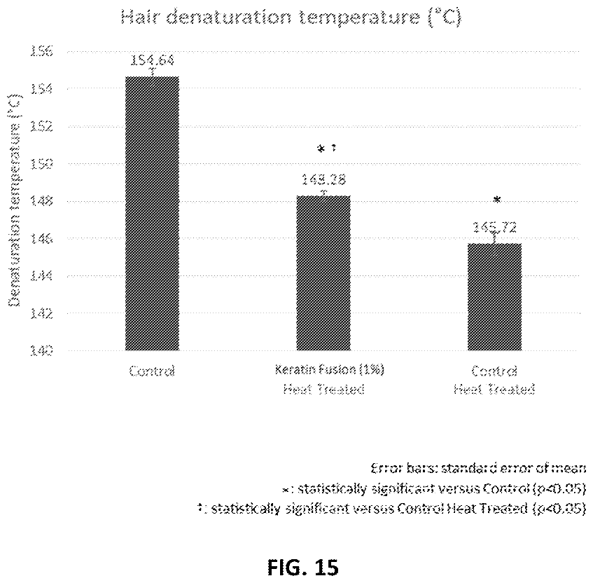

Hair treated with 1% recombinant keratin fusion polypeptide solution had a higher denaturation temperature than untreated hair (control) after heat damage, suggesting that the recombinant keratin fusion polypeptide improves matrix crosslinking density when compared to untreated hair (control) in response to heat damage (FIG. 15). The data clearly demonstrate that hair pre-treatment with a 1% recombinant keratin fusion polypeptide solution can promote significant thermal protection.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 20. A Recombinant Keratin Fusion Polypeptide of the Disclosure Improves Hair Suppleness

This example was performed to evaluate the impact of an exemplary recombinant keratin fusion polypeptide of the disclosure (SEQ ID NO: 71) on hair suppleness. A solution comprising 1% of the recombinant keratin fusion polypeptide formulated at a pH of 4 was used for application to type I hair tresses that were bleached three times (3× bleached type I hair tresses).

Tresses were first cleaned with 20% SLS solution (0.4 g/g_{dry hair}) and then dried for 24 hours at 85% RH. 1% recombinant keratin fusion polypeptide solution was applied to dry tresses (0.2 g/g_{dry hair}), rested for 5 minutes, and then rinsed under running water for 15 seconds. Some tresses were subject to multiple recombinant keratin fusion polypeptide application and drying cycles. Tresses were dried for 24 hours at 85% HR after the 1^{st} and 5^{th} application but otherwise blow dried using the cold setting in between applications. No resting or rinsing was performed on the test condition for the leave-on application condition.

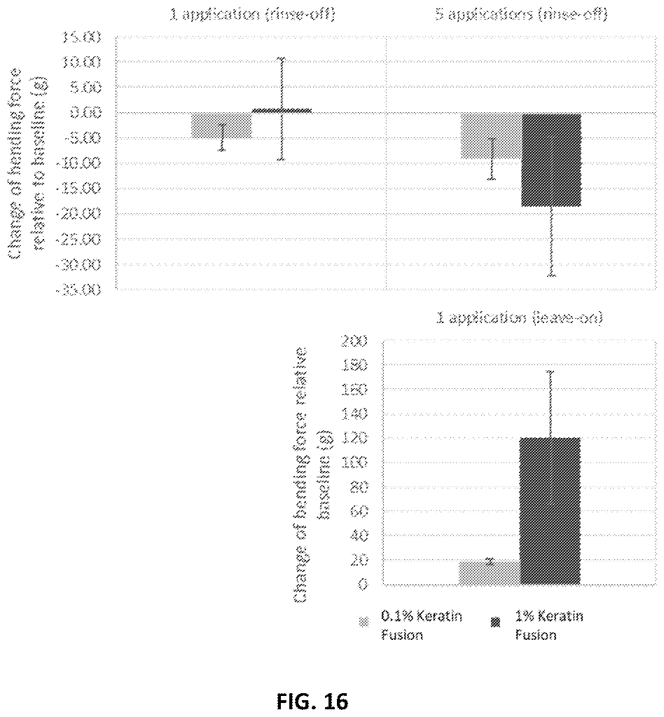

A 3-point bend test was performed on tresses, using the TA XT (Stable Microsystems) with a target distance of 10 mm, after baseline and after 1 and 5 treatment applications. A lower bending force indicates that the hair is more pliable and can translate to supple hair with more natural hair movement. The data show that recombinant keratin fusion polypeptide application as a rinse-off reduced the force necessary to bend tresses, suggesting that a rinse-off application of the recombinant keratin fusion polypeptide rendered the hair more supple (FIG. 16). Increasing the number of rinse-off application iterations further decreased the force necessary to bend tresses, indicating that hair suppleness increases with increasing rinse-off application frequency (FIG. 16). In contrast, tresses conditioned with the recombinant keratin fusion polypeptide as a leave-on increased the force necessary to bend tresses (FIG. 16).

Collectively, it can be seen that the recombinant keratin fusion polypeptide can be versatile in imparting suppleness or stiffness depending on the application regimen. The recombinant keratin fusion polypeptide can be applied to hair and rinsed off when supple hair is desired, and alternatively, the recombinant keratin fusion polypeptide can be applied to hair and left on for styling and hair manageability purposes.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 21. A Recombinant Keratin Fusion Polypeptide of the Disclosure Improves Style Retention

This example was performed to evaluate the effect of an exemplary recombinant keratin fusion polypeptide of the disclosure (SEQ ID NO: 71) on hair style retention. A solution comprising 1% of the recombinant keratin fusion polypeptide formulated at a pH of 4 was used for application to either type I hair tresses that were bleached three times (3× bleached type I hair tresses) or virgin type I hair tresses.

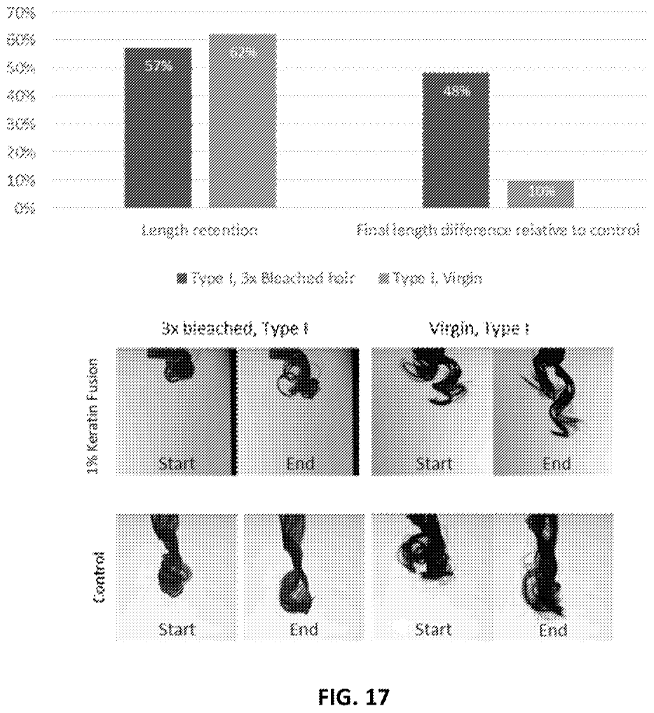

Tresses were first cleaned with a 20% SLS solution (0.4 g/g_{dry hair}). 1% recombinant keratin fusion polypeptide solution was applied to wet tresses (0.2 g/g_{dry hair}); control tresses did not have the recombinant keratin fusion polypeptide applied. Wet tresses were then rolled in 0.2-inch plastic curlers and dried overnight at 85% RH. After drying overnight, the plastic curlers were removed from tresses and the tresses were placed in a hair image acquisition system (Shuffle, Bossa Nova Vision) set at 85% RH, where images were acquired at the initial time of setup and after 24 hours (FIG. 17). Hair style retention was quantified using two metrics in this example. First, length retention was defined as the ratio between the initial hair length and the final hair length. Second, final length difference relative to control was defined as the final length difference between the control tress and the treated tress sample, normalized to the final length of the control tress. The data show that 3× bleached type I hair tresses retained 57% of their initial length, while virgin type I hair tresses retained 62% of their initial length (FIG. 17). Importantly, tresses of both hair types treated with the recombinant keratin fusion polypeptide were found to be shorter than control counterpart tresses (FIG. 17), indicating that recombinant keratin fusion polypeptide treatment can improve style retention. It can also be noted that recombinant keratin fusion polypeptide treatment may potentiate stronger style retention benefits when applied to damaged hair, such as 3× bleached type I hair tresses.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 22. A Recombinant Keratin Fusion Polypeptide of the Disclosure Improves Frizz Control when Applied as a Leave-on

This example was performed to evaluate the impact of an exemplary recombinant keratin fusion polypeptide of the disclosure (SEQ ID NO: 71) on frizz control. A solution comprising 0.1% or 1% of the recombinant keratin fusion polypeptide formulated at a pH of 4 was used for application to either type I hair tresses that were bleached three times (3× bleached type I hair tresses), virgin type I hair tresses, or virgin type IV hair tresses.

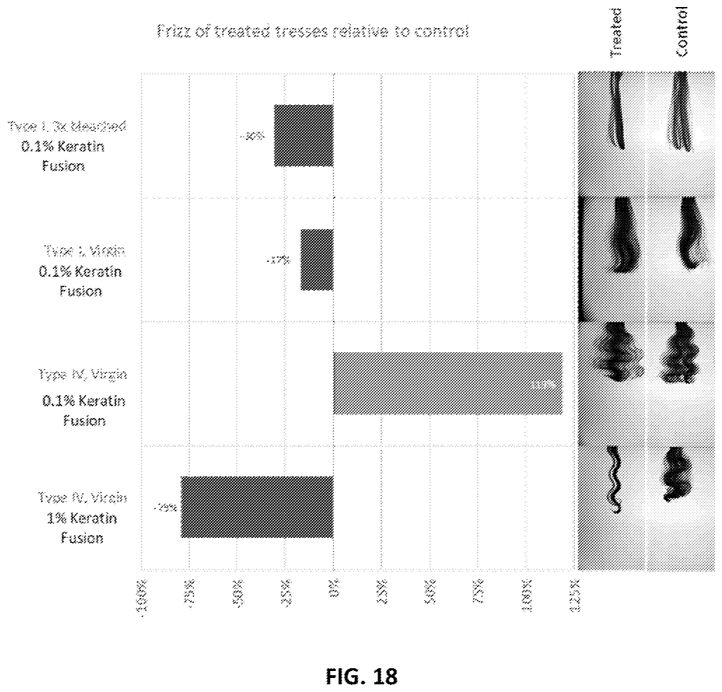

Tresses were first cleaned with a 20% SLS solution (0.4 g/g_{dry hair}). Recombinant keratin fusion polypeptide solutions were applied to wet tresses (0.2 g/g_{dry hair}); control tresses did not have the recombinant keratin fusion polypeptide applied. Tresses were then placed in a hair acquisition system (Shuffle, Bossa Nova Vision) set at 85% RH, where images were acquired at the initial time of setup and after 24 hours (FIG. 18). The change of frizz surface was quantified for each sample and the average change of frizz surface relative to control tresses were calculated such that negative values indicate a reduction of frizz when compared to untreated control tresses.

The data show a reduction of frizz surface for 3× bleached type I hair tresses and virgin type I hair tresses that were treated with a 0.1% recombinant keratin fusion polypeptide solution as a leave-on application (FIG. 18). Application with a higher dose of the solution comprising recombinant keratin fusion polypeptide (1%) on virgin type IV hair tresses also demonstrated improvement in frizz control relative to untreated virgin type IV hair (FIG. 18). The data show that the recombinant keratin fusion polypeptide, when applied as a leave-on, can improve frizz control of various hair types.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 23. A Recombinant Keratin Fusion Polypeptide of the Disclosure Improves Frizz Control when Applied as a Rinse-Off

This example was performed to evaluate the impact of an exemplary recombinant keratin fusion polypeptide of the disclosure (SEQ ID NO: 71) on frizz control. A solution comprising 1% of the recombinant keratin fusion polypeptide formulated at a pH of 4 was used for application to either type I hair tresses that were bleached three times (3× bleached type I hair tresses), virgin type I hair tresses, or virgin type IV hair tresses.

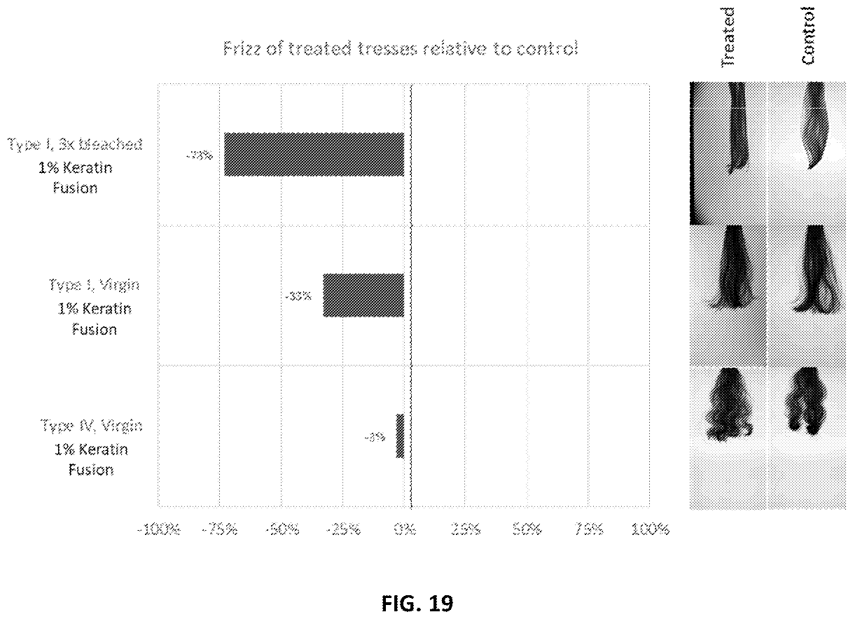

Tresses were first cleaned with a 20% SLS solution (0.4 g/gdr_{y} hair). 1% recombinant keratin fusion polypeptide solution was applied to wet tresses (0.2 g/g_{dry hair}) for 5 minutes and subsequently rinsed with water for 15 seconds; control tresses did not have the recombinant keratin fusion polypeptide applied. Tresses were then placed in a hair acquisition system (Shuffle, Bossa Nova Vision) set at 85% RH, where images were acquired at the initial time of setup and after 24 hours (FIG. 19). The change of frizz surface was quantified for each sample and the average change of frizz surface relative to control tresses were calculated such that negative values indicate a reduction of frizz when compared to untreated control tresses.

The data show a substantial reduction of frizz surface for 3× bleached type I hair tresses and virgin type I hair tresses that were treated with 1% recombinant keratin fusion polypeptide solution as a leave-on application (FIG. 19). The data show that the recombinant keratin fusion polypeptide can also improve frizz control of various hair types when applied as a rinse-off.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 24. A Recombinant Keratin Fusion Polypeptide of the Disclosure Impacts Combing Performance

This example was performed to evaluate the impact of an exemplary recombinant keratin fusion polypeptide of the disclosure (SEQ ID NO: 71) on hair combing performance. A solution comprising 0.1% or 1% of the recombinant keratin fusion polypeptide formulated at a pH of 4 was used for application to type I hair tresses that were bleached three times (3× bleached type I hair tresses). Tresses were first cleaned with a 20% SLS solution (0.4 g/g_{dry hair}). Recombinant keratin fusion polypeptide solution was applied to wet tresses (0.2 g/g_{dry hair}) as a leave-on or rinse-off. Recombinant keratin fusion polypeptide solution was rested on tresses for 5 minutes and subsequently rinsed with water for 15 seconds in the rinse-off condition, while the recombinant keratin fusion polypeptide solution was left on tresses for the leave-on condition. A Texture Analyzer (TA.XT Plus Connect, Stable Micro Systems) equipped with a load cell was used to perform combing tests to measure combing force, the force necessary to run a comb through hair tress. Secured by a clamp attached to the load cell, a hair tress is held in place by a platform attached to the base of the instrument. An automated arm moves the hair tress through a comb while measuring the force required to reach a programmed displacement as the combing force. The test can be performed when hair is dry. The test can also be performed when the hair is damp for a wet combing test; damp hair can also subsequently be dried for a dry combing test. Combing force translates to the ease at which hair can be brushed and untangled, and a lower combing force can indicate hair that is more easily combed and untangled. Baseline combing force measurements were taken for tresses prior to recombinant keratin fusion polypeptide application. The change in combing force after recombinant keratin fusion polypeptide treatment was calculated relative to the baseline combing force such that a positive increase in combing force indicates an increase in combing force after recombinant keratin fusion polypeptide treatment.

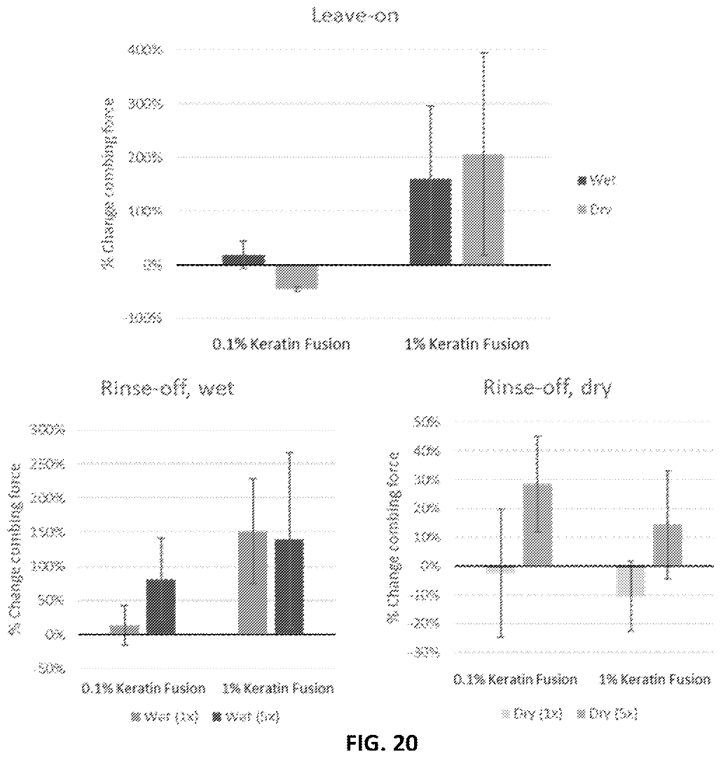

The data show that the application of recombinant keratin fusion polypeptide to hair increases wet combing force, whereas lower doses of the recombinant keratin fusion polypeptide can reduce dry combing force (FIG. 20). In contrast, increasing deposition of the recombinant keratin fusion polypeptide on hair surface, for example through using a composition with a higher concentration or increasing application frequency, can increase dry combing force (FIG. 20).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 25. A Recombinant Keratin Fusion Polypeptide of the Disclosure Exhibits Antioxidant Properties

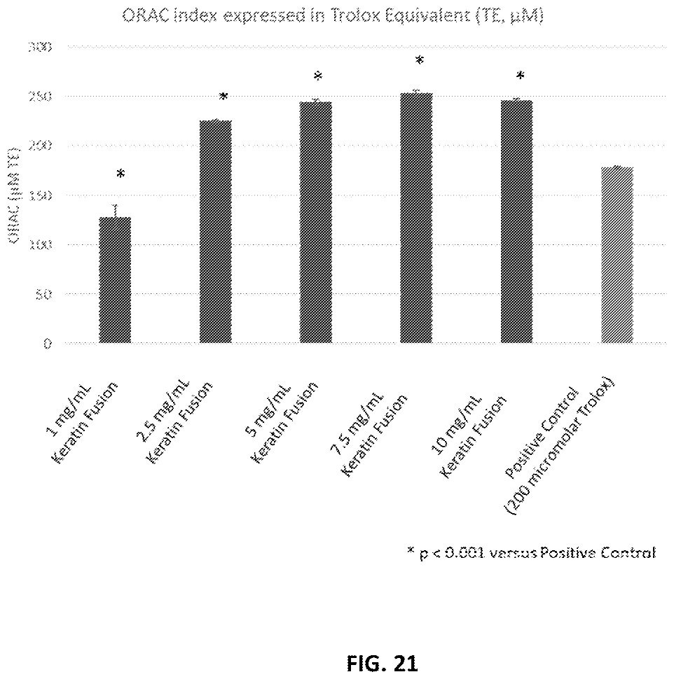

An ORAC assay (OxiSelect™ Oxygen Antioxidant Capacity (ORAC) Activity Assay, Cell Biolabs, Inc.) was performed to evaluate the antioxidant capacity of an exemplary recombinant keratin fusion polypeptide of the disclosure (SEQ ID NO: 71) at various concentrations. Recombinant keratin fusion polypeptide samples were diluted in Assay Diluent (1×) provided in the kit at 1 mg/mL, 2.5 mg/mL, 5 mg/mL, 7.5 mg/mL, and 10 mg/mL; the assay was carried out following the manufacturer's instructions and the data are reported in Trolox (Vitamin E) equivalents. The data show that the recombinant keratin fusion polypeptide, at concentrations greater than 2.5 mg/mL, has a significantly higher ORAC index when compared to the positive control (200 micromolar Trolox; FIG. 21), suggesting that recombinant keratin fusion polypeptide exhibits substantial antioxidant properties.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 26. A Recombinant Keratin Fusion Polypeptide of the Disclosure Improves Viability of Cell Populations Contributing to Hair and Scalp Health

A MTT assay was performed to evaluate the impact of an exemplary recombinant keratin fusion polypeptide of the disclosure (SEQ ID NO: 71) on cell viability for fibroblast and keratinocytes. 5,000 fibroblasts and 10,000 keratinocytes were seeded per well in an incubator (maintained at 37° C. and 5% CO₂). After 24 hours, spent cell culture medium was removed and replenished with fresh culture media containing 0.125-10 mg/mL of the recombinant keratin fusion polypeptide and cultured in the incubator for another 24 hours. Spent cell culture media containing the recombinant keratin fusion polypeptide was removed and the cells were subsequently incubated in MTT solution for 4 hours in the incubator. Dimethyl sulfoxide (DMSO) was added at the end of the MTT incubation period for dissolution of cells and the absorbance reading was performed at 550 nm using a spectrophotometer.

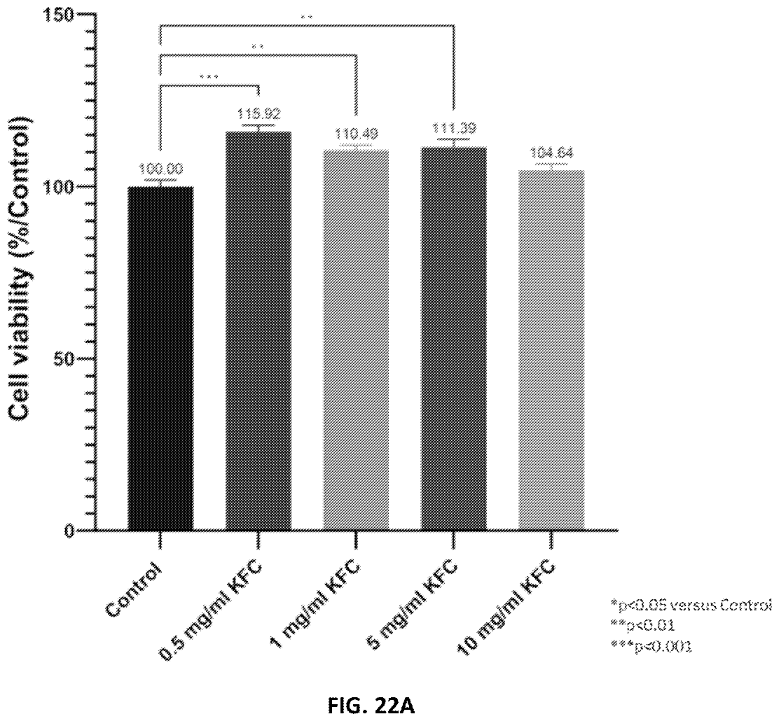

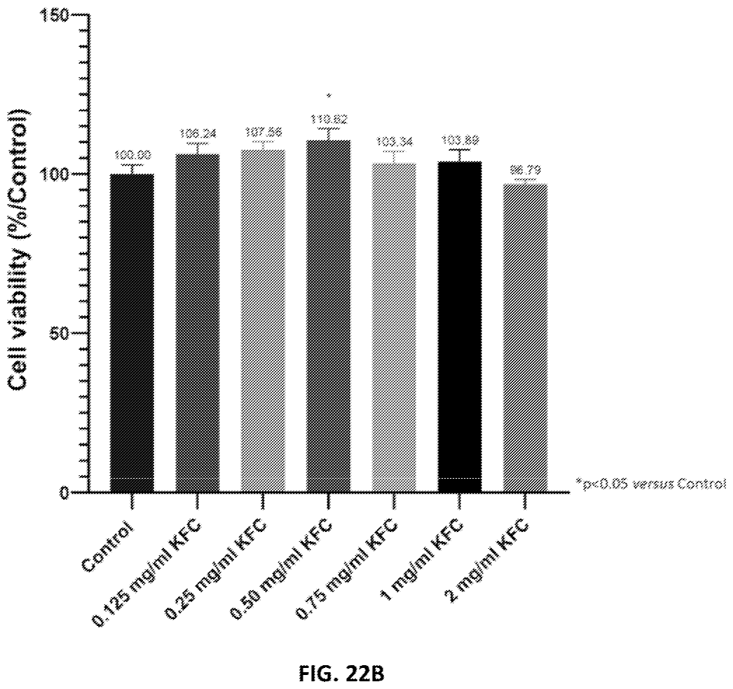

Fibroblast cultures incubated with 0.5 mg/mL, 1 mg/mL, and 5 mg/mL of the recombinant keratin fusion polypeptide resulted in significant increases in cell viability compared to the untreated control (FIG. 22A). Keratinocyte cultures incubated with 0.5 mg/mL of the recombinant keratin fusion polypeptide also resulted in a significant increase in cell viability compared to the untreated control (FIG. 22B). Importantly, the recombinant keratin fusion polypeptide did not result in any detriment to fibroblast and keratinocyte cell viability across all conditions tested (FIG. 22). The data suggest that the recombinant keratin fusion polypeptide is not toxic in vitro and can improve fibroblast and keratinocyte cell viability. Taken together, it can be seen that the recombinant keratin fusion polypeptide can significantly improve the viability of cell populations contributing to hair and scalp health, thereby enhancing desirable hair and scalp properties.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 27. A Recombinant Keratin Fusion Polypeptide of the Disclosure Imparts Significant Wound Healing Benefits

An in vitro wound healing assay as described in Example 12 was performed to evaluate wound healing benefits provide by an exemplary recombinant keratin fusion polypeptide of the disclosure (SEQ ID NO: 71). Wound healing is an important biological process intricately tied to biomedicine (e.g., wound repair) and beauty and personal care (e.g., skin care, scalp care).

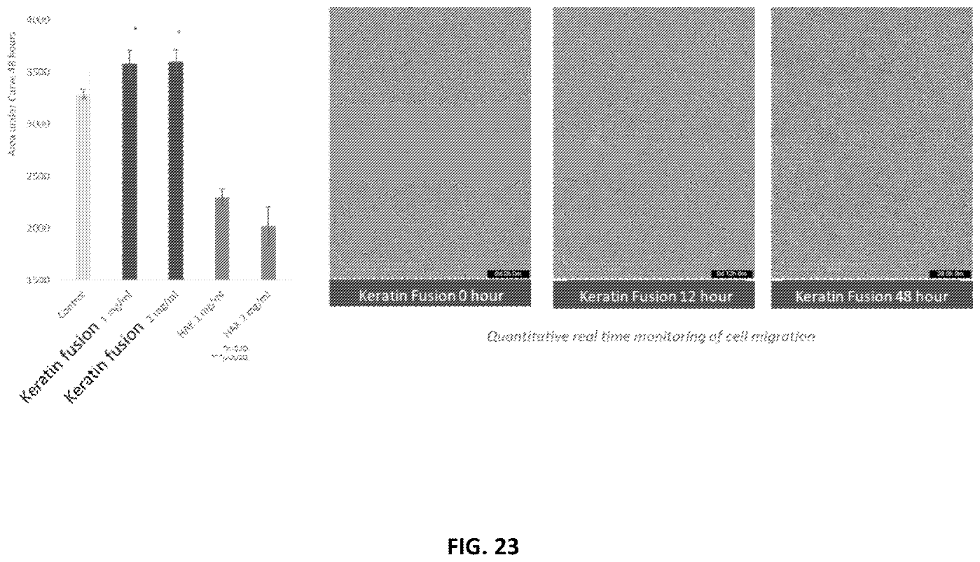

The wound healing assay described herein can also be used to illustrate the wound healing benefits of the recombinant keratin fusion polypeptide on fibroblasts. Collectively, the data show that the recombinant keratin fusion polypeptide demonstrates significant wound healing benefits, measured as cell migration in this assay, outperforming HAK counterparts (FIG. 23). Such wound healing benefits in vitro can translate to benefits in beauty and personal care applications, for example, by enhancing epidermal integrity for skin and scalp care. Moreover, such wound healing benefits may also improve wound repair outcomes in biomedical applications.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 28. Evaluating the Impact of a Recombinant Keratin Fusion Polypeptide of the Disclosure on Hair Durability

The impact of an exemplary recombinant keratin fusion polypeptide of the disclosure (SEQ ID NO: 71) on hair durability will be valuated by fatigue testing. A solution comprising 1% of the recombinant keratin fusion polypeptide formulated at a pH of 4 will be used for application to type I hair that will have been bleached three times (3× bleached type I hair tresses).

Hair strands will be soaked in 1% recombinant keratin fusion polypeptide solution for 5 minutes, with both ends outside of the solution, for 5 or 10 applications. Hair strands will be washed with deionized water for 30 seconds after each application and gently pressed between two layers of laboratory paper wiper for drying between each application cycle. The durability of hair strands is evaluated by a fatigue test using the CYC801 Cyclic Tester (Diastron). In this test, hair is subject to a small deformation (low strain) and the number of strain/relaxation cycles until failure (breakage) is measured for each hair fiber; survival time can also be calculated in this setup. Recombinant keratin fusion polypeptide application is expected to improve hair durability compared to untreated hair.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 29. A Comparison Between a Recombinant Keratin Fusion Polypeptide of the Disclosure and a Representative Collagen for Hair Care

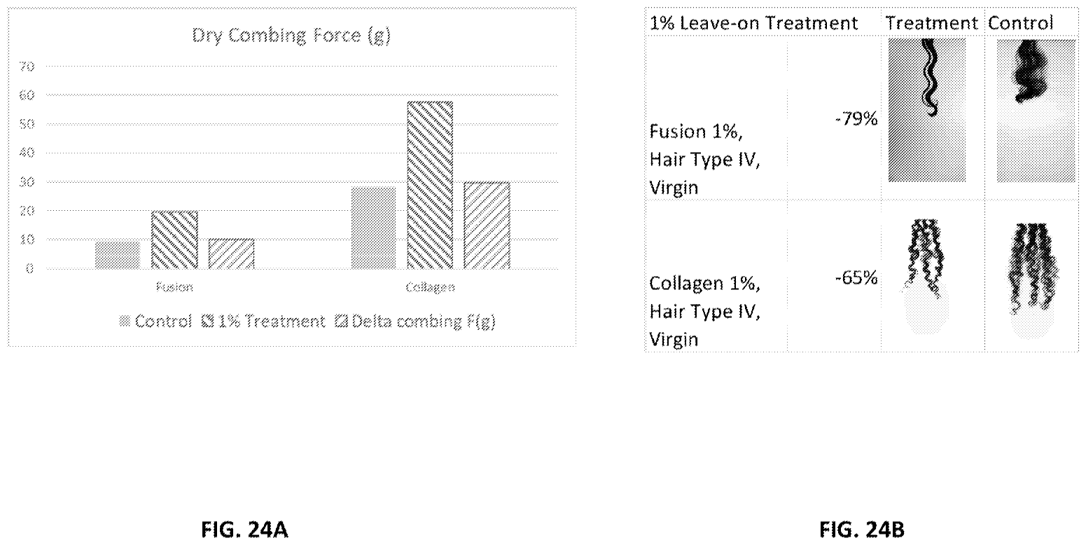

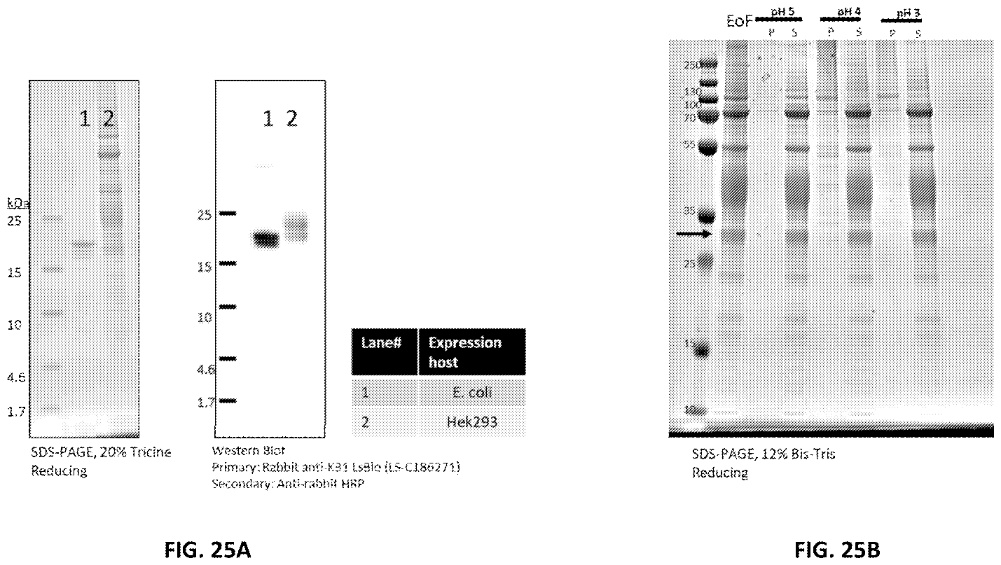

This example compares the effects of an exemplary recombinant keratin fusion polypeptide of the disclosure (SEQ ID NO: 71) and a representative collagen (a truncate of a fibrillar hydrozoan (Podocoryna carnea) collagen) on hair properties. Both the recombinant keratin fusion polypeptide and the representative collagen were applied to hair tresses and subsequently evaluated for their impact on hair. Combing force and frizz control were selected as representative tests, as outlined in Example 24 and Example 22, respectively, to evaluate the impact of the polypeptides on hair. The results illustrate that an increase in combing force (delta combing F) for hair treated with the recombinant keratin fusion polypeptide is lower than the increase in combing force for hair treated with the representative collagen (FIG. 24A). This suggests that hair treatment with the recombinant keratin fusion polypeptide may impart a greater reduction in hair-to-comb friction force when compared to hair treatment with the representative collagen. The results further illustrate that hair tresses treated with the recombinant keratin fusion polypeptide exhibited superior frizz control when compared to hair tresses treated with the representative collagen (FIG. 24B). Taken together, the data collected in this experiment suggest that the recombinant keratin fusion polypeptide compositions impart superior hair benefits when compared to representative collagen compositions. It is also expected that the recombinant keratin fusion polypeptide can confer superior hair tensile strength and resistance to heat damage when compared to representative collagen compositions.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

### Example 30. Expression of a Solubility-Enhancing Peptide Fusion in Prokaryotic and Eukaryotic Host Cells

This example demonstrates the recombinant expression of a representative polypeptide of the disclosure in both prokaryotic and eukaryotic host cells. The “C196-K50” (SEQ ID NO: 71) polypeptide, which is a fusion of human collagen type I, alpha 1 polypeptide fragment of 196 aa (SEQ ID NO: 28) with human keratin type 31 polypeptide of 50 aa (SEQ ID NO: 11), was used to validate recombinant expression in Escherichia coli and human embryonic kidney (HEK293) cells. The “C196-K50” polypeptide was expressed in Escherichia coli following the method of Example 1. HEK293 cells were transiently transfected to express the recombinant “C196-K50” polypeptide.

“C196-K50” expression in Escherichia coli and HEK293 cells was confirmed using SDS-PAGE followed by protein staining with Coomassie blue (FIG. 18A, left), showing a protein band at the predicted size. “C196-K50” expression was further validated by Western blot analysis using a keratin-specific antibody (FIG. 18A, right). HEK293 cell culture extract was subjected to pH treatment to a pH of 5, a pH of 4, or a pH of 3 using H₂SO₄ (FIG. 1B). Insoluble and soluble fractions were examined by SDS-PAGE followed by protein staining with Coomassie blue (FIG. 18B). A thick and clear band was observed at the expected size of “C196-K50” in the soluble fraction across all pH ranges (FIG. 18B), indicating that the “C196-K50” proteins were stable in low pH conditions.

Taken together, the data indicate that the solubility-enhancing peptide fusions described herein, such as representative fusion polypeptide “C196-K50”, can be successfully expressed by both prokaryotic host cells and eukaryotic host cells. The data further exemplify the utility of solubility-enhancing fusion peptides described herein as a platform-enabling technology, which can be employed in both prokaryotic and eukaryotic host cells.

Taken together, the data indicate that the solubility-enhancing peptide fusions described herein, such as representative fusion polypeptide “C196-K50”, can be successfully expressed by both prokaryotic host cells and eukaryotic host cells. The data further exemplify the utility of solubility-enhancing fusion peptides described herein as a platform-enabling technology, which can be employed in both prokaryotic and eukaryotic host cells.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

What is claimed is:

1. A non-naturally occurring polypeptide comprising (a) a solubility-enhancing amino acid sequence; and (b) a target amino acid sequence at least 50 amino acids in length having at least 80% sequence identity to the corresponding region of a keratin polypeptide, wherein the solubility-enhancing amino acid sequence consists of a triplet amino acid pattern of (Gly-X—Y)ₙ, wherein n=8-300, and X and Y are any amino acid residue.

2. The non-naturally occurring polypeptide of claim 1, wherein for at least one (Gly-X—Y) triplet, X and Y are the same amino acid residue.

3. The non-naturally occurring polypeptide of claim 1 or 2, wherein for at least one (Gly-X—Y) triplet, X and Y are different amino acid residues.

4. The non-naturally occurring polypeptide of any one of claims 1-3, wherein at least one (Gly-X—Y) triplet is different from another (Gly-X—Y) triplet.

5. The non-naturally occurring polypeptide of any one of claims 1-4, wherein at least one (Gly-X—Y) triplet is the same as another (Gly-X—Y) triplet.

6. The non-naturally occurring polypeptide of any one of claims 1-5, wherein the non-naturally occurring polypeptide is a fusion protein.

7. The non-naturally occurring polypeptide of claim 6, wherein the solubility-enhancing amino acid sequence is fused to the N-terminus of the target amino acid sequence.

8. The non-naturally occurring polypeptide of claim 6, wherein the solubility-enhancing amino acid sequence is fused to the C-terminus of the target amino acid sequence.

9. The non-naturally occurring polypeptide of claim 6, wherein the solubility-enhancing amino acid sequence comprises a first solubility-enhancing amino acid sequence fused to the N-terminus of the target amino acid sequence, and a second solubility-enhancing amino acid sequence fused to the C-terminus of the target amino acid sequence.

10. The non-naturally occurring polypeptide of any one of claims 1-9, wherein a ratio of the number of amino acid residues present in the solubility-enhancing amino acid sequence to the number of amino acid residues present in the target amino acid sequence is at least 0.5:1.

11. The non-naturally occurring polypeptide of any one of claims 1-10, wherein the keratin polypeptide is a Type I keratin or a Type II keratin.

12. The non-naturally occurring polypeptide of claim 11, wherein the Type I keratin is selected from the group consisting of an epithelial keratin, a hair follicle-specific epithelial keratin, and a hair keratin.

13. The non-naturally occurring polypeptide of claim 12, wherein the keratin polypeptide is a hair keratin selected from the group consisting of K31, K32, K33a, K33b, K34, K35, K36, K37, K38, K39, and K40.

14. The non-naturally occurring polypeptide of claim 12, wherein the keratin polypeptide is an epithelial keratin selected from the group consisting of K9, K10, K12, K13, K14, K15, K16, K17, K18, K19, K20, K23, and K24.

15. The non-naturally occurring polypeptide of claim 17, wherein the Type II keratin is selected from the group consisting of an epithelial keratin, a hair follicle-specific epithelial keratin, and a hair keratin.

16. The non-naturally occurring polypeptide of claim 15, wherein the keratin polypeptide is a hair keratin selected from the group consisting of K81, K82, K83, K84, K85, and K86.

17. The non-naturally occurring polypeptide of claim 15, wherein the keratin polypeptide is an epithelial keratin selected from the group consisting of K1, K2, K3, K4, K5, K6a, K6b, K6c, K7, K8, K76, K77, K78, K79, and K80.

18. The non-naturally occurring polypeptide of any one of claims 1-17, wherein the solubility-enhancing amino acid sequence and the target amino acid sequence are directly linked by a peptide bond.

19. The non-naturally occurring polypeptide of any one of claims 1-17, wherein the solubility-enhancing amino acid sequence and the target amino acid sequence are linked via a linker sequence.

20. The non-naturally occurring polypeptide of claim 19, wherein the linker sequence is selected from the group consisting of a flexible linker, a rigid linker, and a cleavable linker.

21. The non-naturally occurring polypeptide of claim 20, wherein the cleavable linker comprises a protease cleavage site.

22. The non-naturally occurring polypeptide of any one of claims 1-21, wherein the solubility-enhancing sequence comprises or consists of an amino acid sequence of any one of SEQ ID NOS: 18-31 or 119, or comprises or consists of an amino acid sequence having at least about 80% sequence identity to the amino acid sequence of any one of SEQ ID NOS: 18-31 or 119.

23. The non-naturally occurring polypeptide of any one of claims 1-22, wherein the target amino acid sequence comprises or consists of an amino acid sequence of any one of SEQ ID NOS: 2-12, or comprises or consists of an amino acid sequence having at least about 80% sequence identity to the amino acid sequence of any one of SEQ ID NOS: 2-12.

24. The non-naturally occurring polypeptide of any one of claims 1-23, wherein the non-naturally occurring polypeptide comprises or consists of an amino acid sequence of any one of SEQ ID NOS: 56-74, 76, 78, 80-82, 84, 86, 88, 90, 92, 94, 96, 102, 104, 106, 108-113, or 118, or comprises or consists of an amino acid sequence having at least about 80% sequence identity to the amino acid sequence of any one of SEQ ID NOS: 56-74, 76, 78, 80-82, 84, 86, 88, 90, 92, 94, 96, 102, 104, 106, 108-113, or 118.

25. A method of producing a non-naturally occurring polypeptide, the method comprising: (a) culturing a host cell comprising a polynucleotide encoding the non-naturally occurring polypeptide of any one of claims 1-24; and (b) recovering the non-naturally occurring polypeptide, thereby producing the non-naturally occurring polypeptide.

26. A method of producing a target polypeptide, the method comprising: (a) culturing a host cell comprising a polynucleotide encoding the non-naturally occurring polypeptide of any one of claims 1-24, (b) recovering the non-naturally occurring polypeptide, and (c) separating the solubility-enhancing amino acid sequence from the target amino acid sequence, thereby producing the target polypeptide.

27. The method of any one of claim 25 or claim 26, wherein the host cell is a microbial cell or a eukaryotic cell.

28. The method of claim 27, wherein the microbial host cell is a bacteria cell.

29. The method of claim 28, wherein the bacterial cell is E. coli.

30. The method of claim 27, wherein the eukaryotic cell is a mammalian cell.

31. A recombinant polypeptide comprising the amino acid sequence of a full-length human keratin 31 (K31) having a total truncation of from 50 amino acids to 400 amino acids.

32. The recombinant polypeptide of claim 31, wherein the total truncation is from 50 amino acids to 350 amino acids, from 50 amino acids to 300 amino acids, from 50 amino acids to 250 amino acids, from 50 amino acids to 200 amino acids, from 50 amino acids to 150 amino acids, or from 50 amino acids to 100 amino acids.

33. The recombinant polypeptide of claim 31 or 32, wherein the total truncation comprises an N-terminal truncation, a C-terminal truncation, an internal truncation, or any combination thereof.

34. The recombinant polypeptide of claim 33, wherein the total truncation is an N-terminal truncation.

35. The recombinant polypeptide of claim 34, wherein the N-terminal truncation is from 50 amino acids to 250 amino acids, from 50 amino acids to 200 amino acids, from 50 amino acids to 150 amino acids, or from 50 amino acids to 100 amino acids.

36. The recombinant polypeptide of claim 33, wherein the total truncation is a C-terminal truncation.

37. The recombinant polypeptide of claim 36, wherein the C-terminal truncation is from 50 amino acids to 200 amino acids, from 50 amino acids to 150 amino acids, or from 50 amino acids to 100 amino acids.

38. The recombinant polypeptide of claim 33, wherein the total truncation is both an N-terminal truncation and a C-terminal truncation.

39. The recombinant polypeptide of claim 38, wherein the N-terminal truncation is from 50 amino acids to 250 amino acids, from 50 amino acids to 200 amino acids, from 50 amino acids to 150 amino acids, or from 50 amino acids to 100 amino acids, and the C-terminal truncation is from 50 amino acids to 200 amino acids, from 50 amino acids to 150 amino acids, or from 50 amino acids to 100 amino acids.

40. The recombinant polypeptide of any one of claims 31-39, wherein the recombinant polypeptide has less than 75% of the amino acid sequence of the full-length K31.

41. The recombinant polypeptide of any one of claims 31-40, wherein the recombinant polypeptide has less than 70%, less than 65%, less than 60%, less than 55%, less than 50%, less than 45%, less than 40%, less than 35%, less than 30%, less than 25%, less than 20%, or less than 15% of the amino acid sequence of the full-length K31.

42. The recombinant polypeptide of any one of claims 31-41, wherein the recombinant polypeptide comprises the amino acid sequence of SEQ ID NO: 11, or an amino acid sequence having at least 80% sequence identity to SEQ ID NO: 11.

43. The recombinant polypeptide of any one of claims 31-42, wherein the recombinant polypeptide comprises the amino acid sequence of any one of SEQ ID NOs: 7, 9, or 10; or an amino acid sequence having at least 80% sequence identity to any one of SEQ ID NOs: 7, 9, or 10.

44. The recombinant polypeptide of any one of claims 31-41, wherein the recombinant polypeptide consists of the amino acid sequence of any one of SEQ ID NOs: 7 or 9-11; or consists of an amino acid sequence having at least 80% sequence identity to any one of SEQ ID NOs: 7 or 9-11.

45. A non-naturally occurring polypeptide comprising at least 9 contiguous amino acid residues of a collagen polypeptide and at least 25 contiguous amino acid residues of a keratin polypeptide, wherein the at least 9 contiguous amino acid residues of the collagen polypeptide comprise at least three (Gly-X—Y) triplet repeats.

46. A composition comprising from 0.005% to 30% w/w of the non-naturally occurring polypeptide of any one of claims 1-28 or 45, the target polypeptide of any one of claims 26-30, or the recombinant polypeptide of any one of claims 31-44.

47. A composition comprising a recombinant polypeptide, wherein the recombinant polypeptide comprises an amino acid sequence having at least 80% sequence identity to a corresponding region of a full-length keratin polypeptide having a truncation of at least 50 amino acids, wherein the recombinant polypeptide has a purity of at least 80% as measured by high-performance liquid chromatography or mass spectrometry.

48. The composition of claim 46 or 47, wherein the composition is formulated for topical application.

49. The composition of any one of claims 46-48, wherein the composition is a personal care product for application to skin, hair, scalp, or nails.

50. A method for treating or providing a cosmetic benefit to the skin or nails of a subject, the method comprising administering or applying to the skin or nails of a subject the composition of any of claims 46-49, thereby treating or providing the cosmetic benefit to the skin or nails of the subject.

51. The method of claim 50, wherein the administering or applying results in decreasing skin damage, promoting the repair of damaged skin, protecting the skin against UV damage, protecting skin cells against the effects of exposure to urban dust, increasing viability of skin cells, increasing the viability of fibroblast cells, increasing the viability of keratinocyte cells, increasing procollagen synthesis, decreasing the production of inflammatory cytokines, repairing or strengthening the nail plate, repairing dry or damaged nails, preventing damage to nails, improving strength or growth of nails, or any combination thereof.

52. A method for treating or providing a cosmetic benefit to the hair of a subject, the method comprising administering or applying to the hair of a subject the composition of any one of claims 46-49, thereby treating or providing the cosmetic benefit to the hair of the subject.

53. The method of claim 52, wherein the administering or applying results in improving hair strength, improving hair shininess, smoothness, suppleness, combing performance, frizz control, sheen or feel, improving hair combability or manageability, improving hair flexibility, increasing hair strand diameter, improving viability of cell populations contributing to hair and scalp health, providing wound healing benefits for hair care, providing wound healing benefits for skin care, providing wound healing benefits for biomedicine, strengthening hair, repairing split ends, repairing hair damaged by atmospheric agents, repairing hair damaged or weakened by mechanical or chemical treatments, protecting hair structural proteins from oxidative stress, decreasing keratin denaturation, protecting hair from damage by heat, protecting hair from damage by atmospheric agents, mechanical or chemical treatments, or any combination thereof.

54. A method of purifying a recombinant polypeptide from a plurality of host cells, the method comprising:
(a) providing or obtaining a mixture comprising the recombinant polypeptide and host cells or host cell lysate;
(b) separating the mixture into a first soluble fraction comprising the recombinant polypeptide and a first insoluble fraction;
(c) adjusting the pH of the first soluble fraction to a pH of 5 or less to generate a pH-adjusted mixture; and
(d) separating the pH-adjusted mixture into a second soluble fraction comprising the recombinant polypeptide and a second insoluble fraction, wherein the recombinant polypeptide comprises at least 50% of protein in the second soluble fraction.

55. The method of claim 54, wherein the host cell is a microbial cell.

56. The method of claim 55, wherein the microbial cell is a bacterial cell.

57. The method of claim 56, wherein the bacterial cell is a gram-negative bacterium.

58. The method of claim 57, wherein the gram-negative bacterium is E. coli.

59. The method of any one of claims 54-58, wherein separating the mixture comprises centrifugation, ultracentrifugation, filtration, ultrafiltration, or a combination thereof.

60. The method of any one of claims 54-59, wherein the adjusting the pH of (c) comprises adding an acid to the first soluble fraction.

61. The method of claim 60, wherein the acid is a weak acid or a strong acid.

62. The method of claim 60, wherein the acid is H₂SO₄.

63. The method of any one of claims 54-62, wherein the adjusting the pH of (c) comprises adjusting the pH of the first soluble fraction to a pH of 4 or less.

64. The method of any one of claims 54-63, wherein the adjusting the pH of (c) comprises adjusting the pH of the first soluble fraction to a pH of 3 or less.

65. The method of any one of claims 54-64, further comprising (e) adjusting the pH of the second soluble fraction to a pH that is lower than the pH of (c) to generate a second pH-adjusted mixture; and (f) separating the second pH-adjusted mixture into a third soluble fraction comprising the recombinant polypeptide and a third insoluble fraction.

66. The method of claim 65, further comprising (g) adjusting the pH of the third soluble fraction to a pH that is lower than the pH of (e) to generate a third pH-adjusted mixture; and (h) separating the third pH-adjusted mixture into a fourth soluble fraction comprising the recombinant polypeptide and a fourth insoluble fraction.

67. The method of claim 66, wherein a purity of the recombinant polypeptide is higher in the third soluble fraction than in the second soluble fraction; and wherein a purity of the recombinant polypeptide is higher in the fourth soluble fraction than in the third soluble fraction.

68. The method of any one of claims 54-67, wherein the recombinant polypeptide, or portions thereof, is at least 55% of protein present in the second soluble fraction.

69. 16. The method of any one of claims 54-68, wherein the recombinant polypeptide, or portions thereof, is at least 60% of protein present in the second soluble fraction.

70. 17. The method of any one of claims 54-69, wherein the recombinant polypeptide, or portions thereof, is at least 65% of protein present in the second soluble fraction.

71. The method of any one of claims 54-70, wherein the recombinant polypeptide, or portions thereof, is at least 70% of protein present in the second soluble fraction.

72. The method of any one of claims 54-71, wherein the recombinant polypeptide, or portions thereof, is at least 75% of protein present in the second soluble fraction.

73. The method of any one of claims 54-72, wherein the recombinant polypeptide, or portions thereof, is at least 80% of protein present in the second soluble fraction.

74. The method of any one of claims 54-73, wherein the recombinant polypeptide, or portions thereof, is at least 85% of protein present in the second soluble fraction.

75. The method of any one of claims 54-74, wherein the recombinant polypeptide, or portions thereof, is at least 90% of protein present in the second soluble fraction.

76. The method of any one of claims 54-75, wherein the recombinant polypeptide, or portions thereof, is at least 94% of protein present in the second soluble fraction.

77. The method of any one of claims 54-76, wherein the recombinant polypeptide, or portions thereof, is at least 95% of protein present in the second soluble fraction.

78. The method of any one of claims 54-77, wherein the recombinant polypeptide, or portions thereof, is at least 96% of protein present in the second soluble fraction.

79. The method of any one of claims 54-78, wherein the recombinant polypeptide, or portions thereof, is at least 97% of protein present in the second soluble fraction.

80. The method of any one of claims 54-79, wherein the recombinant polypeptide, or portions thereof, is at least 98% of protein present in the second soluble fraction.

81. The method of any one of claims 54-80, wherein the recombinant polypeptide, or portions thereof, is at least 99% of protein present in the second soluble fraction.

82. The method of any one of claims 54-81, wherein the final product comprising the recombinant polypeptide is devoid of host cell debris.

83. The method of any one of claims 54-82, wherein the final product comprising the recombinant polypeptide comprises at most 50% endogenous host cell protein relative to total protein.

84. The method of any one of claims 54-83, wherein the final product comprising the recombinant polypeptide comprises at most 45% endogenous host cell protein relative to total protein.

85. The method of any one of claims 54-84, wherein the final product comprising the recombinant polypeptide comprises at most 40% endogenous host cell protein relative to total protein.

86. The method of any one of claims 54-85, wherein the final product comprising the recombinant polypeptide comprises at most 35% endogenous host cell protein relative to total protein.

87. The method of any one of claims 54-86, wherein the final product comprising the recombinant polypeptide comprises at most 30% endogenous host cell protein relative to total protein.

88. The method of any one of claims 54-87, wherein the final product comprising the recombinant polypeptide comprises at most 25% endogenous host cell protein relative to total protein.

89. The method of any one of claims 54-88, wherein the final product comprising the recombinant polypeptide comprises at most 20% endogenous host cell protein relative to total protein.

90. The method of any one of claims 54-89, wherein the final product comprising the recombinant polypeptide comprises at most 15% endogenous host cell protein relative to total protein.

91. The method of any one of claims 54-90, wherein the final product comprising the recombinant polypeptide comprises at most 10% endogenous host cell protein relative to total protein.

92. The method of any one of claims 54-91, wherein the final product comprising the recombinant polypeptide comprises at most 6% endogenous host cell protein relative to total protein.

93. The method of any one of claims 54-92, wherein the final product comprising the recombinant polypeptide comprises at most 5% endogenous host cell protein relative to total protein.

94. The method of any one of claims 54-93, wherein the final product comprising the recombinant polypeptide comprises at most 4% endogenous host cell protein relative to total protein.

95. The method of any one of claims 54-94, wherein the final product comprising the recombinant polypeptide comprises at most 3% endogenous host cell protein relative to total protein.

96. The method of any one of claims 54-95, wherein the final product comprising the recombinant polypeptide comprises at most 2% endogenous host cell protein relative to total protein.

97. The method of any one of claims 54-96, wherein the final product comprising the recombinant polypeptide comprises at most 1% endogenous host cell protein relative to total protein.

98. The method of any one of claims 54-97, wherein the final product comprising the recombinant polypeptide is devoid of endogenous host cell protein.

99. The method of any one of claims 54-98, wherein the recombinant polypeptide comprises a solubility-enhancing amino acid sequence.

100. The method of any one of claims 54-98, wherein the recombinant polypeptide does not comprise a solubility-enhancing amino acid sequence.
